(19) United States
(12) Patent Application Publication
(10) Pub. No.: US 2020/0179428 A1
(43) Pub. Date: Jun. 11, 2020

(54) CYCLIC PHOSPHATE SUBSTITUTED NUCLEOSIDE DERIVATIVES AND METHODS OF USE THEREOF FOR THE TREATMENT OF VIRAL DISEASES

(71) Applicants: STEPHANE BOGEN, Somerset, NJ (US); David DUKHAN, Montpellier (FR); Francois-Rene ALEXANDRE, Montpellier (FR); Rachid RAHALI, St. Laurent des Arbres (FR); Christophe PARSY, Jacou (FR); Jullen MILHAU, Boujan Sur Libron (FR); Claire Pierra ROUVIERE, Montarnaud (FR); Cyril B. DOUSSON, Canet (FR)

(72) Inventors: STEPHANE BOGEN, Somerset, NJ (US); David DUKHAN, Montpellier (FR); Francois-Rene ALEXANDRE, Montpellier (FR); Rachid RAHALI, St. Laurent des Arbres (FR); Christophe PARSY, Jacou (FR); Jullen MILHAU, Boujan Sur Libron (FR); Claire Pierra ROUVIERE, Montarnaud (FR); Cyril B. DOUSSON, Canet (FR)

(73) Assignees: MERCK SHARP & DOHME CORP., Rahway, NJ (US); IDENIX PHARMACEUTICALS LLC, Cambridge, MA (US)

(21) Appl. No.: 16/311,373

(22) PCT Filed: Jun. 20, 2017
(86) PCT No.: PCT/US2017/038225

§ 371 Date: Dec. 19, 2018

Related U.S. Application Data
(60) Provisional application No. 62/352,138, filed on Jun. 20, 2016.

(51) Int. Cl. A61K 31/7072; C07H 19/24; A61K 45/06; A61K 31/7068; A61K 31/7076; A61P 31/14

(52) U.S. Cl. CPC A61K 31/7072; C07H 19/24; A61P 31/14; A61K 31/7068; A61K 31/7076; A61K 45/06

(57) ABSTRACT

The present invention relates to Cyclic Phosphate Substituted Nucleoside Derivatives of Formula (I), and pharmaceutically acceptable salts thereof, wherein A, B, Q, V, R¹, R² and R³ are as defined herein. The present invention also relates to compositions comprising a Cyclic Phosphate Substituted Nucleoside Derivative, and methods of using the Cyclic Phosphate Substituted Nucleoside Derivatives for treating or preventing HCV infection in a patient.

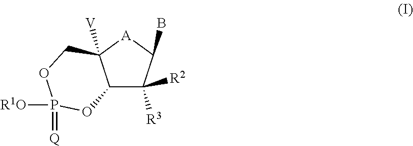

### FIELD OF THE INVENTION

The present invention relates to Cyclic Phosphate Substituted Nucleoside Derivatives, compositions comprising a Cyclic Phosphate Substituted Nucleoside Derivative, and methods of using the Cyclic Phosphate Substituted Nucleoside Derivatives for treating or preventing HCV infection in a patient.

### BACKGROUND OF THE INVENTION

Hepatitis C virus (HCV) infection is a major health problem that leads to chronic liver disease, such as cirrhosis and hepatocellular carcinoma, in a substantial number of infected individuals, estimated to be 2-15% of the world's population. Once infected, about 20% of people clear the virus, but the rest harbor HCV the rest of their lives. Ten to twenty percent of chronically infected individuals eventually develop liver-destroying cirrhosis or cancer. HCV is transmitted parenterally by contaminated blood and blood products, contaminated needles, or sexually and vertically from infected mothers or carrier mothers to their off-spring.

Different approaches to HCV therapy have been taken, which include the inhibition of viral serine proteinase (NS3 protease), helicase, and RNA-dependent RNA polymerase (NS5B), and the development of a vaccine. Current and investigational treatments for HCV infection are reviewed in Poordad et al., Treating hepatitis C: current standard of care. Emerging direct-acting antiviral agents are discussed in Poordad et al., Journal of Viral Hepatitis 19: 449-464 (2012); and Asselah et al., Protease and polymerase inhibitors for the treatment of hepatitis C, Liver International 29(s1): 57-67 (2009). The changing therapeutic landscape for hepatitis C is discussed in Dore, Med. J Australia 196: 629-632 (2012); and Balsano, Mini Rev. Med. Chem. 8(4): 307-318 (2008). Despite the availability of therapeutic treatment options, chronic HCV infection remains a major healthcare concern. Moreover, there is no established vaccine for HCV. Consequently, there is a need for improved therapeutic agents that effectively combat chronic HCV infection.

The HCV virion is an enveloped positive-strand RNA virus with a single oligoribonucleotide genomic sequence of about 9400 bases which encodes a polyprotein of about 3,000 amino acids. The protein products of the HCV gene consist of the structural proteins C, E1, and E2, and the non-structural proteins NS2, NS3, NS4A, NS4B, NS5A and NS5B. The nonstructural (NS) proteins are believed to provide the catalytic machinery for viral replication.

The NS3 protease releases NS5B, the RNA-dependent RNA polymerase from the polyprotein chain. HCV NS5B polymerase is required for the synthesis of a negative-strand RNA intermediate compound from a positive-strand genomic viral RNA that serves as a template in the replication cycle of HCV. NS5B polymerase is an essential component in the HCV replication complex. See K. Ishi, et al., “Expression of Hepatitis C Virus NS5B Protein: Characterization of Its RNA Polymerase Activity and RNA Binding,” Hepatology, 29:1227-1235 (1999) and V. Lohmann, et al., “Biochemical and Kinetic Analyses of NS5B RNA-Dependent RNA Polymerase of the Hepatitis C Virus,” Virology, 249: 108-118 (1998). Inhibition of HCV NS5B polymerase prevents formation of the double-stranded HCV RNA and therefore constitutes an attractive approach to the development of HCV-specific antiviral therapies.

The development of inhibitors of HCV NS5B polymerase with potential for the treatment of HCV infection has been reviewed in Poordad et al. (2012), supra; Asselah et al. (2009), supra; and Chatel-Chaix et al. Direct-acting and host-targeting HCV inhibitors: current and future directions. Current Opinion in Virology, 2:588-598 (2012). The activity of purine ribonucleosides against HCV polymerase was reported by A. E. Eldrup et al., “Structure-Activity Relationship of Purine Ribonucleosides for Inhibition of HCV RNA-Dependent RNA Polymerase,” J. Med. Chem., 47:2283-2295 (2004).

### SUMMARY OF THE INVENTION

In one aspect, the present invention provides Compounds of Formula (I):

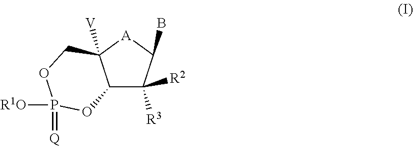

or a pharmaceutically acceptable salt thereof,
wherein:

A is selected from O, S and CH₂;

B is selected from one of the following groups:

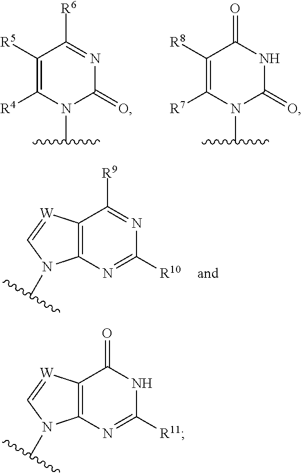

R¹ is —CH(R¹³)—X—Y—Z—R¹⁹;

Q is O or S;

V is H, halo or —N(R¹²)₂;

W is N, CH or CF;

X is a bond or —C(R¹⁴)₂—;

Y is selected from a bond, O, —S(O)₂— and —C(R¹⁵)₂—, such that when Y is O or —S(O)₂—, then X is —C(R¹⁵)₂—;

Z is selected from a bond, —C(R¹⁶)₂— and C₃-C₆ cycloalkylene, such that if X and Y are each a bond, then Z is —C(R¹⁶)₂— or C₃-C₆ cycloalkylene;

R² is selected from H, F, Cl, C₁-C₃ alkyl and C₂-C₃ alkynyl;

R³ is selected from C₁-C₆ alkyl, C₂-C₆ alkenyl, C₂-C₆ alkynyl, —OR¹², F, Cl, —N₃, —CN and —N(R¹²)₂, such that if R² is F or Cl, then R³ is other than F or Cl;

R⁴, R⁵, R⁷ and R⁸ are each independently selected from H, C₁-C₆ alkyl, C₁-C₆ haloalkyl, C₁-C₆ hydroxyalkyl, halo, —OR¹⁸, —SR¹⁸ and —N(R¹⁸)₂;

R⁶, R⁹, R¹⁰ and R¹¹ are each independently selected from H, C₁-C₆ alkyl, C₂-C₆ alkenyl, C₂-C₆ alkynyl, C₃-C₇ cycloalkyl, 5- or 6-membered monocyclic heteroaryl, 9- or 10-membered bicyclic heteroaryl, halo, —OR¹⁸, —SR¹⁸, —S(O)R¹⁸, —S(O)₂R¹⁸, —S(O)₂N(R¹⁸)₂, —NHC(O)OR¹⁸, —NHC(O)N(R¹⁸)₂, C₁-C₆ haloalkyl, C₁-C₆ hydroxyalkyl, —O—(C₁-C₆ haloalkyl), —CN, —NO₂, —N(R¹⁸)₂, —NH(C₁-C₆ alkylene)-(5- or 6-membered monocyclic heteroaryl), —NH(C₁-C₆ alkylene)-(9- or 10-membered bicyclic heteroaryl), —C(O)R¹⁸, —C(O)OR⁸, —C(O)N(R¹⁸)₂ and —NHC(O)R¹⁸, wherein said C₂-C₆ alkenyl group and said C₂-C₆ alkynyl group may be optionally substituted with halo;

each occurrence of R¹² is independently selected from H, C₁-C₆ alkyl, —C(O)R¹⁸ and —C(O)OR¹⁸;

R¹³ is selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)C(O)OR¹⁷ and —C(O)OR¹⁷;

each occurrence of R¹⁴ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)₂, —N(R¹²)C(O)OR¹⁷ and —C(O)OR^{1′}, or both R¹⁴ groups, together with the common carbon atom to which they are attached, can join to form a C₃-C₆ spirocyclic cycloalkyl group;

each occurrence of R¹⁵ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)₂, —N(R¹²)C(O)OR¹⁷ and —C(O)OR¹⁷, or both R¹⁵ groups, together with the common carbon atom to which they are attached, can join to form a C₃-C₆ spirocyclic cycloalkyl group;

each occurrence of R¹⁶ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)C(O)OR¹⁷ and —C(O)OR¹⁷, or both R¹⁶ groups, together with the common carbon atom to which they are attached, can join to form a C₃-C₆ spirocyclic cycloalkyl group;

each occurrence of R¹⁷ is independently selected from H, C₁-C₆ alkyl, C₃-C₇ cycloalkyl and C₆-C₁₀ aryl;

each occurrence of R¹⁸ is independently selected from H, C₁-C₆ alkyl, C₁-C₆ haloalkyl, C₁-C₆ hydroxyalkyl, —(C₁-C₃ alkylene)ₘ-(C₃-C₇ cycloalkyl), —(C₁-C₃ alkylene)ₘ-(C₆-C₁₀ aryl), —(C₁-C₃ alkylene)ₘ-(4 to 7-membered heterocycloalkyl), —(C₁-C₃ alkylene)ₘ-(5- or 6-membered monocyclic heteroaryl) and —(C₁-C₃ alkylene)ₘ-(9- or 10-membered bicyclic heteroaryl);

R¹⁹ is —C(O)OR¹⁷ or:

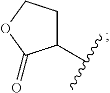 [SMILES: *C1CCOC1=O]

and

each occurrence of m is independently 0 or 1,

such that at least one of R¹³, R¹⁴, R¹⁵ and R¹⁶ is other than H.

The Compounds of Formula (I) (also referred to herein as the “Cyclic Phosphate Substituted Nucleoside Derivatives”) and pharmaceutically acceptable salts thereof may be useful, for example, for inhibiting HCV viral replication or replicon activity, for inhibiting HCV NS5B activity, and for treating or preventing HCV infection in a patient. Without being bound by any specific theory, it is believed that the Cyclic Phosphate Substituted Nucleoside Derivatives inhibit HCV viral replication by inhibiting HCV NS5B.

Accordingly, the present invention provides methods for treating or preventing HCV infection in a patient, comprising administering to the patient an effective amount of at least one Cyclic Phosphate Substituted Nucleoside Derivative.

The details of the invention are set forth in the accompanying detailed description set forth below.

Other embodiments, aspects and features of the present invention are either further described in or will be apparent from the ensuing description, examples and appended claims.

### DETAILED DESCRIPTION OF THE INVENTION

The present invention relates to Cyclic Phosphate Substituted Nucleoside Derivatives, compositions comprising a Cyclic Phosphate Substituted Nucleoside Derivative, and methods of using the Cyclic Phosphate Substituted Nucleoside Derivatives for treating or preventing HCV infection in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “-O-alkyl,” etc. . . . .

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of Cyclic Phosphate Substituted Nucleoside Derivative and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood or severity of HCV infection.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)— alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. In one embodiment, an aryl group can be optionally fused to a cycloalkyl or cycloalkanoyl group. Non-limiting examples of aryl groups include phenyl and naphthyl. In one embodiment, an aryl group is phenyl. Unless otherwise indicated, an aryl group is unsubstituted.

The term “cycloalkyl,” as used herein, refers to a non-aromatic mono- or multicyclic ring system comprising from 3 to about 10 ring carbon atoms. In one embodiment, a cycloalkyl contains from about 5 to about 10 ring carbon atoms. In another embodiment, a cycloalkyl contains from 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. In one embodiment, a cycloalkyl group is unsubstituted. The term “3 to 6-membered cycloalkyl” refers to a cycloalkyl group having from 3 to 6 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

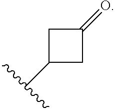 [SMILES: *C1CC(=O)C1]

The term “cycloalkylene,” as used herein, refers to a cycloalkyl group, as defined above, wherein said cycloalkyl group has one or more endocyclic double bonds.

The term “halo,” as used herein, means —F, —Cl, —Br or —I.

The term “haloalkyl,” as used herein, refers to an alkyl group as defined above, wherein one or more of the alkyl group's hydrogen atoms have been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms have been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “5 or 6-membered monocyclic heteroaryl,” as used herein, refers to an aromatic monocyclic ring system comprising about 5 to about 6 ring atoms, wherein from 1 to 4 of the ring atoms is independently O, N or S and the remaining ring atoms are carbon atoms. A 5 or 6-membered monocyclic heteroaryl group is joined via a ring carbon atom, and any nitrogen atom of a heteroaryl can be optionally oxidized to the corresponding N-oxide. The term “5 or 6-membered monocyclic heteroaryl” also encompasses a 5 or 6-membered monocyclic heteroaryl group, as defined above, which is fused to a benzene ring. Non-limiting examples of 5 or 6-membered monocyclic heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, benzofurazanyl, indolyl, azaindolyl, benzimidazolyl, benzothienyl, imidazolyl, benzimidazolyl, thienopyridyl, thienopyrimidyl, pyrrolopyridyl, imidazopyridyl, isoquinolinyl, 1,2,4-triazinyl, benzothiazolyl and the like, and all isomeric forms thereof. Unless otherwise indicated, a 5 or 6-membered monocyclic heteroaryl group is unsubstituted.

The term “9 or 10-membered bicyclic heteroaryl,” as used herein, refers to an aromatic bicyclic ring system comprising about 9 to about 10 ring atoms, wherein from 1 to 4 of the ring atoms is independently O, N or S and the remaining ring atoms are carbon atoms. A 9 or 10-membered bicyclic heteroaryl group is joined via a ring carbon atom, and any nitrogen atom of a heteroaryl can be optionally oxidized to the corresponding N-oxide. Non-limiting examples of 9 or 10-membered bicyclic heteroaryls include imidazo[1,2-a]pyridinyl, imidazo[2,1-b]thiazolyl, benzofurazanyl, indolyl, azaindolyl, benzimidazolyl, benzothienyl, quinolinyl, benzimidazolyl, quinazolinyl, pyrrolopyridyl, imidazopyridyl, isoquinolinyl, benzoazaindolyl, benzothiazolyl, and the like, and all isomeric forms thereof. Unless otherwise indicated, a 9 or 10-membered bicyclic heteroaryl group is unsubstituted.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

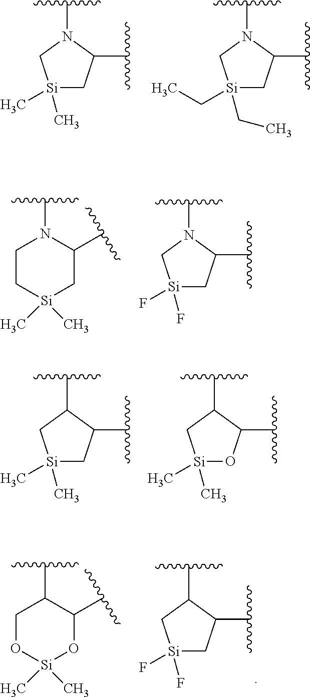

A ring carbon atom of a heterocycloalkyl group may be functionalized as a carbonyl group. An illustrative example of such a heterocycloalkyl group is:

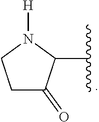 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 6-membered monocyclic cycloalkyl” refers to a monocyclic heterocycloalkyl group having from 3 to 6 ring atoms. The term “4 to 6-membered monocyclic cycloalkyl” refers to a monocyclic heterocycloalkyl group having from 4 to 6 ring atoms. The term “7 to 11-membered bicyclic heterocycloalkyl” refers to a bicyclic heterocycloalkyl group having from 7 to 11 ring atoms. Unless otherwise indicated, an heterocycloalkyl group is unsubstituted.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., C₁-C₆ alkyl, R⁴, R¹⁵, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results directly from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Cyclic Phosphate Substituted Nucleoside Derivative or a pharmaceutically acceptable salt of the compound. The transformation may occur by various mechanisms (e.g., by metabolic or chemical processes), such as, for example, through hydrolysis in blood.

For example, if a Cyclic Phosphate Substituted Nucleoside Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Cyclic Phosphate Substituted Nucleoside Derivative contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate). Other non-limiting example of alcohol-derived prodrugs include —P(O)(OH)₂; —P(O)(—O—C₁-C₆alkyl)₂; —P(O)(—NH-(α-aminoacyl group))(-O-aryl); —P(O)(—O—(C₁-C₆ alkylene)-S-acyl)(—NH-arylalkyl); and those described in U.S. Pat. No. 7,879,815; International Publication Nos. WO2005/003047, WO2008/082602, WO2010/0081628, WO2010/075517 and WO2010/075549; Mehellou, Chem. Med. Chem., 5:1841-1842 (2005); Bobeck et al., Antiviral Therapy 15:935-950 (2010); Furman et al., Future Medicinal Chemistry, 1:1429-1452 (2009); and Erion, Microsomes and Drug Oxidations, Proceedings of the International Symposium, 17th, Saratoga Springs, N.Y., United States, Jul. 6-10, 2008, 7-12 (2008).

If a Cyclic Phosphate Substituted Nucleoside Derivative incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Cyclic Phosphate Substituted Nucleoside Derivatives can form salts which are also within the scope of this invention. The term “salt(s)”, as employed herein, denotes acidic salts formed with inorganic and/or organic acids, as well as basic salts formed with inorganic and/or organic bases. In addition, when a Cyclic Phosphate Substituted Nucleoside Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Cyclic Phosphate Substituted Nucleoside Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures may be separated into their individual diastereomers on the basis of their physical chemical differences by methods well-known to those skilled in the art, such as, for example, by chromatography and/or fractional crystallization. Enantiomers may be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Stereochemically pure compounds may also be prepared by using chiral starting materials or by employing salt resolution techniques. Also, some of the Cyclic Phosphate Substituted Nucleoside Derivatives may be atropisomers (e.g., substituted biaryls) and are considered as part of this invention. Enantiomers can also be directly separated using chiral chromatographic techniques.

It is also possible that the Cyclic Phosphate Substituted Nucleoside Derivatives may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Cyclic Phosphate Substituted Nucleoside Derivative incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples.

Isotopically-enriched Compounds of Formula (I) may be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Cyclic Phosphate Substituted Nucleoside Derivatives, and of the salts, solvates, hydrates, esters and prodrugs of the Cyclic Phosphate Substituted Nucleoside Derivatives, are intended to be included in the present invention.

The following abbreviations are used herein:
Ac acetyl
Ac₂O acetic anhydride
aq aqueous
Bn benzyl
Boc or BOC tert-butoxycarbonyl
Boc₂O tert-butoxycarbonyl anhydride
Bu butyl
n-BuLi n-butyllithium
calc'd calculated
Celite/celite diatomaceous earth
DAST (diethylamino)sulfur trifluoride
DBU 1,8-diazabicyclo(5.4.0)undec-7-ene
DCC dicyclohexylcarbodiimide
DCE 1,2-dichloroethane
dichloromethane dichloromethane
DIBAL or DIBAL-H diisobutylaluminum hydride
DIEA or DIPEA N,N-diisopropylethylamine
DMA 1,2-dimethylacetamide
DMAP 4-dimethylaminopyridine
DMF dimethylformamide
DMSO dimethyl sulfoxide
EDC 1-ethyl-3-(3-dimethylaminopropyl)carbodiimide
EDTA ethylenediamine tetraacetic acid
EGTA ethylene glycol-bis(β-aminoethyl ether)-N,N,N′,N′-tetraacetic acid
ESI electrospray ionization
Et ethyl
Et₂O diethyl ether
EtOH ethanol
EtOAc ethyl acetate
Et₃N triethylamine
iPr isopropyl
LC liquid chromatography
LC/MS liquid chromatography mass spectrometry
Me methyl
MeCN acetonitrile
MeI iodomethane
MeOH methanol
MS mass spectrometry
MTBE methyl tert-butyl ether
NMR nuclear magnetic resonance spectroscopy
NTP nucleoside triphosphate
oxone potassium peroxymonosulfate
Pd/C palladium on carbon
Pd(OH)₂ palladium hydroxide
Pd(OH)₂/C palladium hydroxide on carbon
PE petroleum ether
Ph phenyl
Pr propyl
RT room temperature
Rt retention time
TBME tert-butyl methyl ether
TBDMS tert-butyldimethyl silyl
TBDMSCl tert-butyldimethylsilyl chloride
t-Bu tert-butyl
t-BuOOH tert-butyl peroxide
TFA trifluoroacetic acid
THF tetrahydrofuran
TLC thin layer chromatography
TMS trimethylsilyl

### The Compounds of Formula (I)

The present invention provides Cyclic Phosphate Substituted Nucleoside Derivatives of Formula (I):

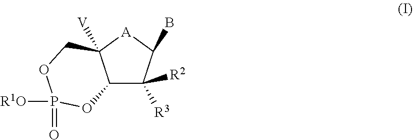

and pharmaceutically acceptable salts thereof, wherein A, B, Q, V, R¹, R² and R³ are defined above for the Compounds of Formula (I).

In one embodiment, for the compounds of formula (I), A is O.

In another embodiment, for the compounds of formula (I), A is S.

In one embodiment, for the compounds of formula (I), Q is O.

In another embodiment, for the compounds of formula (I), Q is S.

In one embodiment, for the compounds of formula (I), R² is C₁-C₃ alkyl.

In another embodiment, for the compounds of formula (I), R² is —CCH.

In another embodiment, for the compounds of formula (I), R² is methyl.

In one embodiment, for the compounds of formula (I), R³ is selected from —OH, F, Cl, —N₃, —CN, —CeCH and —NH₂.

In another embodiment, for the compounds of formula (I), R³ is selected from —Cl, —C≡CH and —NH₂.

In one embodiment, for the compounds of formula (I), R² is methyl and R³ is selected from —OH, F, Cl, —N₃, —CN, —C≡CH and —NH₂.

In one embodiment, for the compounds of formula (I), B is selected from guanine, cytosine, adenine and uracil.

In another embodiment, for the compounds of formula (I), B is selected from adenine and uracil.

In another embodiment, for the compounds of formula (I), B is uracil.

In one embodiment, the compounds of formula (I) have the formula (Ia):

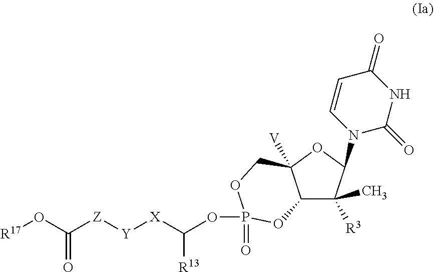

or a pharmaceutically acceptable salt thereof,
wherein:

V is H or F;

X is a bond or —C(R¹⁴)₂—;

Y is selected from a bond, O, S(O)₂ and —C(R¹⁵)₂—; such that when Y is O or —S(O)₂—, then X is —C(R¹⁵)₂—;

Z is selected from a bond, —C(R¹⁶)₂ and C₃-C₆ cycloalkylene, such that if X and Y are each a bond, then Z is —C(R¹⁶)₂— or C₃-C₆ cycloalkylene;

R³ is selected from —OH, F, Cl, N₃, —CN, —C≡CH and —NH₂;

each occurrence of R¹³ is independently selected from H, phenyl and C₁-C₆ alkyl;

each occurrence of R¹⁴ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)₂, —N(R¹²)C(O)OR¹⁷ and —C(O)OR¹⁷;

each occurrence of R¹⁵ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)₂, —N(R¹²)C(O)OR¹⁷ and —C(O)OR¹⁷;

each occurrence of R¹⁶ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)C(O)OR¹⁷ and —C(O)OR¹⁷, or two R¹⁶ groups that are attached to the same carbon atom, together with the common carbon atom to which they are attached, can join to form a C₃-C₆ spirocyclic cycloalkyl group;

each occurrence of R¹⁷ is independently selected from H, C₁-C₆ alkyl, C₃-C₇ cycloalkyl and C₆-C₁₀ aryl; and

each occurrence of m is independently 0 or 1.

In one embodiment, for the compounds of formula (I) or (Ia), V is H or F.

In one embodiment, for the compounds of formula (I) or (Ia), X is a bond.

In another embodiment, for the compounds of formula (I) or (Ia), X is —CH₂—.

In one embodiment, for the compounds of formula (I) or (Ia), Y is a bond.

In another embodiment, for the compounds of formula (I) or (Ia), Y is —CH₂—.

In another embodiment, for the compounds of formula (I) or (Ia), X and Y are each a bond.

In one embodiment, for the compounds of formula (I) or (Ia), Z is —CHR¹⁶—, and R¹⁶ is selected from H, C₁-C₆ alkyl, —O—(C₁-C₆ alkyl), —C(O)O—(C₁-C₆ alkyl) and —O—C(O)—(C₁-C₆ alkyl).

In another embodiment, for the compounds of formula (I) or (Ia), X is a bond; Z is —CHR¹⁶—; and R¹⁶ is selected from H, C₁-C₆ alkyl, —O—(C₁-C₆ alkyl), —C(O)O—(C₁-C₆ alkyl) and —O—C(O)—(C₁-C₆ alkyl).

In another embodiment, for the compounds of formula (I) or (Ia), R¹³ is H; X is a bond; Y is —CH₂— or —CH(CH₃)—; Z is —CHR¹⁶—; and R¹⁶ is selected from H, C₁-C₆ alkyl, —O—(C₁-C₆ alkyl), —C(O)O—(C₁-C₆ alkyl) and —O—C(O)—(C₁-C₆ alkyl).

In one embodiment, for the compounds of formula (I) or (Ia), R³ is F.

In another embodiment, for the compounds of formula (I) or (Ia), R³ is Cl.

In still another embodiment, for the compounds of formula (I) or (Ia), R³ is —C≡CH.

In another embodiment, for the compounds of formula (I) or (Ia), R³ is —NH₂.

In one embodiment, for the compounds of formula (I) or (Ia), R¹³ is H or C₁-C₆ alkyl.

In another embodiment, for the compounds of formula (I) or (Ia), R¹³ is H.

In another embodiment, for the compounds of formula (I) or (Ia), R¹³ is methyl.

In one embodiment, for the compounds of formula (I) or (Ia), R¹⁷ is C₁-C₆ alkyl or C₃-C₇ cycloalkyl.

In another embodiment, for the compounds of formula (I) or (Ia), R¹⁷ is C₁-C₆ alkyl.

In another embodiment, for the compounds of formula (I) or (Ia), R¹⁷ is methyl, ethyl, isopropyl, t-butyl, n-pentyl, cyclopentyl or cyclohexyl, or a pharmaceutically acceptable salt thereof.

In still another embodiment, for the compounds of formula (I) or (Ia), R¹⁷ is selected from methyl, ethyl, isopropyl and n-pentyl.

In another embodiment, for the compounds of formula (I) or (Ia), R¹⁷ is selected from cyclopentyl and cyclohexyl.

In another embodiment, for the compounds of formula (I) or (Ia), R¹⁷ is isopropyl.

In one embodiment, for the compounds of formula (I) or (Ia), R¹ is selected from:

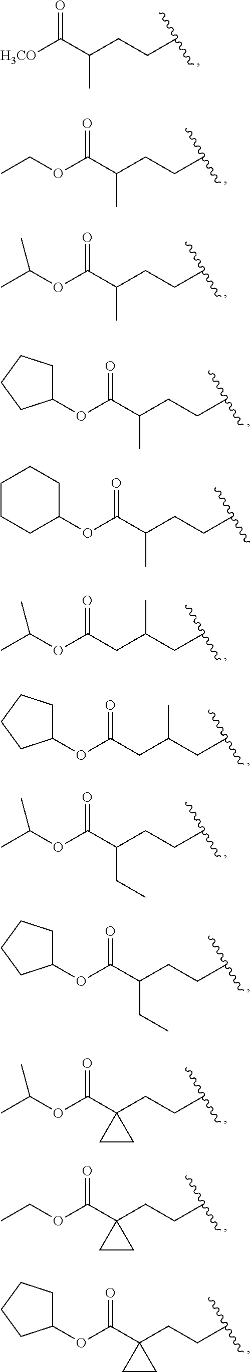

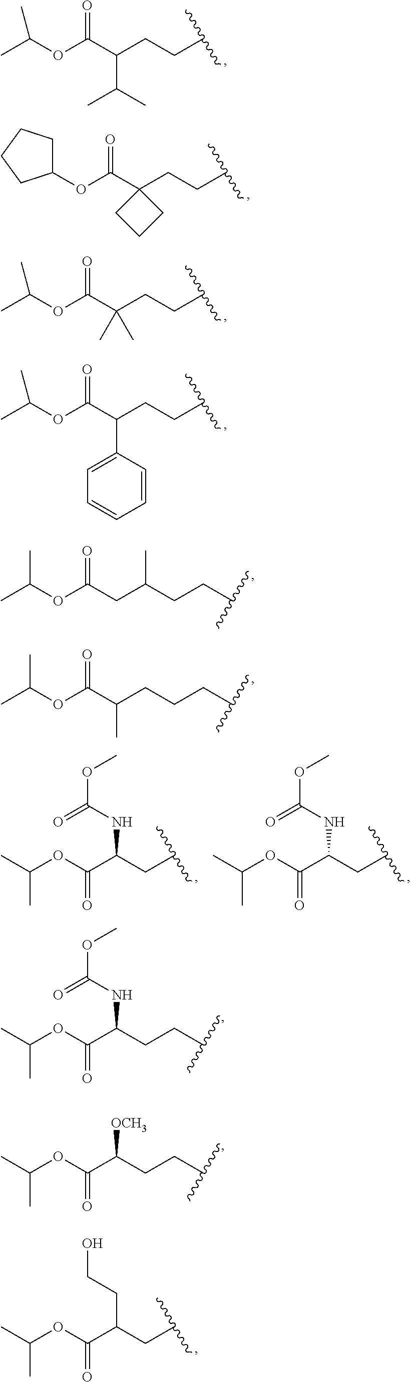

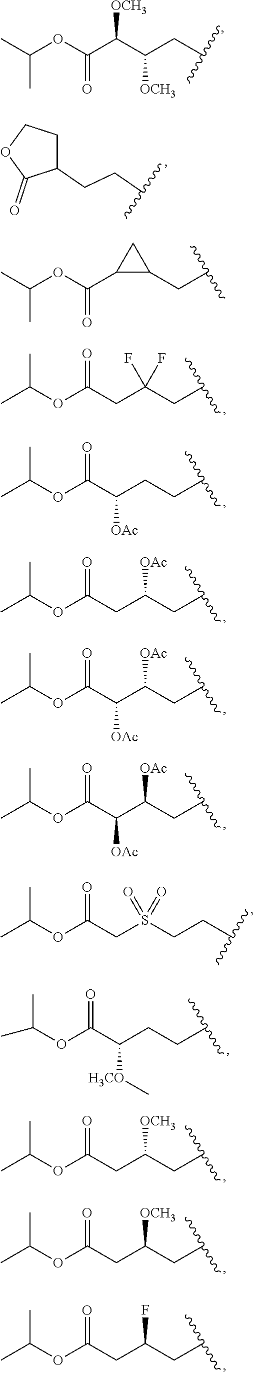

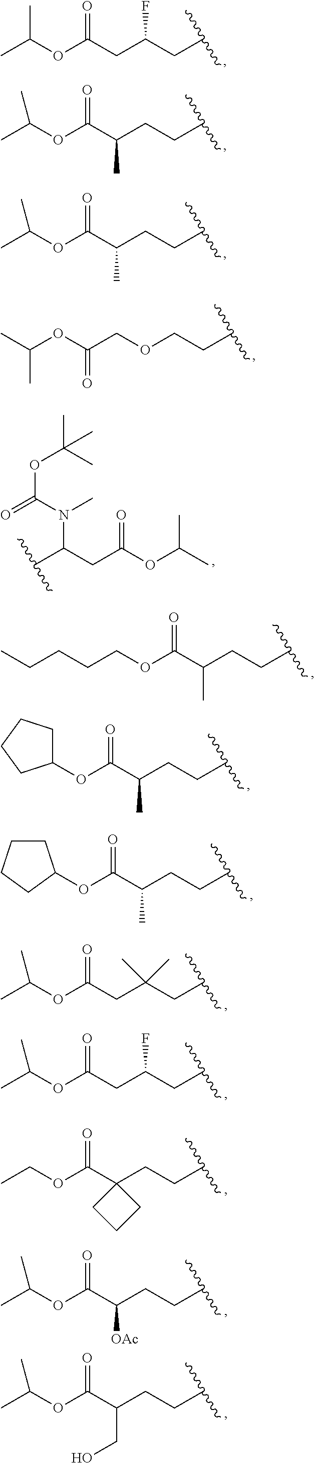

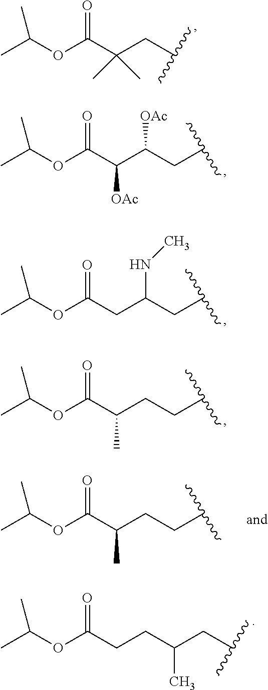

In one embodiment, variables A, B, Q, R¹, R² and R³ for the Compounds of Formula (I) are selected independently of each other.

In another embodiment, the Compounds of Formula (I) are in substantially purified form.

The Compounds of Formula (I) may be referred to herein by chemical structure and/or by chemical name. In the instance that both the structure and the name of a Compound of Formula (I) are provided and a discrepancy is found to exist between the chemical structure and the corresponding chemical name, it is understood that the chemical structure will predominate.

Other embodiments of the present invention include the following:

(a) A pharmaceutical composition comprising an effective amount of a Compound of Formula (I) or a pharmaceutically acceptable salt thereof, and a pharmaceutically acceptable carrier.

(b) The pharmaceutical composition of (a), further comprising a second therapeutic agent selected from the group consisting of HCV antiviral agents, immunomodulators, and anti-infective agents.

(c) The pharmaceutical composition of (b), wherein the HCV antiviral agent is an antiviral selected from the group consisting of HCV protease inhibitors, HCV NS5B polymerase inhibitors and HCV NS5A inhibitors.

(d) A pharmaceutical combination that is (i) a Compound of Formula (I) and (ii) a second therapeutic agent selected from the group consisting of HCV antiviral agents, immunomodulators, and anti-infective agents; wherein the Compound of Formula (I) and the second therapeutic agent are each employed in an amount that renders the combination effective for inhibiting HCV replication, or for treating HCV infection and/or reducing the likelihood or severity of symptoms of HCV infection.

(e) The combination of (d), wherein the HCV antiviral agent is an antiviral selected from the group consisting of HCV protease inhibitors, HCV NS5B polymerase inhibitors and HCV NS5A inhibitors.

(f) A method of inhibiting HCV replication in a subject in need thereof which comprises administering to the subject an effective amount of a Compound of Formula (I) or (Ia).

(g) A method of treating HCV infection and/or reducing the likelihood or severity of symptoms of HCV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HCV antiviral agents, immunomodulators, and anti-infective agents.

(i) The method of (h), wherein the HCV antiviral agent is an antiviral selected from the group consisting of HCV protease inhibitors, HCV NS5B polymerase inhibitors and HCV NS5A inhibitors.

(j) A method of inhibiting HCV replication in a subject in need thereof which comprises administering to the subject the pharmaceutical composition of (a), (b) or (c) or the combination of (d) or (e).

(k) A method of treating HCV infection and/or reducing the likelihood or severity of symptoms of HCV infection in a subject in need thereof which comprises administering to the subject the pharmaceutical composition of (a), (b) or (c) or the combination of (d) or (e).

Additional embodiments of the invention include the pharmaceutical compositions, combinations and methods set forth in (a)-(k) above and the uses set forth in the discussion below, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include the compounds prepared according to Examples 42-65 below and pharmaceutically acceptable salts thereof, and the compounds set forth in Table 1 in the Examples below and pharmaceutically acceptable salts thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes A, B and C below. Alternative synthetic pathways and analogous structures will be apparent to those skilled in the art of organic synthesis.

Scheme A shows a method useful for making nucleoside compounds of formula D, which correspond to the Compounds of Formula (I), wherein A is O; Q is O; and B, R¹, R² and R³ are defined above for the Compounds of Formula (I).

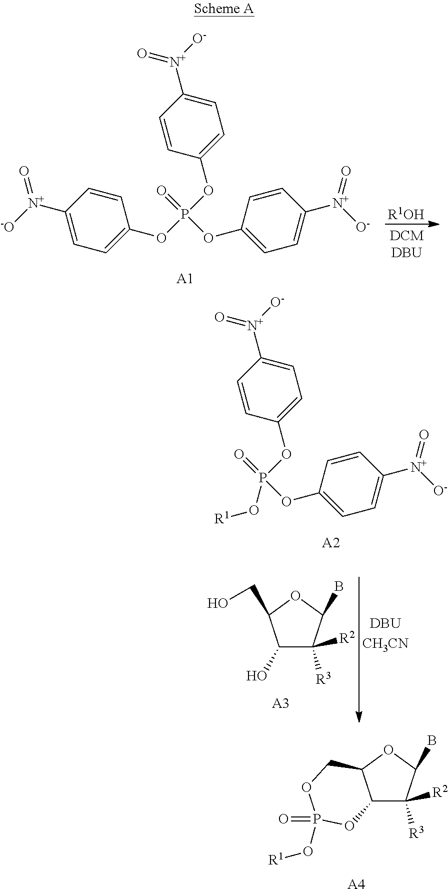

Tris(4-nitrophenyl) phosphate (A1) can be reacted with DBU and an alcohol of formula R¹OH to provide a compound of formula A2. The compound of formula A2 is then reacted with a nucleoside of formula A3 to provide a cyclic phosphate nucleoside prodrug of formula A4.

Scheme B shows an alternate method useful for making nucleoside compounds of formula A4, which correspond to the Compounds of Formula (I), wherein A is O; Q is O; and B, R¹, R² and R³ are defined above for the Compounds of Formula (I).

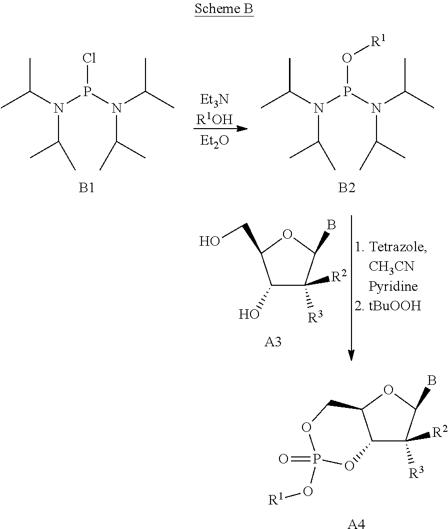

1-chloro-N,N,N′,N′-tetraisopropylphosphinediamine (B1) can be reacted with triethylamine and an alcohol of formula R¹OH to provide a compound of formula B2. The compound of formula B2 is then reacted with a nucleoside of formula A3 to provide a cyclic phosphate nucleoside prodrug of formula A4.

Scheme C shows an alternate method useful for making nucleoside compounds of formula C3, which correspond to the Compounds of Formula (I), wherein A is O or S; Q is O; and B, R¹, R² and R³ are defined above for the Compounds of Formula (I).

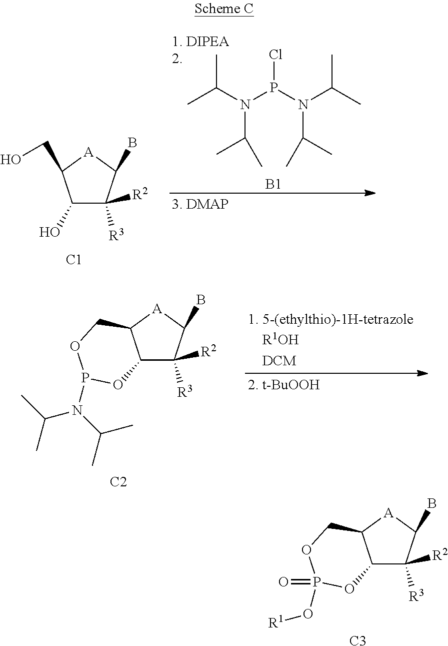

A nucleoside compound of formula C1 is reacted with DIPEA, followed by compound B1, then DMAP to provide a cyclic phosphoramidate of formula C2. The compound of formula C2 can then be reacted with ethylthio tetrazole and an alcohol of formula R¹OH, followed by t-butyl peroxide to provide a cyclic phosphate nucleoside prodrug of formula C3.

### EXAMPLES

### General Procedures

Reactions sensitive to moisture or air were performed under nitrogen or argon atmosphere using anhydrous solvents and reagents. The progress of reactions was determined using either analytical thin layer chromatography (TLC) usually performed with E. Merck pre-coated TLC plates, silica gel 60F-254, layer thickness 0.25 mm or liquid chromatography-mass spectrometry (LC-MS).

The analytical UPLC-MS system used consisted of a Waters SQD2 platform with electrospray ionization in positive and negative detection mode with an Acquity UPLC I-class solvent manager, column manager, sample manager and PDA detector. The column used for standard methods was a CORTECS UPLC C18 1.6 μm, 2.1×30 mm, and the column used for polar methods was an ACQUITY UPLC HSST3 1.8 μm, 2.1×30 mm, the column temperature was 40° C., the flow rate was 0.7 mL/min, and injection volume was 1 μL. UV detection was in the range 210-400 nm. The mobile phase consisted of solvent A (water plus 0.05% formic acid) and solvent B (acetonitrile plus 0.05% formic acid) with different gradients for 4 different methods: 1/ Starting with 99% solvent A for 0.2 minutes changing to 98% solvent B over 1 minutes, maintained for 0.4 minutes, then reverting to 99% solvent A over 0.1 min; 2/ Starting with 99% solvent A for 0.5 minutes changing to 98% solvent B over 3.7 minutes, maintained for 0.4 minutes, then reverting to 99% solvent A over 0.1 min; 3/ Starting with 100% solvent A for 0.4 minutes changing to 98% solvent B over 0.9 minutes, maintained for 0.3 minutes, then reverting to 100% solvent A over 0.1 min; 4/ Starting with 100% solvent A for 0.8 minutes changing to 98% solvent B over 3.4 minutes, maintained for 0.4 minutes, then reverting to 100% solvent A over 0.1 minutes.

The analytical LC-MS system used consisted of an Agilent 6140 quadrupole LC/MS platform with electrospray ionization in positive and negative detection mode with an Agilent 1200 Series solvent manager, column manager, sample manager and PDA detector. The column for standard method was Purospher® STAR RP-18 endcapped 2 μm, Hibar® HR 50-2.1, the column temperature was 60° C., the flow rate was 0.8 mL/min, and injection volume was 0.5-5 μL. UV detection was in the range 210-400 nm. The mobile phase consisted of solvent A (water plus 0.05% formic acid) and solvent B (acetonitrile plus 0.05% formic acid) with different gradients for 2 different methods: 1) Starting with 98% solvent A changing to 100% solvent B over 1.8 minutes, maintained for 0.8 min; 2) Starting with 98% solvent A changing to 100% solvent B over 5.8 minutes, maintained for 0.3 minutes.

Preparative HPLC purifications were usually performed using a mass spectrometry directed system. Usually they were performed on a Waters Chromatography Workstation (MassLynx V4.1) configured with LC-MS System Consisting of: Waters ZQ™ 2000 (quad MS system with Electrospray Ionization), Waters 2545 Gradient Pump, Waters 2767 Injecto/Collector, Waters 2998 PDA Detector, the MS Conditions of: 100-1400 amu, Positive Electrospray, Collection Triggered by MS, and a Waters SUNFIRE® C-18 5 micron, 19 mm (id)×150 mm column. The mobile phases consisted of mixtures of acetonitrile (5-95%) in water containing 0.02% formic acid. Flow rates were maintained at 20 mL/min, the injection volume was 500 to 3000 μL, and the UV detection range was 210-400 nm. Mobile phase gradients were optimized for the individual compounds. Preparative HPLC were also performed on a Gilson system GX-281 (Trilution). The column was a Waters SUNFIRE® Prep C18 5 μm OBD, dimension 50×150 mm. The mobile phase consisted of acetonitrile (5-50%) in water containing 0.02% HCOOH over 60 minutes. Flow rates were maintained at 117 mL/min, the injection volume was 1000 to 7000 μL, and the UV detection range was 260 nm.

Reactions performed using microwave irradiation were normally carried out using an Emrys Optimizer manufactured by Personal Chemistry, or an Initiator manufactured by Biotage. Concentration of solutions was carried out on a rotary evaporator in vacuo. Flash chromatography was usually performed using a Biotage® Flash Chromatography apparatus (Isolera) on silica gel (15-45, 40-63a, or spheric silica) in pre-packed cartridges of the size noted. ¹H NMR spectra were acquired at 400 MHz or 500 MHz spectrometers in CDCl₃ solutions unless otherwise noted. Chemical shifts were reported in parts per million (ppm). Tetramethylsilane (TMS) was used as internal reference in CDCl₃ solutions, and residual CH₃OH peak or TMS was used as internal reference in CD₃OD solutions. Coupling constants (J) were reported in hertz (Hz). Chiral analytical chromatography was performed on one of CHIRALPAK® AS, CHIRALPAK®AD, CHIRALCEL® OD, CHIRALCEL® IA, or CHIRALCEL® OJ columns (250×4.6 mm) (Daicel Chemical Industries, Ltd.) with noted percentage of either ethanol in hexane (% EtOH/Hex) or isopropanol in heptane (% IPA/Hep) as isocratic solvent systems. Chiral preparative chromatography was conducted on one of CHIRALPAK AS, of CHIRALPAK AD, CHIRALCEL® OD, CHIRALCEL®IA, CHIRALCEL® OJ columns (20×250 mm) (Daicel Chemical Industries, Ltd.) with desired isocratic solvent systems identified on chiral analytical chromatography or by supercritical fluid (SFC) conditions.

### Example 1

### Preparation of Intermediate Compound A

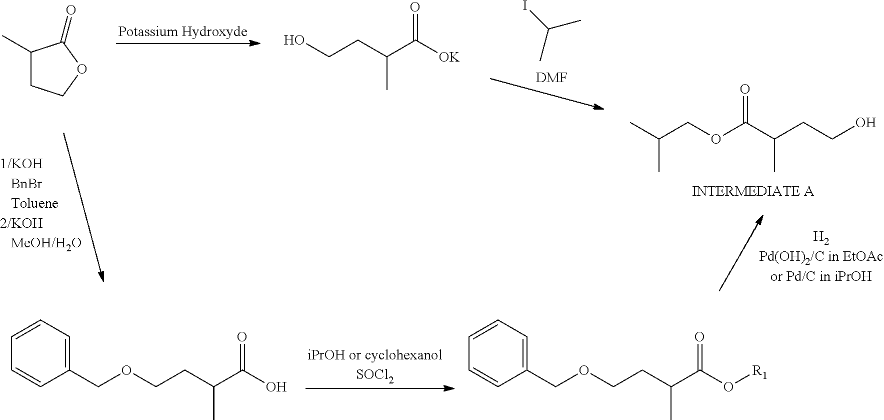

### Preparation of Intermediate Compound A

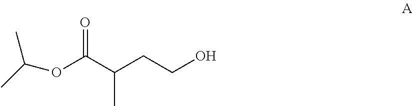

### Method 1

### Step 1:

A solution of α-methyl-γ-butyrolactone (9.90 mL, 88 mmol) in 1M aqueous potassium hydroxide solution (88 mL, 88 mmol) was heated under reflux for 3 hours, then cooled to room temperature and concentrated in vacuo. The crude solid was triturated in diethyl ether, filtered off and washed with diethyl ether. The solid was then dried in vacuo over P₂O₅ at 40° C.

### Step 2:

To a solution of the product of step 1 (10 g, 64.0 mmol) in DMF (80 mL) was added dropwise at room temperature under nitrogen 2-iodopropane (12.78 mL, 128 mmol). The reaction was allowed to stir at room temperature for 5 hours. 2-iodopropane (3.2 mL, 32 mmol) was added, and the reaction mixture was allowed to stir at room temperature for about 15 hours. The mixture was diluted with EtOAc, and the organic layer was washed with a metabisulfite solution and brine. The organic layer was dried, filtered and concentrated in vacuo at 20-30° C. to provide intermediate compound A. ¹H NMR (400 MHz, CDCl₃) δ 5.02 (heptuplet, J=6.30 Hz, 1H), 3.71-3.66 (m, 2H), 2.63-2.54 (m, 1H), 1.96-1.87 (m, 1H), 1.73-1.65 (m, 2H), 1.24 (d, J=6.30 Hz, 3H), 1.235 (d, J=6.30 Hz, 3H), 1.18 (d, J=6.91 Hz, 3H).

### Method 2:

### Step 1:

Crushed KOH (28.0 g, 499 mmol) was added to a solution of α-methyl-γ-butyrolactone (9.43 mL, 100 mmol) and benzyl bromide (47.5 mL, 400 mmol) in toluene (200 mL). The reaction was allowed to stir at 110° C. for 5 hours, then toluene was removed in vacuo. Methanol (200 mL) was added to the reaction mixture followed by KOH (10 g, 178 mmol), and water (100 mL), then the reaction mixture was allowed to stir at reflux for 7 hours. The reaction mixture was extracted with diethyl ether (3×200 mL), the aqueous layer was acidified with concentrated HCl, and extracted with DCM (3×200 mL). The combined organic layers were dried over MgSO₄, filtered, and concentrated in vacuo at room temperature to provide the intermediate compound 4-(benzyloxy)-2-methylbutanoic acid as oil.

### Step 2:

To a solution of 4-(benzyloxy)-2-methylbutanoic acid (21 g, 91 mmol) in 2-propanol (300 mL) was added dropwise thionyl chloride (9.27 mL, 127 mmol) at RT. The reaction was allowed to stir at reflux for 4 hours, then, concentrated in vacuo. The crude residue obtained was purified using flash chromatography on silica gel (PE/EtOAc: 90/10) to provide the intermediate compound isopropyl 4-(benzyloxy)-2-methylbutanoate as oil.

### Step 3:

To a solution of isopropyl 4-(benzyloxy)-2-methylbutanoate (22.15 g, 88 mmol) in 2-propanol (177 mL) in a pressure reactor was added palladium on carbon (2.21 g, 2.077 mmol). The reaction mixture was hydrogenated at room temperature under 7.0 bars for 1 day. The reaction mixture was filtered through a pad of K₂CO₃, and the filtrate was concentrated at RT. The crude residue obtained was further dried for about 15 hours at room temperature under high vacuum to provide intermediate compound A.

### Example 2

### Preparation of Intermediate Compound B

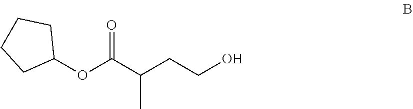

Intermediate B was synthesized using the method described in Example 1, method 1, for the synthesis of intermediate compound A starting for step 2 from iodocyclopentane (1.2 equiv.; no additional iodocyclopentane was necessary for reaction completion.). The reaction was allowed to stir at room temperature for 4 days. ¹H NMR (400 MHz, CDCl₃) δ 5.19-5.15 (m, 1H), 3.73-3.64 (m, 2H), 2.62-2.54 (m, 1H), 1.95-1.82 (m, 4H), 1.74-1.57 (m, 7H), 1.18 (d, J=7.09 Hz, 3H); LC/MS: [(M+1)]⁺=187.0.

### Example 3

### Preparation of Intermediate Compound C

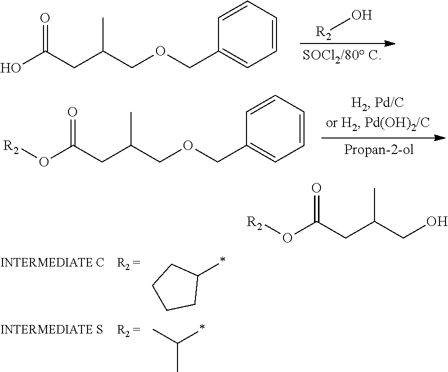

### Step 1:

To a solution of 4-(benzyloxy)-3-methylbutanoic acid (6 g, 28.8 mmol) in cyclopentanol (26.1 mL, 288 mmol) was added dropwise thionyl chloride (2.52 mL, 34.6 mmol). The reaction was allowed to stir at 80° C. for 3 days after further addition of thionyl chloride (5 further additions, each of 0.2 equiv.). The reaction mixture was concentrated in vacuo, and cyclopentanol was distillated in vacuo. The crude residue obtained was purified using silica gel flash chromatography (PE/Et₂O: 0 to 15%).

### Step 2:

To a solution of cyclopentyl 4-(benzyloxy)-3-methylbutanoate (5.7 g, 19.59 mmol) in propan-2-ol (50 mL) purged 3 times in vacuo/nitrogen was added Palladium on Carbon (0.57 g, 5.36 mmol). The reaction mixture was put through a vacuum/hydrogen purge cycle 3 times, then the reaction mixture was allowed to stir at room temperature under hydrogen (5 bars) for about 15 hours. The reaction mixture was filtered through a celite pad, washed with CH₃CN, concentrated in vacuo, and co-evaporated with toluene/CH₃CN to provide intermediate compound C: ¹H NMR (400 MHz, CDCl₃) δ 5.20-5.15 (m, 1H), 3.57 (dd, J=10.73 Hz, 5.13 Hz, 1H), 3.47 (dd, J=10.73 Hz, 6.65 Hz, 1H), 2.42-2.36 (m, 1H), 2.23-2.11 (m, 2H), 1.88-1.82 (m, 2H), 1.74-1.58 (m, 6H), 0.97 (d, J=6.62 Hz, 3H).

### Example 4

### Preparation of Intermediate Compound D

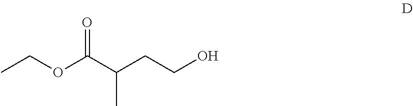

Intermediate D was synthesized using the method described in Example 1, method 1, for the synthesis of intermediate compound A starting for step 2 from iodoethane (1.2 equiv.; no additional iodoethane was necessary for reaction completion). ¹H NMR (400 MHz, CDCl₃) δ 4.14 (q, J=7.11 Hz, 2H), 3.69-3.63 (m, 2H), 2.67-2.58 (m, 1H), 1.98-1.89 (m, 1H), 1.71-1.63 (m, 1H), 1.26 (t, J=7.11 Hz, 3H), 1.19 (d, J=7.10 Hz, 3H); LC/MS: [(M+1)]⁺=147.2.

### Example 5

### Preparation of Intermediate Compounds E and F

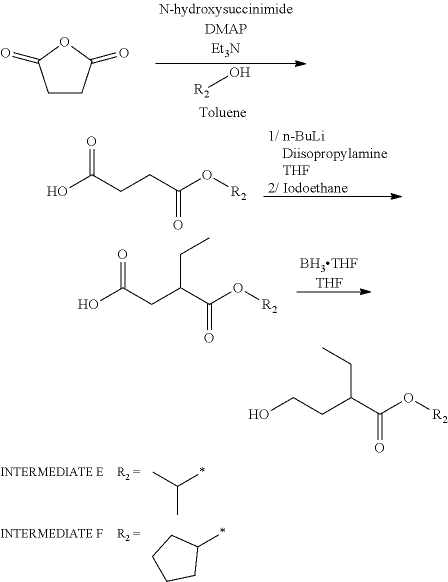

### Step 1:

To a suspension of dihydrofuran-2,5-dione (10 g, 100 mmol) in toluene (60 mL) under nitrogen was added N-hydroxysuccinimide (3.45 g, 30.0 mmol), 4-dimethylaminopyridine (1.221 g, 9.99 mmol), propan-2-ol (22.92 mL, 300 mmol) and triethylamine (4.18 mL, 30.0 mmol). The reaction was allowed to stir at 110° C. for about 15 hours. After cooling to RT, ethyl acetate was added, and the organic layer was washed twice with 10% citric acid solution and brine, dried over Na₂SO₄, filtered and concentrated in vacuo to provide the product as oil.

### Step 2:

To a solution of diisopropylamine (14.56 mL, 103 mmol) in THF (80 mL) (0.8 mL/mmol of DIPEA) at −78° C. was added a solution of n-butyllithium (41.2 mL, 103 mmol). The reaction was allowed to stir at −78° C. for 30 minutes, and then a solution of the product of Step 1 (4-isopropoxy-4-oxobutanoic acid: 7.5 g, 46.8 mmol) in THF (42 mL) (0.9 mL/mmol) was added. The reaction mixture was warmed to 0° C. and allowed to stir at this temperature for 2 hours before cooled to −78° C. Iodoethane (5.27 mL, 65.6 mmol) was added dropwise. The reaction was allowed to warm to room temperature and allowed to stir for about 15 hours, then the reaction mixture was quenched with H₂O (10 mL) and concentrated in vacuo. The crude residue obtained was partitioned between EtOAc (250 mL) and cold HCl 1N (140 mL). The organic layer was dried over Na₂SO₄, filtered and concentrated in vacuo. The crude residue obtained was purified using flash chromatography on silica gel (PE/EtOAc: 0 to 35%) to provide the product as oil. LC/MS: [(M+1)]⁺=189.0.

### Step 3:

To a solution of previous intermediate compound, 3-(isopropoxycarbonyl)pentanoic acid (4.2 g, 22.31 mmol) in THF (170 mL) (8 mL/mmol) cooled at 0° C. was added dropwise borane-tetrahydrofuran complex (33.5 mL, 33.5 mmol) under nitrogen. The reaction was allowed to warm to room temperature and allowed to stir for 2 hours. The reaction mixture was then added slowly to a saturated aqueous NaHCO₃ solution. The aqueous layer was extracted twice with diethyl ether. The organic layers were dried over Na₂SO₄, filtered and concentrated in vacuo at RT. The crude residue obtained was purified using flash chromatography on silica gel (dichloromethane-dichloromethane/MeOH (8/2): 0 to 40%) to provide intermediate compound E: ¹H NMR (400 MHz, CDCl₃) δ 5.04 (heptuplet, J=6.25 Hz, 1H), 3.67-3.63 (m, 2H), 2.45-2.37 (m, 1H), 1.92-1.83 (m, 1H), 1.77-1.61 (m, 2H), 1.59-1.49 (m, 1H), 1.245 (d, J=6.25 Hz, 3H), 1.242 (d, J=6.25 Hz, 3H), 0.91 (t, J=7.45 Hz, 3H); LC/MS: [(M+1)]⁺=175.0.

### Synthesis of Intermediate Compound F

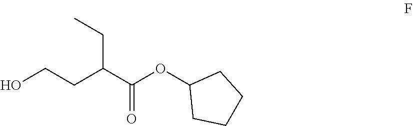

Intermediate F was synthesized using the method described immediately above for the synthesis of intermediate compound E and replacing propan-2-ol with cyclopentanol in step 1. The reaction of step 1 was carried out at 100° C. ¹H NMR (400 MHz, CDCl₃) δ 5.21-5.17 (m, 1H), 3.67-3.64 (m, 2H), 2.44-2.37 (m, 1H), 1.91-1.51 (m, 12H), 0.91 (t, J=7.46 Hz, 3H); LC/MS: [(M+1)]⁺=201.0.

### Example 6

### Preparation of Intermediate Compound G

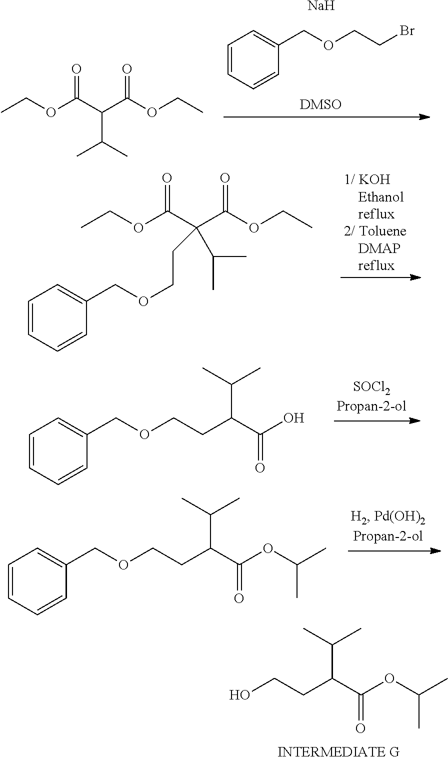

### Step 1:

To a solution of sodium hydride 60% in oil (5.93 g, 148 mmol) in DMSO (124 mL) previously stirred for 10 minutes at room temperature was added dropwise diethyl 2-isopropylmalonate (25.3 mL, 124 mmol). The reaction was allowed to stir for 1 hour at RT, then, ((2-bromoethoxy)methyl)benzene (19.55 mL, 124 mmol) was added, and the reaction mixture was allowed to stir at room temperature for about 15 hours and at 60° C. for 2 hours. The reaction mixture was quenched with saturated aqueous NH₄Cl solution and extracted with Et₂O. The organic layer was washed with deionized water, dried over MgSO₄, filtered, and concentrated in vacuo. The crude residue obtained was purified using flash chromatography on silica gel (hexane/EtOAc: 90/10) to provide the product as oil. LC/MS: [(M+1)]⁺=337.0.

### Step 2:

To a crushed potassium hydroxide (24.42 g, 435 mmol) in ethanol (238 mL) was added the previous intermediate compound diethyl 2-(2-(benzyloxy)ethyl)-2-isopropylmalonate (24 g, 71.3 mmol). The reaction was allowed to stir at reflux for 16 hours. After cooling to RT, the reaction mixture was extracted with diethyl ether, and the collected aqueous layer was acidified with HCl and extracted with dichloromethane (3×200 mL). The combined organic layers were dried over MgSO₄, filtered, and concentrated in vacuo. The crude oil obtained was diluted with toluene (238 mL) and to the resulting solution was added DMAP (2.44 g, 19.97 mmol). The reaction was allowed to stir at reflux for 2 hours, then concentrated under high vacuum, and the crude residue obtained was purified using flash chromatography on silica gel (hexane/acetone: 90/10) to provide the product. LC/MS: [(M+1)]⁺=237.0.

### Step 3:

To a stirred, cooled 0° C. mixture of the product of Step 2 (9.6 g, 40.6 mmol) in propan-2-ol (58.0 mL) under nitrogen was added thionyl chloride (8.90 mL, 122 mmol). The reaction was allowed to stir at reflux for about 15 hours and then concentrated in vacuo. The crude residue obtained was purified using flash chromatography on silica gel (Petroleum ether/Et₂O: 0 to 50%) to provide the product as oil. LC/MS: [(M+1)]⁺=279.2.

### Step 4:

To a stirred mixture of Pd(OH)₂ (2.068 g, 14.73 mmol) in propan-2-ol (36.8 mL) under H₂ was added the product of Step 3 (4.1 g, 14.73 mmol), and the resulting reaction was allowed to stir at room temperature under hydrogen for 30 minutes. The reaction mixture was then filtered through a celite pad, and the filtrate was concentrated in vacuo. The crude residue obtained was purified using flash chromatography on silica gel (CH₂Cl₂/MeOH: 0 to 10%) to provide intermediate compound G: ¹H NMR (400 MHz, CDCl₃) δ 5.04 (heptuplet, J=6.25 Hz, 1H), 3.68-3.58 (m, 2H), 2.26-2.21 (m, 1H), 1.96-1.73 (m, 3H), 1.25-1.23 (m, 6H), 0.94 (d, J=6.76 Hz, 3H), 0.935 (d, J=6.76 Hz, 3H); LC/MS: [(M+1)]⁺=189.2.

### Example 7

### Preparation of Intermediate Compounds H, I and J

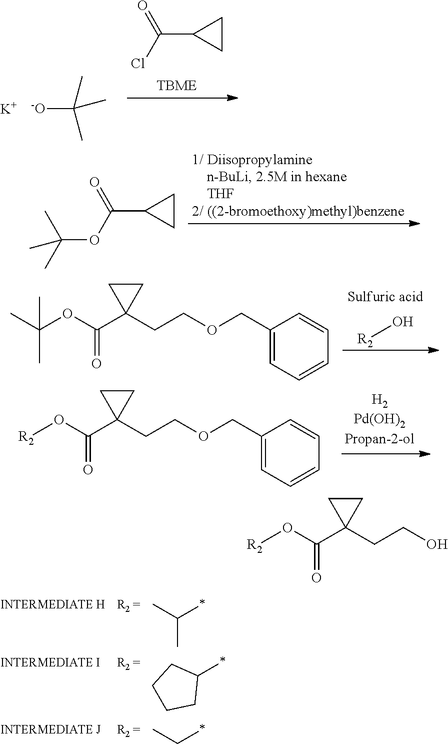

### Preparation of Intermediate H

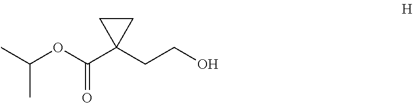

### Step 1:

To a cooled solution of potassium tert-butoxide (32.2 g, 287 mmol) in TBME (478 mL) at 0° C. was added dropwise cyclopropanecarbonyl chloride (21.89 mL, 239 mmol) over a period of 15 minutes. The reaction was warmed to room temperature and allowed to stir for about 15 hours. The reaction mixture was then quenched with a saturated aqueous NaHCO₃ solution and the organic layer was extracted and washed with brine. The organic layer was then dried, filtered and concentrated in vacuo (bath temperature=30° C., 50 mbars because of volatility of product). The crude residue obtained was purified using flash chromatography on silica gel (PE/EtOAc: 0 to 10%) to provide the product as oil: ¹H NMR (400 MHz, CDCl₃) δ 1.53-1.47 (m, 1H), 1.45 (s, 9H), 0.93-0.89 (m, 2H), 0.79-0.74 (m, 2H).

### Step 2:

To a solution of diisopropylamine (15.65 mL, 111 mmol) in THF (105 mL) under nitrogen at −78° C. was slowly added n-butyllithium (44.3 mL, 111 mmol). The reaction was allowed to stir at −78° C. for 30 minutes and the product from step 1 (15 g, 105 mmol) was added dropwise. The reaction was allowed to stir under nitrogen at −78° C. for 30 minutes, then ((2-bromoethoxy)methyl)benzene (16.68 mL, 105 mmol) was added dropwise at −78° C. The reaction was allowed to stir at −78° C. for 1 hour, then was allowed to warm to room temperature, and allowed to stir for about 15 hours. The reaction mixture was then diluted with EtOAc and water, and the collected organic layer was dried, filtered and concentrated in vacuo. The crude residue obtained was purified using flash chromatography on silica gel (PE/EtOAc: 0 to 10%) to provide the product as oil: ¹H NMR (400 MHz, CDCl₃) δ 7.37-7.33 (m, 5H), 4.50 (s, 2H), 3.62 (t, J=7.07 Hz, 2H), 1.85 (t, J=7.07 Hz, 2H), 1.39 (s, 9H), 1.14-1.12 (m, 2H), 0.72-0.69 (m, 2H); LC/MS: [(M+23]⁺=299.2.

### Step 3:

To a solution of the product of Step 2 (2.075 g, 6.38 mmol) in isopropanol (63.8 mL) was added sulfuric acid (1.36 mL, 25.5 mmol). The reaction was allowed to stir at 80° C. for 36 hours, then cooled to room temperature and neutralized with a slow addition of saturated aqueous NaHCO₃ solution. The aqueous layer was extracted with EtOAc, and the organic layer was dried and concentrated in vacuo. The crude residue obtained was purified using flash chromatography on silica gel (PE/EtOAc: 0 to 10%) to provide the product as oil: ¹H NMR (400 MHz, CDCl₃) δ 7.36-7.27 (m, 5H), 4.95 (heptuplet, J=6.26 Hz, 1H), 4.50 (s, 2H), 3.63 (t, J=7.08 Hz, 2H), 1.88 (t, J=7.08 Hz, 2H), 1.19-1.16 (m, 8H), 0.76-0.74 (m, 2H); LC/MS: [(M+1]⁺⁼263.0.

### Step 4:

To a solution of the product of Step 3 (1.41 g, 5.27 mmol) in propan-2-ol (26.3 mL) was added Pd(OH)₂ (350 mg, 2.492 mmol). The reaction was allowed to stir at room temperature for about 15 hours under hydrogen atmosphere then filtered through a celite pad, washed with EtOAc, and concentrated in vacuo to provide the product as oil: ¹H NMR (400 MHz, CDCl₃) δ 4.98 (heptuplet, J=6.33 Hz, 1H), 3.78 (t, J=6.17 Hz, 2H), 1.81 (t, J=6.17 Hz, 2H), 1.22-1.20 (m, 8H), 0.76-0.73 (m, 2H); LC/MS: [(M+1]⁺=173.0.

### Preparation of Intermediate Compound I

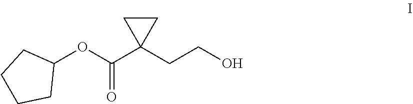 [SMILES: O=C(OC1CCCC1)C1(CCO)CC1]

Intermediate compound I was synthesized using the method described immediately above for the synthesis of intermediate compound H, and substituting cyclopentanol for isopropanol in step 3. The reaction of step 3 allowed to stir at 95° C. for 24 hours. ¹H NMR (400 MHz, CDCl₃) δ 5.16-5.12 (m, 1H), 3.77 (t, J=6.15 Hz, 2H), 1.86-1.79 (m, 4H), 1.71-1.56 (m, 6H), 1.24-1.20 (m, 2H), 0.76-0.73 (m, 2H); LC/MS: [(M+1)]⁺=199.0.

### Preparation of Intermediate Compound J

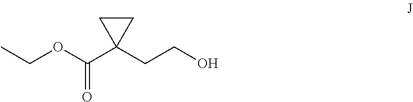

Intermediate compound J was synthesized using the method described immediately above for the synthesis of intermediate compound H, and substituting ethanol for isopropanol in step 3. The reaction of step 3 allowed to stir at 90° C. for 24 hours. ¹H NMR (400 MHz, CDCl₃) δ 4.11 (q, J=7.16 Hz, 2H), 3.78 (t, J=6.12 Hz, 2H), 1.82 (t, J=6.12 Hz, 2H), 1.29-1.20 (m, 5H), 0.78-0.75 (m, 2H); LC/MS: [(M+1)]⁺=159.0.

### Example 8

### Preparation of Intermediate Compound K

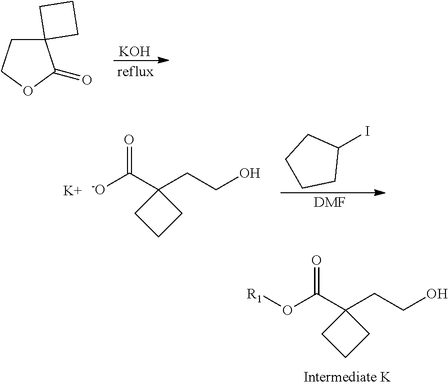

Intermediate K was synthesized in two steps—the first step using the method described in Example 1, method 1, step 1, but using 6-oxaspiro[3.4]octan-5-one as the starting material; and the second step using the method of Example 1, method 1, step 2, but using iodocyclopentane (1.2 eq.). The reaction was allowed to stir at room temperature for about 15 hours and worked up as described to provide compound K. ¹H NMR (400 MHz, CDCl₃) δ 5.21-5.17 (m, 1H), 3.65 (t, J=6.45 Hz, 2H), 2.46-2.39 (m, 2H), 2.05 (t, J=6.45 Hz, 2H), 1.99-1.59 (m, 12H); LC/MS: [(M+1)]⁺=213.0.

### Example 9

### Preparation of Intermediate Compound L

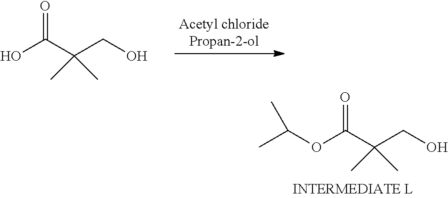

To propan-2-ol (75 mL) was added dropwise acetyl chloride (4.54 mL, 62.21 mmol). The reaction was allowed to stir at room temperature for 5 minutes, then 3-hydroxy-2,2-dimethylpropanoic acid (3 g, 24.89 mmol) was added. The reaction was allowed to stir at 85° C. for about 15 hours, then concentrated in vacuo, co-evaporated with toluene and dried in vacuo to provide the intermediate compound L as a solid. ¹H NMR (400 MHz, CDCl₃) δ 5.03 (heptuplet, J=6.22 Hz, 1H), 3.54 (s, 2H), 1.25 (d, J=6.22 Hz, 6H), 1.18 (s, 6H).

### Example 10

### Preparation of Intermediate Compound 0

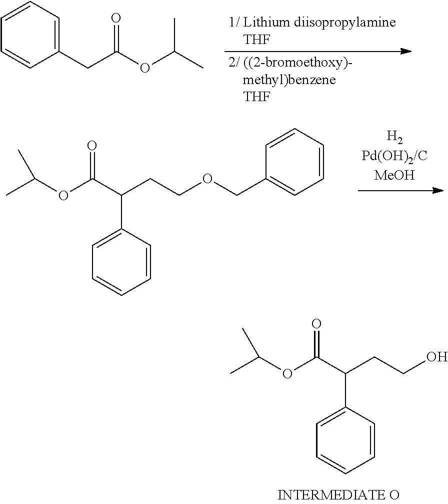 [SMILES: CC(C)OC(=O)Cc1ccccc1 ; CC(C)OC(=O)C(CCOCc1ccccc1)c1ccccc1 ; CC(C)OC(=O)C(CCO)c1ccccc1]

### Step 1:

To a −78° C. solution of lithium diisopropylamine (4.65 mL, 9.30 mmol) in THF (16 mL) was added a solution of isopropyl 2-phenylacetate (1.492 g, 8.37 mmol) in THF (4 mL). The reaction was allowed to stir at 0° C. for 20 minutes, then cooled to −78° C., and a solution of ((2-bromoethoxy)methyl)-benzene (2 g, 9.30 mmol) in THF (4 mL) was added. The reaction was allowed to stir at room temperature for about 15 hours, then the reaction mixture was quenched with a saturated aqueous NH₄Cl solution and concentrated in vacuo. The resulting aqueous mixture was extracted with EtOAc and the organic layer was dried over Na₂SO₄, filtered and concentrated in vacuo. The crude residue obtained was purified using flash chromatography on silica gel (Petroleum ether/EtOAc: 0 to 10%) to provide the product: LC/MS: [(M+1)]⁺=313.2.

### Step 2:

To a solution of isopropyl 4-(benzyloxy)-2-phenylbutanoate (515 mg, 1.65 mmol) in methanol (30 mL, 20 mL/mmol) was added after vacuum-nitrogen (2×) Pd(OH)₂ on carbon (105 mg, 0.75 mmol). After several nitrogen-vacuum purge cycles, the reaction mixture was allowed to stir under hydrogen atmosphere for 3 hours. The reaction mixture was filtered through a celite pad, and concentrated in vacuo to provide compound O: ¹H NMR (400 MHz, CDCl₃) δ 7.32-7.24 (m, 5H), 5.00 (heptuplet, J=6.28 Hz, 1H), 3.75 (t, J=7.59 Hz, 1H), 3.69-3.55 (m, 2H), 2.39-2.30 (m, 1H), 2.04-1.96 (m, 1H), 1.22 (d, J=6.28 Hz, 3H), 1.12 (d, J=6.28 Hz, 3H); LC/MS: [(M+1)]⁺=223.0.

### Example 11

### Preparation of Intermediate Compounds P and Q

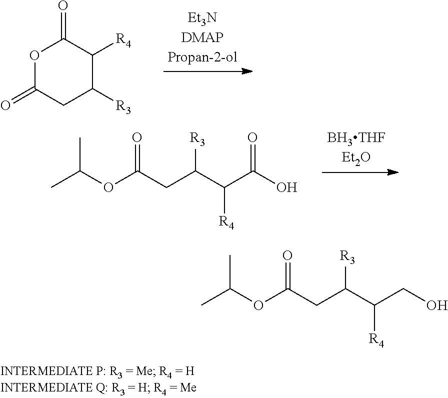

### Synthesis of Intermediate Compound P

### Step 1:

To a solution of 3-methylglutaric anhydride (5.9 g, 46.0 mmol) and DMAP (0.563 g, 4.60 mmol) in propan-2-ol (70.8 mL) at room temperature was added triethylamine (6.41 mL, 46.0 mmol). The reaction was allowed to stir at 95° C. for about 15 hours, then was cooled to RT and concentrated in vacuo. The crude residue obtained was dissolved in EtOAc, and the organic layer was washed with a 1M citric acid solution (2×) and then with brine. The organic layer was dried, filtered and concentrated and dried in vacuo, and the product obtained was used without further purification. LC/MS: [(M+1)]⁺=189.0.

### Step 2:

To a stirred mixture of the product obtained in Step 1 (7.65 g, 40.6 mmol) in diethyl ether (203 mL) at 0° C. under nitrogen was added dropwise borane tetrahydrofuran complex (48.8 mL, 48.8 mmol). The reaction was allowed to stir at room temperature for 90 minutes, then the reaction mixture was then slowly added at 0° C. to a saturated aqueous NaHCO₃ solution. The aqueous layer was extracted with diethyl ether (2×). The combined organic extracts were combined, dried in vacuo, and the crude residue obtained was partitioned between water and dichloromethane. The organic layer was dried, filtered, concentrated in vacuo, and dried in vacuo to provide compound P. ¹H NMR (400 MHz, DMSO-d₆) δ 4.89 (heptuplet, J=6.22 Hz, 1H), 4.38-4.35 (m, 1H), 3.45-3.38 (m, 2H), 2.27 (dd, J=14.17 Hz, 5.27 Hz, 1H), 2.04 (dd, J=14.17 Hz, 8.38 Hz, 1H), 2.03-1.94 (m, 1H), 1.48-1.25 (m, 2H), 1.18 (d, J=6.22 Hz, 6H), 0.88 (d, J=6.43 Hz, 3H); LC/MS: [(M+1)]⁺=175.0.

### Synthesis of Intermediate Compound 0

Intermediate compound Q was synthesized using the method described immediately above for the synthesis of intermediate compound P, and substituting 2-methylglutaric anhydride for 3-methylglutaric anhydride in step 1. ¹H NMR (400 MHz, DMSO-d₆) δ 4.93-4.83 (m, 1H), 4.44-4.38 (m, 1H), 3.41-3.34 (m, 1H), 3.27-3.17 (m, 1H), 2.39-2.18 (m, 2H), 1.68-1.24 (m, 3H), 1.18 (d, J=6.30 Hz, 6H), 1.05 (d, J=6.95 Hz, 1.2H), 0.82 (d, J=6.66 Hz, 1.8H); LC/MS: [(M+1)]⁺=175.3.

### Example 12

### Preparation of Intermediate Compound R

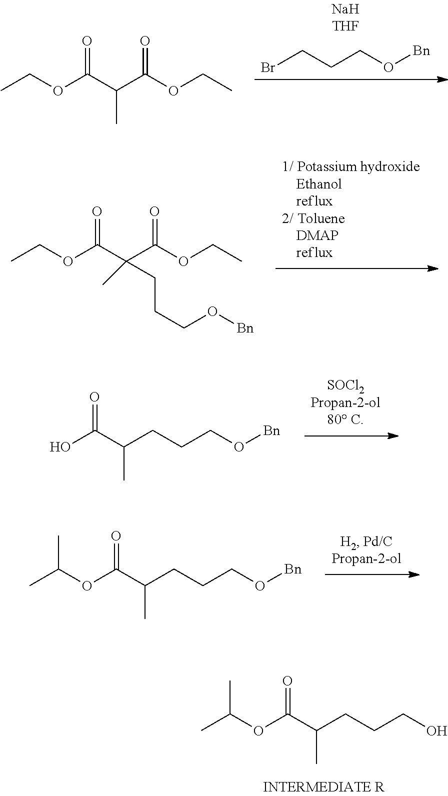 [SMILES: CCOC(=O)C(C)C(=O)OCC ; BrCCCOCc1ccccc1 ; CCOC(=O)C(C)(CCCOCc1ccccc1)C(=O)OCC ; CC(CCCOCc1ccccc1)C(=O)O ; CC(C)OC(=O)C(C)CCCOCc1ccccc1 ; CC(C)OC(=O)C(C)CCCO]

### Step 1:

To a cooled (0° C.) solution of diethyl 2-methylmalonate (10 g, 57.4 mmol) in THF (50 mL) was added portionwise over 5 minutes sodium hydride 60% in oil (2.53 g, 63.1 mmol). After gas evolution ceased, a solution of ((3-bromopropoxy)methyl)benzene (15.78 g, 68.9 mmol) in THF (20 mL) was added dropwise. The reaction was allowed to stir at room temperature for about 15 hours; then the reaction was quenched with few drops of methanol. Saturated aqueous NH₄Cl solution was added to the reaction mixture, and the resulting solution was extracted with EtOAc. The collected organic layer was dried and concentrated in vacuo to provide the product. LC/MS: [(M+1)]⁺=323.0.

### Step 2:

A solution of the product obtained in Step 1 (16.55 g, 25.7 mmol) and crushed potassium hydroxide (8.64 g, 154 mmol) in ethanol (169 mL), was allowed to stir at reflux for 16 hours. After cooling to RT, the reaction mixture was washed with Et₂O (2×), acidified with a concentrated HCl solution to pH 2 and extracted with dichloromethane (3×150 mL). The combined organic extracts were dried and concentrated in vacuo to provide an oil which was heated in toluene (169 mL), and DMAP (0.627 g, 5.13 mmol) for 4 hours. The reaction mixture was concentrated in vacuo, and the crude residue obtained was purified using flash chromatography on silica gel (PE/Et₂O) to provide the product. LC/MS: [(M+1)]⁺=223.0.

### Step 3:

To a solution of the product obtained in Step 2 (3.25 g, 14.61 mmol) in propan-2-ol (48.7 mL) at room temperature was added thionyl chloride (1.49 mL, 20.46 mmol). The reaction was allowed to stir at 80° C. for 6 hours, then cooled to RT. The reaction mixture was concentrated in vacuo, and the crude residue obtained was purified using flash chromatography on silica gel (PE/EtOAc: 0 to 30%) to provide the product. LC/MS: [(M+1)]⁺=265.0.

### Step 4:

To a solution of the product obtained in Step 3 (4.02 g, 14.46 mmol) in propan-2-ol (40 mL) under N₂ was added palladium on carbon (0.80 g, 7.52 mmol). The solution was purged with N₂/vacuum (3 times), then allowed to stir under hydrogen for about 15 hours. The reaction mixture was filtered through a celite pad, and washed with CH₃CN. The filtrate was concentrated in vacuo (bath T°=RT) and co-evaporated with toluene to provide intermediate compound R (stored at 4° C.): ¹H NMR (400 MHz, CDCl₃) δ 5.01 (heptuplet, J=6.25 Hz, 1H), 3.67-3.61 (m, 2H), 2.45-2.36 (m, 1H), 1.79-1.46 (m, 4H), 1.23 (d, J=6.25 Hz, 6H), 1.16 (d, J=7.05 Hz, 3H).

### Example 13

### Preparation of Intermediate Compound S

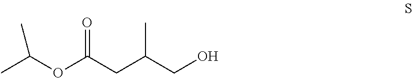

Intermediate compound S was synthesized using the method described in Example 3. In step 1, propan-2-ol was used and the reaction mixture was allowed to stir at reflux for 4 hours, then concentrated in vacuo. The crude compound was used in step 2 without further purification. In step 2, the reaction was carried out with Pd(OH)₂ on carbon, and the reaction mixture was allowed to stir under hydrogen atmosphere for about 15 hours. ¹H NMR (400 MHz, CDCl₃) δ 5.07-4.97 (m, 1H), 3.59-3.46 (m, 2H), 2.43-2.38 (m, 1H), 2.23-2.14 (m, 2H), 1.91 (brs, 1H), 1.24 (d, J=6.18 Hz, 6H), 0.98-0.97 (m, 3H); LC/MS: [(M+1)]⁺=161.0.

### Example 14

### Preparation of Intermediate Compound T

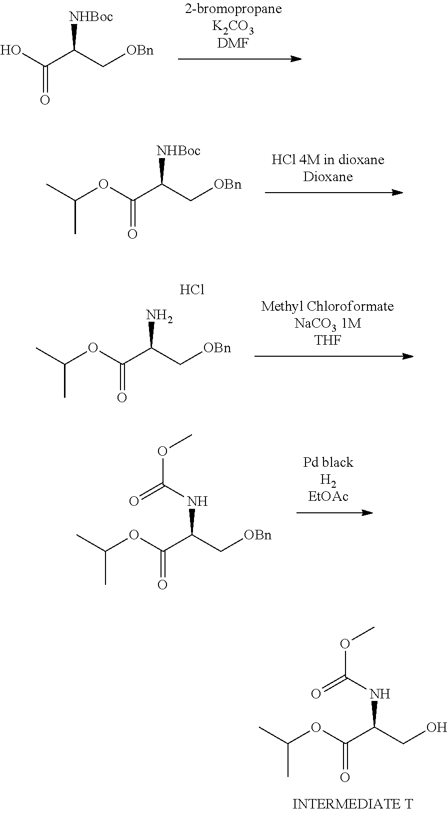 [SMILES: CC(C)(C)OC(=O)N[C@@H](COCc1ccccc1)C(=O)O ; CC(C)OC(=O)[C@H](COCc1ccccc1)NC(=O)OC(C)(C)C ; Cl ; CC(C)OC(=O)[C@@H](N)COCc1ccccc1 ; COC(=O)N[C@@H](COCc1ccccc1)C(=O)OC(C)C ; COC(=O)N[C@@H](CO)C(=O)OC(C)C]

### Step 1:

To a cold (0° C.) solution of O-benzyl-N-(tert-butoxycarbonyl)-L-serine (12 g, 48.76 mmol) and potassium carbonate (6.81 g, 48.76 mmol) in DMF (60 mL) was added 2-bromopropane (4.6 mL, 48.76 mmol). The reaction was allowed to stir at 60° C. for about 15 hours. The reaction mixture was partially concentrated in vacuo, then, ethyl acetate and brine were added. The aqueous layer was extracted once with EtOAc, and the combined organic layers were dried, filtered and concentrated in vacuo to provide the product.

### Step 2:

To a solution of the product obtained in Step 1 (13.5 g, 40 The mmol) in 1,4-dioxane (90 mL) was added a 4M HCl solution in 1,4-dioxane (30 mL). The resulting reaction was allowed to stir at room temperature for about 15 hours, then concentrated in vacuo. EtOAc and Et₂O were added to the resulting residue, and the resulting solution was filtered. The collected solid was washed with Et₂O to provide the product. LC/MS: [(M+1)]⁺=238.0.

### Step 3:

To a solution of the product obtained in Step 2, HCl salt (10.8 g, 39.4 mmol) in THF (120 mL) was added a 1M sodium carbonate solution (60 mL) and methyl chloroformate (3.37 mL, 43.4 mmol). The reaction was allowed to stir at room temperature for about 15 hours, then the reaction mixture was concentrated in vacuo. DCM and water were added to the crude residue, and the collected organic layer was dried, filtered and concentrated in vacuo to provide the product. LC/MS: [(M+23)]⁺=318.0.

### Step 4:

To a solution of the product obtained in Step 3 (12 g, 40.64 mmol) in EtOAc (60 mL) was added a few drops of a 1N HCl solution, followed by palladium black (1.2 g, 11.28 mmol). The reaction was allowed to stir under hydrogen at room temperature for about 15 hours, then filtered through a celite pad, and washed with EtOAc. The filtrate was concentrated in vacuo, and the crude residue obtained was purified using flash chromatography on silica gel to provide intermediate compound T. ¹H NMR (400 MHz, CDCl₃) δ 5.69-5.68 (m, 1H), 5.10 (heptuplet, J=6.23 Hz, 1H), 4.38 (brs, 1H), 3.98-3.89 (m, 2H), 3.71 (s, 3H), 1.28 (d, J=6.23 Hz, 3H), 1.275 (d, J=6.23 Hz, 3H); LC/MS: [(M+1)]⁺=206.0.

### Example 15

### Preparation of Intermediate Compound U

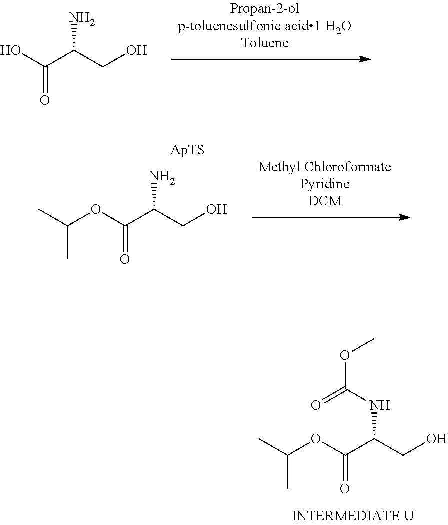

### Step 1:

To a solution of D-serine (10 g, 95.2 mmol) in toluene (190 mL) was added propan-2-ol (66.6 mL, 0.7 mL/mmol) followed by p-toluenesulfonic acid monohydrate (19.85 g, 102.8 mmol). The reaction was fitted with a Dean-Stark trap and allowed to stir at 115° C. for 8 hours. Propan-2-ol (50 mL) and toluene (50 mL) were then added, and the reaction was allowed to stir for an additional 8 hours at 115° C. with the Dean-Stark trap in place. The reaction mixture was cooled to RT, then partially concentrated in vacuo, and Et₂O was added. The resulting solution was filtered, and the collected solid was washed with Et₂O and dried in vacuo to provide the product.

### Step 2:

A suspension of the product obtained in Step 1, tosylate salt (10 g, 21.92 mmol) and pyridine (6.20 mL) in dichloromethane (440 mL) was cooled to 0° C., and methyl chloroformate (2.21 mL, 28.49 mmol) was added dropwise. The reaction was allowed to stir under nitrogen for 5 hours. The reaction mixture was then washed with water (3×50 mL). The organic layer was dried, filtered, concentrated in vacuo, and co-evaporated with toluene to provide intermediate compound U. ¹H NMR (400 MHz, CDCl₃) δ 5.73 (brs, 1H), 5.09 (heptuplet, J=6.20 Hz, 1H), 4.37 (brs, 1H), 3.98-3.88 (m, 2H), 3.70 (s, 3H), 1.28 (d, J=6.20 Hz, 3H), 1.275 (d, J=6.20 Hz, 3H).

### Example 16

### Preparation of Intermediate Compound V

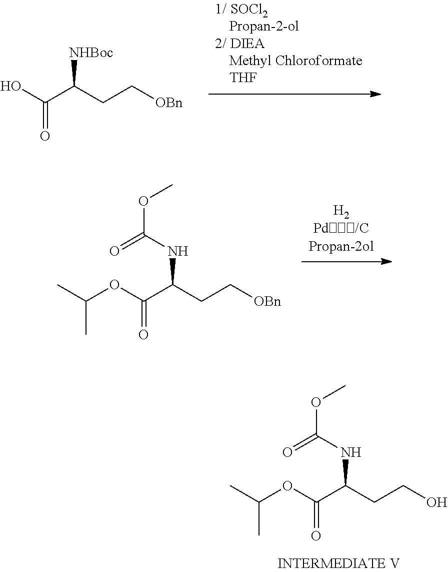 [SMILES: CC(C)(C)OC(=O)N[C@@H](CCOCc1ccccc1)C(=O)O ; COC(=O)N[C@@H](CCOCc1ccccc1)C(=O)OC(C)C ; COC(=O)N[C@@H](CCO)C(=O)OC(C)C]

### Step 1:

To a solution of O-benzyl-N-(tert-butoxycarbonyl)-L-homoserine (5 g, 16.2 mmol) in propan-2-ol (50 mL) was added dropwise thionyl chloride (11.7 mL, 161 mmol). The reaction was allowed to stir at 70° C. for 3 hours, then at room temperature for 16 hours. The reaction mixture was concentrated in vacuo, and the crude residue obtained was dissolved in THF. To the resulting solution was added DIEA (5.34 mL, 32.3 mmol) followed by methylchloroformate. The reaction was allowed to stir at room temperature for 3 hours, then a saturated aqueous NH₄Cl solution was added, and the resulting solution was extracted with dichloromethane. The combined organic layers were dried, filtered and concentrated in vacuo. The crude residue obtained was purified using flash chromatography on silica gel (dichloromethane: 100%) to provide the product. LC/MS: [(M+1)]⁺=310.6.

### Step 2:

To a solution of isopropyl O-benzyl-N-(methoxycarbonyl)-L-homoserinate (10.6 g, 34.3 mmol) in propan-2-ol (300 mL), divided in 3 batches, was added Pd/C (10% mol, 300 mg). The reaction was allowed to stir under hydrogen at room temperature for 16 hours, then filtered through a celite pad. The filtrate was concentrated in vacuo to provide the intermediate compound V. ¹H NMR (400 MHz, DMSO-d₆) δ 7.49-7.47 (m, 1H), 4.88 (heptuplet, J=6.20 Hz, 1H), 4.61-4.58 (m, 1H), 4.07-4.06 (m, 1H), 3.53-3.40 (m, 5H), 1.81-1.76 (m, 1H), 1.70-1.64 (m, 1H), 1.19-1.16 (m, 6H); LC/MS: [(M+1)]⁺=220.5.

### Example 17

### Preparation of Intermediate Compound W

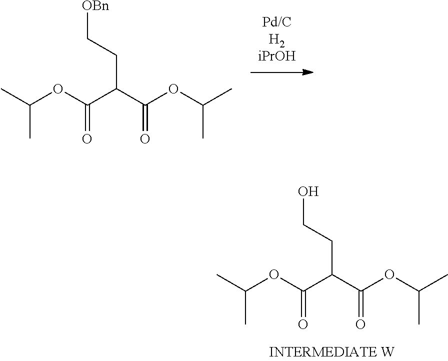 [SMILES: CC(C)OC(=O)C(CCOCc1ccccc1)C(=O)OC(C)C ; CC(C)OC(=O)C(CCO)C(=O)OC(C)C]

A solution of diisopropyl 2-(2-(benzyloxy)ethyl)malonate (see Example 18 for preparation, 10.43 g, 32 mmol) in propan-2-ol (100 mL) was allowed to stir under hydrogen at room temperature for about 15 hours. The reaction mixture was filtered through a celite pad, and the filtrate was concentrated in vacuo to provide intermediate compound W. ¹H NMR (400 MHz, CDCl₃) δ 5.06 (heptuplet, J=6.30 Hz, 2H), 3.72 (t, J=6.05 Hz, 2H), 3.49 (t, J=7.20 Hz, 1H), 2.17-2.12 (m, 2H), 1.255 (d, J=6.30 Hz, 6H), 1.25 (d, J=6.30 Hz, 6H); LC/MS: [(M+1)]⁺⁼233.0.

### Example 18

### Preparation of Intermediate Compound X

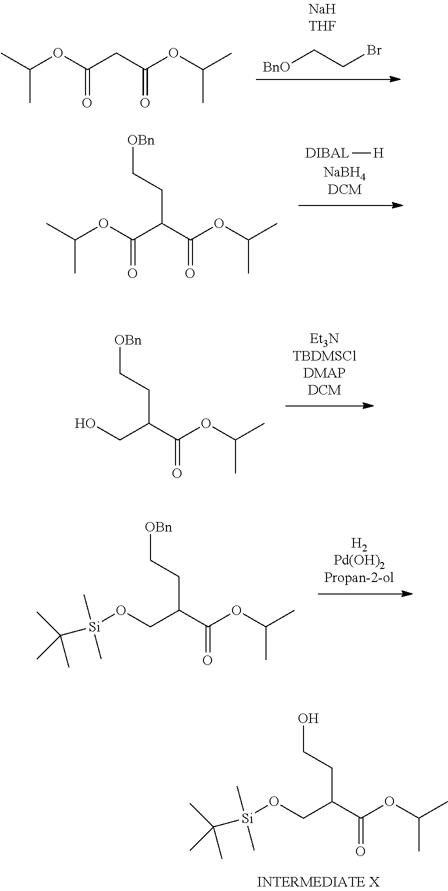

### Step 1:

To a suspension of sodium hydride 60% in oil (4.17 g, 104 mmol) in THF (190 mL) at 0° C. under nitrogen was added dropwise diisopropyl malonate (19.84 mL, 104 mmol). The reaction was allowed to stir at 0° C. for 30 minutes (until gas evolution ceased), then benzyl-2-bromoethylether (15 mL, 95 mmol) was added dropwise. The reaction was allowed to stir under reflux for 6 hours and cooled to RT. EtOAc was added to the reaction mixture and the organic layer was washed with water. The aqueous layer was extracted with EtOAc. The combined organic extracts were dried, filtered and concentrated in vacuo. The crude residue obtained was purified using flash chromatography on silica gel (PE/EtOAc: 0 to 10%) to provide the product. LC/MS: [(M+1)]⁺=323.2.

### Step 2:

A solution of the product obtained in Step 1 (10 g, 31.0 mmol) in dichloromethane (62.0 mL) was cooled to −78° C., then diisobutylaluminium hydride (46.5 mL, 46.5 mmol) was added dropwise under nitrogen. The reaction was allowed to stir at −78° C. for 1 hour, then was allowed to warm to −10° C. and diluted with MeOH (62 mL). Sodium borohydride (2.93 g, 78 mmol) was added, and the reaction was allowed to stir at −10° C. for 1 hour. Additional sodium borohydride (2.347 g, 62.0 mmol) was added, and the reaction mixture was allowed to stir at −10° C. for 30 minutes. The crude reaction mixture was quenched with a saturated aqueous ammonium chloride solution and diluted with dichloromethane. The resulting solution was filtered through a celite pad. The organic layer of the filtrate was extracted and washed with a 1N HCl solution, then dried, filtered and concentrated in vacuo. The crude residue obtained was purified using flash chromatography on silica gel (PE/EtOAc: 0 to 80%) to provide the product. LC/MS: [(M+1)]⁺=267.0.

### Step 3:

A solution of the product obtained in Step 2 (2 g, 7.51 mmol) in dichloromethane (14.30 mL) was cooled to 0° C. and triethylamine (1.15 mL, 8.26 mmol) and 4-dimethylaminopyridine (0.092 g, 0.75 mmol) were added, followed by tert-butyldimethylsilyl chloride (1.245 g, 8.26 mmol) in dichloromethane (7.15 mL) dropwise. The reaction was allowed to warm to RT and allowed to stir at RT for 2 hours under nitrogen. The reaction mixture was then diluted with dichloromethane and the organic layer was washed with a saturated aqueous NaHCO₃ solution. The organic layer was dried, filtered and concentrated in vacuo to provide a crude product which was used in the next step without further purification. LC/MS: [(M+1)]⁺=381.2.

### Step 4:

To a solution of the crude product obtained in Step 3 (2.79 g, 5.86 mmol) in propan-2-ol (29.3 mL) was added Pd(OH)₂ (700 mg, 0.498 mmol). The reaction was allowed to stir at room temperature under hydrogen for 48 hours. The reaction mixture was filtered through a celite pad, and washed with EtOAc. The filtrate was concentrated in vacuo, and the crude residue obtained was purified using 2 successives flash chromatographies on silica gel (dichloromethane/MeOH: 0 to 8%) and (PE/EtOAc: 0 to 50%) to provide intermediate compound X. ¹H NMR (400 MHz, CDCl₃) δ 5.03 (heptuplet, J=6.23 Hz, 1H), 3.86-3.82 (m, 1H), 3.76-3.68 (m, 3H), 2.69-2.63 (m, 1H), 1.96-1.87 (m, 1H), 1.83-1.72 (m, 3H), 1.28-1.20 (m, 7H), 0.88 (s, 9H), 0.06 (s, 6H); LC/MS: [(M+1)]⁺=291.2.

### Example 19

### Preparation of Intermediate Compound Y

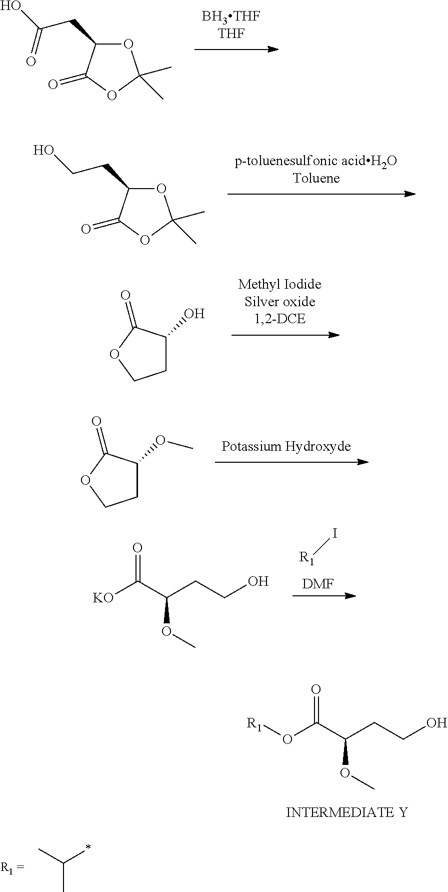

### Step 1:

To an ice-cooled solution of (R)-2-(2,2-dimethyl-5-oxo-1,3-dioxolan-4-yl)acetic acid (9.20 g, 52.8 mmol) in THF (26.4 mL) was added dropwise under nitrogen BH₃.THF (90 mL, 90 mmol). The reaction was allowed to stir at 0° C. for 1 hour, then at room temperature for about 15 hours. The reaction mixture was quenched with MeOH (50 mL) dropwise at 0° C., then the reaction mixture was allowed to stir at room temperature for 1 hour. The reaction mixture was concentrated in vacuo, then taken up in MeOH and concentrated again and taken up in EtOAc and concentrated in vacuo to provide the product.

### Step 2:

To a solution of the product obtained in Step 1 (8.46 g, 52.8 mmol) in toluene (52.8 mL) was added p-toluenesulfonic acid monohydrate (0.50 g, 2.64 mmol). The reaction was allowed to stir at room temperature for 2 days, then the reaction mixture was concentrated in vacuo. The crude residue obtained was purified using flash chromatography on silica gel (Petroleum ether/EtOAc: 60 to 100%) to provide the product.

### Step 3:

To a solution of (R)-3-hydroxydihydrofuran-2(3H)-one (3.63 g, 35.6 mmol) in DCE (71 mL) under nitrogen was added silver oxide (33.0 g, 142 mmol) and methyl iodide (22.3 mL, 356 mmol) at RT. The reaction was allowed to stir at 65° C. for 1 h. The reaction mixture was filtered off and the solution concentrated in vacuo to provide the product.

### Steps 4 and 5:

Steps 4 and 5 were carried out according to the method described in Example 1, method 1, Steps 1 and 2, starting from the product obtained in Step 3 immediately above to provide intermediate compound Y. ¹H NMR (400 MHz, DMSO-d₆) δ 4.95 (heptuplet, J=6.29 Hz, 1H), 4.52 (t, J=5.17 Hz, 1H), 3.85-3.79 (m, 1H), 3.48-3.39 (m, 2H), 3.25 (s, 3H), 1.87-1.63 (m, 2H), 1.22-1.19 (m, 6H); LC/MS: [(M−1)]⁻=175.3.

### Example 20

### Preparation of Intermediate Compound Z

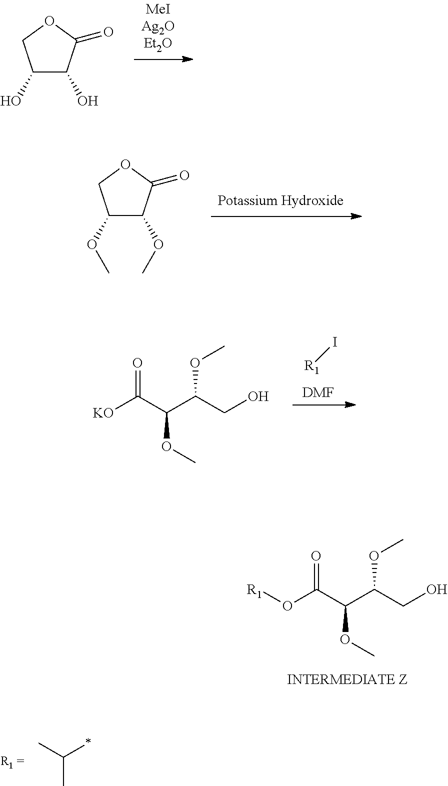

### Step 1:

To a solution of (3R,4R)-3,4-dihydroxydihydrofuran-2(3H)-one (5.7 g, 48.3 mmol) and iodomethane (9.01 mL, 145 mmol) in diethyl ether (193 mL) was added silver oxide (44.7 g, 193 mmol). The reaction was allowed to stir at room temperature for about 15 hours, then filtered and concentrated in vacuo. The crude residue obtained was purified using flash chromatography on silica gel (PE/EtOAc: 10% to 100%) to provide the product.

### Steps 2 and 3:

Steps 2 and 3 were carried out according to the method described in Example 1, method 1, Steps 1 and 2, starting from the product obtained in Step 1 immediately above to provide intermediate compound Z. ¹H NMR (400 MHz, CDCl₃) δ 5.15 (heptuplet, J=6.33 Hz, 1H), 3.93 (d, J=5.00 Hz, 1H), 3.79-3.71 (m, 2H), 3.56-3.52 (m, 1H), 3.45 (s, 3H), 3.43 (s, 3H), 1.30 (d, J=6.33 Hz, 3H), 1.29 (d, J=6.33 Hz, 3H); LC/MS: [(M+1)]⁺=207.0.

### Example 21

### Preparation of Intermediate Compound AA

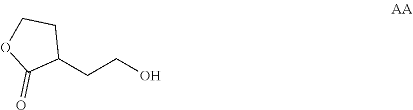

intermediate compound AA is commercially available.

### Example 22

### Preparation of Intermediate Compound BB

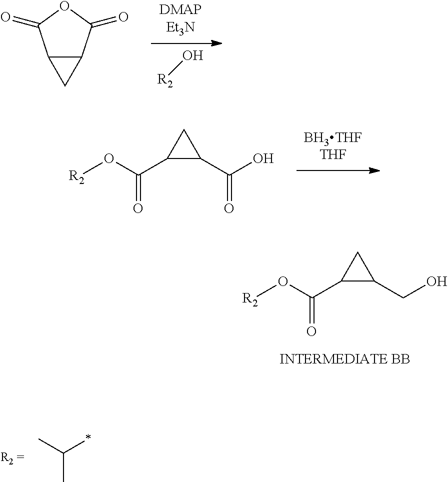

### Step 1:

To a solution of 3-oxabicyclo[3.1.0]hexane-2,4-dione (2.5 g, 22.30 mmol), DMAP (0.27 g, 2.23 mmol) in propan-2-ol (34 mL) was added triethylamine (3.10 mL, 22.30 mmol). The reaction was allowed to stir at 95° C. for about 15 hours, then concentrated in vacuo. The crude residue obtained was dissolved in EtOAc. The resulting solution was washed with a 1M citric acid solution (2×) and then with brine. The organic layer was dried, filtered and concentrated in vacuo. The crude compound obtained was dried in vacuo, and used in the next step without further purification. LC/MS: [(M+1)]=173.0.

### Step 2:

To a 0° C. solution the product obtained in Step 1 (3.6 g, 20.91 mmol) in THF (167 mL) was added dropwise borane-THF complex (31.4 mL, 31.4 mmol) under nitrogen. The reaction was allowed to warm to room temperature and allowed to stir for 1 hour at RT. The reaction mixture was slowly added to a saturated aqueous NaHCO₃ solution. The aqueous layer was extracted with diethyl ether (2×). The combined organic layers were dried and concentrated in vacuo, and the crude residue obtained was purified using flash chromatography on silica gel (dichloromethane/MeOH: 0 to 8%) to provide intermediate compound BB. ¹H NMR (400 MHz, CDCl₃) δ 5.03 (heptuplet, J=6.32 Hz, 1H), 3.95 (dd, J=11.83 Hz and 4.94 Hz, 1H), 3.76 (dd, J=11.83 Hz and 7.92 Hz, 1H), 1.74 (dt, J=8.27 Hz and 5.53 Hz, 1H), 1.63-1.54 (m, 1H), 1.26 (d, J=6.32 Hz, 3H), 1.255 (d, J=6.32 Hz, 3H), 1.18-1.08 (m, 2H); LC/MS: [(M+1)]⁺=159.0.

### Example 23

### Preparation of Intermediate Compound CC

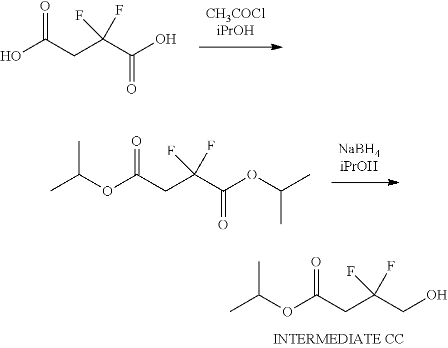

### Step 1:

To a 0° C. solution of propan-2-ol (70 mL) 0° C. under nitrogen was added under acetyl chloride (2.50 mL, 35.3 mmol). The reaction was allowed to stir at 0° C. for 30 minutes, then 2,2-difluorosuccinic acid (1.0 g, 6.49 mmol) was added. The reaction was allowed to stir at 0° C. for 30 minutes, at room temperature for 30 minutes, and at 45° C. for 4 days. The resulting mixture was concentrated in vacuo to provide the product, which was used without further purification.

### Step 2:

To a 0° C. solution of the product obtained in Step 1 (1.3 g, 5.46 mmol) in propan-2-ol (50 mL) was added NaBH₄ (0.247 g, 6.55 mmol). The reaction was allowed to stir at 0° C. for 1.5 hours, then at room temperature for about 15 hours. The reaction mixture was concentrated in vacuo, and the residue obtained was dissolved in a mixture of water (10 mL) and EtOAc (40 mL) and acidified to pH 5 using a 1N HCl solution. The organic layer was extracted, dried and concentrated in vacuo to provide intermediate compound CC. ¹H NMR (400 MHz, CDCl₃) δ 4.96-4.94 (m, 1H), 3.83-3.80 (m, 2H), 3.59-3.58 (m, 1H), 2.92-2.88 (m, 2H), 1.18-1.16 (m, 6H).

### Example 24

### Preparation of Intermediate Compound DD, EE, GG and HH

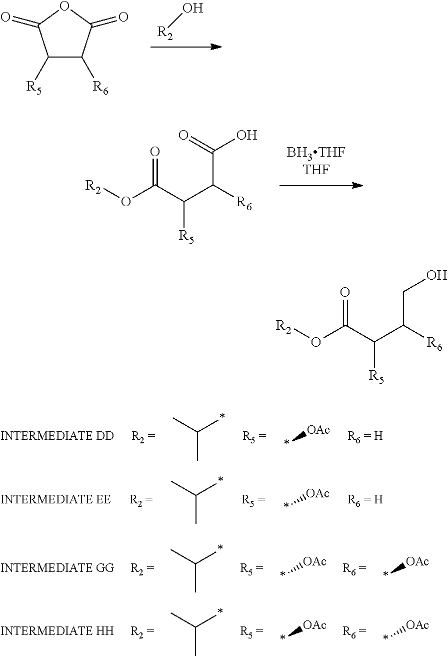

### Preparation of Intermediate Compound DD

### Step 1:

A solution of (R)-(+)-2-acetoxysuccinic anhydride (5 g, 31.6 mmol) in propan-2-ol (14.62 mL, 190 mmol) was allowed to stir at 50° C. for 2 hours and then at room temperature for about 15 hours. The reaction mixture was concentrated in vacuo to provide the product. LC/MS: [(M+23)]⁺=241.2.

### Step 2:

To a cold (−10° C.) solution of the product obtained in Step 1 (1 g, 4.35 mmol) in THF (10 mL) was added borane-THF complex (4.35 mL, 4.35 mmol). The reaction was allowed to stir at −10° C. for about 15 hours and then at 0° C. for 4 hours. Additional borane-THF complex (4.35 mL, 4.35 mmol) was then added at 0° C., and the resulting mixture was allowed to stir for about 15 hours. The reaction mixture was added dropwise to a stirred solution of saturated aqueous NaHCO₃ (2 mL), then diethyl ether (3 mL) was added. The organic layer was extracted. The aqueous layer was further extracted with diethyl ether (4 mL). The combined organic extracts were washed with brine (10 mL), dried and concentrated in vacuo at room temperature to provide intermediate compound DD. ¹H NMR (400 MHz, CDCl₃) δ 5.11 (dd, J=8.19 Hz and 4.67 Hz, 1H), 5.06 (heptuplet, J=6.28 Hz, 1H), 3.80-3.68 (m, 2H), 2.15 (s, 3H), 2.14-1.98 (m, 2H), 1.27 (d, J=6.28 Hz, 3H), 1.25 (d, J=6.28 Hz, 3H).

### Preparation of Intermediate Compound EE

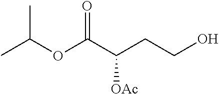

Intermediate compound EE was synthesized using the method described immediately above for the synthesis of intermediate compound DD and using (S)-(−)-2-acetoxysuccinic anhydride as the starting material in Step 1. Step 2 was carried out at 0° C. for 1 hour with 1.6 equiv. of borane-THF complex. ¹H NMR (400 MHz, CDCl₃) δ 5.10 (dd, J=8.10 Hz and 4.73 Hz, 1H), 5.04 (heptuplet, J=6.25 Hz, 1H), 3.79-3.66 (m, 2H), 2.12 (s, 3H), 2.09-2.01 (m, 2H), 1.25 (d, J=6.25 Hz, 3H), 1.22 (d, J=6.25 Hz, 3H).

### Preparation of Intermediate Compound GG

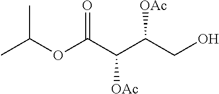 [SMILES: CC(=O)O[C@H](C(=O)OC(C)C)[C@@H](CO)OC(C)=O]

Intermediate compound GG was synthesized using the method described above for the synthesis of intermediate compound DD and using (−)-diacetyl-D-tartaric anhydride as the starting material in Step 1. Step 2 was carried out with 2 equiv. of borane-THF complex. The reaction was allowed to stir at 0° C. for 4 hours, at 4° C. for 2 days and at room temperature for 2 days. ¹H NMR (400 MHz, CDCl₃) δ 5.42-5.39 (m, 1H), 5.33 (d, J=3.27 Hz, 1H), 5.06 (heptuplet, J=6.28 Hz, 1H), 3.79 (dd, J=11.76 Hz and 6.10 Hz, 1H), 3.71 (dd, J=11.76 Hz and 6.10 Hz, 1H), 2.21 (s, 3H), 2.09 (s, 3H), 1.275 (d, J=6.28 Hz, 3H), 1.23 (d, J=6.28 Hz, 3H); LC/MS: [(M+23)]⁺=285.0.

### Preparation of Intermediate Compound HH

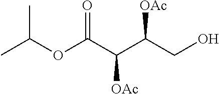 [SMILES: CC(=O)O[C@@H](CO)[C@@H](OC(C)=O)C(=O)OC(C)C]

Intermediate compound HH was synthesized using the method described above for the synthesis of intermediate compound DD and using (+)-diacetyl-L-tartaric anhydride as the starting material in Step 1. Step 2 was carried out with 2 equiv. of borane-THF complex. The reaction was allowed to stir at 35° C. for about 15 hours. The crude residue obtained was purified using flash chromatography on silica gel (dichloromethane/MeOH) to provide intermediate compound HH. ¹H NMR (400 MHz, CDCl₃) δ 5.42-5.39 (m, 1H), 5.33 (d, J=3.25 Hz, 1H), 5.19-5.02 (m, 1H), 3.79 (dd, J=11.67 Hz and 5.95 Hz, 1H), 3.71 (dd, J=11.67 Hz and 5.95 Hz, 1H), 2.21 (s, 3H), 2.09 (s, 3H), 1.275 (d, J=6.24 Hz, 6H); LC/MS: [(M+1)]⁺=263.0.

### Example 25

### Preparation of Intermediate Compound FF

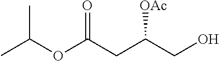

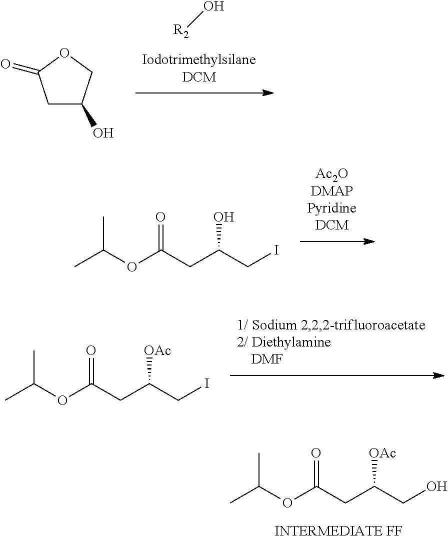

### Step 1:

To a solution of (S)-4-hydroxydihydrofuran-2(3H)-one (5.00 g, 49.0 mmol) and propan-2-ol (11.32 mL, 147 mmol) in dichloromethane (196 mL) under nitrogen was slowly added iodotrimethylsilane (10.00 mL, 73.5 mmol). The reaction was allowed to stir for about 15 hours, then the reaction mixture was concentrated in vacuo. The crude residue obtained was dissolved in diethyl ether, and the resulting solution was washed with sodium thiosulfate, filtered and concentrated in vacuo to provide the product. ¹H NMR (400 MHz, CDCl₃) δ 5.06 (heptuplet, J=6.19 Hz, 1H), 4.01-3.96 (m, 1H), 3.36-3.27 (m, 2H), 2.67-2.54 (m, 2H), 1.26 (d, J=6.19 Hz, 6H).

### Step 2:

To a solution of the product obtained in Step 1 (10.07 g, 37.0 mmol), pyridine (8.98 mL, 111 mmol) and DMAP (0.452 g, 3.70 mmol) in dichloromethane (74.0 mL) was added acetic anhydride (10.48 mL, 111 mmol). The reaction was allowed to stir at room temperature for 3 days. A 1N HCl solution (50 mL) was added to the reaction mixture and it was vigorously stirred for 10 minutes. The reaction mixture was transferred to a separatory funnel, and the organic layer was further washed with saturated aqueous NaHCO₃, dried, concentrated in vacuo, and co-evaporated with toluene to provide the product. LC/MS: [(M+23)]=337.0.

### Step 3:

To a mixture of the product obtained in Step 2 (10.68 g, 34.0 mmol) in DMF (34.0 mL) was added sodium 2,2,2-trifluoroacetate (6.94 g, 51.0 mmol). The reaction was allowed to stir at 90° C. for 2 hours, then the reaction mixture was allowed to cool to room temperature and diethylamine (10.55 mL, 102 mmol) was added. The reaction was allowed to stir at room temperature for 1 hour, then quenched with water and extracted with EtOAc. The organic layer was washed twice with water, dried and concentrated in vacuo, and the crude residue obtained was purified using MS-preparative HPLC (C18, water/MeCN) to provide intermediate compound FF. ¹H NMR (400 MHz, CDCl₃) δ 5.07 (heptuplet, J=6.29 Hz, 1H), 4.30-4.24 (m, 1H), 4.14 (dd, J=11.46 Hz and 4.03 Hz, 1H), 4.08 (dd, J=11.46 Hz and 6.21 Hz, 1H), 2.52-2.50 (m, 2H), 2.10 (s, 3H), 1.26 (d, J=6.29 Hz, 6H); LC/MS: [(M+23)]⁺=227.0.

### Example 26

### Preparation of Intermediate Compound II

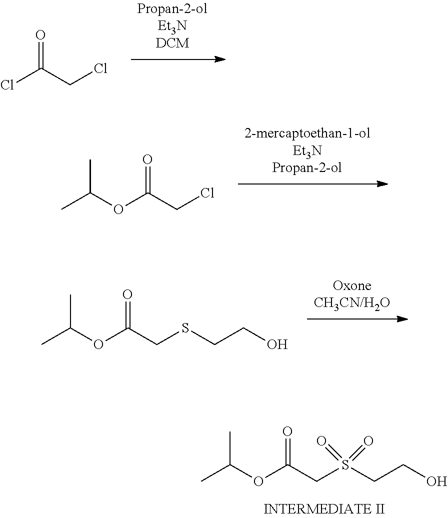

### Step 1:

To a 0° C. solution of triethylamine (12.3 mL, 88.57 mmol) in propan-2-ol (6.43 mL, 84.14 mmol) was slowly added a solution of 2-chloroacetyl chloride (10 g, 88.57 mmol) in dichloromethane (50 mL). The reaction was allowed to stir at 0° C. for 1 hour and at room temperature over 2 days. The reaction mixture was filtered and the resulting solution was washed with water. The organic layer was dried, filtered and concentrated in vacuo to provide the product.

### Step 2:

To a solution of the product obtained in Step 1 (6 g, 44.11 mmol) in propan-2-ol (120 mL) was added triethylamine (6.14 mL, 44.11 mmol). The reaction was cooled to 0° C. and 2-mercaptoethan-1-ol (3.1 mL, 44.11 mmol) was added dropwise. The reaction was allowed to warm to room temperature and allowed to stir at this temperature for about 15 hours. The reaction mixture was then concentrated in vacuo, and the crude residue obtained was dissolved in dichloromethane. The organic layer was washed with water, dried, filtered and concentrated in vacuo to provide a crude residue that was purified using flash chromatography on silica gel (hexane/EtOAc: 50%) to provide the product.

### Step 3:

To a solution of the product obtained in Step 2 (4.4 g, 24.71 mmol) in CH₃CN (220 mL) and water (176 mL) was added oxone (19.36 g, 32.12 mmol) portionwise over 20 minutes at 20° C. The reaction was allowed to stir at room temperature for about 15 hours. Further oxone (10 g) was added portionwise and the reaction mixture was allowed to stir for 2 hours. EtOAc was added to the reaction mixture, and the aqueous layer was extracted twice with EtOAc. The combined organic extracts were dried, filtered and concentrated in vacuo. The crude residue obtained was purified using flash chromatography on silica gel (hexane/EtOAc: 30 to 50%) to provide intermediate compound II. ¹H NMR (400 MHz, CDCl₃) δ 5.12 (heptuplet, J=6.28 Hz, 1H), 4.16 (t, J=4.97 Hz, 2H), 4.10 (s, 2H), 3.52 (t, J=4.97 Hz, 2H), 1.31 (d, J=6.28 Hz, 6H).

### Example 27

### Preparation of Intermediate Compound JJ

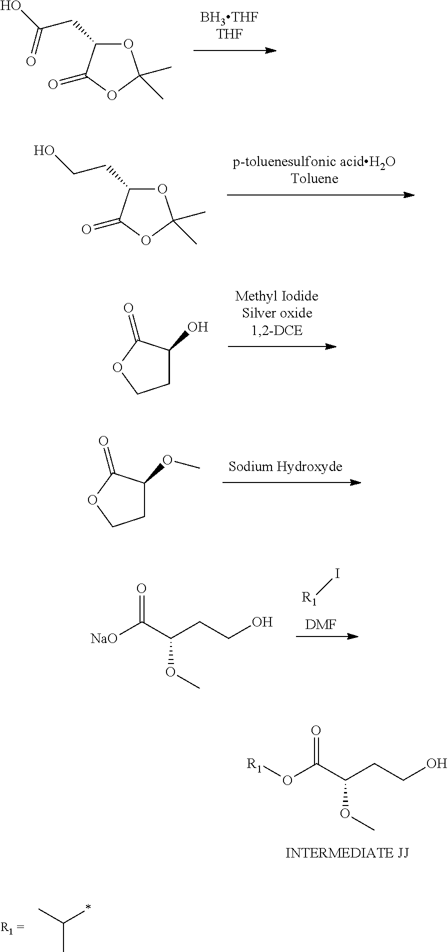

Intermediate compound JJ was synthesized using the methods described in Example 19, and using (S)-2-(2,2-dimethyl-5-oxo-1,3-dioxolan-4-yl)acetic acid as the starting material in Step 1. ¹H NMR (400 MHz, DMSO-d₆) δ 4.94 (heptuplet, J=6.17 Hz, 1H), 3.83 (dd, J=8.36 Hz and 4.37 Hz, 1H), 3.47-3.43 (m, 2H), 3.25 (s, 3H), 1.80-1.63 (m, 2H), 1.22-1.19 (m, 6H).

### Example 28

### Preparation of Intermediate Compound KK

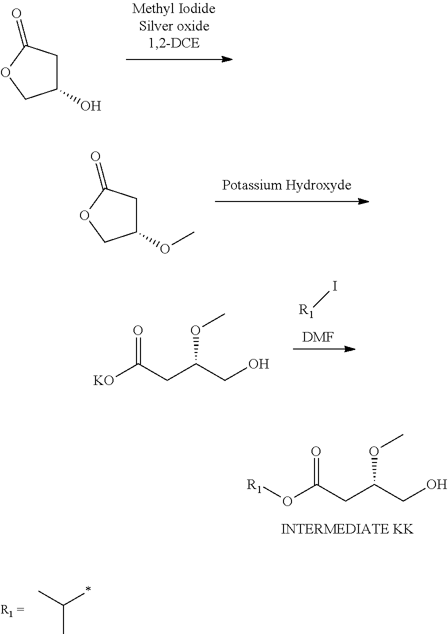

### Step 1:

To a solution of (S)-4-hydroxydihydrofuran-2(3H)-one (5.00 g, 49.0 mmol) in 1,2-dichloroethane (98 mL) under nitrogen and at RT was added silver(I) oxide (17.02 g, 73.5 mmol) and iodomethane (15.31 mL, 245 mmol). The reaction was allowed to stir at 65° C. for 2.5 days, then silver(I) oxide (8.00 g, 34.5 mmol) was added, and the reaction was allowed to stir at 65° C. for 1 additional day. The reaction mixture was filtered and the filtrate was concentrated in vacuo. The crude residue obtained was purified using flash chromatography on silica gel (PE/EtOAc) to provide the product. LC/MS: [(M+23)]⁺=139.0.

### Step 2:

A solution of the product obtained in Step 1 (4.79 g, 41.3 mmol) in 1N aqueous KOH (41.3 mL, 41.3 mmol) was allowed to stir at room temperature for about 15 hours. The reaction mixture was concentrated in vacuo, and the residue obtained was co-evaporated twice with MeCN to provide the product.

### Step 3:

To a mixture of the product obtained in Step 2 (6.64 g, 38.6 mmol) in DMF (38.6 mL) was added 2-iodopropane (4.63 mL, 46.3 mmol). The reaction was allowed to stir at room temperature for 2 days. EtOAc was added, and the resulting solution was washed twice with a sodium metabisulfite solution. The aqueous layer was extracted with EtOAc and the combined organic extracts were washed with brine, dried and concentrated in vacuo to provide intermediate compound KK. ¹H NMR (400 MHz, CDCl₃) δ 5.04 (heptuplet, J=6.28 Hz, 1H), 3.77-3.71 (m, 2H), 3.58-3.54 (m, 1H), 3.43 (s, 3H), 2.59 (dd, J=15.51 Hz and 6.42 Hz, 1H), 2.47 (dd, J=15.51 Hz and 6.15 Hz, 1H), 1.25 (d, J=6.28 Hz, 6H); LC/MS: [(M+1)]⁺=177.0.

### Example 29

### Preparation of Intermediate Compound LL

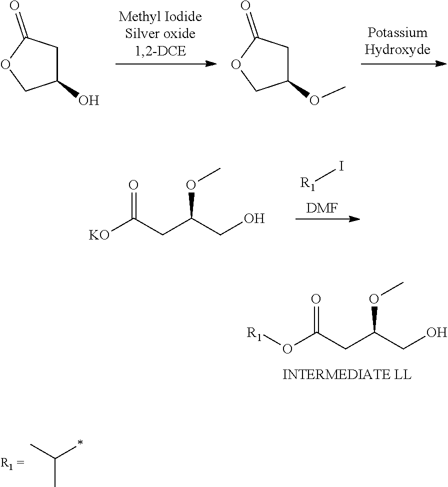

Intermediate compound LL was synthesized using the method described in Example 28, and using (R)-4-hydroxydihydrofuran-2(3H)-one as the starting material in Step 1. ¹H NMR (400 MHz, DMSO-d₆) δ 4.90 (heptuplet, J=6.23 Hz, 1H), 4.71 (t, J=5.59 Hz, 1H), 3.57-3.51 (m, 1H), −3.45-3.36 (m, 2H), 3.27 (s, 3H), 2.49-2.46 (m, 1H), 2.31 (dd, J=15.39 Hz and 8.26 Hz, 1H), 1.18 (d, J=6.23 Hz, 6H).

### Example 30

### Preparation of Intermediate Compound MM

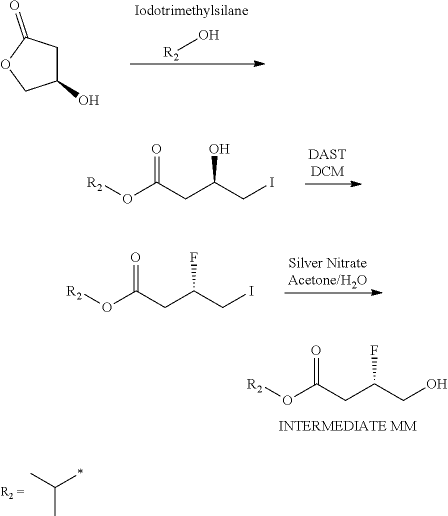

### Step 1:

To a solution of (R)-4-hydroxydihydrofuran-2(3H)-one (10.00 g, 98 mmol) and propan-2-ol (22.64 mL, 294 mmol) in dichloromethane (196 mL) under nitrogen was slowly added iodotrimethylsilane (20.00 mL, 147 mmol). The reaction was allowed to stir at room temperature for about 15 hours, then, a solution of sodium metabisulfite was added, and the reaction mixture was vigorously stirred. The reaction mixture was transferred to a separatory funnel and the collected organic layer was concentrated in vacuo to provide the product. LC/MS: [(M+1)]⁺=273.0.

### Step 2:

To a stirred solution of DAST (6.31 mL, 47.8 mmol) in dichloromethane (119 mL) at −78° C. under nitrogen was added a solution of the product obtained in Step 1 (13.00 g, 47.8 mmol) in dichloromethane (39.8 mL). The reaction was allowed to stir at −78° C. for 10 minutes, then was allowed to warm to room temperature for 1 hour and quenched with saturated aqueous NaHCO₃ solution. The layers were separated and the organic layer was concentrated in vacuo to provide a crude residue that was purified twice using flash chromatography on silica gel (PE/Et₂O) to provide the product. LC/MS: [(M+23)]⁺=296.8.

### Step 3:

To a solution of the product obtained in Step 1 (5.54 g, 20.21 mmol) in acetone (50.5 mL) and water (50.5 mL) was added silver nitrate (6.87 g, 40.4 mmol). The reaction was allowed to stir at room temperature for about 15 hours. The reaction mixture was filtered and the filtrate was partitioned between EtOAc and water, then the aqueous layer was extracted with EtOAc. The combined organic layers were dried, filtered and concentrated in vacuo to provide intermediate compound MM. ¹H NMR (400 MHz, CDCl₃) δ 5.10-4.93 (m, 2H), 3.91-3.70 (m, 2H), 2.85-2.59 (m, 2H), 1.28-1.25 (m, 6H); ¹⁹F NMR (376 MHz, CDCl₃) δ −189.52 (s, 1F); LC/MS: [(M+1)]⁺=165.0.

### Example 31

### Preparation of Intermediate Compound NN

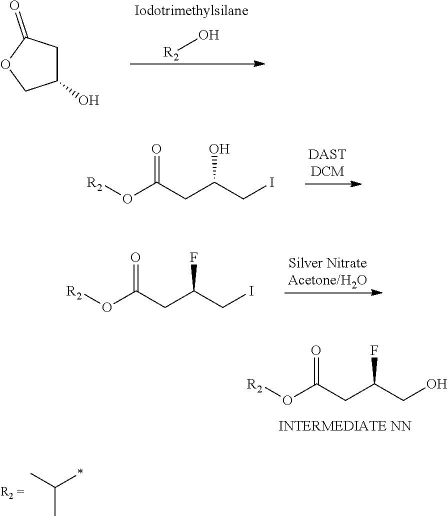

Intermediate compound NN was made using the method described in Example 30 and using (S)-4-hydroxydihydrofuran-2(3H)-one as the starting material in Step 1. ¹H NMR (400 MHz, CDCl₃) δ 5.11-4.93 (m, 2H), 3.91-3.70 (m, 2H), 2.85-2.59 (m, 2H), 1.27-1.25 (m, 6H); ¹⁹F NMR (376 MHz, CDCl₃) δ −189.49 (s, 1F); LC/MS: [(M+1)]⁺=165.0.

### Example 34

### Preparation of Intermediate Compound QQ

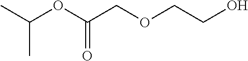

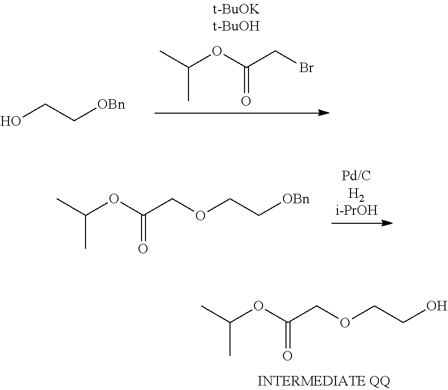

### Step 1:

To a solution of 2-(benzyloxy)ethan-1-ol (5.6 mL, 39.38 mmol) in t-butanol (90 mL) at room temperature was added potassium tert-butoxide (4.96 g, 44.2 mmol). The reaction was allowed to stir for 2.5 hours at RT, then isopropyl 2-bromoacetate (5.1 mL, 39.41 mmol) was added. The reaction was allowed to stir at room temperature for about 15 hours, then concentrated in vacuo. Water was added, and the resulting solution was extracted with dichloromethane. The organic layer was dried, filtered and concentrated in vacuo. The crude residue obtained was purified using flash chromatography on silica gel (n-hexane/EtOAc: 90/10) to provide the product.

### Step 2:

To a solution of the product obtained in Step 1 (2.9 g, 11.51 mmol) in propan-2-ol (30 mL) was added Pd/C (0.25 g). The reaction was allowed to stir at room temperature under hydrogen for 1.5 hours. The reaction mixture was filtered through a celite pad, and the filtrate was concentrated in vacuo to provide intermediate compound QQ.

### Example 35

### Preparation of Intermediate Compounds RR and WW

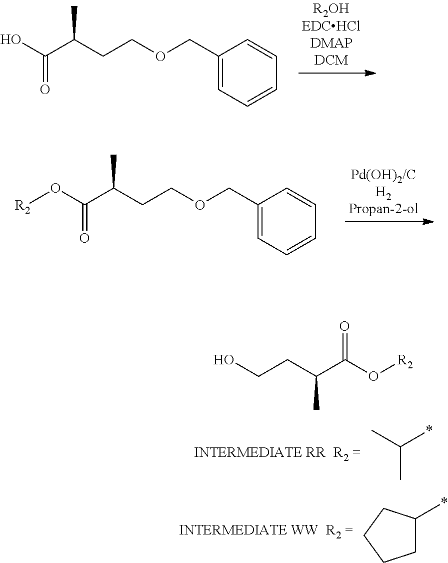

### Preparation of Intermediate Compound RR

### Step 1:

To a solution of (S)-4-(benzyloxy)-2-methylbutanoic acid (12 g, 57.6 mmol) in dichloromethane (222 mL) was added propan-2-ol (22.06 mL, 288 mmol), EDC (13.26 g, 69.1 mmol) and DMAP (0.704 g, 5.76 mmol). The reaction was allowed to stir at room temperature for 20 hours. The reaction mixture was then washed with water, 10% citric acid solution and brine. The organic layer was dried, filtered and concentrated in vacuo. The crude residue obtained was used directly in the next step without further purification.

### Step 2:

To a solution of the product obtained in Step 2 (12.26 g, 49.0 mmol) in propan-2-ol (240 mL) was added palladium hydroxide on carbon (5.16 g, 7.35 mmol). The reaction mixture was flushed 3 times using alternation vacuum and nitrogen and then stirred under hydrogen for 22 hours. The reaction mixture was then filtered through a celite pad, and the filtrate was concentrated in vacuo (T<40° C.) and co-evaporated with toluene to provide intermediate compound RR. ¹H NMR (400 MHz, CDCl₃) δ 5.01 (heptuplet, J=6.28 Hz, 1H), 3.73-3.64 (m, 2H), 2.63-2.54 (m, 1H), 1.96-1.88 (m, 1H), 1.73-1.65 (m, 1H), 1.24 (d, J=6.28 Hz, 3H), 1.23 (d, J=6.28 Hz, 3H), 1.18 (d, J=7.09 Hz, 3H).

### Preparation of Intermediate Compound WW

Intermediate compound WW was made using the method described immediately above for making intermediate compound RR and substituting cyclopentanol for propan-2-ol in Step 1. ¹H NMR (400 MHz, CDCl₃) δ 5.19-5.15 (m, 1H), 3.73-3.64 (m, 2H), 2.64-2.52 (m, 1H), 1.95-1.58 (m, 10H), 1.17 (d, J=7.03 Hz, 3H).

### Example 36

### Preparation of Intermediate Compound SS

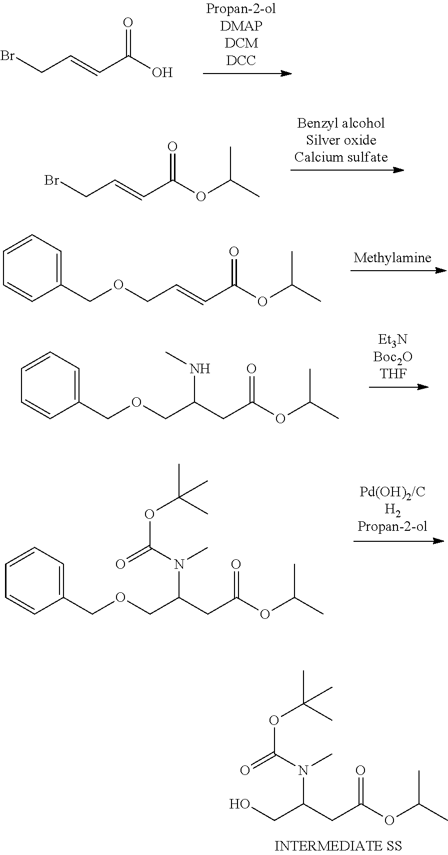

### Step 1:

Step 1 was carried out using the method described in Yamagata et al., Angew. Chem. Int. Ed., 2015, 54:4899-4903.

### Step 2:

To a solution of the product obtained in Step 1 (10.4 g, 50.2 mmol) in benzyl alcohol (47.0 mL, 452 mmol) under N₂ were added silver oxide (13.97 g, 60.3 mmol) and calcium sulfate (29.7 g, 218 mmol). The reaction was allowed to stir at 60° C. in the dark for 60 hours. DCM was then added and the resulting mixture was filtered through a celite pad, and the filtrate was concentrated in vacuo. The crude residue obtained was purified using flash chromatography on silica gel (PE/Et₂O: 4%) to provide the product.

### Step 3:

A solution of the product obtained in Step 2 (4.7 g, 20.06 mmol) in methylamine (201 ml, 401 mmol) was allowed to stir at room temperature for about 15 hours. The reaction mixture was then concentrated in vacuo to provide the product.

### Step 4:

A solution of the product obtained in Step 3 (5.32 g, 20.06 mmol) in anhydrous THF (100 mL) was cooled to 0° C., and triethylamine (3.36 mL, 24.07 mmol) and Boc-Anhydride (5.12 mL, 22.07 mmol) were added. The reaction was allowed to stir at 0° C. to room temperature for about 15 hours, then concentrated in vacuo. The crude residue obtained was dissolved with EtOAc (500 mL), and the resulting mixture was washed sequentially with water (500 mL), HCl 1N (500 mL), water (500 mL) and brine (500 mL). The organic layer was dried over Na₂SO₄, filtered and concentrated in vacuo, and the crude residue obtained was purified using flash chromatography on silica gel (PE/Et₂O: 0 to 20%) to provide the product. LC/MS: [(M+23)]⁺=388.5.

### Step 5:

To a solution of the product obtained in Step 4 (7.12 g, 19.48 mmol) in propan-2-ol (50 mL) was added Pd(OH)₂ on Carbon (4.79 g, 6.82 mmol). The reaction mixture was flushed 3 times with vacuum and nitrogen and then was allowed to stir under hydrogen for 6 hours. The reaction mixture was filtered through a celite pad, and the filtrate was concentrated in vacuo to provide intermediate compound SS. ¹H NMR (400 MHz, CDCl₃) δ 5.01 (heptuplet, J=6.27 Hz, 1H), 4.29-4.25 (m, 1H), 3.72-3.68 (m, 2H), 2.83 (s, 3H), 2.66-2.57 (m, 2H), 1.46 (s, 9H), 1.23 (d, J=6.27 Hz, 6H).

### Example 37

### Preparation of Intermediate Compound TT

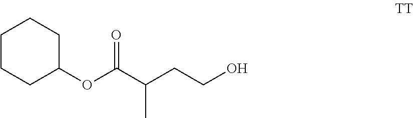

Intermediate compound TT was synthesized using the method described in Example 1, method 2, wherein Step 2 was carried out using 10 equivalents of cyclohexanol in place of 2-propanol and 2 equivalents of thionyl chloride. The reaction was allowed to stir at reflux for 24 hours, then additional thionyl chloride (0.1 equiv.) was added, and the reaction mixture was allowed to stir at reflux for about 15 hours and then concentrated in vacuo. The crude residue obtained in Step 2 was purified using flash chromatography on silica gel (PE/Et₂O: 0 to 15%). Step 3 was carried out using palladium on carbon, and the reaction mixture was hydrogenated at room temperature under 5 bars for about 15 hours to provide intermediate compound TT. ¹H NMR (400 MHz, CDCl₃) δ 4.81-4.75 (m, 1H), 3.73-3.66 (m, 2H), 2.65-2.56 (m, 1H), 1.96-1.81 (m, 4H), 1.74-1.66 (m, 4H), 1.47-1.36 (m, 4H), 1.19 (d, J=7.05 Hz, 3H).

### Example 38

### Preparation of Intermediate Compound UU

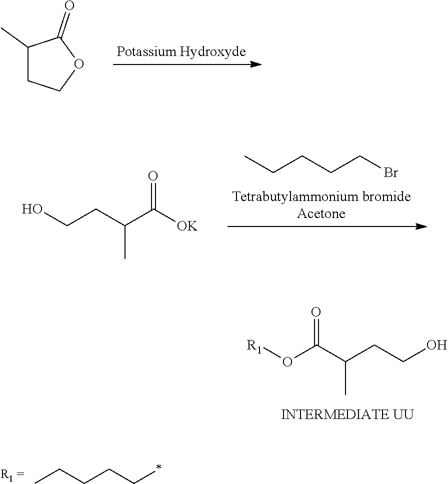

Intermediate compound UU was synthesized using the method described in Example 1, method 1. For Step 2, to a solution of potassium 4-hydroxy-2-methylbutanoate (4.75 g, 30.4 mmol) in acetone (30.4 mL) under nitrogen was added 1-bromopentane (4.07 mL, 32.8 mmol) and tetrabutylammonium bromide (0.49 g, 1.52 mmol). The reaction was allowed to stir at reflux for 24 hours. The reaction was filtered, and the filtrate was concentrated in vacuo at RT. The crude residue obtained was dissolved in EtOAc and washed with NaHCO₃, then brine. The organic layer was dried over Na₂SO₄ and concentrated in vacuo at room temperature to provide intermediate compound UU. ¹H NMR (400 MHz, CDCl₃) δ 4.08 (t, J=6.77 Hz, 2H), 3.71-3.67 (m, 2H), 2.68-2.59 (m, 1H), 1.98-1.89 (m, 1H), 1.74-1.61 (m, 3H), 1.35-1.32 (m, 4H), 1.20 (d, J=7.01 Hz, 3H), 0.93-0.89 (m, 3H).

### Example 39

### Preparation of Intermediate Compound VV

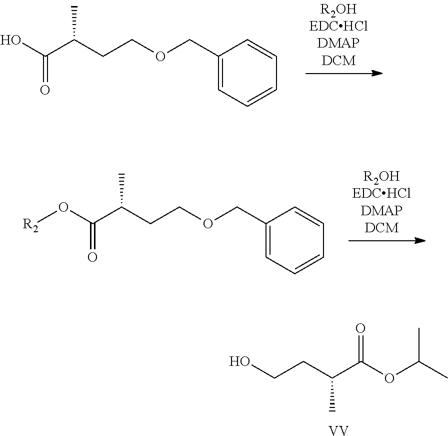

Intermediate compound VV was made using the method described in Example 35 for making intermediate compound RR and substituting (R)-4-(benzyloxy)-2-methylbutanoic acid for (S)-4-(benzyloxy)-2-methylbutanoic acid in step 1. ¹H NMR (400 MHz, CDCl₃) δ 5.02 (heptuplet, J=6.28 Hz, 1H), 3.73-3.64 (m, 2H), 2.63-2.54 (m, 1H), 1.96-1.87 (m, 1H), 1.73-1.65 (m, 1H), 1.24 (d, J=6.28 Hz, 3H), 1.235 (d, J=6.28 Hz, 3H), 1.18 (d, J=7.05 Hz, 3H): LC/MS: [(M+1)]⁺=161.0.

### Example 40

### Preparation of Intermediate Compound XX

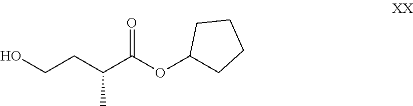

Intermediate compound XX was made using the method described in Example 39 and substituting cyclopentanol for propan-2-ol. ¹H NMR (400 MHz, CDCl₃) δ 5.19-5.15 (m, 1H), 3.73-3.63 (m, 2H), 2.64-2.54 (m, 1H), 1.95-1.57 (m, 10H), 1.18 (d, J=7.03 Hz, 3H).

### Example 41

### Preparation of Intermediate Compounds M and N

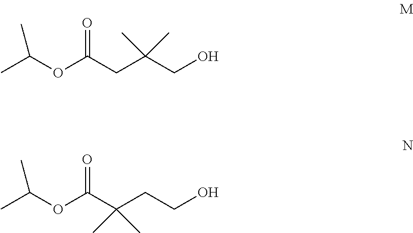

Intermediate compounds M and N are commercially available.

### Example 42

### Preparation of Compounds 1A and 1B

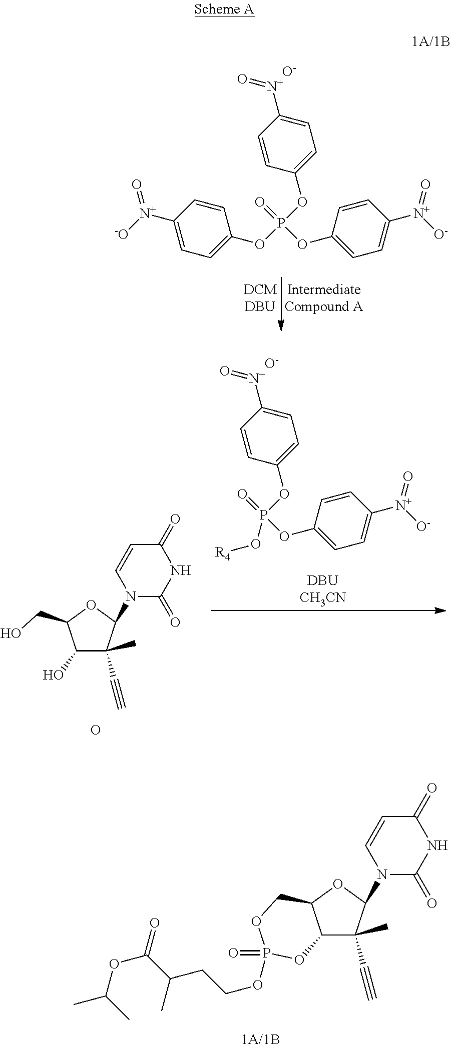

To a solution of intermediate compound A (1 eq., 3.01 g, 16.90 mmol) and tris(4-nitrophenyl) phosphate (1.2 eq., 9.36 g, 20.28 mmol) in dichloromethane (175 mL, 10 mL/mmol) at 0° C. under N₂ was added dropwise 1,8-diazabicyclo[5.4.0]undec-7-ene (DBU, 1.2 eq., 3.03 mL, 20.28 mmol). The reaction was allowed to stir from 0° C. to room temperature over 3 hours. The reaction mixture was then added dropwise to a solution of nucleoside 0 (1 eq., 4.50 g, 16.90 mmol) and DBU (2.4 eq., 6.07 mL, 40.60 mmol) in acetonitrile (300 mL) at RT. The resulting reaction mixture was allowed to stir at room temperature for about 15 hours, then concentrated in vacuo. The crude residue obtained was dissolved in dichloromethane or EtOAc, and the mixture was washed with a saturated aqueous NaHCO₃ solution, and brine. The organic layer was dried and concentrated in vacuo. The crude residue obtained was purified using flash chromatography on silica gel (dichloromethane/MeOH: 0 to 5%) to provide 2 mixtures of separated diastereomers (on the phosphorus atom). Each diastereomer was further purified using preparative HPLC (C18, H₂O/CH₃CN: 0 to 40%) to provide the 2 title compounds, each as a mixture of diastereomers (at the carbon alpha to the isopropyl ester):

Isomer 1A: mixture of diastereomers in an unquantified ratio at the carbon alpha to the isopropyl ester; S_{P}; ¹H NMR (400 MHz, DMSO-d₆) δ 11.58 (s, 1H), 7.76 (d, J=8.15 Hz, 1H), 6.28 (s, 1H), 5.68 (dd, J=8.15 Hz, 2.40 Hz, 1H), 4.89 (heptuplet, J=6.28 Hz, 1H), 4.72-4.57 (m, 2H), 4.24 (t, J=9.22 Hz, 1H), 4.17-4.08 (m, 3H), 3.64 (s, 1H), 2.61-2.52 (m, 1H), 2.06-1.97 (m, 1H), 1.83-1.74 (m, 1H), 1.19-1.17 (m, 9H), 1.11 (d, J=6.95 Hz, 3H); ³¹P NMR (162 MHz, DMSO-d₆) δ −6.72 (s, 2P); LC/MS: [(M+1)]⁺=471.2. The two isomers 1A were separated by Chiral Preparative HPLC with the following conditions: Column: Lux 4 21*250 mm, Mobile Phase A: CO₂: 85%, Mobile Phase B: EtOH 15%+0.2% DEA, to provide: Isomer 1A1 (faster eluting, Rt=18.79 min): ³¹P NMR (202 MHz, DMSO-d₆) δ −6.72 (s, 1P); LC/MS: [(M+1)]⁺=471.0; and Isomer 1A2 (slower eluting, Rt=21.05 min): ³¹P NMR (202 MHz, DMSO-d₆) δ −6.71 (s, 1P); LC/MS: [(M+1)]⁺=471.0.

Isomer 1B: mixture of diastereomers in a ratio 1:1 at the carbon alpha to the isopropyl ester; R_{P}; ¹H NMR (400 MHz, DMSO-d₆) δ 11.57 (s, 1H), 7.80 (d, J=8.14 Hz, 1H), 6.29 (s, 1H), 5.65 (d, J=8.14 Hz, 1H), 4.90 (heptuplet, J=6.20 Hz, 1H), 4.75-4.65 (m, 2H), 4.62 (d, J=9.60 Hz, 1H), 4.32-4.24 (m, 1H), 4.13-4.07 (m, 2H), 3.59 (d, J=2.40 Hz, 1H), 2.56-2.52 (m, 1H), 2.01-1.92 (m, 1H), 1.77-1.68 (m, 1H), 1.20-1.18 (m, 9H), 1.10 (d, J=7.03 Hz, 3H); ³¹P NMR (162 MHz, DMSO-d₆) δ −5.62 (s, 1P), −5.71 (s, 1P); LC/MS: [(M+23)]⁺=493.1. The two isomers 1B were separated by Chiral Preparative HPLC with the following conditions: Column: Chiral AS-H 30*250 mm, Mobile Phase A: Heptane+0.1% DEA, Mobile Phase B: MeOH/EtOH: 1/1, to provide: Isomer 1B1: ³¹P NMR (202 MHz, DMSO-d₆) δ −5.70 (s, 1P); LC/MS: [(M+1)]⁺=471.1; and Isomer 1B2: ³¹P NMR (202 MHz, DMSO-d₆) δ −5.61 (s, 1P); LC/MS: [(M+1)]⁺=471.1.

### Example 43

### Preparation of Compounds 2A and 2B

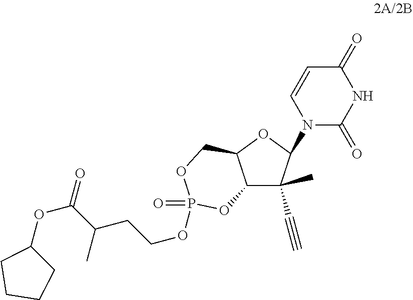

Compounds 2A/2B were made using the method described in Example 42 and substituting intermediate compound B for intermediate compound A. Each of the 2 mixtures of separated diastereomers (on the phosphorus atom) was further purified using preparative HPLC (C18, H₂O/CH₃CN: 0 to 35%) to provide the 2 title compounds (2A and 2B), each as a mixture of diastereomers (at the carbon alpha to the cyclopentyl ester):

Isomer 2A: mixture of diastereomers in an unquantified ratio at the carbon alpha to the cyclopentyl ester; S_{P}; ¹H NMR (400 MHz, DMSO-d₆) δ 11.59 (s, 1H), 7.76 (d, J=8.17 Hz, 1H), 6.28 (s, 1H), 5.68 (dd, J=8.17 Hz, 1.45 Hz, 1H), 5.09-5.05 (m, 1H), 4.72-4.57 (m, 2H), 4.24 (t, J=9.21 Hz, 1H), 4.17-4.08 (m, 3H), 3.64 (s, 1H), 2.59-2.52 (m, 1H), 2.06-1.96 (m, 1H), 1.84-1.75 (m, 3H), 1.66-1.53 (m, 6H), 1.19-1.18 (m, 3H), 1.11 (d, J=7.03 Hz, 3H); ³¹P NMR (162 MHz, DMSO-d₆) δ −6.72 (s, 2P); LC/MS: [(M+1)]⁺=497.2.

Isomer 2B: mixture of diastereomers in a ratio 1:1 at the carbon alpha to the cyclopentyl ester; R_{P}; ¹H NMR (400 MHz, DMSO-d₆) δ 11.57 (s, 1H), 7.80 (d, J=8.16 Hz, 1H), 6.29 (s, 1H), 5.65 (d, J=8.16 Hz, 1H), 5.09-5.06 (m, 1H), 4.75-4.65 (m, 2H), 4.62 (d, J=9.69 Hz, 1H), 4.32-4.24 (m, 1H), 4.13-4.07 (m, 2H), 3.59 (m, 1H), 2.55-2.52 (m, 1H), 2.00-1.92 (m, 1H), 1.85-1.77 (m, 2H), 1.75-1.63 (m, 7H), 1.17 (s, 3H), 1.10 (d, J=7.06 Hz, 3H); ³¹P NMR (162 MHz, DMSO-d₆) δ −5.61 (s, 1P), −5.69 (s, 1P); LC/MS: [(M+1)]⁺=497.2.

### Example 44

### Preparation of Compounds 3A and 3B

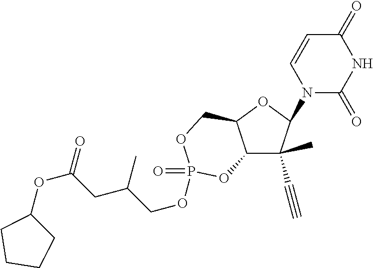

Compounds 3A/3B were made using the method described in Example 42 and substituting intermediate compound C for intermediate compound A. Each of the 2 mixtures of separated diastereomers (on the phosphorus atom) was further purified using preparative HPLC (C18, H₂O/CH₃CN: 0 to 40%) to provide the 2 title compounds, each as a mixture of diastereomers (at the carbon beta to the cyclopentyl ester):

Isomer 3A: mixture of diastereomers in a ratio 1:1 at the carbon beta to the cyclopentyl ester; S_{P}; ¹H NMR (400 MHz, DMSO-d₆) δ 11.58 (s, 1H), 7.75 (d, J=8.16 Hz, 1H), 6.28 (s, 1H), 5.67-5.65 (m, 1H), 5.10-5.05 (m, 1H), 4.72-4.64 (m, 2H), 4.21-4.12 (m, 2H), 3.98-3.94 (m, 2H), 3.64 (s, 1H), 2.48-2.42 (m, 1H), 2.28-2.18 (m, 2H), 1.84-1.77 (m, 2H), 1.66-1.52 (m, 6H), 1.18 (s, 3H), 0.96 (d, J=6.33 Hz, 3H); ³¹P NMR (162 MHz, DMSO-d₆) δ −6.71 (s, 1P), −6.77 (s, 1P); LC/MS: [(M+1)]⁺=497.2.

Isomer 3B: mixture of diastereomers in a ratio 1:1 at the carbon beta to the cyclopentyl ester; R_{P}; ¹H NMR (400 MHz, DMSO-d₆) δ 11.57 (s, 1H), 7.80 (d, J=8.13 Hz, 1H), 6.29 (s, 1H), 5.65 (d, J=8.13 Hz, 1H), 5.10-5.07 (m, 1H), 4.73-4.66 (m, 2H), 4.63 (d, J=9.69 Hz, 1H), 4.34-4.27 (m, 1H), 3.98-3.94 (m, 2H), 3.62-3.61 (m, 1H), 2.41-2.36 (m, 1H), 2.25-2.13 (m, 2H), 1.84-1.78 (m, 2H), 1.66-1.53 (m, 6H), 1.17 (s, 3H), 0.93 (d, J=6.27 Hz, 3H); ³¹P NMR (162 MHz, DMSO-d₆) δ −5.67 (s, 1P), −5.69 (s, 1P); LC/MS: [(M+1)]⁺=497.2.

### Example 45

### Preparation of Compounds 4A and 4B

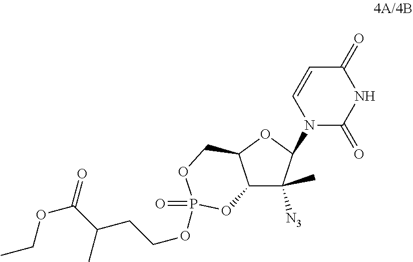

Compounds 4A and 4B were made using the method described in Example 42 and substituting intermediate compound D for intermediate compound A, and nucleoside P for nucleoside O:

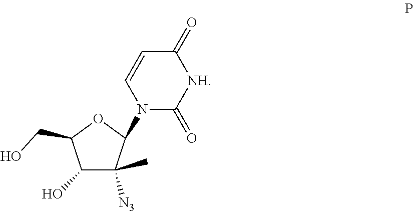 [SMILES: C[C@@]1(N=[N+]=[N-])[C@H](O)[C@@H](CO)O[C@H]1n1ccc(=O)[nH]c1=O]

The crude residue obtained was purified using flash chromatography on silica gel (dichloromethane/MeOH: 0 to 10%) to provide a mixture of diastereomers: LC/MS: [(M+1)]⁺=474.2.

### Example 46

### Preparation of Compounds 5A and 5B

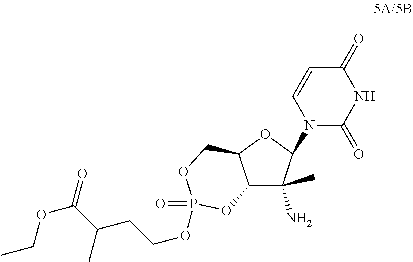

To a solution of compounds 4A/4B (310 mg, 0.65 mmol) in methanol (12 mL) under nitrogen atmosphere was added Pd(OH)₂ on carbon (92 mg, 0.65 mmol). After several hydrogen-vacuum purges, the reaction mixture was allowed to stir under hydrogen atmosphere at room temperature for 30 minutes. The reaction mixture was then filtered through a celite pad, and the filtrate was concentrated in vacuo. The crude residue obtained was purified using flash chromatography on silica gel (dichloromethane/MeOH: 0 to 10%) to provide a mixture of diastereomers:

Isomer 5A: mixture of diastereomers at the carbon alpha to the ethyl ester; S_{P}; ¹H NMR (400 MHz, DMSO-d₆) δ 11.49 (s, 1H), 7.73-7.71 (m, 1H), 5.89 (s, 1H), 5.66-5.64 (m, 1H), 4.68-4.50 (m, 2H), 4.30-4.23 (m, 1H), 4.11-4.04 (m, 5H), 2.64-2.58 (m, 1H), 2.05-1.98 (m, 3H), 1.83-1.76 (m, 1H), 1.18 (t, J=7.08 Hz, 3H), 1.13 (d, J=6.95 Hz, 3H), 1.02 (s, 3H); ³¹P NMR (162 MHz, DMSO-d₆) δ −5.98 (s, 1P); LC/MS: [(M+1)]⁺=448.2.

Isomer 5B: mixture of diastereomers at the carbon alpha to the ethyl ester; R_{P}; ¹H NMR (400 MHz, DMSO-d₆) δ 7.79-7.77 (m, 1H), 5.90 (s, 1H), 5.62 (d, J=8.13 Hz, 1H), 4.68-4.61 (m, 2H), 4.46-4.37 (m, 1H), 4.11-4.05 (m, 5H), 2.58-2.53 (m, 1H), 2.01-1.94 (m, 3H), 1.76-1.68 (m, 1H), 1.21-1.11 (m, 6H), 1.02 (s, 3H); ³¹P NMR (162 MHz, DMSO-d₆) δ −4.76 (s, 1P); LC/MS: [(M+1)]⁺=448.2.

### Example 47

### Preparation of Compounds 6A and 6B

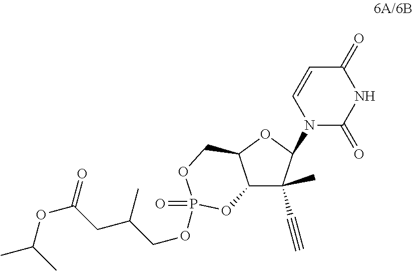

Compounds 6A/6B were made using the method described in Example 42 and substituting intermediate compound S for intermediate compound A. Each of the 2 mixtures of separated diastereomers (on the phosphorus atom) was further purified using preparative HPLC (C18, H₂O/CH₃CN: 0 to 40%) to provide the 2 title compounds, each as a mixture of diastereomers (at the carbon beta to the isopropyl ester):

Isomer 6A: mixture of diastereomers in a ratio 1:1 at the carbon beta to the isopropyl ester; S_{P}; ¹H NMR (400 MHz, DMSO-d₆) δ 11.59 (s, 1H), 7.75 (d, J=8.09 Hz, 1H), 6.28 (s, 1H), 5.68-5.66 (m, 1H), 4.93-4.86 (m, 1H), 4.73-4.65 (m, 2H), 4.22-4.12 (m, 2H), 4.04-3.95 (m, 2H), 3.64 (s, 1H), 2.46-2.43 (m, 1H), 2.29-2.18 (m, 2H), 1.19-1.17 (m, 9H), 0.96 (d, J=6.01 Hz, 3H); ³¹P NMR (162 MHz, DMSO-d₆) δ −6.71 (s, 1P), −6.77 (s, 1P); LC/MS: [(M+1)]⁺=471.0. The two isomers 6A were separated by Chiral Preparative HPLC with the following conditions: Column: OD-H 20*250 mm, Mobile Phase A: CO₂: 88%, Mobile Phase B: EtOH 12%, to provide: Isomer 6A1 (faster eluting, Rt=7.95 min): ³¹P NMR (202 MHz, DMSO-d₆) δ −6.70 (s, 1P); LC/MS: [(M+23)]⁺=493.2; and Isomer 6A2 (slower eluting, Rt=8.83 min): ³¹P NMR (202 MHz, DMSO-d₆) δ −6.76 (s, 1P); LC/MS: [(M+1)]⁺=471.2.

Isomer 6B: mixture of diastereomers at the carbon beta to the isopropyl ester; R_{P}; ¹H NMR (400 MHz, DMSO-d₆) 11.57 (s, 1H), 7.80 (d, J=8.17 Hz, 1H), 6.29 (s, 1H), 5.65 (d, J=8.17 Hz, 1H), 4.91 (heptuplet, J=6.31 Hz, 1H), 4.72-4.67 (m, 2H), 4.63 (d, J=9.75 Hz, 1H), 4.34-4.28 (m, 1H), 3.98-3.95 (m, 2H), 3.62-3.61 (m, 1H), 2.41-2.36 (m, 1H), 2.26-2.08 (m, 2H), 1.20-1.17 (m, 9H), 0.94 (d, J=6.31 Hz, 3H); ³¹P NMR (162 MHz, DMSO-d₆) δ −5.67 (s, 1P), −5.69 (s, 1P); LC/MS: [(M+1)]⁺=471.2. The two isomers 6B were separated by Chiral Preparative HPLC with the following conditions: Column: OJ-H: 20*250 mm, Mobile Phase A: CO₂: 85%, Mobile Phase B: EtOH 15%, to provide: Isomer 6B1 (faster eluting, Rt=2.19 min): ³¹P NMR (202 MHz, DMSO-d₆) δ −5.69 (s, 1P); LC/MS: [(M+23]⁺=493.2; and Isomer 6B2 (slower eluting, Rt=2.61 min): ³¹P NMR (202 MHz, DMSO-d₆) δ −5.67 (s, 1P); LC/MS: [(M+23)]⁺=493.2.

### Example 48

### Preparation of Compounds 7A1, 7A2 and 7B

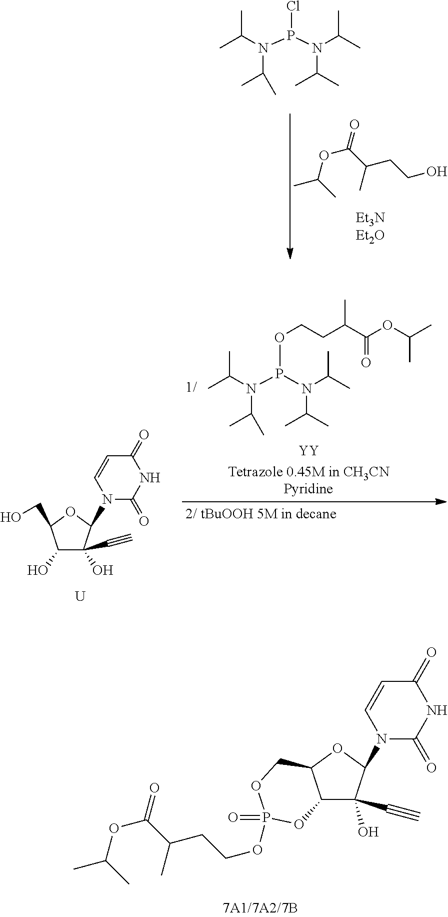

### Preparation of Intermediate compound YY

### Step 1:

To a −15° C. solution of 1-chloro-N,N,N′,N′-tetraisopropylphosphinediamine (1 eq., 1.409 g, 5.28 mmol) in diethyl ether (28.2 mL, 5.3 mL/mmol), under nitrogen, was added triethylamine (3 eq., 2.21 mL, 15.84 mmol). A solution of isopropyl 4-hydroxy-2-methylbutanoate (1 eq, 0.846 g, 5.28 mmol) in diethyl ether (14.08 mL, 2.7 mL/mmol) was added, and the reaction was allowed to stir at −15° C. for 1 hour and then at room temperature for 2 hours. The suspension was filtered under nitrogen and washed with diethyl ether. The filtrate was concentrated in vacuo at room temperature under nitrogen to provide a crude intermediate compound of formula YY, which was stored at −20° C. under nitrogen and was directly used in the next step without further purification: ³¹P NMR (162 MHz, CDCl₃) δ 124.00 (s, 1P).

### Step 2:

To a solution of nucleoside compound U (1 eq., 1 g, 3.73 mmol) in pyridine (24.85 mL, 6.7 mL/mmol) was added 1H-tetrazole 0.45 M in CH₃CN (3.1 eq., 26 mL, 11.70 mmol). The reaction mixture was cooled to −5° C. and a solution of intermediate compound YY (1.1 eq., 2.2 g, 3.66 mmol) in acetonitrile (12.43 mL, 3.3 mL/mmol) was added dropwise. The reaction was allowed to stir at −5° C. for 1.5 hours, then at room temperature for 2 hours. The reaction was monitored by LC/MS. A solution of tert-butylhydroperoxide, 5M in decane (2.7 eq., 2 mL, 10.00 mmol) was then added dropwise and the resulting reaction mixture was allowed to stir for 20 minutes at RT. The crude mixture was concentrated in vacuo, and co-evaporated with toluene (2×). The crude compound was purified using flash chromatography on silica gel (dichloromethane/MeOH: 0 to 10%) to provide a mixture of diastereomers as a solid. This mixture of diastereomers was further purified using preparative HPLC (C18, H₂O/CH₃CN: 0 to 50%) to provide 3 title compounds (isolated diastereomers at P).

Isomer 7A1: pure diastereomer at the carbon alpha to the isopropyl ester; S_{P}; ¹H NMR (400 MHz, DMSO-d₆) 11.50 (s, 1H), 7.78 (d, J=8.09 Hz, 1H), 7.20 (s, 1H), 6.06 (s, 1H), 5.67 (d, J=8.09 Hz, 1H), 4.91 (heptuplet, J=6.29 Hz, 1H), 4.70-4.61 (m, 1H), 4.58-4.53 (m, 1H), 4.40-4.37 (m, 1H), 4.16-4.08 (m, 3H), 3.81 (s, 1H), 2.60-2.55 (m, 1H), 2.08-1.99 (m, 1H), 1.87-1.79 (m, 1H), 1.19 (d, J=6.29 Hz, 6H), 1.12 (d, J=7.06 Hz, 3H); ³¹P NMR (162 MHz, DMSO-d₆) δ −6.43 (s, 1P); LC/MS: [(M+1)]⁺=473.0.

Isomer 7A2: pure diastereomer at the carbon alpha to the isopropyl ester; S_{P}; ¹H NMR (400 MHz, DMSO-d₆) 11.52 (s, 1H), 7.77 (d, J=8.12 Hz, 1H), 7.20 (s, 1H), 6.06 (s, 1H), 5.67 (d, J=8.12 Hz, 1H), 4.91 (heptuplet, J=6.30 Hz, 1H), 4.70-4.57 (m, 2H), 4.37-4.34 (m, 1H), 4.16-4.09 (m, 3H), 3.82 (s, 1H), 2.63-2.57 (m, 1H), 2.07-1.99 (m, 1H), 1.86-1.79 (m, 1H), 1.19 (d, J=6.30 Hz, 3H), 1.185 (d, J=6.30 Hz, 3H), 1.12 (d, J=7.13 Hz, 3H); ³¹P NMR (162 MHz, DMSO-d₆) δ −6.39 (s, 1P); LC/MS: [(M+1)]⁺=473.0.

The two isomers 7A1 and 7A2 were combined to provide the title compound 7A.

Isomer 7B: mixture of diastereomers in a ratio 1:1 at the carbon alpha to the isopropyl ester; R_{P}; ¹H NMR (400 MHz, DMSO-d₆) δ 11.49 (s, 1H), 7.84 (d, J=8.12 Hz, 1H), 7.14 (s, 1H), 6.06 (s, 1H), 5.64 (d, J=8.12 Hz, 1H), 4.89 (heptuplet, J=6.28 Hz, 1H), 4.76-4.74 (m, 1H), 4.68-4.60 (m, 2H), 4.29-4.22 (m, 1H), 4.13-4.05 (m, 2H), 3.83 (s, 1H), 2.53-2.52 (m, 1H), 2.00-1.91 (m, 1H), 1.76-1.67 (m, 1H), 1.19 (d, J=6.28 Hz, 3H), 1.185 (d, J=6.28 Hz, 3H), 1.10 (d, J=7.04 Hz, 3H); ³¹P NMR (162 MHz, DMSO-d₆) δ −5.23, −5.37 (s, 2P); LC/MS: [(M+1)]⁺=473.0.

### Example 49

### Preparation of Compounds 8A and 8B

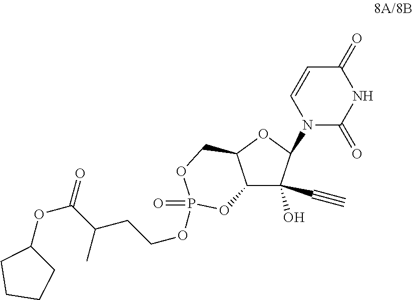

Compounds 8A/8B were made using the method described in Example 48 and substituting intermediate compound B for isopropyl 4-hydroxy-2-methylbutanoate in Step 1. The crude compound was purified using flash chromatography on silica gel (dichloromethane/MeOH: 0 to 10%) to provide a mixture of diastereomers as a solid. This mixture of diastereomers was further purified using preparative HPLC (C18, H₂O/CH₃CN: 0 to 50%) to provide the 2 title compounds, each as a mixture of diastereomers (at the carbon alpha to the cyclopentyl ester):

Isomer 8A: mixture of diastereomers in a ratio 1:1 at the carbon alpha to the cyclopentyl ester; S_{P}; ¹H NMR (400 MHz, DMSO-d₆) δ 11.52 (s, 1H), 7.79-7.76 (m, 1H), 7.20 (s, 1H), 6.06 (s, 1H), 5.67 (d, J=8.00 Hz, 1H), 5.11-5.07 (m, 1H), 4.70-4.53 (m, 2H), 4.40-4.34 (m, 1H), 4.16-4.08 (m, 3H), 3.81 (s, 1H), 2.62-2.54 (m, 1H), 2.07-1.99 (m, 1H), 1.85-1.78 (m, 3H), 1.67-1.53 (m, 6H), 1.11 (d, J=7.03 Hz, 3H); ³¹P NMR (162 MHz, DMSO-d₆) δ −6.39 (s, 1P), −6.41 (s, 1P); LC/MS: [(M+1)]⁺=499.2. The two isomers 8A were separated by Chiral Preparative HPLC with the following conditions: Column: AS-H: 20*250 cm, Mobile Phase A: CO₂: 65%, Mobile Phase B: isopropanol 35%, to provide: Isomer 8A1 (faster eluting, Rt=3.24 min): ³¹P NMR (202 MHz, DMSO-d₆) δ −6.38 (s, 1P); LC/MS: [(M+23]⁺=521.2; and Isomer 8A2 (slower eluting, Rt=4.22 min): ³¹P NMR (202 MHz, DMSO-d₆) δ −6.41 (s, 1P); LC/MS: [(M+23)]⁺=521.0.

Isomer 8B: mixture of diastereomers in a ratio 1:1 at the carbon alpha to the cyclopentyl ester; R_{P}; ¹H NMR (400 MHz, DMSO-d₆) δ 11.47 (s, 1H), 7.84 (d, J=8.09 Hz, 1H), 7.14 (s, 1H), 6.06 (s, 1H), 5.65 (d, J=8.09 Hz, 1H), 5.09-5.05 (m, 1H), 4.76-4.74 (m, 1H), 4.68-4.60 (m, 2H), 4.29-4.22 (m, 1H), 4.13-4.05 (m, 2H), 3.83 (s, 1H), 1.99-1.90 (m, 1H), 1.85-1.77 (m, 2H), 1.75-1.53 (m, 7H), 1.10 (d, J=6.94 Hz, 3H); ³¹P NMR (162 MHz, DMSO-d₆) δ −5.21 (s, 1P), −5.34 (s, 1P); LC/MS: [(M+1)]⁺=499.0.

### Example 50

### Preparation of Compounds 9A and 9B

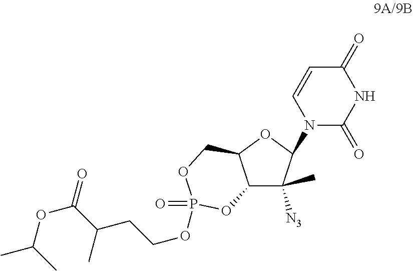

Compounds 9A/9B were made using the method described in Example 42 and substituting nucleoside Q for nucleoside O:

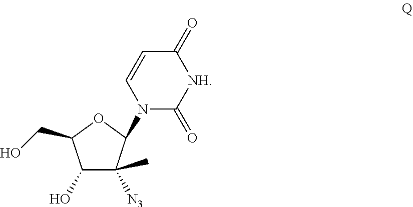 [SMILES: C[C@@]1(N=[N+]=[N-])[C@H](O)[C@@H](CO)O[C@H]1n1ccc(=O)[nH]c1=O]

The crude residue obtained was purified using flash chromatography on silica gel (dichloromethane/MeOH: 0 to 5%) to provide 2 mixtures of separated diastereomers (on the phosphorus atom).

Isomer 9A: mixture of diastereomers; LC/MS: [(M+1)]⁺=488.2.

Isomer 9B: mixture of diastereomers; LC/MS: [(M+1)]⁺=488.0.

### Example 51

### Preparation of Compounds 10A and 10B

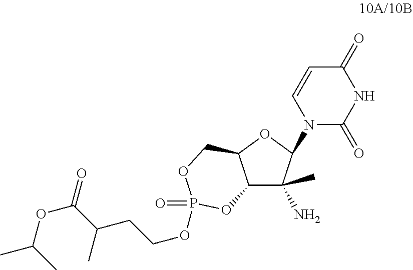

Compounds 10A/10B were made using the method described in Example 46 starting from compounds 9A/9B. Each crude residue obtained was purified using flash chromatography on silica gel (dichloromethane/MeOH: 0 to 3%) to provide a mixture of diastereomers (at the carbon alpha to the isopropyl ester).

Isomer 10A: mixture of diastereomers at the carbon alpha to the isopropyl ester; S_{P}; ¹H NMR (400 MHz, DMSO-d₆) δ 11.48 (s, 1H), 7.73-7.71 (m, 1H), 5.89 (s, 1H), 5.65 (d, J=7.71 Hz, 1H), 4.89 (heptuplet, J=6.22 Hz, 1H), 4.68-4.50 (m, 2H), 4.30-4.23 (m, 1H), 4.12-4.06 (m, 3H), 2.59-2.54 (m, 1H), 2.02-1.98 (m, 3H), 1.82-1.75 (m, 1H), 1.18 (d, J=6.22 Hz, 6H), 1.11 (d, J=7.04 Hz, 3H), 1.02 (brs, 3H); ³¹P NMR (162 MHz, DMSO-d₆) δ −5.97 (s, 1P); LC/MS: [(M+1)]⁺=462.2. The two isomers 10A were separated by Chiral Preparative HPLC with the following conditions: Column: AS-H: 20*250 mm, Mobile Phase A: CO₂: 80%, Mobile Phase B: iPrOH 20%, to provide: Isomer 10A1 (faster eluting, Rt=11.49 min): ³¹P NMR (202 MHz, DMSO-d₆) δ −5.96 (s, 1P); LC/MS: [(M+23)]⁺=484.3; and Isomer 10A2 (slower eluting, Rt=13.30 min): ³¹P NMR (202 MHz, DMSO-d₆) δ −5.95 (s, 1P); LC/MS: [(M+23)]⁺=484.2.

Isomer 10B: mixture of diastereomers in a ratio 1:1 at the carbon alpha to the isopropyl ester; R_{P}; ¹H NMR (400 MHz, DMSO-d₆) δ 11.46 (s, 1H), 7.77 (d, J=8.15 Hz, 1H), 5.90 (s, 1H), 5.61 (d, J=8.15 Hz, 1H), 4.90 (heptuplet, J=6.18 Hz, 1H), 4.65-4.61 (m, 2H), 4.46-4.35 (m, 2H), 4.13-4.04 (m, 2H), 2.54-2.52 (m, 1H), 1.99-1.91 (m, 3H), 1.75-1.67 (m, 1H), 1.19 (d, J=6.18 Hz, 3H), 1.185 (d, J=6.18 Hz, 3H), 1.10 (d, J=7.11 Hz, 3H), 1.02 (brs, 3H); ³¹P NMR (162 MHz, DMSO-d₆) δ −4.72 (s, 1P), −4.75 (s, 1P); LC/MS: [(M+1)]⁺=462.2. The two isomers 10B were separated by Chiral Preparative HPLC with the following conditions: Column: AD-H: 20*250 mm, Mobile Phase A: CO₂: 85%, Mobile Phase B: MeOH 15%, to provide: Isomer 10B1 (faster eluting, Rt=4.23 min): ³¹P NMR (202 MHz, DMSO-d₆) δ −4.72 (s, 1P); LC/MS: [(M+23)]⁺=484.1; and

Isomer 10B2 (slower eluting, Rt=4.40 min): ³¹P NMR (202 MHz, DMSO-d₆) δ −4.75 (s, 1P); LC/MS: [(M+23)]⁺=484.2.

Compounds 10A/10B were also made using the method described below in Example 52.

### Example 52

### Preparation of Compounds 10A, 10B, 12A and 12B

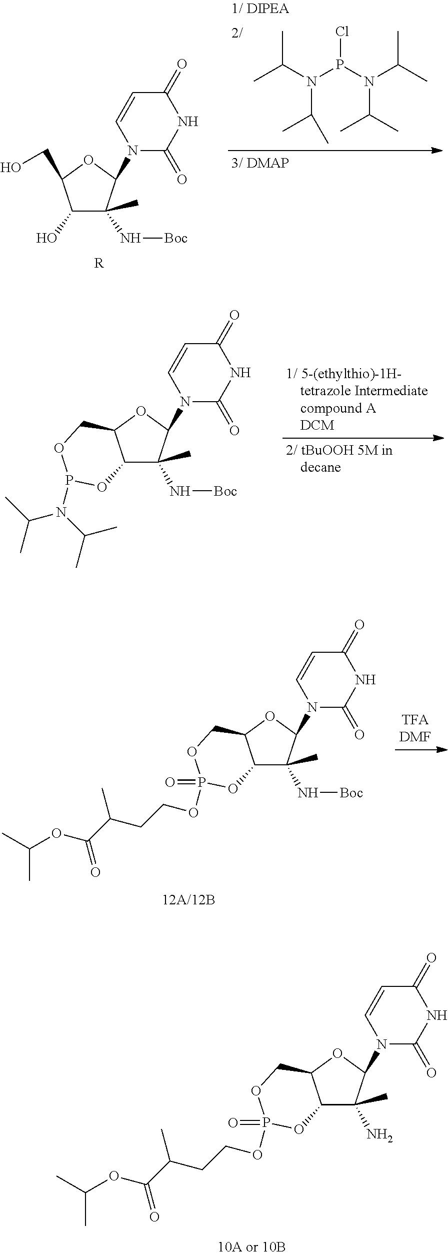

### Preparation of Nucleoside Starting Material R

### Step 1:

To a solution of 1-((2R,3R,4S,5R)-3-amino-4-hydroxy-5-(hydroxymethyl)-3-methyltetrahydrofuran-2-yl)pyrimidine-2,4(1H,3H)-dione (24.34 g, 95 mmol) in MeOH (189 mL) under nitrogen was added di-tert-butyl dicarbonate (26.8 g, 123 mmol). The reaction was allowed to stir at room temperature for 3 days. Further di-tert-butyl dicarbonate (4.6 g, 21 mmol) was added, and the reaction mixture was allowed to stir at room temperature for 24 hours. The resulting reaction mixture was concentrated in vacuo. The crude residue obtained was purified using flash chromatography on silica gel (dichloromethane/MeOH: 0 to 5%) to provide the expected protected nucleoside.

### Step 2:

To a cold (0° C.) solution of the protected nucleoside R (1 eq., 17.8 g, 49.8 mmol), DIPEA (3 eq., 26.1 mL, 149 mmol) and molecular sieve in anhydrous dichloromethane (199 mL) was added 1-chloro-N,N,N′,N′-tetraisopropylphosphinediamine (1.2 eq., 15.95 g, 59.8 mmol). The reaction was allowed to stir at 0° C. for 30 min and at room temperature for 3 h. The reaction was monitored using ³¹P NMR in CD₃CN. Then N,N-dimethylpyridin-4-amine (0.5 eq., 3.04 g, 24.90 mmol) was added, and the reaction mixture was allowed to stir at room temperature for about 15 hours. The reaction mixture was filtered and concentrated in vacuo. The obtained solid was dissolved in acetone, and then salts were filtered off. The mixture was concentrated in vacuo under nitrogen to provide the product 11, tert-butyl ((4aR,6R,7R,7aS)-2-(diisopropylamino)-6-(2,4-dioxo-3,4-dihydropyrimidin-1(2H)-yl)-7-methyltetrahydro-4H-furo[3,2-d][1,3,2]dioxaphosphinin-7-yl)carbamate: ³¹P NMR (162 MHz, DMSO-d₆) δ 149.84 (s, 1P).

### Step 3:

To a solution of the product of step 2 (1 eq., 6.5 g, 13.36 mmol) in dichloromethane (40 mL) at 0° C. under nitrogen were added 5-(ethylthio)-1H-tetrazole (2 eq., 3.48 g, 26.7 mmol) followed by the corresponding alcohol intermediate compound A (1.7 eq., 3.64 g, 22.71 mmol). The reaction was allowed to stir at 0° C. for 15 minutes then at room temperature for 3 h. Tert-butylhydroperoxide 5M in decane (4 eq., 10.69 mL, 53.4 mmol) was then added and the reaction mixture was allowed to stir at room temperature for about 15 hours. The reaction mixture was washed with 1N HCl and brine, the organic layer was extracted and concentrated in vacuo. The crude residue obtained was purified using flash chromatography on silica gel (dichloromethane/MeOH: 0 to 5%) to provide the 2 expected compounds, each as a mixture of diastereomers (at the carbon alpha to the isopropylester).

Isomer 12A: The mixture of diastereomers was further purified using RP-18 chromatography (H₂O/CH₃CN: 0 to 100%): mixture of diastereomers at the carbon alpha to the isopropyl ester; S_{P}; ³¹P NMR (162 MHz, DMSO-d₆) δ −6.35 (s, 1P); LC/MS: [(M+23)]⁺=584.4.

Isomer 12B: mixture of diastereomers at the carbon alpha to the isopropyl ester; R_{P}; LC/MS: [(M+23)]⁺=584.4.

### Step 4:

To a solution of compound 12A (1 eq., 4.2 g, 7.48 mmol) in anhydrous dichloromethane (74.8 mL) under nitrogen was added trifluoroacetic acid (11.53 mL, 150 mmol). The reaction was allowed to stir at room temperature for 3 hours, then solvents were removed under nitrogen flow and purified using flash chromatography on silica gel SNAP HP-SIL 100+50 g (dichloromethane/MeOH: 10%). The obtained solid was solubilized in a solution dichloromethane/EtOH 20%, filtered through PL-HCO₃ resin in order to remove TFA, and concentrated in vacuo to provide as a mixture of S_{P} diastereomers at the carbon alpha to the isopropyl ester (compounds 10A/10B). ³¹P NMR (162 MHz, DMSO-d₆) δ −5.96 (s, 1P); LC/MS: [(M+1)]⁺=462.4.

### Example 53

### Preparation of Compounds 13A and 13B

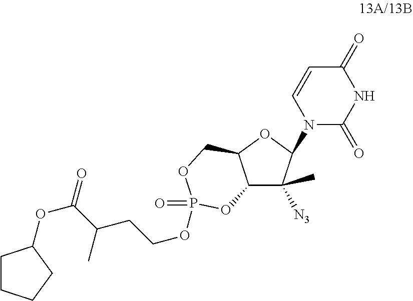

Compounds 13A/13B were made using the method described in Example 42 and substituting intermediate compound C for intermediate compound A, and nucleoside P for nucleoside 0:

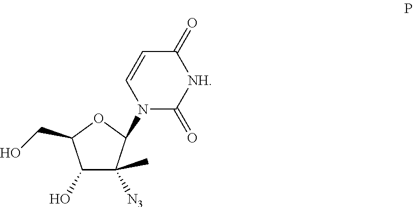 [SMILES: C[C@@]1(N=[N+]=[N-])[C@H](O)[C@@H](CO)O[C@H]1n1ccc(=O)[nH]c1=O]

The crude residue obtained was purified using flash chromatography on silica gel (dichloromethane/MeOH: 0 to 5%) to provide 2 mixtures of separated diastereomers (on the phosphorus atom).

Isomer 13A: mixture of diastereomers; LC/MS: [(M+1)]⁺=514.0.

Isomer 13B: mixture of diastereomers; LC/MS: [(M+1)]⁺=514.0.

### Example 54

### Preparation of Compounds 14A and 14B

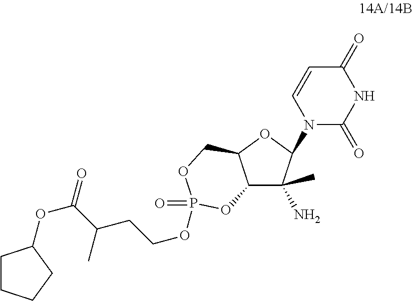

Compounds 14A/14B were made using the method described in Example 46 starting from compound 13A or 13B in ethanol. Each crude residue obtained was purified using flash chromatography on silica gel (dichloromethane/MeOH: 0 to 5%) to provide a mixture of diastereomers (at the carbon alpha to the cyclopentyl ester).

Isomer 14A: mixture of diastereomers at the carbon alpha to the cyclopentyl ester; S_{P}; ¹H NMR (400 MHz, DMSO-d₆) δ 11.49 (s, 1H), 7.72 (d, J=8.00 Hz, 1H), 5.89 (s, 1H), 5.65 (d, J=8.00 Hz, 1H), 5.09-5.06 (m, 1H), 4.68-4.51 (m, 2H), 4.30-4.24 (m, 1H), 4.13-4.05 (m, 3H), 2.59-2.54 (m, 1H), 2.02-1.96 (m, 3H), 1.82-1.74 (m, 3H), 1.66-1.53 (m, 6H), 1.11 (d, J=7.08 Hz, 3H), 1.02 (brs, 3H); ³¹P NMR (162 MHz, DMSO-d₆) δ −5.96 (s, 1P); LC/MS: [(M+1)]⁺=488.2. The two isomers 14A were separated by Chiral Preparative HPLC with the following conditions: Column: OD-H: 20*250 mm, Mobile Phase A: CO₂: 88%, Mobile Phase B: MeOH 12%, to provide: Isomer 14A1 (faster eluting, Rt=18.73 min): ³¹P NMR (202 MHz, DMSO-d₆) δ −5.95 (s, 1P); LC/MS: [(M+23)]⁺=510.2; and Isomer 14A2 (slower eluting, Rt=20.52 min): ³¹P NMR (202 MHz, DMSO-d₆) δ −5.96 (s, 1P); LC/MS: [(M+23)]⁺=510.3.

Isomer 14B: mixture of diastereomers in a ratio 1:1 at the carbon alpha to the cyclopentyl ester; R_{P}; ¹H NMR (400 MHz, DMSO-d₆) δ 11.47 (s, 1H), 7.78 (d, J=8.03 Hz, 1H), 5.90 (s, 1H), 5.61 (d, J=8.03 Hz, 1H), 5.09-5.06 (m, 1H), 4.65-4.61 (m, 2H), 4.46-4.35 (m, 2H), 4.13-4.03 (m, 2H), 2.56-2.54 (m, 1H), 1.99-1.90 (m, 3H), 1.85-1.77 (m, 2H), 1.74-1.53 (m, 7H), 1.10 (d, J=7.11 Hz, 3H), 1.02 (brs, 3H); ³¹P NMR (162 MHz, DMSO-d₆) δ −4.71 (s, 1P), −4.73 (s, 1P); LC/MS: [(M+1)]⁺=488.2. The two isomers 14B were separated by Chiral Preparative HPLC with the following conditions: Column: OJ-H: 20*250 mm, Mobile Phase A: CO₂: 88%, Mobile Phase B: MeOH 12%, to provide: Isomer 14B1 (faster eluting, Rt=4.90 min): ³¹P NMR (202 MHz, DMSO-d₆) δ −4.73 (s, 1P); LC/MS: [(M+1)]⁺=488.2; and Isomer 14B2 (slower eluting, Rt=5.45 min) which was further purified using Chiral Preparative HPLC with the following conditions: Column: IA: 20*250 mm, Mobile Phase A: CO₂: 80%, Mobile Phase B: MeOH 20%: ³¹P NMR (202 MHz, DMSO-d₆) δ −4.71 (s, 1P); LC/MS: [(M+1)]⁺=488.1.

### Example 55

### Preparation of Compounds 35A and 35B

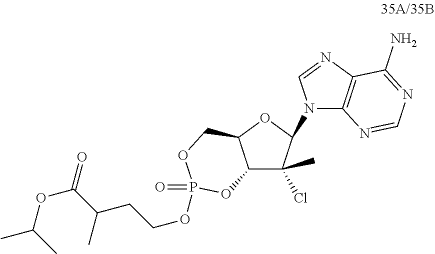

Compounds 35A/35B were made using the method described in Example 42 as follows: To a solution of intermediate compound A (1.1 g, 6.87 mmol) and tris(4-nitrophenyl) phosphate (3.17 g, 6.87 mmol) in dichloromethane (63 mL) at room temperature was added DBU (1.035 mL, 6.87 mmol) dropwise. The reaction was allowed to stir at room temperature for 2 hours. This mixture was added dropwise to a solution of nucleoside S (1.543 g, 5.15 mmol):

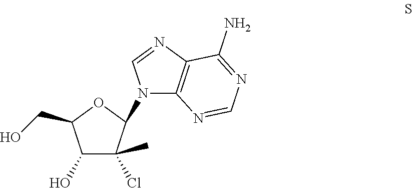 [SMILES: C[C@@]1(Cl)[C@H](O)[C@@H](CO)O[C@H]1n1cnc2c(N)ncnc21]

and DBU (2.07 mL, 13.73 mmol) in dichloromethane (20 mL)/Acetonitrile (20 mL)/THF (20 mL). The resulting reaction mixture was allowed to stir at room temperature for about 15 hours, then concentrated in vacuo. The crude residue obtained was directly purified using flash chromatography on silica gel (dichloromethane/MeOH) to provide 2 mixtures of separated diastereomers (on the phosphorus atom). Each diasteromeric mixture was further purified using C18 flash chromatography (water/acetonitrile) to provide the title compounds, each as a mixture of diastereomers (at the carbon alpha to the isopropyl ester).

Isomer 35A: mixture of diastereomers at the carbon alpha to the isopropyl ester; S_{P}; ¹H NMR (400 MHz, DMSO-d₆) δ 8.39 (s, 1H), 8.19-8.18 (m, 1H), 7.48 (s, 2H), 6.68 (s, 1H), 5.95 (brs, 1H), 4.88 (heptuplet, J=6.27 Hz, 1H), 4.76-4.67 (m, 1H), 4.62-4.57 (m, 1H), 4.36-4.30 (m, 1H), 4.21-4.15 (m, 2H), 2.67-2.59 (m, 1H), 2.12-2.03 (m, 1H), 1.86-1.76 (m, 1H), 1.40-1.39 (m, 3H), 1.16-1.10 (m, 9H); ³¹P NMR (162 MHz, DMSO-d₆) δ −6.51 (s, 1P); LC/MS: [(M+1)]⁺=504.4. The two isomers 35A were separated by Chiral Preparative HPLC with the following conditions: Column: AD-H: 20*250 mm, Mobile Phase A: CO₂: 85%, Mobile Phase B: iPrOH 15%, to provide: Isomer 35A1 (faster eluting, Rt=7.96 min): ³¹P NMR (202 MHz, DMSO-d₆) δ −6.48 (s, 1P); LC/MS: [(M+1)]⁺=504.2; and Isomer 35A2 (slower eluting, Rt=8.85 min): ³¹P NMR (202 MHz, DMSO-d₆) δ −6.50 (s, 1P); LC/MS: [(M+1)]⁺=504.2.

Isomer 35B: mixture of diastereomers at the carbon alpha to the isopropyl ester; R_{P}; ¹H NMR (400 MHz, DMSO-d₆) δ 8.43 (s, 1H), 8.20 (s, 1H), 7.46 (s, 2H), 6.67 (s, 1H), 5.63 (brs, 1H), 4.90 (heptuplet, J=6.25 Hz, 1H), 4.79-4.70 (m, 2H), 4.53-4.46 (m, 1H), 4.19-4.12 (m, 2H), 2.58-2.53 (m, 1H), 2.04-1.95 (m, 1H), 1.80-1.71 (m, 1H), 1.36 (brs, 3H), 1.20 (d, J=6.25 Hz, 3H), 1.19 (d, J=6.25 Hz, 3H), 1.12 (d, J=7.02 Hz, 3H); ³¹P NMR (162 MHz, DMSO-d₆) δ −5.55 (s, 1P); LC/MS: [(M+1)]⁺=504.4. The two isomers 35B were separated by Chiral Preparative HPLC with the following conditions: Column: AD-H: 30*250 mm, Mobile Phase A: CO₂: 75%, Mobile Phase B: EtOH 25%, to provide: Isomer 35B1 (faster eluting, Rt=7.34 min): ³¹P NMR (202 MHz, DMSO-d₆) δ −5.57 (s, 1P); LC/MS: [(M+1)]⁺=504.2; and Isomer 35B2 (slower eluting, Rt=8.32 min): ³¹P NMR (202 MHz, DMSO-d₆) δ −5.54 (s, 1P); LC/MS: [(M+1)]⁺=504.2.

### Example 56

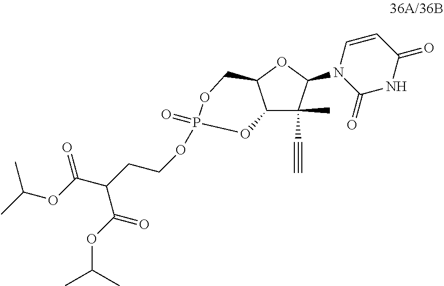

Compounds 36A/36B were made using the method described in Example 52 and substituting intermediate compound W for intermediate compound A and nucleoside compound O for nucleoside compound R. For Step 2: after the addition of tertbutylhydroperoxide 5M in decane (2 eq.) at 0° C., the reaction mixture was allowed to stir at 0° C. for 30 min, and at room temperature for 1 hour. The reaction mixture was then concentrated in vacuo, and the crude residue obtained was purified using flash chromatography on silica gel (dichloromethane/EtOAc: 50 to 100%) to provide the 2 separated isomers. Each of them was further purified using flash chromatography on silica gel (EtOAc: 100%) to provide pure compounds 36A and 36B.

Isomer 36A: S_{P}; ¹H NMR (400 MHz, DMSO-d₆) δ 11.61 (s, 1H), 7.75 (d, J=8.05 Hz, 1H), 6.29 (s, 1H), 5.67 (d, J=8.05 Hz, 1H), 4.99-4.91 (m, 2H), 4.73-4.59 (m, 2H), 4.25 (d, J=9.49 Hz, 1H), 4.18-4.10 (m, 3H), 3.64 (s, 1H), 3.64-3.60 (m, 1H), 2.22-2.17 (m, 2H), 1.21-1.18 (m, 15H); ³¹P NMR (162 MHz, DMSO-d₆) δ −6.82 (s, 1P); LC/MS: [(M+1)]⁺=543.2.

Isomer 36B: R_{P}; ¹H NMR (400 MHz, DMSO-d₆) δ 11.58 (s, 1H), 7.80 (d, J=8.07 Hz, 1H), 6.29 (s, 1H), 5.67-5.64 (m, 1H), 4.95 (heptuplet, J=6.28 Hz, 2H), 4.73-4.62 (m, 3H), 4.33-4.26 (m, 1H), 4.15-4.10 (m, 2H), 3.56 (s, 1H), 3.51 (t, J=7.12 Hz, 1H), 2.17-2.12 (m, 2H), 1.22-1.18 (m, 15H); ³¹P NMR (162 MHz, DMSO-d₆) δ −5.77 (s, 1P); LC/MS: [(M+1)]⁺=543.2.

### Example 57

### Preparation of Compounds 37A and 37B

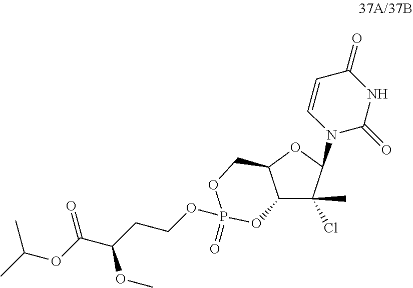

Compounds 37A/37B were made using the method described in Example 42 and substituting intermediate compound Y for intermediate compound A and nucleoside compound T:

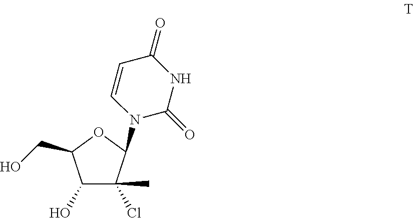 [SMILES: C[C@@]1(Cl)[C@H](O)[C@@H](CO)O[C@H]1n1ccc(=O)[nH]c1=O]

for nucleoside compound O. For Step 1, the addition of DBU was done at room temperature, and the reaction mixture was then stirred at room temperature for 5 hours. After Step 2, the crude residue obtained was directly purified using flash chromatography on silica gel (dichloromethane/MeOH) to provide 2 separated diastereomers (on the phosphorus atom). Each of them was further purified using C18 flash chromatography (water/acetonitrile) to provide pure compounds 37A and 37B.

Isomer 37A: S_{P}; ¹H NMR (400 MHz, DMSO-d₆) δ 11.66 (s, 1H), 7.79 (d, J=8.11 Hz, 1H), 6.46 (s, 1H), 5.71 (d, J=8.11 Hz, 1H), 5.01 (heptuplet, J=6.28 Hz, 1H), 4.77-4.68 (m, 1H), 4.62-4.57 (m, 1H), 4.51 (d, J=9.49 Hz, 1H), 4.28-4.22 (m, 1H), 4.19-4.11 (m, 2H), 3.97 (t, J=5.69 Hz, 1H), 3.30 (s, 3H), 2.18-2.04 (m, 2H), 1.55 (s, 3H), 1.24-1.21 m, 6H); ³¹P NMR (162 MHz, DMSO-d₆) δ −7.06 (s, 1P); LC/MS: [(M+1)]⁺=497.2.

Isomer 37B: R_{P}; ¹H NMR (400 MHz, DMSO-d₆) δ 11.62 (s, 1H), 7.79 (d, J=8.13 Hz, 1H), 6.45 (s, 1H), 5.68 (d, J=8.13 Hz, 1H), 4.97 (heptuplet, J=6.27 Hz, 1H), 4.90 (d, J=9.52 Hz, 1H), 4.76-4.72 (m, 2H), 4.41-4.35 (m, 1H), 4.21-4.12 (m, 2H), 3.88 (dd, J=8.67 Hz and 4.34 Hz, 1H), 3.29 (s, 3H), 2.10-2.02 (m, 1H), 1.96-1.88 (m, 1H), 1.52 (s, 3H), 1.225 (d, J=6.27 Hz, 3H), 1.215 (d, J=6.27 Hz, 3H); ³¹P NMR (162 MHz, DMSO-d₆) δ −5.83 (s, 1P); LC/MS: [(M+1)]⁺=497.2.

### Example 58

### Preparation of Compounds 38A and 38B

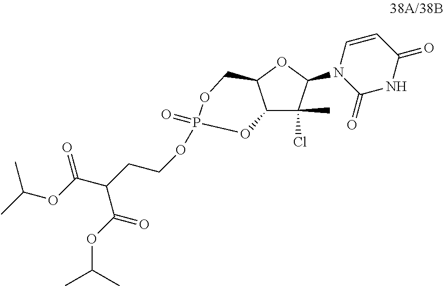

Compounds 38A/38B were made using the method described in Example 52 and substituting intermediate compound W for intermediate compound A and nucleoside compound T for nucleoside compound R. The crude residue obtained was purified using flash chromatography on silica gel (dichloromethane/MeOH: 0 to 10%) to provide the 2 separated diastereomers. Each of them was further purified using C18 flash chromatography (water/acetonitrile) to provide compounds 38A and 38B.

Isomer 38A: S_{P}; ¹H NMR (400 MHz, DMSO-d₆) δ 11.65 (s, 1H), 7.75 (d, J=8.16 Hz, 1H), 6.44 (s, 1H), 5.71-5.68 (m, 1H), 4.98-4.91 (m, 2H), 4.76-4.62 (m, 2H), 4.47-4.44 (m, 1H), 4.27-4.21 (m, 1H), 4.17-4.12 (m, 2H), 3.62 (t, J=7.11 Hz, 1H), 2.22-2.17 (m, 2H), 1.54 (s, 3H), 1.21-1.17 (m, 12H); ³¹P NMR (162 MHz, DMSO-d₆) δ −7.05 (s, 1P); LC/MS: [(M+1)]⁺=553.2.

Isomer 38B: R_{P}; ¹H NMR (400 MHz, DMSO-d₆) δ 11.63 (brs, 1H), 7.79 (d, J=8.16 Hz, 1H), 6.44 (s, 1H), 5.69-5.67 (m, 1H), 4.94 (heptuplet, J=6.31 Hz, 2H), 4.89 (d, J=9.55 Hz, 1H), 4.75-4.69 (m, 2H), 4.41-4.34 (m, 1H), 4.16-4.11 (m, 2H), 3.52 (t, J=7.36 Hz, 1H), 2.17-2.12 (m, 2H), 1.51 (s, 3H), 1.20 (d, J=6.31 Hz, 6H), 1.18 (d, J=6.31 Hz, 6H); ³¹P NMR (162 MHz, DMSO-d₆) δ −6.07 (s, 1P); LC/MS: [(M+1)]⁺=553.2.

### Example 59

### Preparation of Compounds 39A and 39B

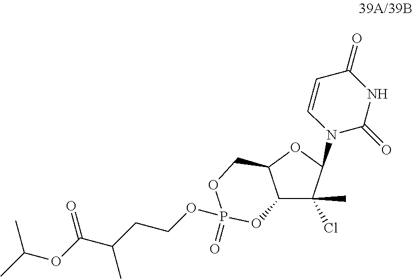

Compounds 39A/39B were made using the method described in Example 42 and substituting nucleoside compound T for nucleoside compound O. The reaction was carried out at room temperature for 1 hour for the first step. After step 2 and described work-up, the crude residue obtained was purified using flash chromatography on silica gel (dichloromethane/MeOH) to provide 2 mixtures of separated diastereomers (on the phosphorus atom). Each of them was further purified using preparative MS/HPLC (C18, water/acetonitrile) to provide compounds 39A and 39B, each as a mixture of diastereomers (at the carbon alpha to the isopropyl ester).

Isomer 39A: mixture of diastereomers at the carbon alpha to the isopropyl ester; S_{P}; ¹H NMR (400 MHz, DMSO-d₆) δ 11.63 (s, 1H), 7.76 (d, J=8.16 Hz, 1H), 6.44 (s, 1H), 5.72-5.69 (m, 1H), 4.89 (heptuplet, J=6.28 Hz, 1H), 4.75-4.60 (m, 2H), 4.45 (t, J=8.79 Hz, 1H), 4.26-4.20 (m, 1H), 4.16-4.11 (m, 2H), 2.60-2.52 (m, 1H), 2.08-1.98 (m, 1H), 1.84-1.75 (m, 1H), 1.53-1.52 (m, 3H), 1.185 (d, J=6.28 Hz, 3H), 1.18 (d, J=6.28 Hz, 3H), 1.12 (d, J=7.08 Hz, 3H); ³¹P NMR (162 MHz, DMSO-d₆) δ −6.93 (s, 1P); LC/MS: [(M+1)]⁺=481.0. The two isomers 39B were separated by Chiral Preparative HPLC with the following conditions: Column: Lux Cellulose-4: 20*250 mm, Mobile Phase A: CO₂: 80%, Mobile Phase B: iPrOH 20%, to provide: Isomer 39A1 (faster eluting, Rt=15.98 min): ³¹P NMR (202 MHz, DMSO-d₆) δ −6.92 (s, 1P); LC/MS: [(M+23)]⁺=503.2; and Isomer 39A2 (slower eluting, Rt=16.80 min): ³¹P NMR (202 MHz, DMSO-d₆) δ −6.92 (s, 1P); LC/MS: [(M+23)]⁺=503.2.

Isomer 39B: mixture of diastereomers at the carbon alpha to the isopropyl ester; R_{P}; 1H NMR (400 MHz, DMSO-d₆) δ 11.62 (s, 1H), 7.79 (d, J=8.12 Hz, 1H), 6.45 (s, 1H), 5.68 (d, J=8.12 Hz, 1H), 4.93-4.86 (m, 2H), 4.76-4.69 (m, 2H), 4.41-4.34 (m, 1H), 4.15-4.09 (m, 2H), 2.57-2.53 (m, 1H), 2.02-1.93 (m, 1H), 1.78-1.70 (m, 1H), 1.52 (s, 3H), 1.195 (d, J=6.25 Hz, 3H), 1.185 (d, J=6.25 Hz, 3H), 1.11 (d, J=7.00 Hz, 3H); ³¹P NMR (162 MHz, DMSO-d₆) δ −5.90 (s, 1P), −5.93 (s, 1P); LC/MS: [(M+1)]⁺=481.0. The two isomers 39B were separated by Chiral Preparative HPLC with the following conditions: Column: AD-H: 20*250 mm, Mobile Phase A: CO₂: 80%, Mobile Phase B: EtOH 20%, to provide: Isomer 39B1 (faster eluting, Rt=4.38 min): ³¹P NMR (202 MHz, DMSO-d₆) δ −5.89 (s, 1P); LC/MS: [(M+23)]⁺=503.2; and Isomer 39B2 (slower eluting, Rt=5.33 min): ³¹P NMR (202 MHz, DMSO-d₆) δ −5.93 (s, 1P); LC/MS: [(M+23)]⁺⁼503.2.

All compounds with a chiral R¹ group could also be synthesized according to same methods using chiral intermediates as described below in Examples 60-62.

### Example 60

### Preparation of Compounds 8A1 and 8B2

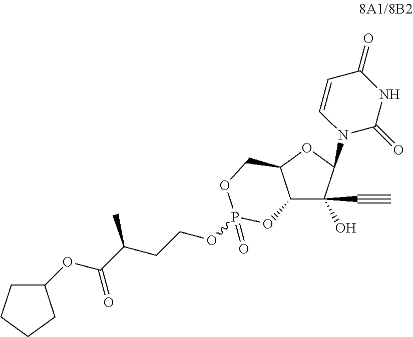

### Step 1:

To a cold (−15° C.) solution of 1-chloro-N,N,N′,N′-tetraisopropylphosphinediamine (8.59 g, 32.2 mmol) in diethyl ether (85 mL) were added triethylamine (13.47 mL, 97 mmol) under nitrogen, then a solution of intermediate compound WW (6 g, 32.2 mmol) in diethyl ether (42 mL) was slowly added. The reaction was allowed to stir at −15° C. for 1 hour and then at room temperature for 2 hours. The suspension was filtered under nitrogen and washed with diethyl ether. The filtrate was concentrated in vacuo at room temperature under nitrogen to provide the product. The crude intermediate compound was stored at −20° C. under nitrogen and was directly used in the next step without further purification: ³¹P NMR (162 MHz, CDCl₃) δ 123.8 (s, 1P).

### Step 2:

To a solution of nucleoside U (6.5 g, 24.23 mmol) in pyridine (150 mL, 6.5 mL/mmol) was added 1H-tetrazole 0.45 M in CH₃CN (162 mL, 72.7 mmol). The reaction mixture was cooled to −5° C. and a solution of the intermediate compound from step 1 (13.12 g, 31.5 mmol) in acetonitrile (70 mL) was added dropwise. The reaction was allowed to stir at −15° C. for 1 h and at room temperature for 3 hours. The reaction was monitored by LC/MS. A solution of tert-butylhydroperoxide, 5M in decane (11.5 mL, 24.23 mmol) was then added dropwise, and the resulting reaction mixture was allowed to stir for about 15 hours at RT. The crude mixture was concentrated in vacuo, and co-evaporated with toluene (2×). The crude compound was purified using flash chromatography on silica gel (dichloromethane/MeOH: 0 to 10%) to provide a mixture of diastereomers at P. This mixture of diastereomers was further purified using preparative HPLC (C18, H₂O/CH₃CN: 0 to 50%) to provide the compounds 8A1 and 8B2 (isolated diastereomers at P).

Isomer 8A1: ¹H NMR (400 MHz, DMSO-d₆) δ 11.52 (s, 1H), 7.77 (d, J=8.19 Hz, 1H), 7.20 (s, 1H), 6.05 (s, 1H), 5.67 (d, J=8.19 Hz, 1H), 5.10-5.07 (m, 1H), 4.70-4.58 (m, 2H), 4.36-4.34 (m, 1H), 4.16-4.08 (m, 3H), 3.81 (s, 1H), 2.64-2.55 (m, 1H), 2.05-1.98 (m, 1H), 1.83-1.78 (m, 3H), 1.67-1.53 (m, 6H), 1.115 (d, J=7.06 Hz, 3H); ³¹P NMR (162 MHz, DMSO-d₆) δ −6.39 (s, 1P); LC/MS: [(M+1)]⁺=499.2.

Isomer 8B2: ¹H NMR (400 MHz, DMSO-d₆) δ 11.50 (s, 1H), 7.84 (d, J=8.16 Hz, 1H), 7.14 (s, 1H), 6.06 (s, 1H), 5.65 (d, J=8.16 Hz, 1H), 5.09-5.05 (m, 1H), 4.76-4.74 (m, 1H), 4.69-4.60 (m, 2H), 4.28-4.22 (m, 1H), 4.13-4.05 (m, 2H), 3.83 (s, 1H), 1.99-1.90 (m, 1H), 1.85-1.77 (m, 2H), 1.75-1.53 (m, 8H), 1.10 (d, J=7.07 Hz, 3H); ³¹P NMR (162 MHz, DMSO-d₆) δ −5.34 (s, 1P); LC/MS: [(M+23)]⁺=521.3.

### Alternate Method for Making 8A2/8B1

Compounds 8A2/8B1 were also made using the method described in Example 48 using intermediate compound XX as a starting material.

Isomer 8A2: ³¹P NMR (162 MHz, DMSO-d₆) δ −6.42 (s, 1P).

Isomer 8B1: ³¹P NMR (162 MHz, DMSO-d₆) δ −5.21 (s, 1P).

### Example 61

### Preparation of Compounds 35B1 and 35A2

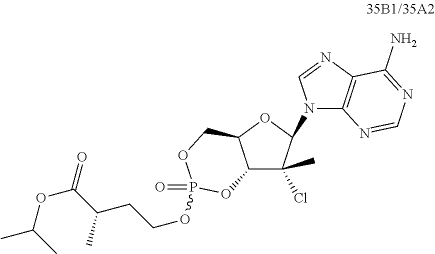

Compounds 35B1/35A2 were made using the method described in Example 42 using intermediate compound RR as the alcohol starting material and substituting nucleoside compound S for nucleoside compound O. Final compounds were further purified using C18 flash chromatography (water/acetonitrile) to provide compounds 35B1 and 35A2 (isolated diastereomers at P).

Isomer 35B1: ¹H NMR (400 MHz, DMSO-d₆) δ 8.45 (s, 1H), 8.20 (s, 1H), 7.49 (s, 2H), 6.67 (s, 1H), 4.90 (heptuplet, J=6.28 Hz, 1H), 4.79-4.72 (m, 2H), 4.52-4.46 (m, 1H), 4.18-4.12 (m, 2H), 2.58-2.55 (m, 1H), 2.04-1.95 (m, 1H), 1.79-1.71 (m, 1H), 1.36 (brs, 3H), 1.20 (d, J=6.28 Hz, 3H), 1.19 (d, J=6.28 Hz, 3H), 1.12 (d, J=7.03 Hz, 3H); ³¹P NMR (162 MHz, DMSO-d₆) δ −5.58 (s, 1P); LC/MS: [(M+1)]⁺=504.2.

Isomer 35A2: ¹H NMR (400 MHz, DMSO-d₆) δ 8.39 (s, 1H), 8.18 (s, 1H), 7.50 (s, 2H), 6.68 (s, 1H), H), 4.88 (heptuplet, J=6.28 Hz, 1H), 4.76-4.67 (m, 1H), 4.61-4.56 (m, 1H), 4.36-4.30 (m, 1H), 4.19-4.14 (m, 2H), 2.68-2.60 (m, 1H), 2.11-2.02 (m, 1H), 1.86-1.77 (m, 1H), 1.40 (s, 3H), 1.155 (d, J=6.28 Hz, 3H), 1.15 (d, J=6.28 Hz, 3H), 1.11 (d, J=7.08 Hz, 3H); ³¹P NMR (162 MHz, DMSO-d₆) δ −6.50 (s, 1P); LC/MS: [(M+1)]⁺=504.2.

### Alternate Method for Making 35B2/35A1

Compounds 35B2/35A1 were also made using the method described in Example 42 using intermediate compound VV as the alcohol starting material.

Isomer 35B2: ³¹P NMR (162 MHz, DMSO-d₆) δ −5.54 (s, 1P); LC/MS: [(M+1)]⁺=504.2.

Isomer 35A1: ³¹P NMR (162 MHz, DMSO-d₆) δ −6.49 (s, 1P); LC/MS: [(M+1)]⁺=504.2.

### Example 62

### Preparation of Compounds 39A1 and 39B2

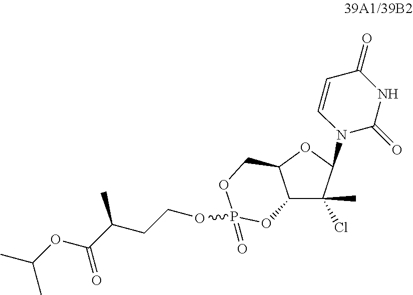

Compounds 39A1/39B2 were made using the method described in Example 42 using intermediate compound RR as starting material and substituting nucleoside compound T for nucleoside compound O.

Isomer 39A1: ¹H NMR (400 MHz, DMSO-d₆) δ 11.64 (s, 1H), 7.76 (d, J=8.18 Hz, 1H), 6.43 (s, 1H), 5.72 (d, J=8.18 Hz, 1H), 4.89 (heptuplet, J=6.24 Hz, 1H), 4.75-4.61 (m, 2H), 4.45-4.43 (m, 1H), 4.26-4.20 (m, 1H), 4.16-4.11 (m, 2H), 2.59-2.52 (m, 1H), 2.08-1.99 (m, 1H), 1.84-1.75 (m, 1H), 1.52 (s, 3H), 1.185 (d, J=6.24 Hz, 3H), 1.18 (d, J=6.24 Hz, 3H), 1.12 (d, J=7.06 Hz, 3H); ³¹P NMR (162 MHz, DMSO-d₆) δ −6.93 (s, 1P); LC/MS: [(M+1)]⁺=481.0.

Isomer 39B2: ¹H NMR (400 MHz, DMSO-d₆) δ 11.62 (s, 1H), 7.79 (d, J=8.15 Hz, 1H), 6.44 (s, 1H), 5.68 (d, J=8.15 Hz, 1H), 4.92-4.86 (m, 2H), 4.75-4.67 (m, 2H), 4.40-4.33 (m, 1H), 4.15-4.09 (m, 2H), 2.54-2.52 (m, 1H), 2.02-1.93 (m, 1H), 1.77-1.69 (m, 1H), 1.52 (s, 3H), 1.19 (d, J=6.26 Hz, 3H), 1.18 (d, J=6.26 Hz, 3H), 1.10 (d, J=7.04 Hz, 3H); ³¹P NMR (162 MHz, DMSO-d₆) δ −5.93 (s, 1P); LC/MS: [(M+1)]⁺=481.0.

### Example 63

### Preparation of Compounds 7A1 and 7B1

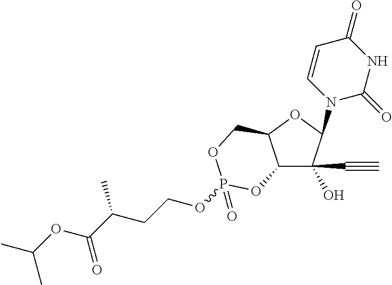

Compounds 7A1/7B1 were also made using the method described in Example 48 and using intermediate compound VV as the alcohol starting material.

Compounds 7A1/7B1 were also made using the following alternate method.

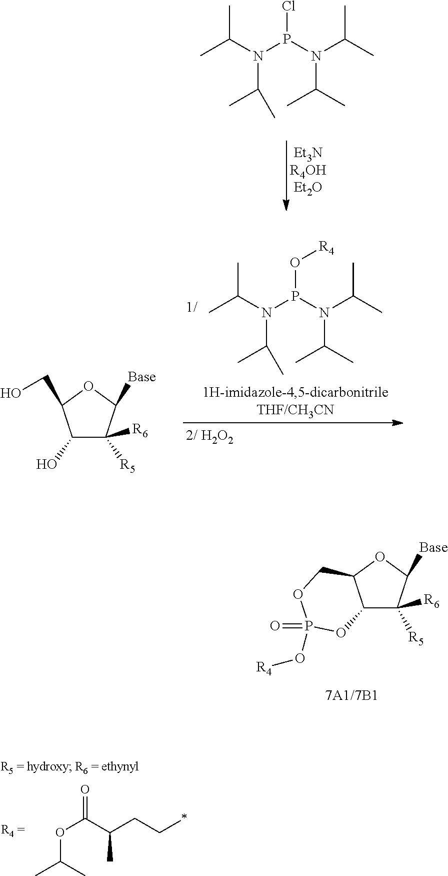

### Step 1:

carried out using the method described in Example 48.

### Step 2:

To a solution of THF (117 mL) under nitrogen at RT were simultaneously and slowly added a solution of appropriate nucleoside (13.87 g, 51.7 mmol) and 1H-imidazole-4,5-dicarbonitrile (15.26 g, 129 mmol) (coevaporated 3 times with CH₃CN and THF) in THF (374 mL) and CH₃CN (187 mL), and a solution of the intermediate compound of step 1 (20.19 g, 51.7 mmol) in THF (117 mL). The reaction mixture was stirred at RT for about 15 hours. Hydrogen peroxide (22.26 mL, 259 mmol) was then added dropwise at RT for 15 min. The reaction mixture was stirred at RT for 3 hours, and then, diluted with EtOAc and water. The aqueous layer was washed twice with EtOAc. The combined organic layers were washed with brine, dried, and concentrated under reduced pressure. The crude residue was purified by flash chromatographies on silica gel (DCM/MeOH: 0 to 10%) followed by RP-18 chromatography (H₂O/CH₃CN) and preparative HPLC to afford the 2 title compounds (isolated diasteromers at P).

Isomer 7A1: ¹H NMR (400 MHz, DMSO-d₆) δ 11.55 (s, 1H), 7.79 (d, J=8.05 Hz, 1H), 7.22 (s, 1H), 6.07 (s, 1H), 5.67 (d, J=8.05 Hz, 1H), 4.92 (heptuplet, J=6.17 Hz, 1H), 4.71-4.62 (m, 1H), 4.58-4.54 (m, 1H), 4.40-4.38 (m, 1H), 4.17-4.07 (m, 3H), 3.83 (s, 1H), 2.60-2.55 (m, 1H), 2.09-2.02 (m, 1H), 1.88-1.75 (m, 1H), 1.19 (d, J=6.17 Hz, 6H), 1.12 (d, J=7.03 Hz, 3H); ³¹P NMR (162 MHz, DMSO-d₆) δ −6.41 (s, 1P); LC/MS: [(M+23)]⁺=495.2.

Isomer 7B1: ¹H NMR (400 MHz, DMSO-d₆) δ 11.53 (s, 1H), 7.84 (d, J=8.06 Hz, 1H), 7.17 (s, 1H), 6.07 (s, 1H), 5.65 (d, J=8.06 Hz, 1H), 4.90 (heptuplet, J=6.14 Hz, 1H), 4.77-4.74 (m, 1H), 4.69-4.63 (m, 2H), 4.30-4.23 (m, 1H), 4.12-4.07 (m, 2H), 3.84 (s, 1H), 2.00-1.92 (m, 1H), 1.76-1.68 (m, 1H), 1.20 (d, J=6.14 Hz, 3H), 1.19 (d, J=6.14 Hz, 3H), 1.12 (d, J=7.05 Hz, 3H); ³¹P NMR (162 MHz, DMSO-d₆) δ −5.23 (s, 1P); LC/MS: [(M+23)]⁺=495.1.

### Example 64

### Preparation of Compounds 7A2 and 7B2

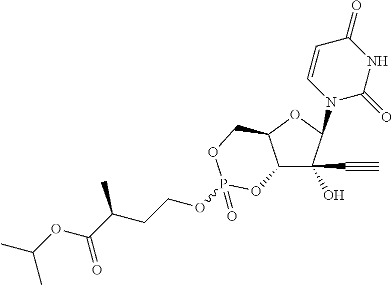

Compounds 7A2/7B2 were also made using the method described in Example 48 using Intermediate RR as the alcohol starting material.

Compounds 7A2/7B2 were also made using the alternate method described in Example 63.

Isomer 7A2: ¹H NMR (400 MHz, DMSO-d₆) δ 11.55 (s, 1H), 7.78 (d, J=8.12 Hz, 1H), 7.22 (s, 1H), 6.06 (s, 1H), 5.68 (d, J=8.12 Hz, 1H), 4.91 (heptuplet, J=6.24 Hz, 1H), 4.70-4.58 (m, 2H), 4.37-4.34 (m, 1H), 4.16-4.09 (m, 3H), 3.83 (s, 1H), 2.63-2.58 (m, 1H), 2.07-1.99 (m, 1H), 1.86-1.78 (m, 1H), 1.19 (d, J=6.24 Hz, 3H), 1.185 (d, J=6.24 Hz, 3H), 1.12 (d, J=7.06 Hz, 3H); ³¹P NMR (162 MHz, DMSO-d₆) δ −6.38 (s, 1P); LC/MS: [(M+1)]⁺=473.0.

Isomer 7B2: ¹H NMR (400 MHz, DMSO-d₆) δ 11.50 (s, 1H), 7.84 (d, J=8.00 Hz, 1H), 7.14 (s, 1H), 6.06 (s, 1H), 5.64 (d, J=8.00 Hz, 1H), 4.89 (heptuplet, J=6.18 Hz, 1H), 4.76-4.74 (m, 1H), 4.69-4.60 (m, 2H), 4.28-4.22 (m, 1H), 4.13-4.05 (m, 2H), 3.82 (s, 1H), 2.00-1.91 (m, 1H), 1.76-1.68 (m, 1H), 1.19 (d, J=6.18 Hz, 3H), 1.185 (d, J=6.18 Hz, 3H), 1.10 (d, J=7.07 Hz, 3H); ³¹P NMR (162 MHz, DMSO-d₆) δ −5.37 (s, 1P); LC/MS: [(M+1)]⁺=473.0.

### Example 65

### Preparation of Compound 83A1

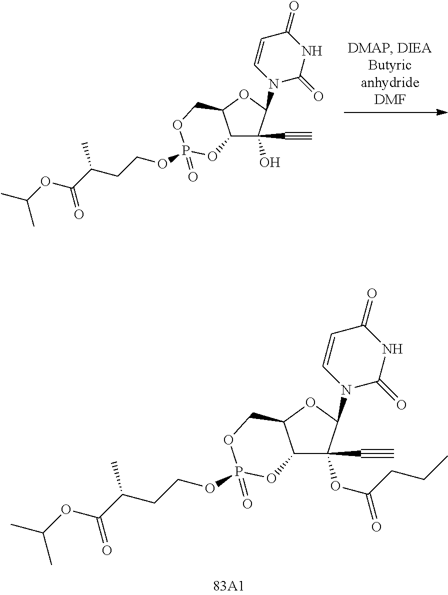 [SMILES: C#C[C@@]1(O)[C@@H]2O[P@@](=O)(OCC[C@@H](C)C(=O)OC(C)C)OC[C@H]2O[C@H]1n1ccc(=O)[nH]c1=O ; C#C[C@@]1(OC(=O)CCC)[C@@H]2O[P@@](=O)(OCC[C@@H](C)C(=O)OC(C)C)OC[C@H]2O[C@H]1n1ccc(=O)[nH]c1=O]

To a solution of compound 7A1 (374 mg, 0.79 mmol), DIEA (0.28 mL, 1.58 mmol) and butyric anhydride (0.19 mL, 1.19 mmol) in DMF (2 mL) was added DMAP (9.67 mg, 0.08 mmol). The reaction mixture was allowed to stir at room temperature for 1 hour. Then additional butyric anhydride (0.13 mL, 0.79 mmol) was added and the reaction mixture was allowed to stir for about 15 hours. The reaction mixture was concentrated in vacuo and the crude residue obtained was purified using flash chromatography on silica gel (DCM/MeOH: 0 to 10%) followed by RP-18 chromatography (H₂O/CH₃CN) to provide compound 83A1. ¹H NMR (400 MHz, DMSO-d₆) δ 11.53 (s, 1H), 7.88-7.87 (m, 0.65H), 7.45 (brs, 0.35H), 6.41 (s, 0.65H), 5.92 (brs, 0.35H), 5.71 (d, J=8.03 Hz, 1H), 5.29 (brs, 0.35H), 4.91 (heptuplet, J=6.21 Hz, 1H), 4.82-4.80 (m, 0.65H), 4.73-4.65 (m, 1H), 4.44 (brs, 0.65H), 4.19-4.11 (m, 4H), 3.94 (brs, 0.35H), 2.58-2.54 (m, 1H), 2.45-2.41 (m, 2H), 2.07-2.02 (m, 1H), 1.85-1.79 (m, 1H), 1.59-1.54 (m, 2H), 1.20 (d, J=6.21 Hz, 3H), 1.19 (d, J=6.21 Hz, 3H), 1.12 (d, J=7.07 Hz, 3H), 0.91 (t, J=7.36 Hz, 3H); ³¹P NMR (162 MHz, DMSO-d₆) δ −6.90 (s, 1P); LC/MS: [(M+23)]⁺=565.3.

### Example 66

### Preparation of Compound 84A1

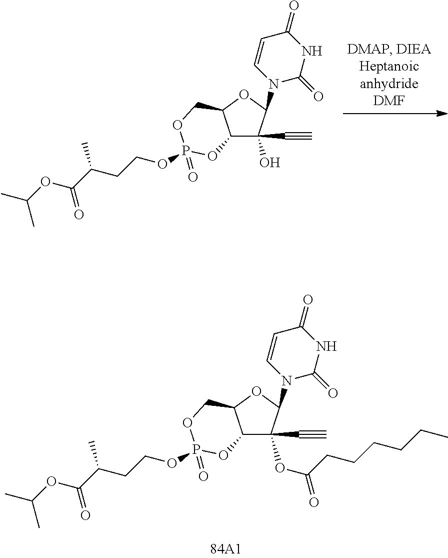 [SMILES: C#C[C@@]1(O)[C@@H]2O[P@@](=O)(OCC[C@@H](C)C(=O)OC(C)C)OC[C@H]2O[C@H]1n1ccc(=O)[nH]c1=O ; C#C[C@@]1(OC(=O)CCCCCC)[C@@H]2O[P@@](=O)(OCC[C@@H](C)C(=O)OC(C)C)OC[C@H]2O[C@H]1n1ccc(=O)[nH]c1=O]

Compound 84A1 was made as described below, using the method described in Example 65 and substituting the corresponding anhydride. ¹H NMR (400 MHz, DMSO-d₆) δ 11.50 (s, 1H), 7.88-7.85 (m, 0.6H), 7.45 (brs, 0.4H), 6.41 (brs, 0.6H), 5.92 (brs, 0.4H), 5.70 (d, J=8.04 Hz, 1H), 5.28 (brs, 0.4H), 4.91 (heptuplet, J=6.21 Hz, 1H), 4.82-4.65 (m, 1.6H), 4.43 (brs, 0.6H), 4.18-4.09 (m, 4H), 3.94 (brs, 0.4H), 2.58-2.54 (m, 1H), 2.46-2.41 (m, 2H), 2.07-2.02 (m, 1H), 1.86-1.79 (m, 1H), 1.56-1.52 (m, 2H), 1.32-1.24 (m, 6H), 1.20 (d, J=6.21 Hz, 3H), 1.19 (d, J=6.21 Hz, 3H), 1.13 (d, J=7.01 Hz, 3H), 0.86 (t, J=6.86 Hz, 3H); ³¹P NMR (162 MHz, DMSO-d₆) δ −6.90 (s, 1P); LC/MS: [(M+1)]⁺=585.3.

### Example 67

### Preparation of Compound 85A2

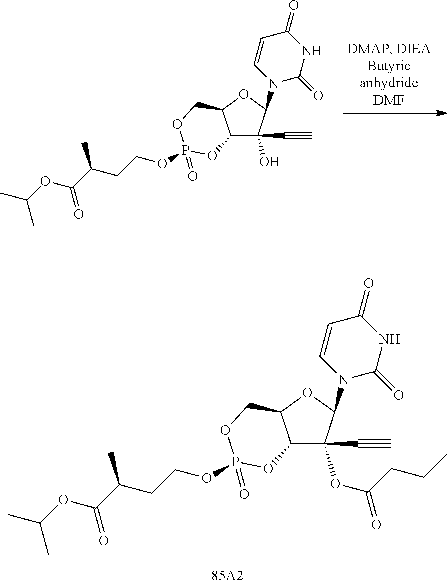 [SMILES: C#C[C@@]1(O)[C@@H]2O[P@@](=O)(OCC[C@H](C)C(=O)OC(C)C)OC[C@H]2O[C@H]1n1ccc(=O)[nH]c1=O ; C#C[C@@]1(OC(=O)CCC)[C@@H]2O[P@@](=O)(OCC[C@H](C)C(=O)OC(C)C)OC[C@H]2O[C@H]1n1ccc(=O)[nH]c1=O]

Compound 85A2 was made using the method described in Example 65 starting from Compound 7A2. ¹H NMR (400 MHz, DMSO-d₆) δ 11.51 (s, 1H), 7.86 (brs, 0.6H), 7.45 (brs, 0.4H), 6.40 (brs, 0.6H), 5.92 (brs, 0.4H), 5.71 (d, J=8.03 Hz, 1H), 5.30 (brs, 0.4H), 4.91 (heptuplet, J=6.25 Hz, 1H), 4.80-4.65 (m, 1.6H), 4.47 (brs, 0.6H), 4.16-4.10 (m, 4H), 3.95 (brs, 0.4H), 2.60-2.58 (m, 1H), 2.45-2.41 (m, 2H), 2.06-2.00 (m, 1H), 1.84-1.79 (m, 1H), 1.60-1.55 (m, 2H), 1.195 (d, J=6.25 Hz, 3H), 1.19 (d, J=6.25 Hz, 3H), 1.13 (d, J=7.09 Hz, 3H), 0.91 (t, J=7.36 Hz, 3H); ³¹P NMR (162 MHz, DMSO-d₆) δ −6.88 (s, 1P); LC/MS: [(M+23)]⁺=565.4.

### Example 68

### Preparation of Compound 86A

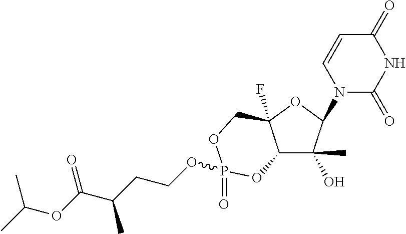

Compounds 86A/86B were made using the method described in Example 63 and substituting nucleoside compound V for nucleoside compound O:

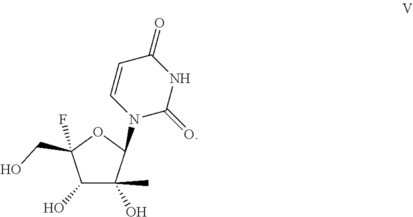 [SMILES: C[C@]1(O)[C@H](n2ccc(=O)[nH]c2=O)O[C@](F)(CO)[C@H]1O]

Isomer 86A: ¹H NMR (400 MHz, DMSO-d₆) δ 11.62 (brs, 1H), 7.74 (d, J=8.11 Hz, 1H), 6.36 (s, 1H), 6.01 (s, 1H), 5.73 (d, J=8.11 Hz, 1H), 4.89 (heptuplet, J=6.24 Hz, 1H), 4.82-4.52 (m, 3H), 4.16-4.11 (m, 2H), 2.60-2.55 (m, 1H), 2.08-1.97 (m, 1H), 1.84-1.75 (m, 1H), 1.19-1.16 (m, 9H), 1.12 (d, J=6.98 Hz, 3H); ³¹P NMR (162 MHz, DMSO-d₆) δ −7.54 (s, 1P); ¹⁹F NMR (376 MHz, DMSO-d₆) δ −122.70 (s, 1F); LC/MS: [(M+1)]=481.2.

### Example 69

### Preparation of Compound 87A/87B

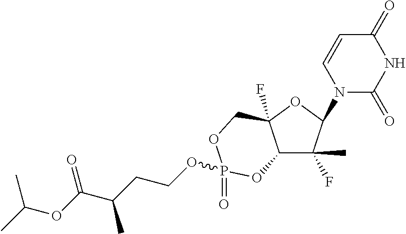

Compounds 87A/87B were made using the method described in Example 42 using intermediate compound RR as the alcohol starting material and substituting nucleoside compound W for nucleoside compound O:

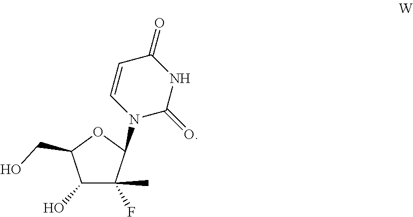 [SMILES: C[C@@]1(F)[C@H](O)[C@@H](CO)O[C@H]1n1ccc(=O)[nH]c1=O]

Isomer 87A: ¹H NMR (400 MHz, DMSO-d₆) δ 11.64 (brs, 1H), 7.74 (d, J=8.10 Hz, 1H), 6.24 (d, J=22.17 Hz, 1H), 5.72 (d, J=8.10 Hz, 1H), 4.89 (heptuplet, J=6.29 Hz, 1H), 4.70 (ddd, J=21.85 Hz, 8.95 Hz, 4.69 Hz, 1H), 4.58-4.54 (m, 1H), 4.39 (dd, J=23.03 Hz, 9.91 Hz, 1H), 4.22-4.10 (m, 3H), 2.60-2.55 (m, 1H), 2.05-2.00 (m, 1H), 1.84-1.76 (m, 1H), 1.37 (d, J=22.60 Hz, 3H), 1.19 (d, J=6.29 Hz, 3H), 1.18 (d, J=6.29 Hz, 3H), 1.12 (d, J=7.10 Hz, 3H); ³¹P NMR (162 MHz, DMSO-d₆) δ −6.68 (s, 1P); ¹⁹F NMR (376 MHz, DMSO-d₆) δ −156.70 (s, 1F); LC/MS: [(M+1)]-=465.2.

Isomer 87B: ¹H NMR (400 MHz, DMSO-d₆) δ 11.64 (brs, 1H), 7.77 (d, J=8.03 Hz, 1H), 6.24 (d, J=22.08 Hz, 1H), 5.69 (d, J=8.03 Hz, 1H), 4.89 (heptuplet, J=6.29 Hz, 1H), 4.82-4.63 (m, 3H), 4.35-4.29 (m, 1H), 4.15-4.03 (m, 2H), 1.99-1.90 (m, 1H), 1.76-1.68 (m, 1H), 1.36 (d, J=22.74 Hz, 3H), 1.19 (d, J=6.29 Hz, 3H), 1.18 (d, J=6.29 Hz, 3H), 1.10 (d, J=7.09 Hz, 3H); ³¹P NMR (162 MHz, DMSO-d₆) δ −5.15 (s, 1P); ¹⁹F NMR (376 MHz, DMSO-d₆) δ −157.25 (s, 1F); LC/MS: [(M+1)]⁺=465.2.

### Example 70

### Preparation of Compound 88A/88B

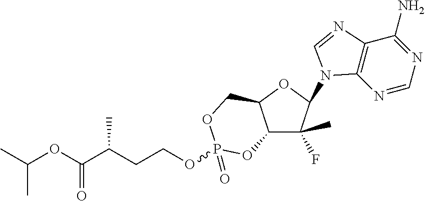

Compounds 88A/88B were made using the method described in Example 42 using intermediate compound RR as the alcohol starting material and substituting nucleoside compound X for nucleoside compound O:

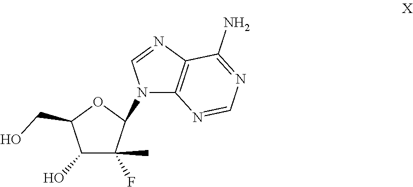 [SMILES: C[C@@]1(F)[C@H](O)[C@@H](CO)O[C@H]1n1cnc2c(N)ncnc21]

Isomer 88A: ¹H NMR (400 MHz, DMSO-d₆) δ 8.37 (s, 1H), 8.18 (s, 1H), 7.50 (s, 2H), 6.47 (d, J=20.62 Hz, 1H), 5.63 (brs, 1H), 4.88 (heptuplet, J=6.22 Hz, 1H), 4.73 (ddd, J=22.50 Hz, 9.07 Hz, 4.70 Hz, 1H), 4.48-4.43 (m, 1H), 4.32-4.26 (m, 1H), 4.21-4.16 (m, 2H), 2.68-2.61 (m, 1H), 2.14-2.06 (m, 1H), 1.86-1.78 (m, 1H), 1.28 (d, J=22.83 Hz, 3H), 1.17-1.14 (m, 9H); ³¹P NMR (162 MHz, DMSO-d₆) δ −6.38 (s, 1P); ¹⁹F NMR (376 MHz, DMSO-d₆) δ −159.13 (s, 1F); LC/MS: [(M+1)]⁺=488.6.

Isomer 88B: ¹H NMR (400 MHz, DMSO-d₆) δ 8.42 (s, 1H), 8.20 (s, 1H), 7.48 (s, 2H), 6.45 (d, J=20.89 Hz, 1H), 5.48 (brs, 1H), 4.91 (heptuplet, J=6.28 Hz, 1H), 4.80-4.73 (m, 1H), 4.63-4.56 (m, 1H), 4.47-4.40 (m, 1H), 4.19-4.07 (m, 2H), 2.57-2.54 (m, 1H), 2.02-1.93 (m, 1H), 1.79-1.70 (m, 1H), 1.25 (d, J=22.80 Hz, 3H), 1.20 (d, J=6.28 Hz, 3H), 1.19 (d, J=6.28 Hz, 3H), 1.11 (d, J=7.02 Hz, 3H); ³¹P NMR (162 MHz, DMSO-d₆) δ −4.86 (s, 1P); ¹⁹F NMR (376 MHz, DMSO-d₆) δ −158.75 (s, 1F); LC/MS: [(M+1)]⁺=488.5.

### Example 71

### Preparation of Compound 89A/89B

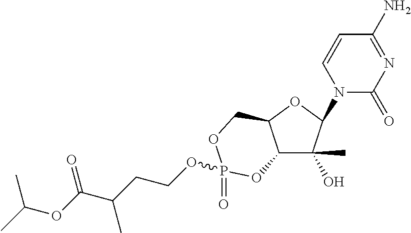

Compounds 89A/89B were made using the method described in Example 63 using appropriate nucleoside and intermediate compound A as starting material, followed by deprotection with TFA.

Isomer 89A: ¹H NMR (400 MHz, DMSO-d₆) δ 7.62 (dd, J=7.46 Hz, 4.73 Hz, 1H), 7.44 (brs, 1H), 7.34 (brs, 1H), 6.07 (brs, 1H), 5.99-5.98 (m, 1H), 5.81-5.79 (m, 1H), 4.89 (heptuplet, J=6.21 Hz, 1H), 4.70-4.61 (m, 1H), 4.56-4.49 (m, 1H), 4.19-4.07 (m, 3H), 4.00-3.95 (m, 1H), 2.60-2.55 (m, 1H), 2.06-1.97 (m, 1H), 1.85-1.75 (m, 1H), 1.19 (d, J=6.21 Hz, 6H), 1.12 (d, J=7.01 Hz, 3H), 1.06-1.05 (m, 3H); ³¹P NMR (162 MHz, DMSO-d₆) δ −5.99 (s, 1P); LC/MS: [(M+1)]⁺=462.4.

### Example 72

### Preparation of Compound 90A/90B

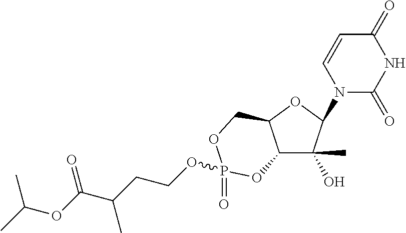

Compounds 90A/90B were made using the method described in Example 63 using appropriate nucleoside and intermediate compound A as starting material.

Isomer 90A: ¹H NMR (400 MHz, DMSO-d₆) δ 11.49 (brs, 1H), 7.76-7.69 (m, 1H), 6.08 (brs, 1H), 6.00-5.96 (m, 1H), 5.67-5.61 (m, 1H), 4.89 (heptuplet, J=6.31 Hz, 1H), 4.69-4.52 (m, 2H), 4.18-4.04 (m, 3H), 2.60-2.53 (m, 1H), 2.06-1.92 (m, 1H), 1.84-1.68 (m, 1H), 1.20-1.17 (m, 6H), 1.12-1.10 (m, 6H); ³¹P NMR (162 MHz, DMSO-d₆) δ −6.06 (s, 1P); LC/MS: [(M+1)]⁺=463.1.

The following compounds of the invention, set forth below in Table 1, were prepared using the methods described above in Examples 42 (Method A), 48 (Method B) and 52 (Method C), and substituting the appropriate reactants and/or reagents. Stereoisomeric products were separated using either Prep-HPLC or preparative Chiral-HPLC. The column having the heading “INT” identifies the intermediate compound used to make each exemplified compound.

TABLE 1

| | | | | | LC/ | | |
|---|---|---|---|---|---|---|---|
| | | | NMR | | MS | | |
| | | | ³¹P | Stereo | (M + | | METH- |
| No. | Structure | NMR ¹H (ppm) | (ppm) | P | 1)⁺ | INT | OD |
| 15A | 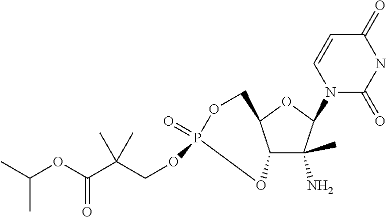 [SMILES: CC(C)OC(=O)C(C)(C)CO[P@@]1(=O)OC[C@H]2O[C@@H](n3ccc(=O)[nH]c3=O)[C@](C)(N)[C@@H]2O1] | | CDCl₃: −7.16 | S_{P} | 462 | L | A |
| 15B | 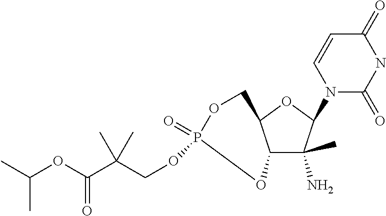 [SMILES: CC(C)OC(=O)C(C)(C)CO[P@]1(=O)OC[C@H]2O[C@@H](n3ccc(=O)[nH]c3=O)[C@](C)(N)[C@@H]2O1] | | CDCl₃: −3.90 | R_{P} | 462 | L | A |
| 16A | 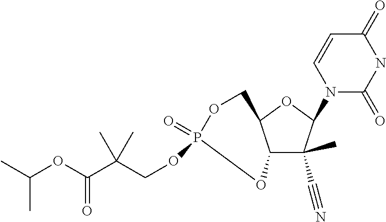 [SMILES: CC(C)OC(=O)C(C)(C)CO[P@@]1(=O)OC[C@H]2O[C@@H](n3ccc(=O)[nH]c3=O)[C@](C)(C#N)[C@@H]2O1] | | CDCl₃: −8.89 | S_{P} | 472 | L | A |
| 16B | 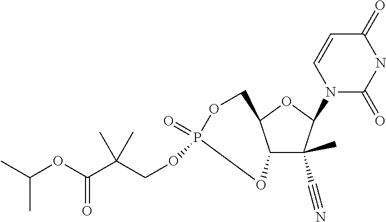 [SMILES: CC(C)OC(=O)C(C)(C)CO[P@]1(=O)OC[C@H]2O[C@@H](n3ccc(=O)[nH]c3=O)[C@](C)(C#N)[C@@H]2O1] | | CDCl₃: −5.38 | R_{P} | 472 | L | A |
| 17A | 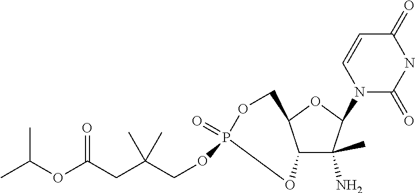 [SMILES: CC(C)OC(=O)CC(C)(C)CO[P@@]1(=O)OC[C@H]2O[C@@H](n3ccc(=O)[nH]c3=O)[C@](C)(N)[C@@H]2O1] | DMSO: 11.45 (1H, s), 7.65 (1H, d, J = 8.1 Hz), 5.90 (1H, s), 5.62 (1H, d, J = 8.1 Hz), 4.92- 4.85 (1H, m), 4.72- 4.55 (2H, m), 4.33- 4.25 (1H, m), 4.06 (1H, d, J = 9.5 Hz), 3.87- 3.84 (2H, m), 2.30- 2.29 (2H, m), 2.03 (2H, s), 1.18 (6H, d, J = 6.2 Hz), 1.02 (9H, d, | DMSO: −6.13 | S_{P} | 476 | M | A |
| | | J = 6.8 Hz) | | | | | |
| 17B | 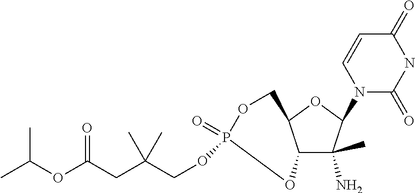 [SMILES: CC(C)OC(=O)CC(C)(C)CO[P@]1(=O)OC[C@H]2O[C@@H](n3ccc(=O)[nH]c3=O)[C@](C)(N)[C@@H]2O1] | DMSO: 11.46 (1H, s), 7.78 (1H, d, J = 8.2 Hz), 5.91 (1H, s), 5.61 (1H, d, J = 8.1 Hz), 4.95- 4.85 (1H, m), 4.68- 4.60 (2H, m), 4.48- 4.38 (2H, m), 3.88 (2H, ddd, J = 5.2, 9.4, 13.4 Hz), 2.24 (2H, s), 2.00 (2H, s), 1.19 (6H, d, J = 6.2 Hz), 1.02 (3 hours, s), 0.98 (6H, s) | DMSO: −4.67 | R_{P} | 476 | M | A |
| 18A | 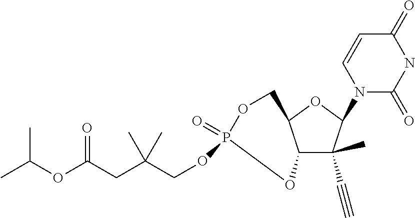 [SMILES: C#C[C@]1(C)[C@@H]2O[P@@](=O)(OCC(C)(C)CC(=O)OC(C)C)OC[C@H]2O[C@H]1n1ccc(=O)[nH]c1=O] | DMSO: 11.61 (1H, s), 7.69 (1H, d, J = 8.1 Hz), 6.27 (1H, s), 5.65 (1H, d, J = 8.1 Hz), 4.93- 4.83 (1H, m), 4.74- 4.65 (2H, m), 4.17 (2H, dd, J = 5.2, 5.2 Hz), 3.88 (2H, dd, J = 1.4, 4.8 Hz), 3.65 (1H, s), 2.32 (2H, dd, J = 14.5, 32.2 Hz), 1.21- 1.16 (9H, m), 1.01 (6H, d, J = 10.8 Hz) | DMSO: −5.79 | S_{P} | 485 | M | A |
| 18B | 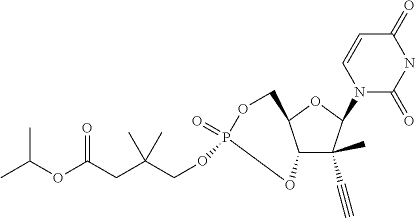 [SMILES: C#C[C@]1(C)[C@@H]2O[P@](=O)(OCC(C)(C)CC(=O)OC(C)C)OC[C@H]2O[C@H]1n1ccc(=O)[nH]c1=O] | DMSO: 11.58 (1H, s), 7.79 (1H, d, J = 8.1 Hz), 6.29 (1H, s), 5.65 (1H, d, J = 8.2 Hz), 4.95- 4.85 (1H, m), 4.74- 4.62 (3 hours, m), 4.32 (1H, dd, J = 9.4, 16.3 Hz), 3.89 (2H, dd, J = 1.1, 5.2 Hz), 3.63 (1H, s), 2.24 (2H, s), 1.20-1.16 (9H, m), 0.99 (6H, s) | DMSO: −5.79 | R_{P} | 485 | M | A |
| 19A | 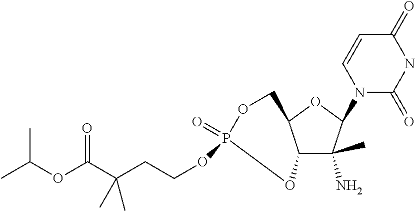 [SMILES: CC(C)OC(=O)C(C)(C)CCO[P@@]1(=O)OC[C@H]2O[C@@H](n3ccc(=O)[nH]c3=O)[C@](C)(N)[C@@H]2O1] | DMSO: 11.51 (1H, s), 7.70 (1H, d, J = 7.9 Hz), 5.90 (1H, s), 5.66 (1H, d, J = 8.1 Hz), 4.93- 4.83 (1H, m), 4.69- 4.59 (1H, m), 4.48 (1H, dd, J = 9.5, 9.5 Hz), 4.31-4.24 (1H, m), 4.10-4.03 (3 hours, m), 2.03 (2H, s), 1.96 (2H, t, J = 6.9 Hz), 1.18 (6H, d, J = 6.6 Hz), 1.16 (6H, d, J = 4.9 Hz), 1.02 (3 hours, s) | DMSO: −6.05 | S_{P} | 476 | N | A |
| 19B | 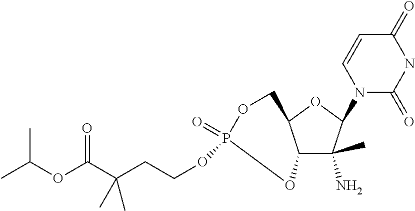 [SMILES: CC(C)OC(=O)C(C)(C)CCO[P@]1(=O)OC[C@H]2O[C@@H](n3ccc(=O)[nH]c3=O)[C@](C)(N)[C@@H]2O1] | DMSO: 11.47 (1H, s), 7.78 (1H, d, J = 8.1 Hz), 5.90 (1H, s), 5.61 (1H, d, J = 8.1 Hz), 4.92- 4.82 (1H, m), 4.67- 4.58 (2H, m), 4.47- 4.33 (2H, m), 4.11- 4.04 (2H, m), 1.99 (2H, s), 1.89 (2H, dd, J = 7.1, 7.1 Hz), 1.18 (6H, d, J = 6.2 Hz), 1.14 (6H, s), 1.02 (3 hours, s) | DMSO: −4.89 | R_{P} | 476 | N | A |
| 20A | 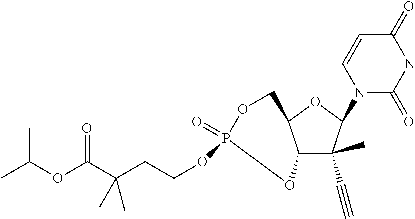 [SMILES: C#C[C@]1(C)[C@@H]2O[P@@](=O)(OCCC(C)(C)C(=O)OC(C)C)OC[C@H]2O[C@H]1n1ccc(=O)[nH]c1=O] | DMSO: 11.60 (1H, s), 7.74 (1H, d, J = 8.1 Hz), 6.28 (1H, s), 5.69 (1H, d, J = 8.1 Hz), 4.92- 4.82 (1H, m), 4.73- 4.63 (1H, m), 4.55 (1H, dd, J = 9.6, 9.6 Hz), 4.24-4.07 (4H, m), 3.64 (1H, s), 1.96 (2H, dd, J = 7.0, 7.0 Hz), 1.19-1.14 (15H, m) | DMSO: −6.82 | S_{P} | 485 | N | A |
| 20B | 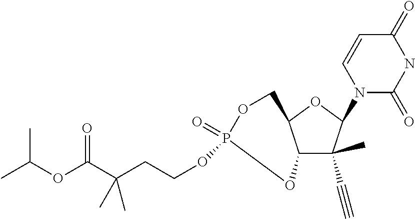 [SMILES: C#C[C@]1(C)[C@@H]2O[P@](=O)(OCCC(C)(C)C(=O)OC(C)C)OC[C@H]2O[C@H]1n1ccc(=O)[nH]c1=O] | DMSO: 11.57 (1H, s), 7.80 (1H, d, J = 8.2 Hz), 6.29 (1H, s), 5.65 (1H, d, J = 8.1 Hz), 4.92- 4.82 (1H, m), 4.74- 4.60 (3 hours, m), 4.31- 4.22 (1H, m), 4.09 (2H, q, J = 7.2 Hz), 3.60 (1H, s), 1.91 (2H, dd, J = 7.0, 7.0 Hz), 1.19 (3 hours, s), 1.18 (6H, s), 1.14 (6H, s) | DMSO: −5.79 | R_{P} | 485 | N | A |
| 21A | 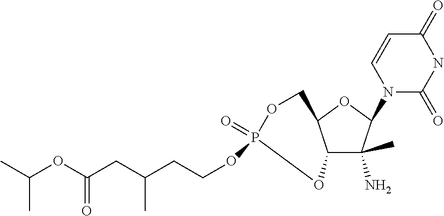 [SMILES: CC(CCO[P@@]1(=O)OC[C@H]2O[C@@H](n3ccc(=O)[nH]c3=O)[C@](C)(N)[C@@H]2O1)CC(=O)OC(C)C] | DMSO: 11.48 (1H, s), 7.71 (1H, d, J = 7.9 Hz), 5.89 (1H, s), 5.64 (1H, d, J = 8.1 Hz), 4.93- 4.85 (1H, m), 4.68- 4.52 (2H, m), 4.29- 4.23 (1H, m), 4.14- 4.06 (3 hours, m), 2.31 (1H, ddd, J = 2.1, 5.8, 14.9 Hz), 2.18-2.11 (1H, m), 2.00 (3 hours, s), 1.79-1.69 (1H, m), 1.60-1.55 (1H, m), 1.19-1.16 (6H, m), | DMSO: −5.91; −5.94 | S_{P} | 476 | P | A |
| | | 1.02 (3 hours, s), 0.94- | | | | | |
| | | 0.92 (3 hours, m) | | | | | |
| 21B | 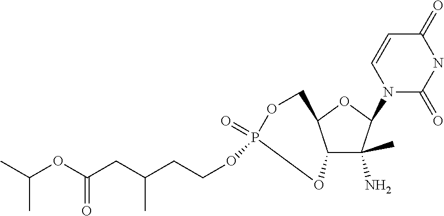 [SMILES: CC(CCO[P@]1(=O)OC[C@H]2O[C@@H](n3ccc(=O)[nH]c3=O)[C@](C)(N)[C@@H]2O1)CC(=O)OC(C)C] | DMSO: 11.46 (1H, s), 7.77 (1H, d, J = 8.2 Hz), 5.90 (1H, s), 5.61 (1H, d, J = 8.1 Hz), 4.94- 4.86 (1H, m), 4.67- 4.59 (2H, m), 4.47- 4.36 (2H, m), 4.11 (2H, dd, J = 6.9, 13.6 Hz), 2.36-2.29 (1H, m), 2.14-1.97 (4H, m), 1.73-1.64 (1H, m), 1.57-1.48 (1H, m), 1.18 (6H, d, J = 6.2 Hz), 1.02 (3 hours, s), | DMSO: −4.67 | R_{P} | 476 | P | A |
| | | 0.91 (3 hours, d, J = 6.5 | | | | | |
| | | Hz) | | | | | |
| 22A | 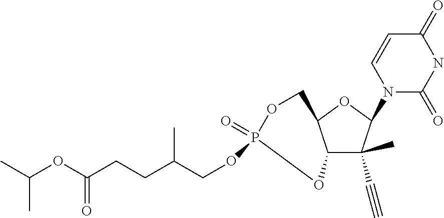 [SMILES: C#C[C@]1(C)[C@@H]2O[P@@](=O)(OCC(C)CCC(=O)OC(C)C)OC[C@H]2O[C@H]1n1ccc(=O)[nH]c1=O] | DMSO: 11.58 (1H, s), 7.76 (1H, dd, J = 8.3, 8.3 Hz), 6.28 (1H, s), 5.67 (1H, d, J = 8.1 Hz), 4.92-4.84 (1H, m), 4.72-4.61 (2H, m), 4.25-4.05 (3 hours, m), 3.97-3.89 (1H, m), 3.64 (1H, s), 2.45- 2.27 (2H, m), 1.71- 1.61 (2H, m), 1.50- 1.37 (1H, m), 1.18 (9H, d, J = 6.2 Hz), 1.08 (3 hours, d, J = 7.0 Hz) | DMSO: −6.66; −6.69 | -S_{P} | 485 | Q | A |
| 22B | 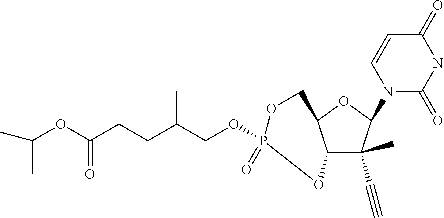 [SMILES: C#C[C@]1(C)[C@@H]2O[P@](=O)(OCC(C)CCC(=O)OC(C)C)OC[C@H]2O[C@H]1n1ccc(=O)[nH]c1=O] | DMSO: 11.60-11.56 (1H, m), 7.80 (1H, d, J = 8.2 Hz), 6.29 (1H, s), 5.65 (1H, d, J = 8.1 Hz), 4.93-4.85 (1H, m), 4.74-4.60 (3 hours, m), 4.34-4.25 (1H, m), 4.11-4.04 (1H, m), 3.96-3.90 (1H, m), 3.60 (1H, dd, J = 1.9, 7.6 Hz), 2.43- 2.27 (2H, m), 1.67- 1.61 (2H, m), 1.47- 1.33 (1H, m), 1.19 | DMSO: −5.52; −5.65 | R_{P} | 485 | Q | A |
| | | (9H, d, J = 6.2 Hz), 1.07 | | | | | |
| | | (2H, d, J = 7.0 Hz), 0.90 | | | | | |
| | | (1H, d, J = 6.8 Hz) | | | | | |
| 23A | 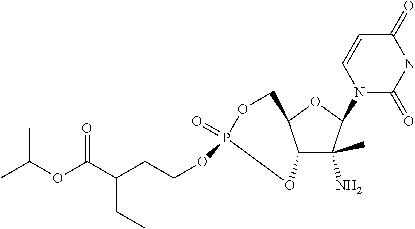 [SMILES: CCC(CCO[P@@]1(=O)OC[C@H]2O[C@@H](n3ccc(=O)[nH]c3=O)[C@](C)(N)[C@@H]2O1)C(=O)OC(C)C] | DMSO: 11.48 (1H, s), 7.74-7.70 (1H, m), 5.89 (1H, s), 5.64 (1H, d, J = 8.1 Hz), 4.95- 4.88 (1H, m), 4.68- 4.51 (2H, m), 4.30- 4.23 (1H, m), 4.13- 4.02 (3 hours, m), 2.44- 2.37 (1H, m), 2.02- 1.94 (3 hours, m), 1.87- 1.80 (1H, m), 1.57- 1.50 (2H, m), 1.19 (6H, d, J = 6.2 Hz), 1.02- 1.02 (3 hours, m), 0.85 (3 hours, dd, | DMSO: −6.01 | S_{P} | 476 | E | A |
| | | J = 7.4, 7.4 Hz); | | | | | |
| 23B | 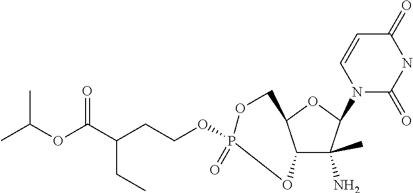 [SMILES: CCC(CCO[P@]1(=O)OC[C@H]2O[C@@H](n3ccc(=O)[nH]c3=O)[C@](C)(N)[C@@H]2O1)C(=O)OC(C)C] | DMSO: 11.46 (1H, s), 7.77 (1H, d, J = 7.9 Hz), 5.90 (1H, s), 5.61 (1H, d, J = 8.1 Hz), 4.96- 4.89 (1H, m), 4.66- 4.60 (2H, m), 4.47- 4.36 (2H, m), 4.12- 3.99 (2H, m), 2.40- 2.34 (1H, m), 1.99- 1.98 (2H, m), 1.95- 1.72 (2H, m), 1.58- 1.49 (2H m), 1.19 | DMSO: −4.76; −4.81 | R_{P} | 476 | E | A |
| | | (6H, dd, J = 2.7, 6.3 | | | | | |
| | | Hz), 1.02 (3 hours, s), | | | | | |
| | | 0.84 (3 hours, dd, | | | | | |
| | | J = 7.4, 7.4 Hz) | | | | | |
| 24A | 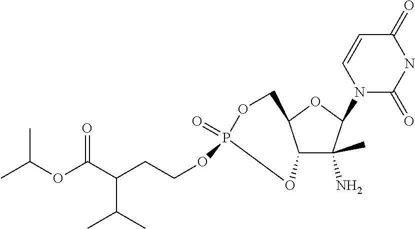 [SMILES: CC(C)OC(=O)C(CCO[P@@]1(=O)OC[C@H]2O[C@@H](n3ccc(=O)[nH]c3=O)[C@](C)(N)[C@@H]2O1)C(C)C] | DMSO: 11.48 (1H, s), 7.72 (1H, d, J = 7.9 Hz), 5.90 (1H, s), 5.63 (1H, d, J = 8.1 Hz), 4.98- 4.88 (1H, m), 4.63 (1H, d, J = 36.1 Hz), 4.52 (1H, ddd, J = 8.5, 8.5, 8.5 Hz), 4.31- 4.24 (1H, m), 4.15- 4.01 (3 hours, m), 2.29- 2.23 (1H, m), 2.02 (2H, s), 1.99-1.82 (3 hours, m), 1.19 (6H, d, J = 6.3 Hz), 1.05-1.00 | DMSO: −6.03 | S_{P} | 490 | G | A |
| | | (3 hours, m), 0.89 (6H, | | | | | |
| | | dd, J = 7.1, 7.1 Hz) | | | | | |
| 24B | 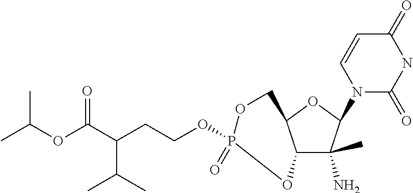 [SMILES: CC(C)OC(=O)C(CCO[P@]1(=O)OC[C@H]2O[C@@H](n3ccc(=O)[nH]c3=O)[C@](C)(N)[C@@H]2O1)C(C)C] | DMSO: 11.46 (1H, s), 7.80-7.75 (1H, m), 5.90 (1H, s), 5.61 (1H, d, J = 8.1 Hz), 4.98- 4.88 (1H, m), 4.66- 4.59 (2H, m), 4.47- 4.35 (2H, m), 4.11- 3.93 (2H, m), 2.28- 2.20 (1H, m), 1.98 (2H, d, J = 5.9 Hz), 1.89- 1.79 (3 hours, m), 1.19 (6H, dd, J = 3.2, | DMSO: −4.77; −4.84 | R_{P} | 490 | G | A |
| | | 6.2 Hz), 1.02 (3 hours, | | | | | |
| | | s), 0.89 (6H, dd, J = 6.9, | | | | | |
| | | 8.6 Hz) | | | | | |
| 25A | 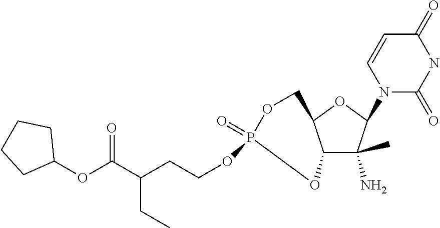 [SMILES: CCC(CCO[P@@]1(=O)OC[C@H]2O[C@@H](n3ccc(=O)[nH]c3=O)[C@](C)(N)[C@@H]2O1)C(=O)OC1CCCC1] | DMSO: 11.48 (1H, s), 7.73 (1H, d, J = 7.6 Hz), 5.90 (1H, d, J = 0.6 Hz), 5.64 (1H, d, J = 8.1 Hz), 5.11-5.07 (1H, m), 4.59-4.51 (2H, m), 4.30-4.24 (1H, m), 4.10-4.02 (3 hours, m), 2.43-2.36 (1H, m), 2.03 (2H, s), 1.86- 1.78 (3 hours, m), 1.65- 1.51 (9H, m), 1.02- 1.02 (3 hours, m), 0.84 (3 hours, dd, J = 7.4, 7.4 | DMSO: −6.01 | S_{P} | 502 | F | A |
| | | Hz) | | | | | |
| 25B | 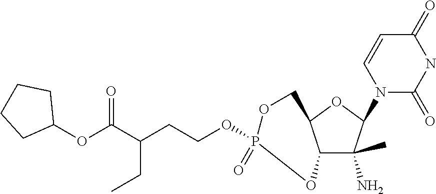 [SMILES: CCC(CCO[P@]1(=O)OC[C@H]2O[C@@H](n3ccc(=O)[nH]c3=O)[C@](C)(N)[C@@H]2O1)C(=O)OC1CCCC1] | DMSO: 11.46 (1H, s), 7.77 (1H, d, J = 8.2 Hz), 5.90 (1H, s), 5.61 (1H, d, J = 8.2 Hz), 5.11- 5.07 (1H, m), 4.66- 4.59 (2H, m), 4.47- 4.36 (2H, m), 4.11- 4.01 (2H, m), 2.40- 2.35 (1H, m), 1.99 (2H, d, J = 3.3 Hz), 1.93- 1.73 (4H, m), 1.67- 1.50 (8H, m), 1.02 (3 | DMSO: −4.75; −4.78 | R_{P} | 502 | F | A |
| | | hours, s), 0.84 (3 | | | | | |
| | | hours, dd, J = 7.4, 7.4 | | | | | |
| | | Hz) | | | | | |
| 26A | 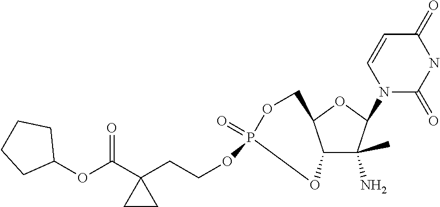 [SMILES: C[C@@]1(N)[C@@H]2O[P@@](=O)(OCCC3(C(=O)OC4CCCC4)CC3)OC[C@H]2O[C@H]1n1ccc(=O)[nH]c1=O] | DMSO: 11.48 (1H, s), 7.69 (1H, d, J = 7.9 Hz), 5.89 (1H, s), 5.65 (1H, d, J = 8.1 Hz), 5.07- 5.02 (1H, m), 4.63 (1H, ddd, J = 4.4, 8.6, 21.7 Hz), 4.50 (1H, dd, J = 9.7, 9.7 Hz), 4.28- 4.05 (4H, m), 2.02 (2H, s), 1.98-1.90 (2H, m), 1.82-1.72 (2H, m), 1.66-1.51 (6H, m), 1.11-1.06 | DMSO: −6.03 | S_{P} | 500 | I | A |
| | | (2H, m), 1.01 (3 hours, | | | | | |
| | | s), 0.88-0.83 (2H, m); | | | | | |
| 26B | 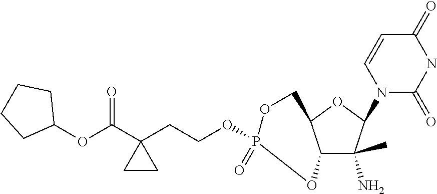 [SMILES: C[C@@]1(N)[C@@H]2O[P@](=O)(OCCC3(C(=O)OC4CCCC4)CC3)OC[C@H]2O[C@H]1n1ccc(=O)[nH]c1=O] | DMSO: 11.47 (1H, s), 7.78 (1H, d, J = 8.1 Hz), 5.90 (1H, s), 5.61 (1H, d, J = 8.1 Hz), 5.08- 5.03 (1H, m), 4.65- 4.58 (2H, m), 4.46- 4.33 (2H, m), 4.17 (2H, q, J = 7.5 Hz), 1.98 (2H, s), 1.91-1.73 (4H, m), 1.67-1.52 (6H, m), 1.06 (2H, dd, J = 3.6, 6.1 Hz), 1.03 (3 | DMSO: −4.71 | R_{P} | 500 | I | A |
| | | hours, s), 0.87-0.83 | | | | | |
| | | (2H, m) | | | | | |
| 27A | 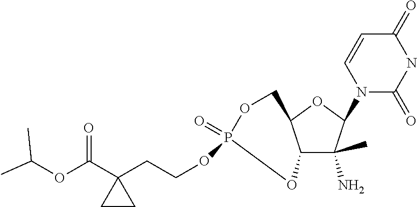 [SMILES: CC(C)OC(=O)C1(CCO[P@@]2(=O)OC[C@H]3O[C@@H](n4ccc(=O)[nH]c4=O)[C@](C)(N)[C@@H]3O2)CC1] | DMSO: 11.51 (1H, s), 7.70 (1H, d, J = 8.1 Hz), 5.89 (1H, s), 5.65 (1H, d, J = 8.2 Hz), 4.90- 4.81 (1H, m), 4.69- 4.58 (1H, m), 4.54- 4.48 (1H, m), 4.28- 4.06 (4H, m), 2.02 (2H, s), 1.96-1.92 (2H, m), 1.16 (6H, d, J = 6.2 Hz), 1.12-1.07 (2H, m), 1.01 (3 hours, s), 0.88-0.83 (2H, m) | DMSO: −6.04 | S_{P} | 474 | H | A |
| 27B | 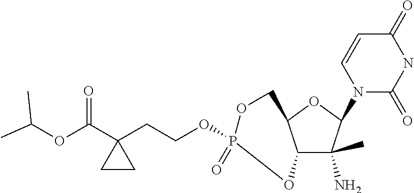 [SMILES: CC(C)OC(=O)C1(CCO[P@]2(=O)OC[C@H]3O[C@@H](n4ccc(=O)[nH]c4=O)[C@](C)(N)[C@@H]3O2)CC1] | DMSO: 11.46 (1H, s), 7.78 (1H, d, J = 8.1 Hz), 5.90 (1H, s), 5.61 (1H, dd, J = 2.0, 8.0 Hz), 4.89-4.82 (1H, m), 4.66-4.59 (2H, m), 4.47-4.34 (2H, m), 4.18 (2H, q, J = 7.5 Hz), 1.99 (2H, s), 1.92- 1.87 (2H, m), 1.17 (6H, d, J = 6.3 Hz), 1.07 (2H, dd, J = 3.9, 6.9 | DMSO: −4.78 | R_{P} | 474 | H | A |
| | | Hz), 1.02 (3 hours, s), | | | | | |
| | | 0.87-0.83 (2H, m) | | | | | |
| 28A | 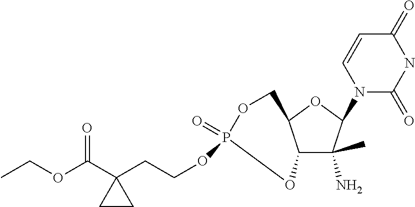 [SMILES: CCOC(=O)C1(CCO[P@@]2(=O)OC[C@H]3O[C@@H](n4ccc(=O)[nH]c4=O)[C@](C)(N)[C@@H]3O2)CC1] | DMSO: 11.48 (1H, s), 7.70 (1H, d, J = 7.9 Hz), 5.89 (1H, s), 5.67- 5.63 (1H, m), 4.69- 4.58 (1H, m), 4.51 (1H, dd, J = 9.5, 9.5 Hz), 4.26 (1H, qdd, J = 5.2, 4.9, 4.9 Hz), 4.18 (2H, dd, J = 7.0, 15.3 Hz), 4.10-4.01 (3 hours, m), 2.01 (2H, s), 1.99-1.93 (2H, m), 1.18-1.10 (5H, m), | DMSO: −6.04 | S_{P} | 460 | J | A |
| | | 1.01 (3 hours, s), 0.90- | | | | | |
| | | 0.85 (2H, m) | | | | | |
| 28B | 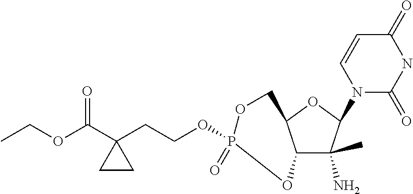 [SMILES: CCOC(=O)C1(CCO[P@]2(=O)OC[C@H]3O[C@@H](n4ccc(=O)[nH]c4=O)[C@](C)(N)[C@@H]3O2)CC1] | DMSO: 11.46 (1H, s), 7.78 (1H, d, J = 8.1 Hz), 5.90 (1H, s), 5.61 (1H, d, J = 8.1 Hz), 4.67- 4.58 (2H, m), 4.47- 4.34 (2H, m), 4.22- 4.15 (2H, m), 4.05 (2H, q, J = 7.1 Hz), 1.99 (2H, s), 1.90 (2H, dd, J = 7.1, 7.1 Hz), 1.17 (3 hours, t, J = 7.2 Hz), 1.10 (2H, dd, J = 3.5, | DMSO: −4.82 | R_{P} | 460 | J | A |
| | | 7.0 Hz), 1.02 (3 hours, | | | | | |
| | | s), 0.89-0.85 (2H, m) | | | | | |
| 29A | 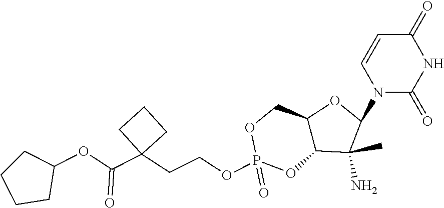 | DMSO: 11.50 (1H, s), 7.72 (1H, d, J = 8.1 Hz), 5.91 (1H, s), 5.68 (1H, d, J = 7.9 Hz), 5.11 (1H, dd, J = 5.6, 5.6 Hz), 4.70-4.59 (1H, m), 4.47-4.40 (1H, m), 4.31-4.23 (1H, m), 4.08-3.94 (3 hours, m), 2.35-2.27 (2H, m), 2.23-2.16 (2H, m), 2.06 (2H, s), 2.00- 1.89 (3 hours, m), 1.86- | DMSO: −6.13 | S_{P} | 514 | K | A |
| | | 1.76 (3 hours, m), | | | | | |
| | | 1.68-1.53 (6H, m), | | | | | |
| | | 1.03 (3 hours, s) | | | | | |
| 29B | 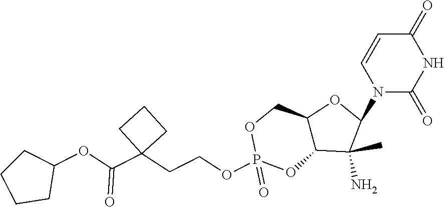 | DMSO: 11.45 (1H, s), 7.77 (1H, d, J = 8.2 Hz), 5.90 (1H, s), 5.61 (1H, d, J = 8.1 Hz), 5.12- 5.07 (1H, m), 4.66- 4.58 (2H, m), 4.46- 4.33 (2H, m), 4.01 (2H, q, J = 6.9 Hz), 2.31- 2.25 (2H, m), 2.12 (2H, dd, J = 6.7, 6.7 Hz), 1.99-1.78 (8H, m), 1.68-1.53 (6H, m), 1.02 (3 hours, s) | DMSO: −4.87 | R_{P} | 514 | K | A |
| 30A | 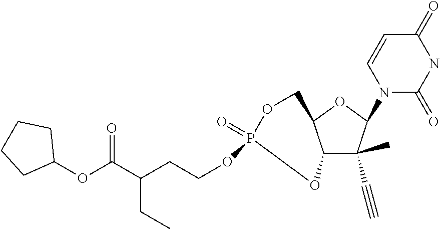 [SMILES: C#C[C@]1(C)[C@@H]2O[P@@](=O)(OCCC(CC)C(=O)OC3CCCC3)OC[C@H]2O[C@H]1n1ccc(=O)[nH]c1=O] | DMSO: 11.59 (1H, s), 7.77-7.75 (1H, m), 6.28 (1H, s), 5.66 (1H, d, J = 8.2 Hz), 5.11- 5.07 (1H, m), 4.72- 4.57 (2H, m), 4.27- 4.04 (4H, m), 3.64 (1H, s), 2.41-2.36 (1H, m), 2.00-1.93 (1H, m), 1.85-1.78 (3 hours, m), 1.65-1.51 (8H, m), 1.18 (3 hours, d, J = 3.0 Hz), 0.84 (3 hours, dd, J = 7.4, 7.4 | DMSO: −6.77 | S_{P} | 511 | F | A |
| | | Hz) | | | | | |
| 30B | 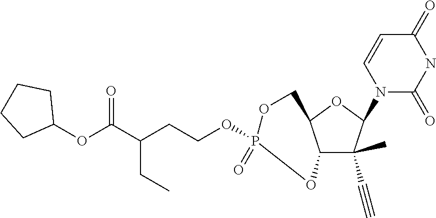 [SMILES: C#C[C@]1(C)[C@@H]2O[P@](=O)(OCCC(CC)C(=O)OC3CCCC3)OC[C@H]2O[C@H]1n1ccc(=O)[nH]c1=O] | 11.58 (1H, s), 7.80 (1H, d, J = 8.1 Hz), 6.29 (1H, s), 5.65 (1H, dd, J = 1.9, 8.1 Hz), 5.12- 5.07 (1H, m), 4.73- 4.60 (3 hours, m), 4.32- 4.24 (1H, m), 4.12- 4.02 (2H, m), 3.59 (1H, d, J = 2.1 Hz), 2.39- 2.31 (1H, m), 1.93- 1.78 (4H, m), 1.67- 1.50 (8H, m), 1.17 (3 hours, s), 0.84 (3 hours, dd, J = 7.5, 7.5 | DMSO: −5.63; −5.70 | R_{P} | 511 | F | A |
| | | Hz) | | | | | |
| 31A | 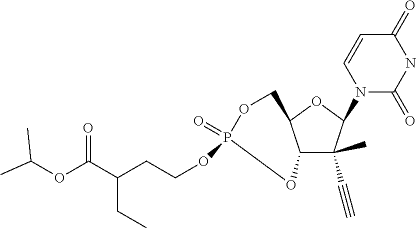 [SMILES: C#C[C@]1(C)[C@@H]2O[P@@](=O)(OCCC(CC)C(=O)OC(C)C)OC[C@H]2O[C@H]1n1ccc(=O)[nH]c1=O] | DMSO: 11.58 (1H, s), 7.74 (1H, d, J = 8.1 Hz), 6.29 (1H, s), 5.66 (1H, d, J = 8.2 Hz), 4.95- 4.88 (1H, m), 4.72- 4.56 (2H, m), 4.23 (1H, dd, J = 8.2, 19.9 Hz), 4.16 (1H, dd, J = 4.1, 10.0 Hz), 4.08 (2H, ddd, J = 7.4, 7.4, 7.4 Hz), 3.64 (1H, s), 2.43-2.39 (1H, m), 1.97-1.94 (1H, m), 1.86 (1H, s), 1.59- | DMSO: −6.79 | S_{P} | 485 | E | A |
| | | 1.50 (2H, m), 1.19 | | | | | |
| | | (9H, d, J = 6.2 Hz), 0.85 | | | | | |
| | | (3 hours, dd, J = 7.4, 7.4 | | | | | |
| | | Hz) | | | | | |
| 31B | 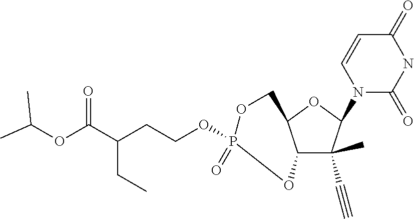 [SMILES: C#C[C@]1(C)[C@@H]2O[P@](=O)(OCCC(CC)C(=O)OC(C)C)OC[C@H]2O[C@H]1n1ccc(=O)[nH]c1=O] | DMSO: 11.56 (1H, s), 7.79 (1H, d, J = 8.2 Hz), 6.29 (1H, s), 5.64 (1H, d, J = 8.1 Hz), 4.96- 4.89 (1H, m), 4.74- 4.60 (3 hours, m), 4.31- 4.23 (1H, m), 4.12- 4.01 (2H, m), 3.58 (1H, d, J = 3.0 Hz), 2.39- 2.34 (1H, m), 1.96- 1.75 (2H, m), 1.57- 1.49 (2H, m), 1.21- 1.16 (9H, m), 0.84 (3 hours, dd, J = 7.5, 7.5 | DMSO: −5.63; −5.70 | R_{P} | 485 | E | A |
| | | Hz) | | | | | |
| 32A | 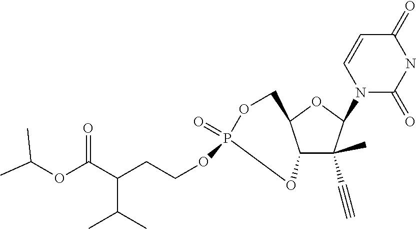 [SMILES: C#C[C@]1(C)[C@@H]2O[P@@](=O)(OCCC(C(=O)OC(C)C)C(C)C)OC[C@H]2O[C@H]1n1ccc(=O)[nH]c1=O] | DMSO: 11.58 (1H, s), 7.78-7.74 (1H, m), 6.28 (1H, s), 5.68- 5.64 (1H, m), 4.97- 4.87 (1H, m), 4.73- 4.55 (2H, m), 4.28- 4.01 (4H, m), 3.64 (1H, s), 2.30-2.22 (1H, m), 1.99-1.82 (3 hours, m), 1.19 (9H, dd, J = 2.1, 6.2 Hz), 0.89 (6H, dd, J = 7.4, 7.4 Hz) | DMSO: −6.79 | S_{P} | 499 | G | A |
| 32B | 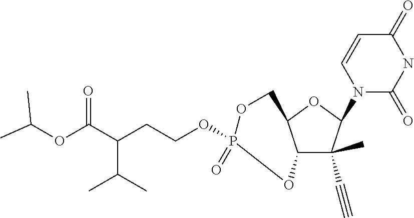 [SMILES: C#C[C@]1(C)[C@@H]2O[P@](=O)(OCCC(C(=O)OC(C)C)C(C)C)OC[C@H]2O[C@H]1n1ccc(=O)[nH]c1=O] | DMSO: 11.57 (1H, s), 7.80 (1H, d, J = 8.1 Hz), 6.29 (1H, s), 5.65 (1H, dd, J = 2.2, 8.1 Hz), 4.99-4.88 (1H, m), 4.74-4.60 (3 hours, m), 4.31-4.22 (1H, m), 4.13-3.94 (2H, m), 3.58 (1H, d, J = 3.0 Hz), 2.28-2.20 (1H, m), 1.91-1.80 (3 hours, m), 1.22-1.16 (9H, m), 0.92-0.86 (6H, m) | DMSO: −5.63; −5.69 | R_{P} | 499 | G | A |
| 33A | 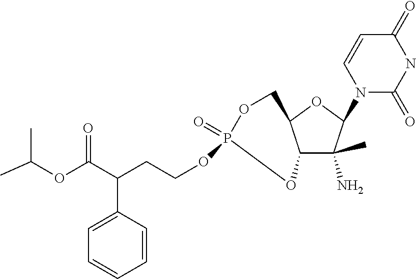 [SMILES: CC(C)OC(=O)C(CCO[P@@]1(=O)OC[C@H]2O[C@@H](n3ccc(=O)[nH]c3=O)[C@](C)(N)[C@@H]2O1)c1ccccc1] | DMSO: 11.51 (1H, s), 7.74 (1H, dd, J = 7.8, 7.8 Hz), 7.37-7.27 (5H, m), 5.88 (1H, d, J = 2.1 Hz), 5.67 (1H, dd, J = 7.8, 7.8 Hz), 4.93-4.85 (1H, m), 4.69-4.51 (2H, m), 4.30-4.21 (1H, m), 4.13-3.92 (3 hours, m), 3.85-3.74 (1H, m), 2.42 (1H, dt, J = 5.9, 15.2 Hz), 2.15- 2.05 (1H, m), 2.01 (2H, s), 1.17 (3 hours, dd, J = 3.6, 6.3 Hz), 1.06 (3 hours, d, J = 6.2 | DMSO: −6.00 | S_{P} | 524 | O | A |
| | | Hz), 0.98 (3 hours, d, | | | | | |
| | | J = 15.9 Hz) | | | | | |
| 33B | 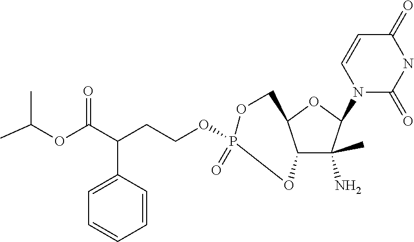 | 11.47 (1H, s), 7.77 (1H, d, J = 8.1 Hz), 7.39- 7.34 (2H, m), 7.29 (3 hours, d, J = 6.7 Hz), 5.90 (1H, s), 5.61 (1H, d, J = 8.1 Hz), 4.95- 4.84 (1H, m), 4.66- 4.57 (2H, m), 4.48- 4.33 (2H, m), 4.07- 3.92 (2H, m), 3.77- 3.70 (1H, m), 2.41- 2.31 (1H, m), 2.07- 1.91 (3 hours, m), 1.18 (3 hours, d, J = 6.2 Hz), 1.08-1.00 (6H, m) | DMSO: −4.75; −4.83 | R_{P} | 524 | O | A |
| 34A | 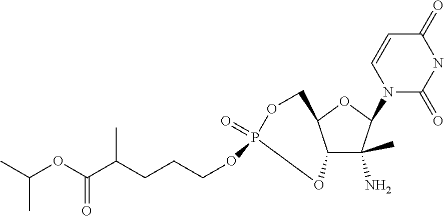 [SMILES: CC(C)OC(=O)C(C)CCCO[P@@]1(=O)OC[C@H]2O[C@@H](n3ccc(=O)[nH]c3=O)[C@](C)(N)[C@@H]2O1] | 11.50 (1H, s), 7.73 (1H, d, J = 7.9 Hz), 5.89 (1H, s), 5.64 (1H, d, J = 8.1 Hz), 4.93-4.83 (1H, m), 4.68-4.54 (2H, m), 4.29-4.22 (1H, m), 4.13-4.01 (3 hours, m), 2.40 (1H, dd, J = 7.0, 13.6 Hz), 2.06-1.99 (2H, m), 1.70-1.59 (3 hours, m), 1.51-1.43 (1H, m), 1.17 (6H, d, J = 6.3 Hz), 1.07 (3 hours, d, | DMSO: −5.92 | S_{P} | 476 | R | A |
| | | J = 6.9 Hz), 1.02 (3 | | | | | |
| | | hours, s) | | | | | |
| 34B | 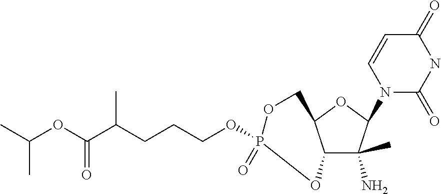 [SMILES: CC(C)OC(=O)C(C)CCCO[P@]1(=O)OC[C@H]2O[C@@H](n3ccc(=O)[nH]c3=O)[C@](C)(N)[C@@H]2O1] | DMSO: 11.46 (1H, s), 7.78 (1H, d, J = 8.1 Hz), 5.90 (1H, s), 5.61 (1H, d, J = 8.1 Hz), 4.94- 4.84 (1H, m), 4.67- 4.59 (2H, m), 4.47- 4.34 (2H, m), 4.06 (2H, d, J = 4.1 Hz), 2.44- 2.37 (1H, m), 1.99 (2H, s), 1.61 (3 hours, d, J = 3.0 Hz), 1.48- 1.40 (1H, m), 1.18 | DMSO: −4.62 | R _{P} | 476 | R | A |
| | | (6H, d, J = 6.2 Hz), 1.07 | | | | | |
| | | (3 hours, d, J = 6.9 Hz), | | | | | |
| | | 1.03 (3 hours, s) | | | | | |
| 40A | 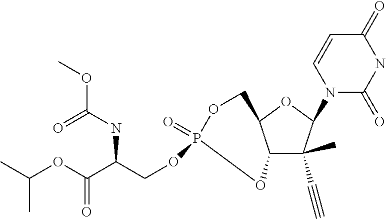 [SMILES: C#C[C@]1(C)[C@@H]2O[P@@](=O)(OC[C@H](NC(=O)OC)C(=O)OC(C)C)OC[C@H]2O[C@H]1n1ccc(=O)[nH]c1=O] | DMSO: 11.63 (1H, s), 7.79 (1H, d, J = 8.2 Hz), 7.61 (1H, d, J = 8.2 Hz), 6.28 (1H, s), 5.71 (1H, J = 8.2 Hz), 4.97- 4.90 (1H, m), 4.75- 4.57 (2H, m), 4.50- 4.43 (1H, m), 4.33- 4.13 (4H, m), 3.65 (1H, s), 3.57 (3 hours, s), 1.22-1.17 (9H, m) | DMSO: −7.04 | S_{P} | 516 | T | C |
| 40B | 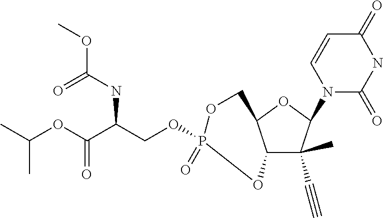 [SMILES: C#C[C@]1(C)[C@@H]2O[P@](=O)(OC[C@H](NC(=O)OC)C(=O)OC(C)C)OC[C@H]2O[C@H]1n1ccc(=O)[nH]c1=O] | DMSO: 11.57 (1H, s), 7.80 (2H, dd, J = 7.7, 7.7 Hz), 6.29 (1H, s), 5.65 (1H, d, J = 8.1 Hz), 4.99-4.88 (1H, m), 4.73-4.64 (3 hours, m), 4.41-4.22 (4H, m), 3.58 (3 hours, s), 3.54 (1H, s), 1.22- 1.16 (9H, m) | DMSO: −5.88 | R_{P} | 516 | T | C |
| 41A | 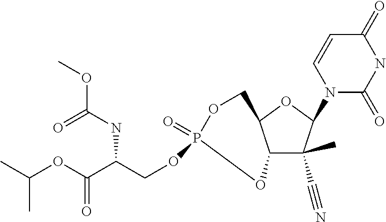 [SMILES: COC(=O)N[C@H](CO[P@@]1(=O)OC[C@H]2O[C@@H](n3ccc(=O)[nH]c3=O)[C@](C)(C#N)[C@@H]2O1)C(=O)OC(C)C] | DMSO: 11.73 (1H, s), 7.86 (1H, d, J = 8.6 Hz), 7.71 (1H, d, J = 8.1 Hz), 6.50 (1H, s), 5.78 (1H, d, J = 8.1 Hz), 5.01- 4.90 (1H, m), 4.78- 4.60 (2H, m), 4.54- 4.44 (2H, m), 4.39- 4.18 (3 hours, m), 3.56 (3 hours, s), 1.34 (3 hours, s), 1.21 (6H, d, J = 6.2 Hz) | DMSO: −7.84 | S_{P} | 517 | U | B |
| 41B | 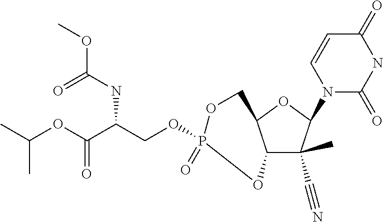 [SMILES: COC(=O)N[C@H](CO[P@]1(=O)OC[C@H]2O[C@@H](n3ccc(=O)[nH]c3=O)[C@](C)(C#N)[C@@H]2O1)C(=O)OC(C)C] | DMSO: 11.68 (1H, s), 7.81 (2H, dd, J = 8.1, 8.1 Hz), 6.49 (1H, s), 5.71 (1H, dd, J = 1.9, 8.1 Hz), 4.96-4.91 (2H, m), 4.77-4.66 (2H, m), 4.43-4.27 (4H, m), 3.32 (3 hours, s), 1.34 (3 hours, s), 1.22-1.18 (6H, m) | DMSO: −6.18 | R_{P} | 517 | U | B |
| 42A | 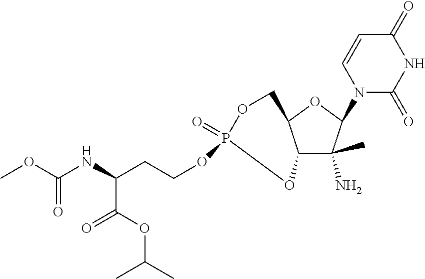 [SMILES: COC(=O)N[C@@H](CCO[P@@]1(=O)OC[C@H]2O[C@@H](n3ccc(=O)[nH]c3=O)[C@](C)(N)[C@@H]2O1)C(=O)OC(C)C] | 11.48 (1H, s), 7.67 (2H, d, J = 7.9 Hz), 5.89 (1H, s), 5.65 (1H, d, J = 8.2 Hz), 4.96-4.86 (1H, m), 4.70-4.53 (2H, m), 4.31-4.23 (1H, m), 4.14-4.05 (4H, m), 3.32 (3 hours, s), 2.18-2.08 (1H, m), 2.06-1.97 (3 hours, m), 1.19 (6H, dd, J = 3.2, 6.2 Hz), 1.02 (3 hours, s) | DMSO: −6.14 | S_{P} | 521 | V | A |
| 42B | 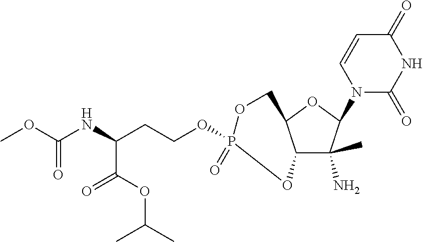 [SMILES: COC(=O)N[C@@H](CCO[P@]1(=O)OC[C@H]2O[C@@H](n3ccc(=O)[nH]c3=O)[C@](C)(N)[C@@H]2O1)C(=O)OC(C)C] | 11.47 (1H, s), 7.77 (1H, d, J = 8.1 Hz), 7.64 (1H, d, J = 8.1 Hz), 5.90 (1H, s), 5.62 (1H, d, J = 8.1 Hz), 4.95-4.87 (1H, m), 4.65-4.60 (2H, m), 4.49-4.35 (2H, m), 4.14-4.05 (3 hours, m), 3.55 (3 hours, s), 2.12-2.05 (1H, m), 1.99 (2H, s), 1.96-1.88 (1H, m), 1.20 (6H, dd, J = 3.7, 6.3 Hz), 1.02 (3 hours, s) | DMSO: −4.87 | R_{P} | 521 | V | A |
| 43A | 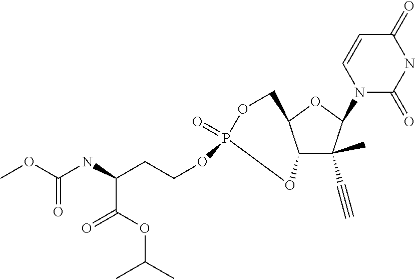 [SMILES: C#C[C@]1(C)[C@@H]2O[P@@](=O)(OCC[C@H](NC(=O)OC)C(=O)OC(C)C)OC[C@H]2O[C@H]1n1ccc(=O)[nH]c1=O] | 11.59 (1H, s), 7.72 (1H, d, J = 7.8 Hz), 7.68 (1H, d, J = 6.6 Hz), 6.28 (1H, s), 5.68 (1H, d, J = 8.1 Hz), 4.96-4.86 (1H, m), 4.73-4.60 (2H, m), 4.24-4.10 (5H, m), 3.64 (1H, s), 3.54 (3 hours, s), 2.21- 2.12 (1H, m), 2.04- 1.95 (1H, m), 1.19 (9H, dd, J = 2.9, 6.2 Hz) | DMSO: −6.90 | S_{P} | 530 | V | A |
| 43B | 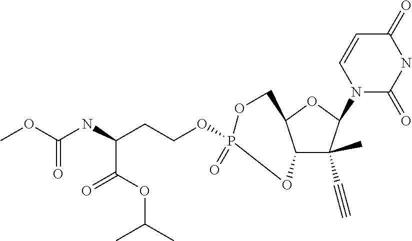 [SMILES: C#C[C@]1(C)[C@@H]2O[P@](=O)(OCC[C@H](NC(=O)OC)C(=O)OC(C)C)OC[C@H]2O[C@H]1n1ccc(=O)[nH]c1=O] | 11.58 (1H, s), 7.80 (1H, d, J = 8.2 Hz), 7.63 (1H, d, J = 8.1 Hz), 6.29 (1H, s), 5.65 (1H, d, J = 8.1 Hz), 4.95-4.87 (1H, m), 4.74-4.62 (3 hours, m), 4.35-4.27 (1H, m), 4.17-4.05 (3 hours, m), 3.55 (3 hours, s), 2.15-2.06 (1H, m), 1.96-1.90 (1H, m), 1.21-1.16 (9H, m) | DMSO: −5.70 | R_{P} | 530 | V | A |
| 44A | 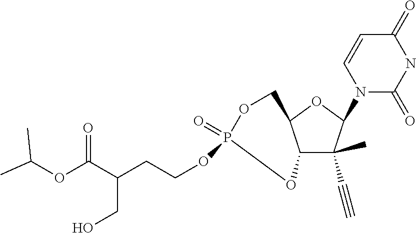 [SMILES: C#C[C@]1(C)[C@@H]2O[P@@](=O)(OCCC(CO)C(=O)OC(C)C)OC[C@H]2O[C@H]1n1ccc(=O)[nH]c1=O] | | DMSO: −6.76; −6.78 | S_{P} | 487 | X | A |
| 44B | 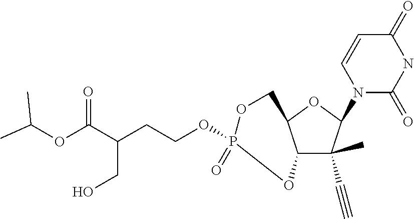 [SMILES: C#C[C@]1(C)[C@@H]2O[P@](=O)(OCCC(CO)C(=O)OC(C)C)OC[C@H]2O[C@H]1n1ccc(=O)[nH]c1=O] | | DMSO: −5.65; −5.70 | R_{P} | 487 | X | A |
| 45A | 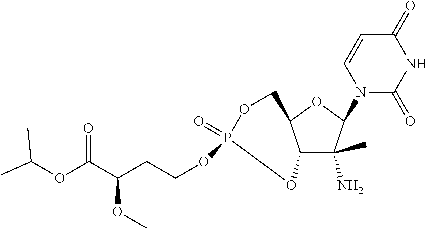 [SMILES: CO[C@H](CCO[P@@]1(=O)OC[C@H]2O[C@@H](n3ccc(=O)[nH]c3=O)[C@](C)(N)[C@@H]2O1)C(=O)OC(C)C] | DMSO: 11.50 (s, 1H), 7.76 (d, J = 8.13Hz, 1H), 5.91 (s, 1H), 5.65 (d, J = 8.13Hz, 1H), 5.01 (heptuplet, J = 6.24Hz, 1H), 4.69-4.60 (m, 1H), 4.50 (t, J = 9.73Hz, 1H), 4.30-4.24 (m, 1H), 4.19-4.17 (m, 1H), 4.13-4.06 (m, 2H), 3.96 (t, J = 5.64Hz, 1H), 3.30 (s, 3H), 2.12-2.07 (m, 2H), 2.04 (s, 2H), | DMSO: −6.05 | S_{P} | 478 | Y | C |
| | | 1.24-1.21 (m, 6H), | | | | | |
| | | 1.04 (brs, 3H) | | | | | |
| 45B | 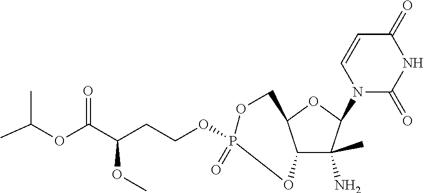 [SMILES: CO[C@H](CCO[P@]1(=O)OC[C@H]2O[C@@H](n3ccc(=O)[nH]c3=O)[C@](C)(N)[C@@H]2O1)C(=O)OC(C)C] | DMSO: 11.46 (s, 1H), 7.78 (d, J = 8.16Hz, 1H), 5.91 (s, 1H), 5.61 (d, J = 8.16Hz, 1H), 4.98 (heptuplet, J = 6.23Hz, 1H), 4.67-4.62 (m, 2H), 4.47-4.36 (m, 2H), 4.16-4.11 (m, 2H), 3.86 (dd, J = 8.71Hz, 4.22Hz, 1H), 3.30 (s, 3H), 2.08-2.02 (m, 1H), 2.00 (s, 2H), | DMSO: −4.71 | R_{P} | 478 | Y | C |
| | | 1.94-1.86 (m, 1H), | | | | | |
| | | 1.23 (d, J = 6.23Hz, | | | | | |
| | | 3H), 1.22 (d, J = | | | | | |
| | | 6.23Hz, 3H), 1.02 (brs, | | | | | |
| | | 3H) | | | | | |
| 46A | 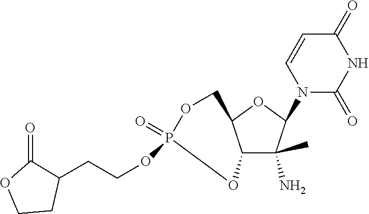 [SMILES: C[C@@]1(N)[C@@H]2O[P@@](=O)(OCCC3CCOC3=O)OC[C@H]2O[C@H]1n1ccc(=O)[nH]c1=O] | 11.48 (1H, s), 7.70 (1H, dd, J = 8.2, 11.3 Hz), 5.89 (1H, s), 5.67- 5.62 (1H, m), 4.69- 4.52 (2H, m), 4.35- 4.07 (6H, m), 2.85- 2.77 (1H, m), 2.42- 2.34 (1H, m), 2.17- 2.07 (1H, m), 2.02 (2H, s), 2.00-1.81 (2H, m), 1.02 (3 hours, s) | DMSO: −5.96; −5.98 | S_{P} | 432 | AA | A |
| 46B | 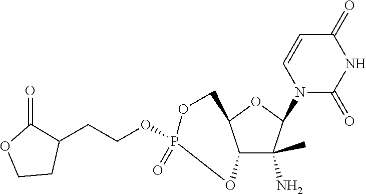 [SMILES: C[C@@]1(N)[C@@H]2O[P@](=O)(OCCC3CCOC3=O)OC[C@H]2O[C@H]1n1ccc(=O)[nH]c1=O] | 11.47 (1H, s), 7.78 (1H, d, J = 8.1 Hz), 5.91 (1H, s), 5.62 (1H, d, J = 8.2 Hz), 4.69-4.61 (2H, m), 4.49-4.13 (6H, m), 2.78-2.70 (1H, m), 2.43-2.34 (1H, m), 2.12-1.91 (4H, m), 1.80-1.70 (1H, m), 1.02 (3 hours, s) | DMSO: −4.76 | R_{P} | 432 | AA | A |
| 47A | 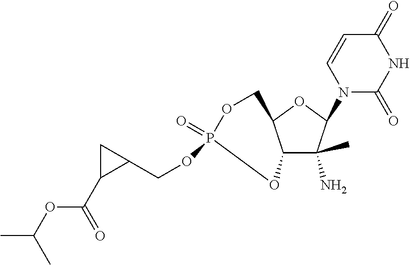 [SMILES: CC(C)OC(=O)C1CC1CO[P@@]1(=O)OC[C@H]2O[C@@H](n3ccc(=O)[nH]c3=O)[C@](C)(N)[C@@H]2O1] | 11.49 (1H, s), 7.68 (1H, d, J = 8.2 Hz), 5.91- 5.87 (1H, m), 5.65 (1H, d, J = 8.1 Hz), 4.95- 4.88 (1H, m), 4.67- 4.47 (2H, m), 4.39- 4.23 (2H, m), 4.17- 4.06 (2H, m), 2.03- 1.99 (2H, m), 1.89- 1.75 (2H, m), 1.22- 1.16 (7H, m), 1.05- 0.97 (4H, m) | DMSO: −6.15; −6.20 | S_{P} | 460 | BB | A |
| 47B | 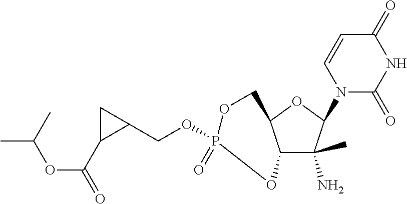 [SMILES: CC(C)OC(=O)C1CC1CO[P@]1(=O)OC[C@H]2O[C@@H](n3ccc(=O)[nH]c3=O)[C@](C)(N)[C@@H]2O1] | 11.46 (1H, s), 7.78 (1H, d, J = 8.1 Hz), 5.90 (1H, s), 5.61 (1H, d, J = 8.1 Hz), 4.96-4.86 (1H, m), 4.67-4.58 (2H, m), 4.47-4.33 (3 hours, m), 4.09-3.97 (1H, m), 2.02-1.98 (2H, m), 1.88-1.71 (2H, m), 1.22-1.17 (7H, m), 1.02 (3 hours, s), 0.99-0.90 (1H, m | DMSO: −4.95; −5.15 | R_{P} | 460 | BB | A |
| 48A | 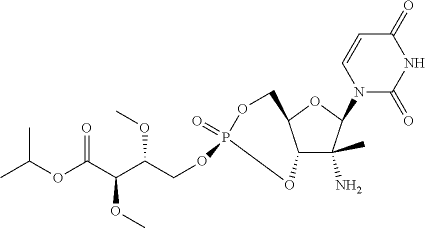 [SMILES: CO[C@H](CO[P@@]1(=O)OC[C@H]2O[C@@H](n3ccc(=O)[nH]c3=O)[C@](C)(N)[C@@H]2O1)[C@@H](OC)C(=O)OC(C)C] | 11.52 (1H, s), 7.70 (1H, d, J = 7.9 Hz), 5.91 (1H, s), 5.65 (1H, d, J = 8.1 Hz), 5.07-4.99 (1H, m), 4.72-4.60 (1H, m), 4.53 (1H, dd, J = 9.8, 9.8 Hz), 4.34- 4.25 (1H, m), 4.18- 4.01 (4H, m), 3.75 (1H, d, J = 4.6 Hz), 3.36 (3 hours, s), 3.33 (3 hours, s), 2.05 (2H, s), 1.23 (6H, dd, J = 6.3, 11.0 Hz), 1.04 (3 | DMSO: −6.24 | S_{P} | 508 | Z | C |
| | | hours, s) | | | | | |
| 48B | 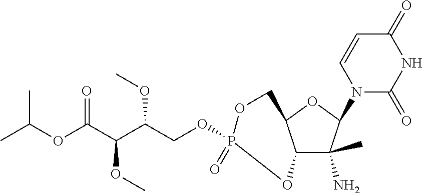 [SMILES: CO[C@H](CO[P@]1(=O)OC[C@H]2O[C@@H](n3ccc(=O)[nH]c3=O)[C@](C)(N)[C@@H]2O1)[C@@H](OC)C(=O)OC(C)C] | 11.46 (1H, s), 7.78 (1H, d, J = 8.1 Hz), 5.92 (1H, s), 5.62 (1H, d, J = 8.1 Hz), 5.04-4.97 (1H, m), 4.69-4.61 (2H, m), 4.50-4.36 (2H, m), 4.28-4.21 (1H, m), 4.06 (1H, ddd, J = 5.5, 8.3, 11.2 Hz), 3.86 (1H, d, J = 5.9 Hz), 3.60 (1H, ddd, J = 4.1, 4.1, 4.1 Hz), | DMSO: −4.46 | R_{P} | 508 | Z | C |
| | | 3.34 (3 hours, s), 3.31 | | | | | |
| | | (3 hours, s), 1.99 (2H, | | | | | |
| | | s), 1.23 (6H, dd, J = 6.4, | | | | | |
| | | 6.4 Hz), 1.03 (3 hours, | | | | | |
| | | s) | | | | | |
| 49A | 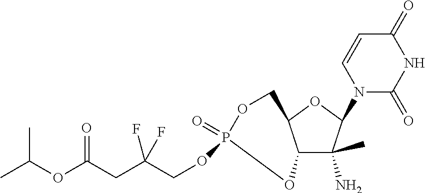 [SMILES: CC(C)OC(=O)CC(F)(F)CO[P@@]1(=O)OC[C@H]2O[C@@H](n3ccc(=O)[nH]c3=O)[C@](C)(N)[C@@H]2O1] | 11.59 (1H, s), 7.74 (1H, d, J = 8.2 Hz), 6.29 (1H, s), 5.67 (1H, d, J = 8.1 Hz), 4.99-4.89 (1H, m), 4.77-4.70 (2H, m), 4.55-4.44 (2H, m), 4.29-4.16 (2H, m), 3.65 (1H, s), 3.24 (2H, dd, J = 2.1, 16.1 Hz), 1.22-1.16 (9H, m) | DMSO: −7.17 | R_{P} | 484 | CC | C |
| 49B | 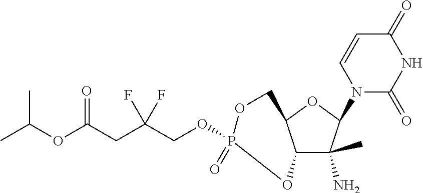 [SMILES: CC(C)OC(=O)CC(F)(F)CO[P@]1(=O)OC[C@H]2O[C@@H](n3ccc(=O)[nH]c3=O)[C@](C)(N)[C@@H]2O1] | 11.58 (1H, s), 7.78 (1H, d, J = 8.2 Hz), 6.30 (1H, s), 5.66 (1H, d, J = 8.1 Hz), 5.01-4.91 (1H, m), 4.78-4.71 (3 hours, m), 4.52-4.32 (3 hours, m), 3.61 (1H, s), 3.18 (2H, dd, J = 16.1, 16.1 Hz), 1.23- 1.17 (9H, m) | DMSO: −5.98 | S_{P} | 484 | CC | C |
| 50A | 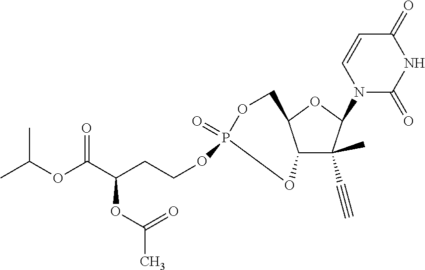 [SMILES: C#C[C@]1(C)[C@@H]2O[P@@](=O)(OCC[C@@H](OC(C)=O)C(=O)OC(C)C)OC[C@H]2O[C@H]1n1ccc(=O)[nH]c1=O] | 11.59 (1H, s), 7.73 (1H, d, J = 8.2 Hz), 6.29 (1H, s), 5.66 (1H, d, J = 8.1 Hz), 5.03-4.92 (2H, m), 4.68 (1H, ddt, J = 4.8, 10.3, 10.9 Hz), 4.58 (1H, t, J = 9.5 Hz), 4.28-4.13 (4H, m), 3.65 (1H, s), 2.28- 2.15 (2H, m), 2.08 (3 hours, s), 1.20 (9H, dd, J = 6.7, 6.7 Hz) | DMSO: −6.85 | S_{P} | 515 | DD | C |
| 50B | 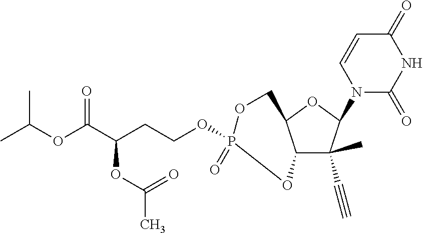 [SMILES: C#C[C@]1(C)[C@@H]2O[P@](=O)(OCC[C@@H](OC(C)=O)C(=O)OC(C)C)OC[C@H]2O[C@H]1n1ccc(=O)[nH]c1=O] | 11.57 (1H, s), 7.80 (1H, d, J = 8.2 Hz), 6.29 (1H, s), 5.65 (1H, d, J = 8.1 Hz), 4.97-4.90 (2H, m), 4.75-4.62 (3 hours, m), 4.33-4.15 (3 hours, m), 3.57 (1H, s), 2.23-2.11 (2H, m), 2.10 (3 hours, s), 1.22-1.18 (9H, m) | DMSO: −5.64 | R_{P} | 515 | DD | C |
| 51A | 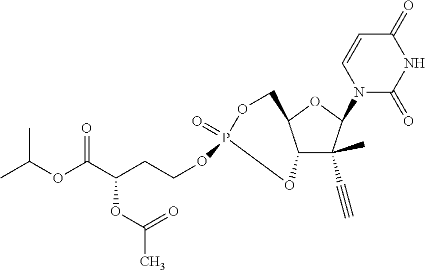 [SMILES: C#C[C@]1(C)[C@@H]2O[P@@](=O)(OCC[C@H](OC(C)=O)C(=O)OC(C)C)OC[C@H]2O[C@H]1n1ccc(=O)[nH]c1=O] | 11.61 (1H, s), 7.72 (1H, d, J = 8.2 Hz), 6.29 (1H, s), 5.67 (1H, d, J = 8.2 Hz), 5.04-4.90 (2H, m), 4.74-4.59 (2H, m), 4.26-4.14 (4H, m), 3.64 (1H, s), 2.28-2.15 (2H, m), 2.09 (3 hours, s), 1.22- 1.16 (9H, m) | DMSO: −6.79 | S_{P} | 515 | EE | C |
| 51B | 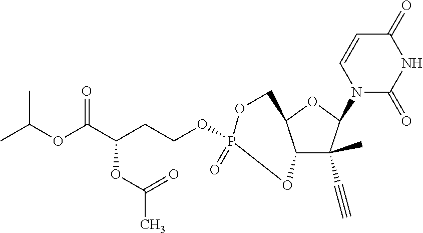 [SMILES: C#C[C@]1(C)[C@@H]2O[P@](=O)(OCC[C@H](OC(C)=O)C(=O)OC(C)C)OC[C@H]2O[C@H]1n1ccc(=O)[nH]c1=O] | 11.57 (1H, s), 7.80 (1H, d, J = 8.1 Hz), 6.29 (1H, s), 5.65 (1H, d, J = 8.1 Hz), 4.97-4.90 (2H, m), 4.75-4.62 (3 hours, m), 4.34-4.16 (3 hours, m), 3.59 (1H, s), 2.25-2.13 (2H, m), 2.10 (3 hours, s), 1.20 (9H, dd, J = 5.9, 12.1 Hz) | DMSO: −5.71 | R_{P} | 515 | EE | C |
| 52A | 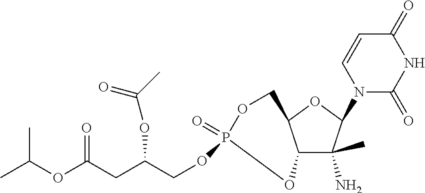 [SMILES: CC(=O)O[C@H](CO[P@@]1(=O)OC[C@H]2O[C@@H](n3ccc(=O)[nH]c3=O)[C@](C)(N)[C@@H]2O1)CC(=O)OC(C)C] | 11.49 (1H, s), 7.66 (1H, d, J = 8.1 Hz), 5.89 (1H, s), 5.65 (1H, d, J = 8.1 Hz), 4.95-4.84 (2H, m), 4.70-4.58 (1H, m), 4.50 (1H, dd, J = 9.6, 9.6 Hz), 4.32- 4.15 (4H, m), 2.84- 2.66 (2H, m), 2.06 (3 hours, s), 1.20 (6H, d, J = 6.3 Hz), 1.02 (3 hours, s) | DMSO: −7.60 | S_{P} | 506 | FF | C |
| 52B | 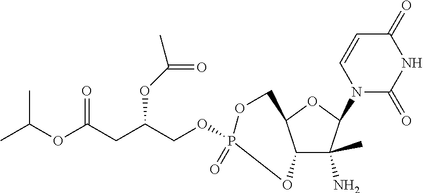 [SMILES: CC(=O)O[C@H](CO[P@]1(=O)OC[C@H]2O[C@@H](n3ccc(=O)[nH]c3=O)[C@](C)(N)[C@@H]2O1)CC(=O)OC(C)C] | 11.47 (1H, s), 7.77 (1H, d, J = 8.1 Hz), 5.90 (1H, s), 5.76 (1H, s), 5.62 (1H, dd, J = 1.2, 8.2 Hz), 4.96-4.87 (2H, m), 4.66-4.54 (2H, m), 4.48 (1H, d, J = 9.5 Hz), 4.42-4.34 (1H, m), 4.21 (2H, d, J = 5.9 Hz), 2.80-2.65 (2H, m), 2.05 (3 hours, s), 2.02-1.93 (1H, m), | DMSO: −5.24 | R_{P} | 506 | FF | C |
| | | 1.21 (6H, d, J = 6.3 Hz), | | | | | |
| | | 1.02 (3 hours, s) | | | | | |
| 53A | 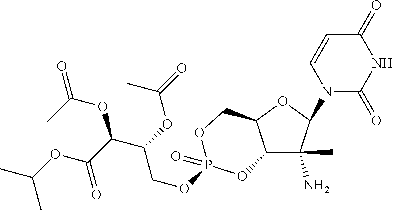 [SMILES: CC(=O)O[C@H](C(=O)OC(C)C)[C@@H](CO[P@@]1(=O)OC[C@H]2O[C@@H](n3ccc(=O)[nH]c3=O)[C@](C)(N)[C@@H]2O1)OC(C)=O] | | DMSO: −6.19 | R_{P} | 564 | GG | C |
| 53B | 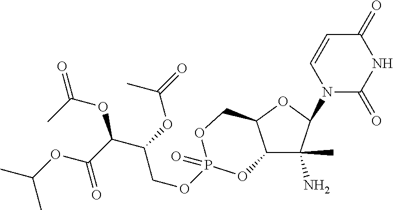 | | DMSO: −4.99 | S_{P} | 564 | GG | C |
| 54A | 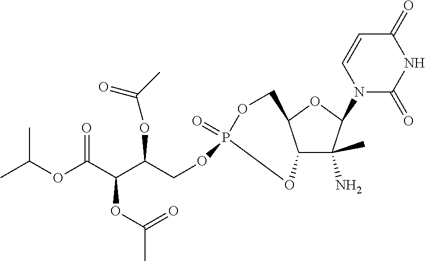 [SMILES: CC(=O)O[C@@H](CO[P@@]1(=O)OC[C@H]2O[C@@H](n3ccc(=O)[nH]c3=O)[C@](C)(N)[C@@H]2O1)[C@@H](OC(C)=O)C(=O)OC(C)C] | 11.50 (1H, s), 7.66 (1H, d, J = 8.1 Hz), 5.89 (1H, s), 5.67 (1H, d, J = 7.8 Hz), 5.60-5.54 (1H, m), 5.33 (1H, d, J = 2.7 Hz), 4.96-4.88 (1H, m), 4.71-4.54 (2H, m), 4.36-4.05 (4H, m), 2.14 (3 hours, s), 2.04 (5H, 1s), 1.20 (3 hours, d, J = 5.1 Hz), 1.14 (3 hours, d, J = 6.8 Hz), 1.02 (3 hours, s) | DMSO: −6.27 | R_{P} | 564 | HH | C |
| 54B | 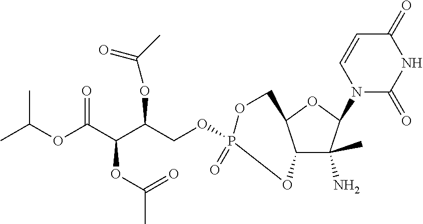 [SMILES: CC(=O)O[C@@H](CO[P@]1(=O)OC[C@H]2O[C@@H](n3ccc(=O)[nH]c3=O)[C@](C)(N)[C@@H]2O1)[C@@H](OC(C)=O)C(=O)OC(C)C] | 11.47 (1H, s), 7.76 (1H, d, J = 7.9 Hz), 5.91 (1H, s), 5.62 (1H, d, J = 8.1 Hz), 5.56-5.50 (1H, m), 5.27 (1H, d, J = 2.5 Hz), 4.94-4.87 (1H, m), 4.68-4.59 (2H, m), 4.51 (1H, d, J = 8.8 Hz), 4.45-4.38 (1H, m), 4.34-4.26 (2H, m), 4.19 (2H, d, J = 25.3 Hz), 2.15 (3 hours, s), 2.04 (3 hours, s), 2.02-1.98 | HMSO: −4.91 | S_{P} | 564 | HH | C |
| | | (2H, m), 1.20 (3 hours, | | | | | |
| | | d, J = 6.4 Hz), 1.15 (3 | | | | | |
| | | hours, d, J = 6.1 Hz), | | | | | |
| | | 1.02 (3 hours, s) | | | | | |
| 55A | 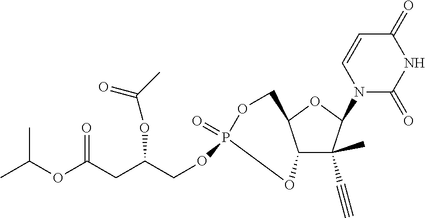 [SMILES: C#C[C@]1(C)[C@@H]2O[P@@](=O)(OC[C@H](CC(=O)OC(C)C)OC(C)=O)OC[C@H]2O[C@H]1n1ccc(=O)[nH]c1=O] | 11.61 (1H, s), 7.71 (1H, d, J = 8.2 Hz), 6.28 (1H, s), 5.67 (1H, d, J = 8.1 Hz), 4.95-4.87 (2H, m), 4.73-4.55 (2H, m), 4.34-4.12 (4H, m), 3.64 (1H, s), 2.84-2.67 (2H, m), 2.06 (3 hours, s), 1.22- 1.17 (9H, m) | DMSO: −8.33 | S_{P} | 515 | FF | C |
| 55B | 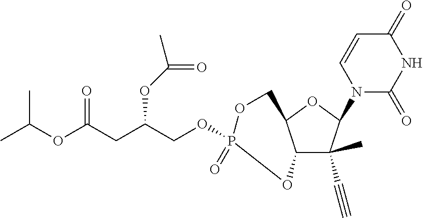 [SMILES: C#C[C@]1(C)[C@@H]2O[P@](=O)(OC[C@H](CC(=O)OC(C)C)OC(C)=O)OC[C@H]2O[C@H]1n1ccc(=O)[nH]c1=O] | 11.57 (1H, s), 7.79 (1H, d, J = 8.1 Hz), 6.28 (1H, s), 5.65 (1H, d, J = 8.1 Hz), 4.97-4.89 (2H, m), 4.73-4.61 (3 hours, m), 4.30-4.22 (1H, m), 4.20 (2H, d, J = 5.4 Hz), 3.52 (1H, s), 2.82-2.66 (2H, m), 2.04 (3 hours, s), 1.22- 1.16 (9H, m) | DMSO: −5.53 | R_{P} | 515 | FF | C |
| 56A | 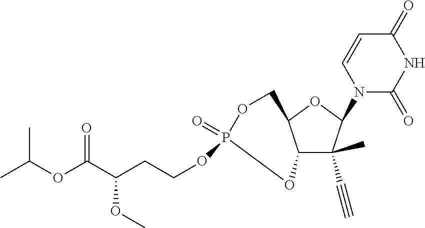 [SMILES: C#C[C@]1(C)[C@@H]2O[P@@](=O)(OCC[C@H](OC)C(=O)OC(C)C)OC[C@H]2O[C@H]1n1ccc(=O)[nH]c1=O] | DMSO: 11.60 (s, 1H), 7.75 (d, J = 8.09Hz, 1H), 6.29 (s, 1H), 5.68 (d, J = 8.09Hz, 1H), 4.97 (heptuplet, J = 6.25Hz, 1H), 4.73-4.62 (m, 2H), 4.25 (d, J = 9.53Hz, 1H), 4.18-4.12 (m, 3H), 3.98 (dd, J = 7.54Hz, 4.67Hz, 1H), 3.64 (s, 1H), 3.30 (s, 3H), 2.16-1.98 (m, 2H), 1.23-1.18 (m, 9H) | DMSO: −6.68 | S_{P} | 487 | JJ | C |
| 56B | 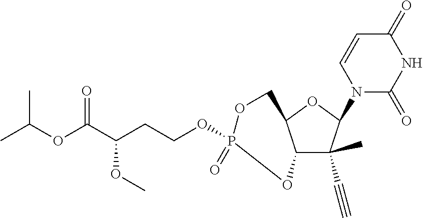 [SMILES: C#C[C@]1(C)[C@@H]2O[P@](=O)(OCC[C@H](OC)C(=O)OC(C)C)OC[C@H]2O[C@H]1n1ccc(=O)[nH]c1=O] | DMSO: 11.57 (s, 1H), 7.80 (d, J = 8.10Hz, 1H), 6.29 (s, 1H), 5.66.5.64 (m, 1H), 4.97 (heptuplet, J = 6.22Hz, 1H), 4.73-4.67 (m, 2H), 4.63 (d, J = 9.69Hz, 1H), 4.34-4.26 (m, 1H), 4.19-4.11 (m, 2H), 3.86 (dd, J = 8.49Hz, 4.25Hz, 1H), 3.59 (s, 1H), 3.30 (s, 3H), 2.09-2.01 (m, 1H), 1.96-1.88 (m, | DMSO: −5.61 | R_{P} | 487 | JJ | C |
| | | 1H), 1.23 (d, J = | | | | | |
| | | 6.22Hz, 3H), 1.22 (d, | | | | | |
| | | J = 6.22Hz, 3H), 1.18 | | | | | |
| | | (s, 3H) | | | | | |
| 57A | 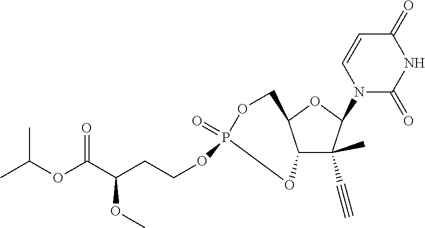 [SMILES: C#C[C@]1(C)[C@@H]2O[P@@](=O)(OCC[C@@H](OC)C(=O)OC(C)C)OC[C@H]2O[C@H]1n1ccc(=O)[nH]c1=O] | DMSO: 11.60 (s, 1H), 7.79 (d, J = 8.07Hz, 1H), 6.30 (s, 1H), 5.68 (d, J = 8.07Hz, 1H), 5.01 (heptuplet, J = 6.22Hz, 1H), 4.73-4.64 (m, 1H), 4.56 (t, J = 9.78Hz, 1H), 4.31 (d, J = 9.58Hz, 1H), 4.18- 4.08 (m, 3H), 3.97 (t, J = 5.84Hz, 1H), 3.65 (s, 1H), 3.30 (s, 3H), 2.17-2.03 (m, 2H), 1.24-1.20 (m, 9H) | DMSO: −6.83 | S_{P} | 487 | Y | C |
| 57B | 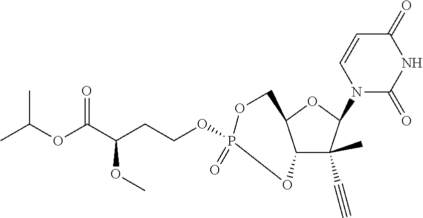 [SMILES: C#C[C@]1(C)[C@@H]2O[P@](=O)(OCC[C@@H](OC)C(=O)OC(C)C)OC[C@H]2O[C@H]1n1ccc(=O)[nH]c1=O] | DMSO: 11.57 (s, 1H), 7.80 (d, J = 8.09Hz, 1H), 6.29 (s, 1H), 5.65 (d, J = 8.09Hz, 1H), 4.97 (heptuplet, J = 6.26Hz, 1H), 4.74-4.67 (m, 2H), 4.64 (d, J = 9.60Hz, 1H), 4.34-4.26 (m, 1H), 4.20-4.11 (m, 2H), 3.87 (dd, J = 8.73Hz, 4.27Hz, 1H), 3.57 (s, 1H), 3.30 (s, 3H), 2.10-2.02 (m, 1H), 1.95-1.86 (m, | DMSO: −5.54 | R_{P} | 487.2 | Y | C |
| | | 1H), 1.23 (d, J = | | | | | |
| | | 6.26Hz, 3H), 1.22 (d, | | | | | |
| | | J = 6.26Hz, 3H), 1.18 | | | | | |
| | | (s, 3H) | | | | | |
| 58A | 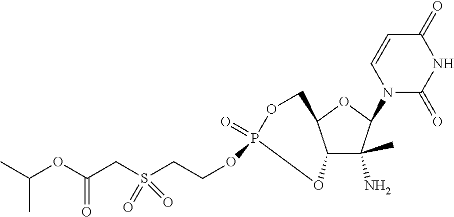 [SMILES: CC(C)OC(=O)CS(=O)(=O)CCO[P@@]1(=O)OC[C@H]2O[C@@H](n3ccc(=O)[nH]c3=O)[C@](C)(N)[C@@H]2O1] | 11.50 (1H, s), 7.48 (1H, d, J = 8.1 Hz), 5.91 (1H, s), 5.76-5.67 (1H, m), 5.03-4.95 (1H, m), 4.74-4.63 (1H, m), 4.56-4.24 (7H, m), 3.80 (2H, q, J = 5.7 Hz), 2.10-2.07 (2H, m), 1.23 (6H, d, J = 6.2 Hz), 1.03 (3 hours, s) | DMSO: −6.95 | R_{P} | 512 | II | C |
| 59A | 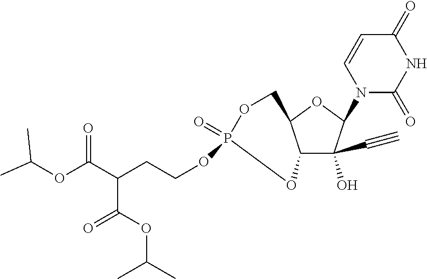 [SMILES: C#C[C@@]1(O)[C@@H]2O[P@@](=O)(OCCC(C(=O)OC(C)C)C(=O)OC(C)C)OC[C@H]2O[C@H]1n1ccc(=O)[nH]c1=O] | DMSO: 11.54 (s, 1H), 7.75 (d, J = 8.11Hz, 1H), 7.20 (s, 1H), 6.06 (s, 1H), 5.66 (d, J = 8.11Hz, 1H), 4.96 (heptuplet, J = 6.26Hz, 2H), 4.70-4.61 (m, 1H), 4.58-4.54 (m, 1H), 4.38-4.36 (m, 1H), 4.15-4.12 (m, 3H), 3.82 (s, 1H), 3.63 (t, J = 6.83Hz, 1H), 2.21 (q, J = 6.83Hz, 2H), 1.21-1.18 (m, 12H) | DMSO: −6.49 | S_{P} | 545 | W | C |
| 59B | 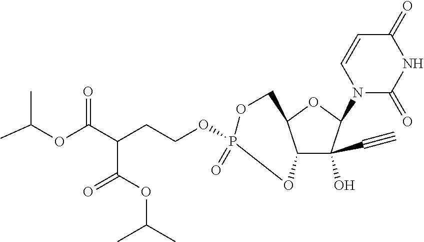 [SMILES: C#C[C@@]1(O)[C@@H]2O[P@](=O)(OCCC(C(=O)OC(C)C)C(=O)OC(C)C)OC[C@H]2O[C@H]1n1ccc(=O)[nH]c1=O] | DMSO: 11.51 (s, 1H), 7.84 (d, J = 8.09Hz, 1H), 7.14 (s, 1H), 6.06 (s, 1H), 5.65 (d, J = 8.09Hz, 1H), 4.95 (heptuplet, J = 6.21Hz, 2H), 4.77-4.74 (m, 1H), 4.68-4.60 (m, 2H), 4.29-4.23 (m, 1H), 4.13-4.08 (m, 2H), 3.83 (s, 1H), 3.50 (t, J = 7.23Hz, 1H), 2.12 (q, J = 6.64Hz, 2H), 1.21-1.18 (m, 12H) | DMSO: −5.44 | R_{P} | 545 | W | C |
| 60A | 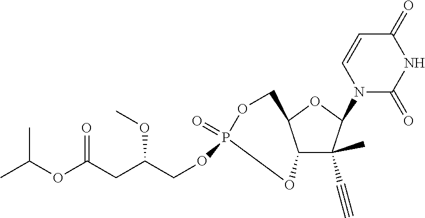 [SMILES: C#C[C@]1(C)[C@@H]2O[P@@](=O)(OC[C@H](CC(=O)OC(C)C)OC)OC[C@H]2O[C@H]1n1ccc(=O)[nH]c1=O] | CDCl₃: 8.46 (s, 1H), 7.28-7.26 (m, 1H), 6.46 (s, 1H), 5.80-5.78 (m, 1H), 5.03 (heptuplet, J = 6.29Hz, 1H), 4.68-4.61 (m, 1H), 4.43-4.35 (m, 2H), 4.28-4.16 (m, 2H), 4.12-4.10 (m, 1H), 3.91-3.86 (m, 1H), 3.41 (s, 3H), 2.65 (dd, J = 15.42Hz and 6.29Hz, 1H), 2.54 (dd, J = 15.42Hz and | CDCl₃: −7.29 | S_{P} | 487.2 | KK | A |
| | | 6.29Hz, 1H), 2.54 (s, | | | | | |
| | | 1H), 1.33 (s, 3H), 1.26 | | | | | |
| | | (d, J = 6.29Hz, 3H), | | | | | |
| | | 1.25 (d, J = 6.29Hz, | | | | | |
| | | 3H) | | | | | |
| 60B | 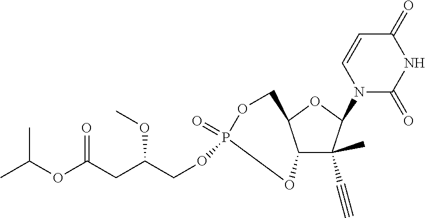 [SMILES: C#C[C@]1(C)[C@@H]2O[P@](=O)(OC[C@H](CC(=O)OC(C)C)OC)OC[C@H]2O[C@H]1n1ccc(=O)[nH]c1=O] | CDCl₃: 8.58 (s, 1H), 7.23 (d, J = 8.04Hz, 1H), 6.46 (s, 1H), 5.80 (d, J = 8.04Hz, 1H), 5.04 (heptuplet, J = 6.26Hz, 1H), 4.72-4.64 (m, 1H), 4.58-4.51 (m, 1H), 4.46-4.40 (m, 1H), 4.34-4.28 (m, 1H), 4.23-4.17 (m, 2H), 3.92-3.86 (m, 1H), 3.45 (s, 3H), 2.57 (s, 1H), 2.56-2.54 (m, 2H), 1.33 (s, 3H), | CDCl₃: −4.05 | R_{P} | 487.2 | KK | A |
| | | 1.255 (d, J = 6.26Hz, | | | | | |
| | | 3H), 1.25 (d, J = | | | | | |
| | | 6.26Hz, 3H) | | | | | |
| 61A | 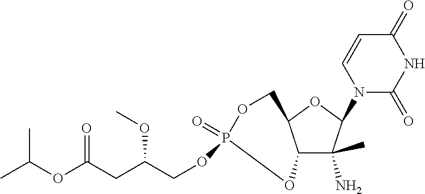 [SMILES: CO[C@H](CO[P@@]1(=O)OC[C@H]2O[C@@H](n3ccc(=O)[nH]c3=O)[C@](C)(N)[C@@H]2O1)CC(=O)OC(C)C] | DMSO: 11.49 (s, 1H), 7.66 (d, J = 8.17Hz, 1H), 5.89 (s, 1H), 5.65 (d, J = 8.17Hz, 1H), 4.92 (heptuplet, J = 6.36Hz, 1H), 4.69-4.57 (m, 2H), 4.32-4.26 (m, 1H), 4.20-4.14 (m, 1H), 4.11-4.03 (m, 2H), 3.84-3.81 (m, 1H), 3.31 (s, 3H), 2.65-2.52 (m, 2H), | DMSO: −5.98 | S_{P} | 478.2 | KK | A |
| | | 1.19 (d, J = 6.36Hz, | | | | | |
| | | 6H), 1.02 (s, 3H) | | | | | |
| 61B | 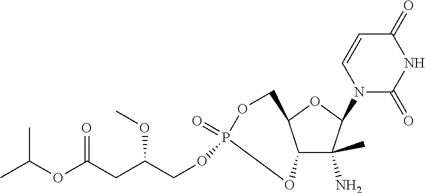 [SMILES: CO[C@H](CO[P@]1(=O)OC[C@H]2O[C@@H](n3ccc(=O)[nH]c3=O)[C@](C)(N)[C@@H]2O1)CC(=O)OC(C)C] | DMSO: 11.46 (s, 1H), 7.77 (d, J = 8.13Hz, 1H), 5.91 (s, 1H), 5.62 (d, J = 8.13Hz, 1H), 4.92 (heptuplet, J = 6.34Hz, 1H), 4.67-4.62 (m, 2H), 4.48-4.38 (m, 2H), 4.22-4.16 (m, 1H), 4.07-4.01 (m, 1H), 3.81-3.75 (m, 1H), 3.31 (s, 3H), 2.58-2.53 (m, 1H), | DMSO: −4.57 | R_{P} | 478.2 | KK | A |
| | | 2.49-2.42 (m, 1H), | | | | | |
| | | 2.01 (brs, 2H), 1.20- | | | | | |
| | | 1.19 (m, 6H), 1.02 (s, | | | | | |
| | | 3H) | | | | | |
| 62A | 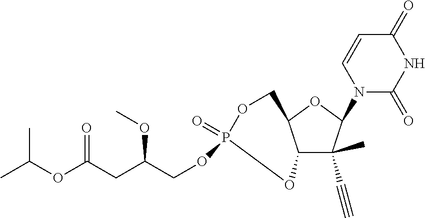 [SMILES: C#C[C@]1(C)[C@@H]2O[P@@](=O)(OC[C@@H](CC(=O)OC(C)C)OC)OC[C@H]2O[C@H]1n1ccc(=O)[nH]c1=O] | CDCl₃: 8.36 (s, 1H), 7.30-7.28 (m, 1H), 6.46 (s, 1H), 5.80-5.78 (m, 1H), 5.01 (heptuplet, J = 6.36Hz, 1H), 4.69-4.60 (m, 1H), 4.47-4.35 (m, 2H), 4.29-4.24 (m, 1H), 4.19-4.13 (m, 1H), 4.09-4.07 (m, 1H), 3.91-3.86 (m, 1H), 3.45 (s, 3H), 2.64 (dd, J = 15.31Hz and 5.89Hz, 1H), 2.55 (s, | CDCl₃: −7.20 | S_{P} | 487.2 | LL | A |
| | | 1H), 2.53 (dd, J = | | | | | |
| | | 15.31Hz and 6.83Hz, | | | | | |
| | | 1H), 1.33 (s, 3H), | | | | | |
| | | 1.255 (d, J = 6.36Hz, | | | | | |
| | | 3H), 1.25 (d, J = | | | | | |
| | | 6.36Hz, 3H) | | | | | |
| 62B | 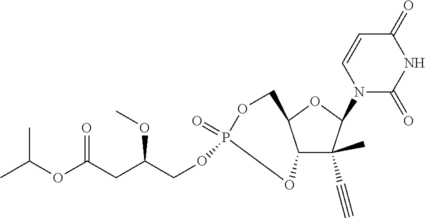 [SMILES: C#C[C@]1(C)[C@@H]2O[P@](=O)(OC[C@@H](CC(=O)OC(C)C)OC)OC[C@H]2O[C@H]1n1ccc(=O)[nH]c1=O] | CDCl₃: 8.44 (s, 1H), 7.21 (d, J = 8.15Hz, 1H), 6.46 (s, 1H), 5.81-5.79 (m, 1H), 5.04 (heptuplet, J = 6.30Hz, 1H), 4.72-4.64 (m, 1H), 4.57-4.50 (m, 1H), 4.46-4.39 (m, 1H), 4.29-4.17 (m, 3H), 3.92-3.87 (m, 1H), 3.45 (s, 3H), 2.57 (s, 1H), 2.56-2.54 (m, 2H), 1.33 (s, 3H), 1.26-1.24 (m, 6H) | CDCl₃: −4.07 | R_{P} | 487.2 | LL | A |
| 63A | 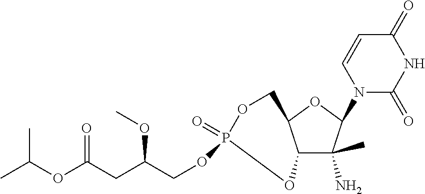 [SMILES: CO[C@@H](CO[P@@]1(=O)OC[C@H]2O[C@@H](n3ccc(=O)[nH]c3=O)[C@](C)(N)[C@@H]2O1)CC(=O)OC(C)C] | DMSO: 11.50 (s, 1H), 7.68 (d, J = 8.06Hz, 1H), 5.90 (s, 1H), 5.65 (d, J = 8.06Hz, 1H), 4.91 (heptuplet, J = 6.32Hz, 1H), 4.70-4.58 (m, 2H), 4.32-4.25 (m, 1H), 4.18-4.13 (m, 1H), 4.11-4.01 (m, 2H), 3.84-3.81 (m, 1H), 3.33 (s, 3H), 2.63-2.52 (m, 2H), | DMSO: −6.02 | S_{P} | 478.2 | LL | A |
| | | 1.19 (d, J = 6.32Hz, | | | | | |
| | | 3H), 1.18 (d, J = | | | | | |
| | | 6.32Hz, 3H), 1.02 (s, | | | | | |
| | | 3H) | | | | | |
| 63B | 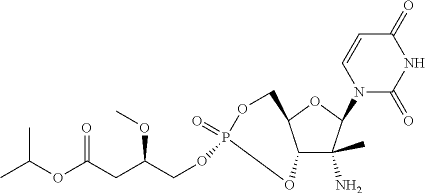 [SMILES: CO[C@@H](CO[P@]1(=O)OC[C@H]2O[C@@H](n3ccc(=O)[nH]c3=O)[C@](C)(N)[C@@H]2O1)CC(=O)OC(C)C] | DMSO: 11.47 (s, 1H), 7.77 (d, J = 8.03Hz, 1H), 5.91 (s, 1H), 5.62 (d, J = 8.03Hz, 1H), 4.92 (heptuplet, J = 6.33Hz, 1H), 4.67-4.62 (m, 2H), 4.49-4.39 (m, 2H), 4.19-4.14 (m, 1H), 4.09-4.03 (m, 1H), 3.81-3.75 (m, 1H), 3.31 (s, 3H), 2.58-2.52 (m, 1H), | DMSO: −4.63 | R_{P} | 478.2 | LL | A |
| | | 2.48-2.42 (m, 1H), | | | | | |
| | | 2.02 (brs, 2H), 1.195 | | | | | |
| | | (d, J = 6.33Hz, 6H), | | | | | |
| | | 1.02 (s, 3H) | | | | | |
| 64A | 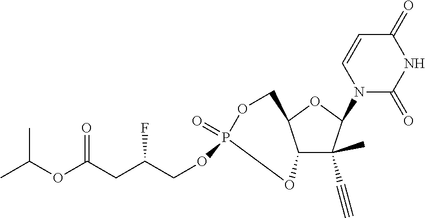 [SMILES: C#C[C@]1(C)[C@@H]2O[P@@](=O)(OC[C@@H](F)CC(=O)OC(C)C)OC[C@H]2O[C@H]1n1ccc(=O)[nH]c1=O] | DMSO: 11.59 (s, 1H), 7.77 (d, J = 8.17Hz, 1H), 6.28 (s, 1H), 5.68 (d, J = 8.17Hz, 1H), 5.22-5.07 (m, 1H), 4.94 (heptuplet, J = 6.25Hz, 1H), 4.73-4.65 (m, 2H), 4.38-4.25 (m, 3H), 4.19-4.13 (m, 1H), 3.64 (s, 1H), 2.90-2.71 (m, 2H), 1.21-1.17 (m, 9H) | DMSO: −6.83 | R_{P} | 475.2 | MM | C |
| 64B | 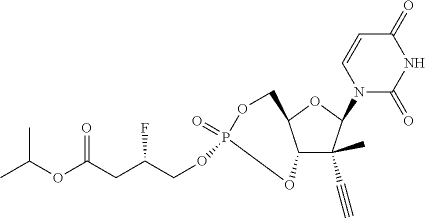 [SMILES: C#C[C@]1(C)[C@@H]2O[P@](=O)(OC[C@@H](F)CC(=O)OC(C)C)OC[C@H]2O[C@H]1n1ccc(=O)[nH]c1=O] | DMSO: 11.58 (s, 1H), 7.79 (d, J = 8.06Hz, 1H), 6.30 (s, 1H), 5.66 (d, J = 8.06Hz, 1H), 5.15-5.03 (m, 1H), 4.94 (heptuplet, J = 6.20Hz, 1H), 4.74-4.67 (m, 3H), 4.38-4.18 (m, 3H), 3.59 (s, 1H), 2.87-2.64 (m, 2H), 1.21-1.18 (m, 9H) | DMSO: −5.61 | S_{P} | 475.2 | MM | C |
| 65A | 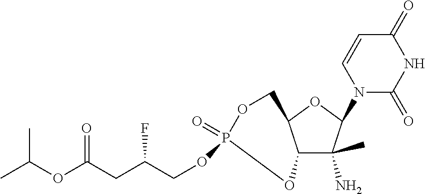 [SMILES: CC(C)OC(=O)C[C@H](F)CO[P@@]1(=O)OC[C@H]2O[C@@H](n3ccc(=O)[nH]c3=O)[C@](C)(N)[C@@H]2O1] | DMSO: 11.65 (s, 1H), 7.81 (d, J = 8.07Hz, 1H), 6.15 (s, 1H), 5.75-7.73 (m, 1H), 5.24-5.11 (m, 1H), 4.94 (heptuplet, J = 6.23Hz, 1H), 4.81-4.73 (m, 1H), 4.64-4.52 (m, 1H), 4.40-4.23 (m, 3H), 2.91-2.71 (m, 2H), 1.21-1.19 (m, 9H) | DMSO: −6.93 | R_{P} | 466 | MM | C |
| 66A | 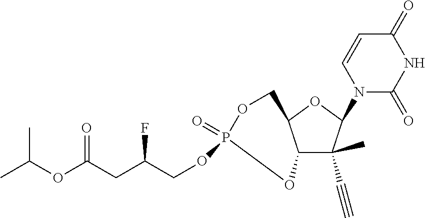 [SMILES: C#C[C@]1(C)[C@@H]2O[P@@](=O)(OC[C@H](F)CC(=O)OC(C)C)OC[C@H]2O[C@H]1n1ccc(=O)[nH]c1=O] | DMSO: 11.59 (s, 1H), 7.77 (d, J = 8.16Hz, 1H), 6.28 (s, 1H), 5.68 (d, J = 8.16Hz, 1H), 5.21-5.08 (m, 1H), 4.94 (heptuplet, J = 6.23Hz, 1H), 4.73-4.65 (m, 2H), 4.39-4.22 (m, 3H), 4.20-4.14 (m, 1H), 3.64 (s, 1H), 2.92-2.72 (m, 2H), 1.195 (d, J = 6.23Hz, 6H), 1.17 (s, 3H) | DMSO: −6.74 | R_{P} | 475 | NN | C |
| 66B | 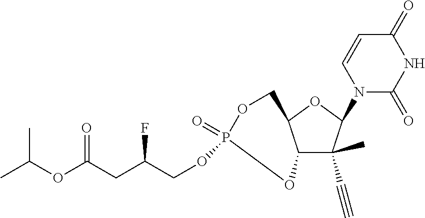 [SMILES: C#C[C@]1(C)[C@@H]2O[P@](=O)(OC[C@H](F)CC(=O)OC(C)C)OC[C@H]2O[C@H]1n1ccc(=O)[nH]c1=O] | DMSO: 11.58 (s, 1H), 7.79 (d, J = 8.16Hz, 1H), 6.30 (s, 1H), 5.66 (d, J = 8.16Hz, 1H), 5.16-5.03 (m, 1H), 4.94 (heptuplet, J = 6.22Hz, 1H), 4.75-4.67 (m, 3H), 4.38-4.19 (m, 3H), 3.59 (s, 1H), 2.86-2.65 (m, 2H), 1.21-1.19 (m, 6H), 1.18 (s, 3H) | DMSO: −5.68 | S_{P} | 475 | NN | C |
| 67A | 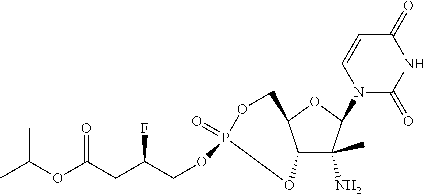 [SMILES: CC(C)OC(=O)C[C@@H](F)CO[P@@]1(=O)OC[C@H]2O[C@@H](n3ccc(=O)[nH]c3=O)[C@](C)(N)[C@@H]2O1] | DMSO: 11.66 (s, 1H), 7.81 (d, J = 8.01Hz, 1H), 6.17 (s, 1H), 5.75-7.73 (m, 1H), 5.23-5.11 (m, 1H), 4.95 (heptuplet, J = 6.32Hz, 1H), 4.82-4.73 (m, 1H), 4.66-4.52 (m, 1H), 4.40-4.22 (m, 3H), 2.95-2.73 (m, 2H), 1.23 (s, 3H), 1.20 (d, J = 6.32Hz, 6H) | DMSO: −6.92 | R_{P} | 466 | NN | C |
| 68A | 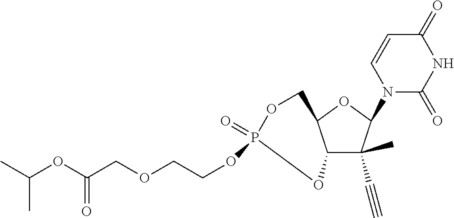 [SMILES: C#C[C@]1(C)[C@@H]2O[P@@](=O)(OCCOCC(=O)OC(C)C)OC[C@H]2O[C@H]1n1ccc(=O)[nH]c1=O] | | DMSO: −6.89 | S_{P} | 473.2 | QQ | A |
| 68B | 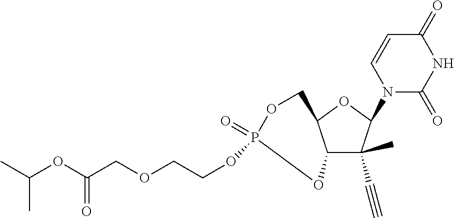 [SMILES: C#C[C@]1(C)[C@@H]2O[P@](=O)(OCCOCC(=O)OC(C)C)OC[C@H]2O[C@H]1n1ccc(=O)[nH]c1=O] | | DMSO: −5.51 | R_{P} | 473.2 | QQ | A |
| 69A | 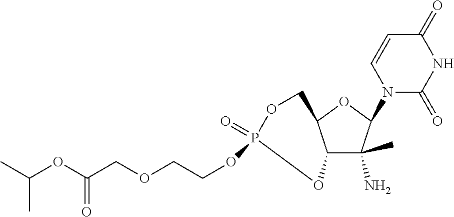 [SMILES: CC(C)OC(=O)COCCO[P@@]1(=O)OC[C@H]2O[C@@H](n3ccc(=O)[nH]c3=O)[C@](C)(N)[C@@H]2O1] | | DMSO: −6.14 | S_{P} | 464.2 | QQ | A |
| 69B | 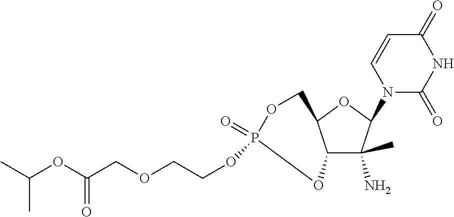 [SMILES: CC(C)OC(=O)COCCO[P@]1(=O)OC[C@H]2O[C@@H](n3ccc(=O)[nH]c3=O)[C@](C)(N)[C@@H]2O1] | | DMSO: −4.72 | R_{P} | 464.2 | QQ | A |
| 70A | 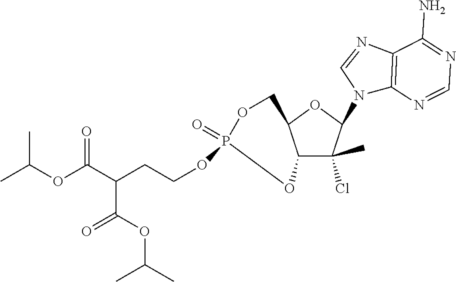 [SMILES: CC(C)OC(=O)C(CCO[P@@]1(=O)OC[C@H]2O[C@@H](n3cnc4c(N)ncnc43)[C@](C)(Cl)[C@@H]2O1)C(=O)OC(C)C] | DMSO: 8.38 (s, 1H), 8.22 (s, 1H), 7.47 (s, 2H), 6.68 (s, 1H), 5.97 (brs, 1H), 4.97-4.89 (m, 2H), 4.76-4.67 (m, 1H), 4.59 (t, J = 9.60Hz, 1H), 4.33 (dt, J = 9.81Hz, J = 4.78Hz, 1H), 4.20-4.16 (m, 2H), 3.64 (t, J = 7.13Hz, 1H), 2.26-2.21 (m, 2H), 1.40 (s, 3H), 1.17-1.14 (m, 12H) | DMSO: −6.64 | S_{P} | 576.2 | W | B |
| 70B | 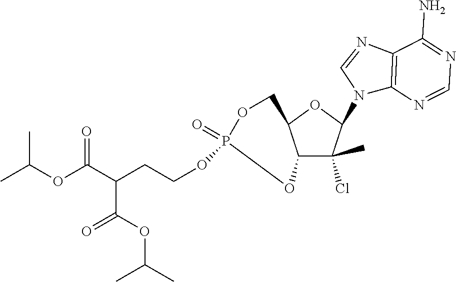 [SMILES: CC(C)OC(=O)C(CCO[P@]1(=O)OC[C@H]2O[C@@H](n3cnc4c(N)ncnc43)[C@](C)(Cl)[C@@H]2O1)C(=O)OC(C)C] | DMSO 8.44 (s, 1H), 8.20 (s, 1H), 7.46 (s, 2H), 6.67 (s, 1H), 5.63 (brs, 1H), 4.95 (heptuplet, J = 6.20Hz, 2H), 4.79-4.70 (m, 2H), 4.54-4.47 (m, 1H), 4.17 (dd, J = 13.95Hz, J = 6.55Hz, 2H), 3.54 (t, J = 7.19Hz, 1H), 2.16 (q, J = 6.55Hz, 2H), 1.36 (s, 3H), 1.22-1.18 (m, 12H) | DMSO: −5.73 | R_{P} | 576.2 | W | B |
| 71A | 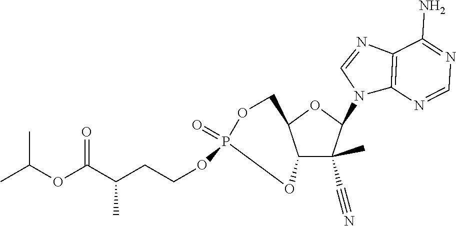 [SMILES: CC(C)OC(=O)[C@@H](C)CCO[P@@]1(=O)OC[C@H]2O[C@@H](n3cnc4c(N)ncnc43)[C@](C)(C#N)[C@@H]2O1] | DMSO: 8.31 (s, 1H), 8.18 (s, 1H), 7.49 (s, 2H), 6.86 (s, 1H), 5.85 (brs, 1H), 4.89 (heptuplet, J = 6.21Hz, 1H), 4.78-4.69 (m, 1H), 4.54 (t, J = 9.78Hz, 1H), 4.26 (dt, J = 9.85Hz, J = 4.59Hz, 1H), 4.18 (q, J = 6.65Hz, 2H), 2.67-2.62 (m, 1H), 2.11-2.03 (m, 1H), 1.86-1.77 (m, 1H), 1.24 (s, 3H), 1.16 | DMSO: −7.05 | S_{P} | 495.2 | RR | A |
| | | (d, J = 6.21Hz, 3H), | | | | | |
| | | 1.155 (d, J = 6.21Hz, | | | | | |
| | | 3H), 1.115 (d, J = | | | | | |
| | | 7.06Hz, 3H) | | | | | |
| 71B | 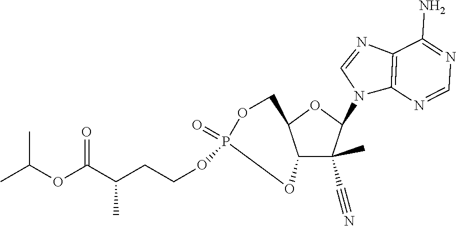 [SMILES: CC(C)OC(=O)[C@@H](C)CCO[P@]1(=O)OC[C@H]2O[C@@H](n3cnc4c(N)ncnc43)[C@](C)(C#N)[C@@H]2O1] | DMSO: 8.37 (s, 1H), 8.21 (s, 1H), 7.48 (s, 2H), 6.84 (s, 1H), 5.60 (brs, 1H), 4.90 (heptuplet, J = 6.25Hz, 1H), 4.81-4.73 (m, 1H), 4.67-4.65 (m, 1H), 4.42 (dt, J = 9.83Hz, J = 5.59Hz, 1H), 4.20-4.15 (m, H), 2.58-2.52 (m, 1H), 2.05-1.96 (m, 1H), 1.81-1.73 (m, 1H), 1.20-1.18 (m, | DMSO: −5.89 | R_{P} | 495.2 | RR | A |
| | | 9H), 1.12 (d, J = | | | | | |
| | | 7.26Hz, 3H) | | | | | |
| 72A | 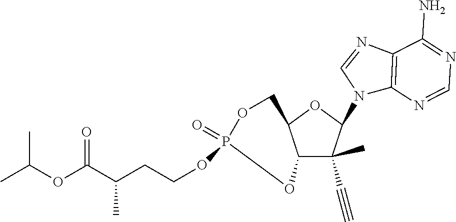 [SMILES: C#C[C@]1(C)[C@@H]2O[P@@](=O)(OCC[C@H](C)C(=O)OC(C)C)OC[C@H]2O[C@H]1n1cnc2c(N)ncnc21] | DMSO: 8.37 (s, 1H), 8.17 (s, 1H), 7.43 (s, 2H), 6.50 (s, 1H), 5.72 (brs, 1H), 4.88 (heptuplet, J = 6.23Hz, 1H), 4.73-4.64 (m, 1H), 4.61-4.56 (m, 1H), 4.26 (dt, J = 9.86Hz, J = 4.76Hz, 1H), 4.14 (q, J = 6.78Hz, 2H), 3.73 (s, 1H), 2.67-2.62 (m, 1H), 2.10-2.01 (m, 1H), 1.84-1.76 (m, | DMSO: −6.34 | S_{P} | 494.2 | RR | A |
| | | 1H), 1.16 (d, J = | | | | | |
| | | 6.23Hz, 3H), 1.155 (d, | | | | | |
| | | J = 6.23Hz, 3H), 1.105 | | | | | |
| | | (d, J = 7.08Hz, 3H), | | | | | |
| | | 1.02 (s, 3H) | | | | | |
| 72B | 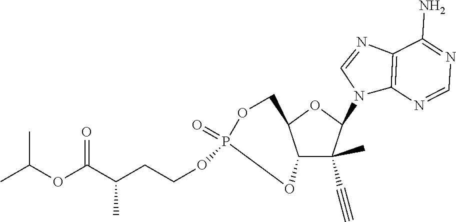 [SMILES: C#C[C@]1(C)[C@@H]2O[P@](=O)(OCC[C@H](C)C(=O)OC(C)C)OC[C@H]2O[C@H]1n1cnc2c(N)ncnc21] | DMSO 8.44 (s, 1H), 8.19 (s, 1H), 7.42 (s, 2H), 6.49 (s, 1H), 5.25 (brs, 1H), 4.90 (heptuplet, J = 6.25Hz, 1H), 4.74-4.69 (m, 2H), 4.45-4.39 (m, 1H), 4.16-4.10 (m, 2H), 3.69 (s, 1H), 2.57-2.52 (m, 1H), 2.03-1.94 (m, 1H), 1.79-1.71 (m, 1H), 1.20 (d, J = 6.25Hz, 3H), 1.19 (d, J = | DMSO: −5.45 | R_{P} | 494.2 | RR | A |
| | | 6.25Hz, 3H), 1.12 (d, | | | | | |
| | | J = 7.08Hz, 3H), 0.99 | | | | | |
| | | (s, 3H) | | | | | |
| 73A | 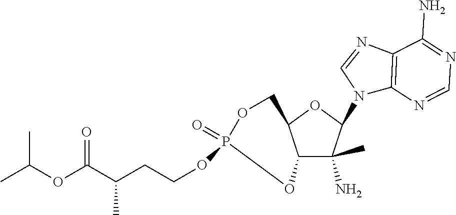 [SMILES: CC(C)OC(=O)[C@@H](C)CCO[P@@]1(=O)OC[C@H]2O[C@@H](n3cnc4c(N)ncnc43)[C@](C)(N)[C@@H]2O1] | DMSO: 9.50 (s, 2H), 8.37 (s, 1H), 8.17 (s, 1H), 7.40 (s, 2H), 6.30 (s, 1H), 4.89 (heptuplet, J = 6.33Hz, 1H), 4.72-4.63 (m, 2H), 4.58-4.54 (m, 1H), 4.35-4.29 (m, 1H), 4.11 (q, J = 6.22Hz, 2H), 2.65-2.63 (m, 1H), 2.10-2.01 (m, 1H), 1.84-1.76 (m, 1H), 1.165 (d, J = 6.33Hz, 3H), 1.155 (d, | DMSO: −5.99 | S_{P} | 485.2 | RR | A |
| | | J = 6.33Hz, 3H), 1.11 | | | | | |
| | | (d, J = 7.04Hz, 3H), | | | | | |
| | | 0.96 (s, 3H) | | | | | |
| 73B | 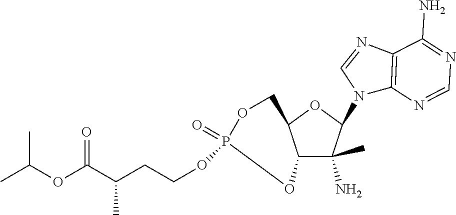 [SMILES: CC(C)OC(=O)[C@@H](C)CCO[P@]1(=O)OC[C@H]2O[C@@H](n3cnc4c(N)ncnc43)[C@](C)(N)[C@@H]2O1] | DMSO: 8.35 (s, 1H), 8.17 (s, 1H), 7.36 (s, 2H), 6.01 (s, 1H), 5.12 (brs, 1H), 4.91 (heptuplet, J = 6.19Hz, 1H), 4.70-4.59 (m, 2H), 4.56-4.50 (m, 1H), 4.17-4.05 (m, 2H), 2.58-2.53 (m, 1H), 2.15 (s, 2H), 2.01-1.93 (m, 1H), 1.77-1.69 (m, 1H), 1.20 (d, J = 6.19Hz, 3H), 1.195 (d, J = | DMSO: −4.56 | R_{P} | 485.2 | RR | A |
| | | 6.19Hz, 3H), 1.12 (d, | | | | | |
| | | J = 7.03Hz, 3H), 0.86 | | | | | |
| | | (s, 3H) | | | | | |
| 74A1 | 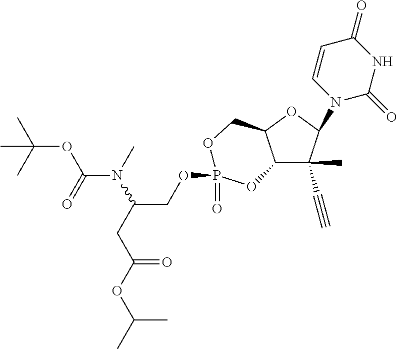 [SMILES: C#C[C@]1(C)[C@@H]2O[P@@](=O)(OCC(CC(=O)OC(C)C)N(C)C(=O)OC(C)(C)C)OC[C@H]2O[C@H]1n1ccc(=O)[nH]c1=O] | | −6.91 | S_{P} | (M + 23)⁺ 608.4 | SS | C |
| 74A2 | 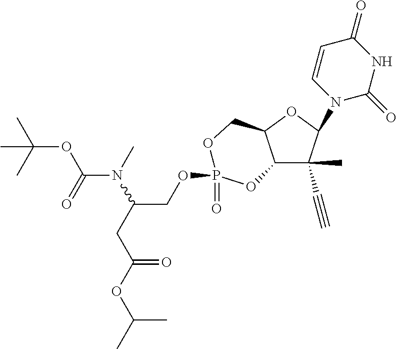 [SMILES: C#C[C@]1(C)[C@@H]2O[P@@](=O)(OCC(CC(=O)OC(C)C)N(C)C(=O)OC(C)(C)C)OC[C@H]2O[C@H]1n1ccc(=O)[nH]c1=O] | | −6.89; −6.93 | S_{P} | (M + 23)⁺ 608.4 | SS | C |
| 74B | 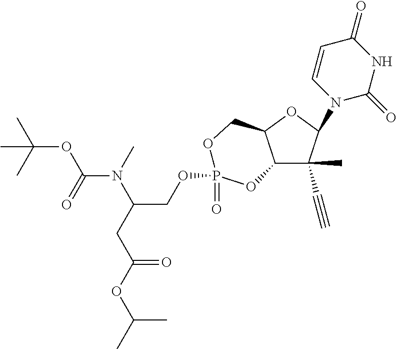 [SMILES: C#C[C@]1(C)[C@@H]2O[P@](=O)(OCC(CC(=O)OC(C)C)N(C)C(=O)OC(C)(C)C)OC[C@H]2O[C@H]1n1ccc(=O)[nH]c1=O] | | −5.68; −5.74; −5.81 | R_{P} | (M + 23)⁺ 608.4 | SS | C |
| 75A1 | 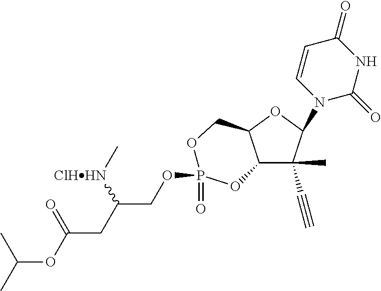 | DMSO: 11.57 (s, 1H), 9.70 (brs, 1H), 9.58 (brs, 1H), 8.20 (d, J = 8.06Hz, 1H), 6.31 (s, 1H), 5.58-5.56 (m, 1H), 5.10-5.05 (m, 1H), 4.95 (heptuplet, J = 6.32Hz, 1H), 4.87- 4.85 (m, 1H), 4.68 (ddd, J = 22.91Hz, 8.85Hz, 4.74Hz, 1H), 4.45-4.42 (m, 1H), 4.32-4.26 (m, 1H), 4.18-4.12 (m, 1H), 3.76 (brs, 1H), 3.64 (s, 1H), 2.95 (dd, J = 17.11Hz, 5.15Hz, 1H), 2.82 (dd, J = 17.27Hz, | −6.81 | S_{P} | 486.7 | SS | Via C |
| | | 8.33Hz, 1H), 2.60-2.58 | | | | | |
| | | (m, 3H), 1.23-1.20 (m, | | | | | |
| | | 9H) | | | | | |
| 75A2 | 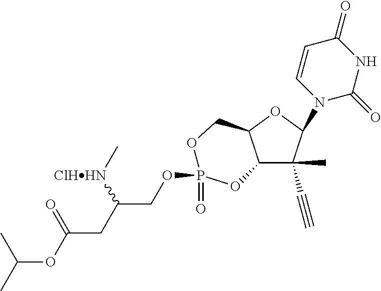 | DMSO: 11.57 (s, 1H), 9.66 (brs, 1H), 9.56 (brs, 1H), 8.22 (d, J = 8.06Hz, 1H), 6.32 (s, 1H), 5.58-5-55 (m, 1H), 5.10-5.05 (m, 1H), 4.94 (heptuplet, J = 6.20Hz, 1H), 4.92- 4.89 (m, 1H), 4.68 (ddd, J = 23.00Hz, 8.96Hz, 4.75Hz, 1H), 4.46-4.41 (m, 1H), 4.33-4.27 (m, 1H), 4.18-4.12 (m, 1H), 3.78 (brs, 1H), 3.64 (s, 1H), 2.97 (dd, J = 17.28Hz, 5.18Hz, 1H), 2.77 (dd, J = 17.28Hz, | −6.75 | S_{P} | 486.8 | SS | Via C |
| | | 8.42Hz, 1H), 2.62-2.60 | | | | | |
| | | (m, 3H), 1.21-1.19 (m, | | | | | |
| | | 9H) | | | | | |
| 76 | 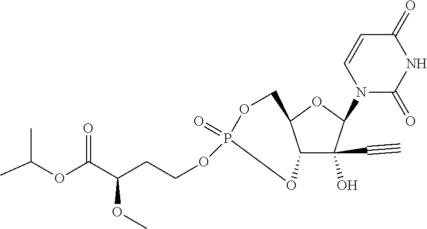 | DMSO: 11.53 (s, 1H), 7.84 (d, J = 8.01Hz, 0.4H), 7.80 (d, J = 8.01Hz, 0.6H), 7.23 (s, 0.6H), 7.18 (s, 0.4H), 6.08-6.07 (m, 1H), 5.69-5.64 (m, 1H), 5.05-4.95 (m, 1H), 4.77-4.62 (m, 2H), 4.57-4.45 (m, 1H), 4.30-4.10 (m, 3H), 3.99-3.96 (m, 0.6H), 3.88-3.85 (m, 0.4H), 3.82 (s, 1H), 3.32 (s, | −5.22 (s, 0.4P); −6.55 (s, 0.6P) | Mix- ture R_{P}/S_{P} | 489.0 | Y | B |
| | | 1.8H), 3.30 (s, 1.2H), | | | | | |
| | | 2.22-1.88 (m, 2H), | | | | | |
| | | 1.25-1.22 (m, 6H). | | | | | |
| 77A | 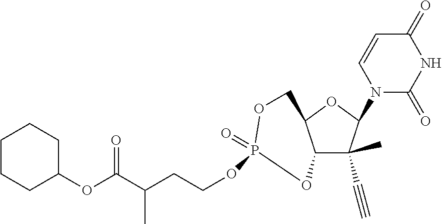 [SMILES: C#C[C@]1(C)[C@@H]2O[P@@](=O)(OCCC(C)C(=O)OC3CCCCC3)OC[C@H]2O[C@H]1n1ccc(=O)[nH]c1=O] | | −6.73 | S_{P} | 511.2 | TT | A |
| 77B | 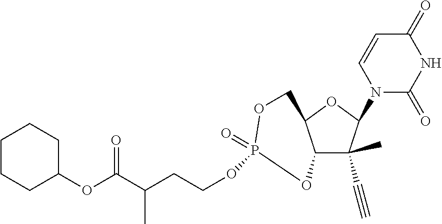 [SMILES: C#C[C@]1(C)[C@@H]2O[P@](=O)(OCCC(C)C(=O)OC3CCCCC3)OC[C@H]2O[C@H]1n1ccc(=O)[nH]c1=O] | | −5.61; −5.69 | R_{P} | 511.2 | TT | A |
| 78A | 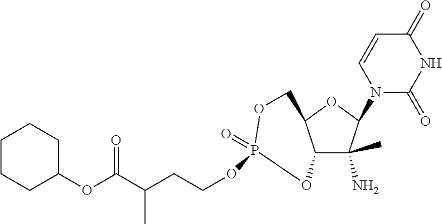 [SMILES: CC(CCO[P@@]1(=O)OC[C@H]2O[C@@H](n3ccc(=O)[nH]c3=O)[C@](C)(N)[C@@H]2O1)C(=O)OC1CCCCC1] | | −5.97 | S_{P} | 502.2 | TT | A |
| 78B | 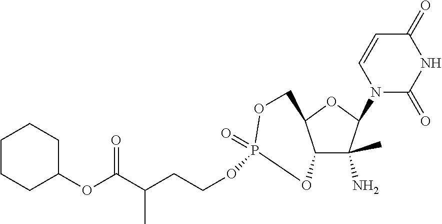 [SMILES: CC(CCO[P@]1(=O)OC[C@H]2O[C@@H](n3ccc(=O)[nH]c3=O)[C@](C)(N)[C@@H]2O1)C(=O)OC1CCCCC1] | | −4.72 | R_{P} | 502.2 | TT | A |
| 79A | 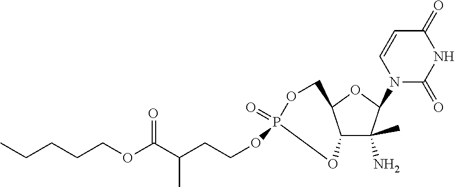 [SMILES: CCCCCOC(=O)C(C)CCO[P@@]1(=O)OC[C@H]2O[C@@H](n3ccc(=O)[nH]c3=O)[C@](C)(N)[C@@H]2O1] | | −5.99 | S_{P} | 490.4 | UU | A |
| 79B | 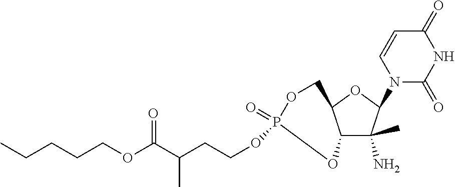 [SMILES: CCCCCOC(=O)C(C)CCO[P@]1(=O)OC[C@H]2O[C@@H](n3ccc(=O)[nH]c3=O)[C@](C)(N)[C@@H]2O1] | | −4.77 | R_{P} | 490.5 | UU | A |
| 80A | 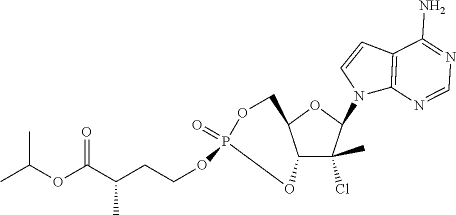 [SMILES: CC(C)OC(=O)[C@@H](C)CCO[P@@]1(=O)OC[C@H]2O[C@@H](n3ccc4c(N)ncnc43)[C@](C)(Cl)[C@@H]2O1] | | −6.32; −6.78 | S_{P} | 503.4 | RR | A |
| 80B | 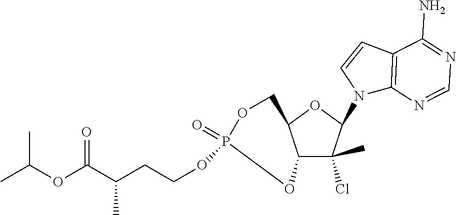 [SMILES: CC(C)OC(=O)[C@@H](C)CCO[P@]1(=O)OC[C@H]2O[C@@H](n3ccc4c(N)ncnc43)[C@](C)(Cl)[C@@H]2O1] | | −5.98 | R_{P} | 503.5 | RR | A |
| 81A | 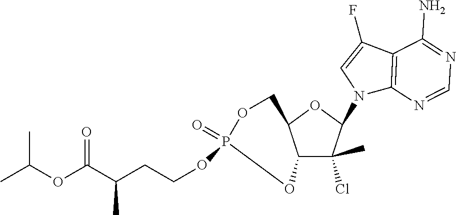 [SMILES: CC(C)OC(=O)[C@H](C)CCO[P@@]1(=O)OC[C@H]2O[C@@H](n3cc(F)c4c(N)ncnc43)[C@](C)(Cl)[C@@H]2O1] | | −6.83 | S_{P} | 521.0 | VV | A |
| 81B | 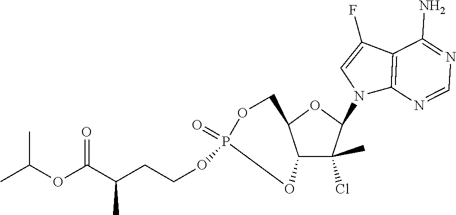 [SMILES: CC(C)OC(=O)[C@H](C)CCO[P@]1(=O)OC[C@H]2O[C@@H](n3cc(F)c4c(N)ncnc43)[C@](C)(Cl)[C@@H]2O1] | | −5.98 | R_{P} | 521.0 | VV | A |
| 82A | 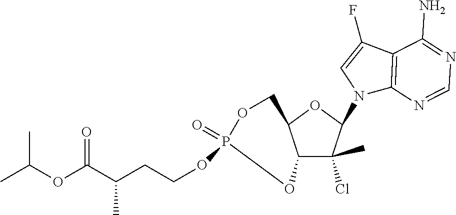 [SMILES: CC(C)OC(=O)[C@@H](C)CCO[P@@]1(=O)OC[C@H]2O[C@@H](n3cc(F)c4c(N)ncnc43)[C@](C)(Cl)[C@@H]2O1] | | −6.81 | S_{P} | 521.0 | RR | A |
| 82B | 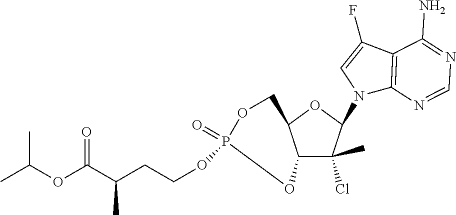 [SMILES: CC(C)OC(=O)[C@H](C)CCO[P@]1(=O)OC[C@H]2O[C@@H](n3cc(F)c4c(N)ncnc43)[C@](C)(Cl)[C@@H]2O1] | | −6.01 | R_{P} | 521.0 | RR | A |

### Example 73

### Replicon Activity and Cytotoxicity Assays

To measure cell-based anti-HCV activity of the compounds of the present invention, replicon cells (1b-Con1) are seeded at 5000 cells/well in 96-well plates one day prior to treatment with a compound of the invention. Various concentrations of a test compound of the invention in DMSO are then added to the replicon cells, with the final concentration of DMSO at 0.5% and fetal bovine serum at 10% in the assay media. Cells are harvested three days post-dosing, and the replicon RNA level is determined using real-time RT-PCR (Taqman assay) with GAPDH RNA as endogenous control. EC₅₀ values are calculated from experiments with 10 serial twofold dilutions of the inhibitor in triplicate. To measure cytotoxicity in replicon cells of an inhibitor, an MTS assay is performed according to the manufacturer's protocol for CellTiter 96 Aqueous One Solution Cell Proliferation Assay (Promega, Cat # G3580) three days post dosing on cells treated identically as in replicon activity assays. CC₅₀ is the concentration of inhibitor that yields 50% inhibition compared to vehicle-treated cells. Cytotoxicity in other types of cells may be measured using the same MTS protocol.

Data were obtained using this method for selected compounds of the present invention, and are set forth below in Table 2. These data indicate that the compounds possess significant cytotoxicity windows over replicon activity.

### Example 74

### Determination of In Vivo Conversion of Prodrug to Nucleoside Triphosphate

The degree of conversion of a prodrug compound of the present invention to its corresponding nucleoside triphosphate (NTP) was measured in vivo using the procedure described below.

Liver samples were collected from either Wistar Hannover Rats or Beagle Dogs dosed with the prodrug via the freeze clamp procedure (animals anesthetized via isofluorane, the liver was clamped with modified clamps that are frozen in liquid nitrogen, then the clamped liver piece was placed in liquid nitrogen to ensure frozen completely; the liver clamp procedure was repeated to get a second piece of liver sample; samples stored at −80° C.). Liver samples were then homogenized using a Spex Sample Prep Freezer/Mill (Cryomill); settings for the cryomill operation are 1 Cycle, 2 minute pre-chill time, 2 minute run time, 1 minute cool time, and a rate of 15 cycles/second (cps). Control liver samples collected from rats dosed with vehicle were cryomilled in the same manner. During this process it is imperative that anything that will come into contact with the liver samples remain frozen on dry ice at all times, such as all Cryomill sample containers/lids and spatulas.

The cryomilled control liver sample was used to generate the standard curve. An appropriate amount of cryomilled control liver sample was weighed out into a conical tube, depending on how many standard curves are needed, placed on wet ice and suspended with cold (approx. 0° C.) 70% Methanol/30% (20 mM EDTA/EGTA) that had been adjusted to pH 8 with sodium hydroxide at a ratio of 1:4 (liver:MeOH/EDTA-EGTA). The suspended liver homogenate was vortexed until a homogenous suspension was obtained. The standard curve ranged from 10 ng/mL to 50,000 ng/mL of NTP standard, as well as a QC sample at 10,000 ng/mL. A 500 μL aliquot of suspended control liver homogenate per each point on the standard curve and each QC was removed and placed into a 1.5 mL centrifuge tube, and 125 μL of each corresponding standard curve or QC standard solution was added to each individual control aliquot and re-vortexed. Liver sample aliquots were centrifuged at 4° C., 3645×g, for 10 minutes, and 450 μL of the supernatant was aliquoted into a 2 mL Square 96 well bioanalytical plate. Single and double blank samples are also generated from the suspended control liver homogenate using the procedure above, substituting the 125 μL of standard solution with 125 μL of water.

Approximately 1-2 grams of the cryomilled liver sample was weighed out into a 50 mL conical tube and placed on wet ice and suspended with cold 70% Methanol/30% (20 mM EDTA/EGTA) that had been adjusted to pH 8 with sodium hydroxide at a ratio of 1:4 (liver:MeOH/EDTA-EGTA); the remaining cryomilled liver sample was stored at −80° C. for possible re-assay if needed. The suspended liver homogenate was vortexed until a homogenous suspension was obtained. A 500 μL aliquot of each unknown liver sample was removed and placed into a 1.5 mL centrifuge tube, and 125 μL of water was added to each aliquot and re-vortexed. Standard curve/QC liver sample aliquots were centrifuged at 4° C., 3645×g, for 10 minutes, and 450 μL of the supernatant was aliquoted into a 2 mL square 96 well bioanalytical plate, and an appropriate internal standard was added to all sample wells, standard curve/QC wells, and the single blank well. The sample plate was stored at −80° C. until analysis and results were reported in μM of NTP measured.

Results are provided in Table 2 below.

TABLE 2

| | | | NTP Mouse |
|---|---|---|---|
| Compound | EC50 (1b) | CC50 (EDU) | AUC0-24 1 mpk |
| No | Range | Range | Range |
| 1A | ++ | ++ | +++ |
| 1A1 | +++ | ++ | ++ |
| 1A2 | ++ | ++ | ++ |
| 1B | ++ | ++ | +++ |
| 1B1 | ++ | ++ | ND |
| 1B2 | +++ | ++ | ND |
| 2A | ++ | ++ | +++ |
| 2B | ++++ | ++ | +++ |
| 3A | + | ++ | ND |
| 3B | ++++ | ++ | ++ |
| 5A | ++ | ++ | ND |
| 5B | + | ++ | ND |
| 6A | ++ | ++ | + |
| 6A1 | + | ++ | ND |
| 6A2 | + | ++ | ND |
| 6B | ++ | ++ | ND |
| 6B1 | ++ | ++ | ND |
| 6B2 | ++ | ++ | ND |
| 7A | +++ | ++ | + |
| 7A1 | +++ | ++ | + |
| 7A2 | +++ | ++ | + |
| 7B | ++++ | ++ | + |
| 7B1 | +++ | ++ | ++ |
| 7B2 | ++ | ++ | ++ |
| 8A | +++ | ++ | + |
| 8A1 | +++ | ++ | + |
| 8A2 | ++++ | ++ | + |
| 8B | ++++ | ++ | + |
| 8B1 | ++++ | ++ | ++ |
| 8B2 | +++ | ++ | ++ |
| 10A | + | ++ | ++ |
| 10A1 | + | ++ | ++ |
| 10A2 | + | ++ | ++ |
| 10B | + | ++ | +++ |
| 10B1 | + | ++ | ++ |
| 10B2 | + | ++ | ++ |
| 14A | + | ++ | +++ |
| 14A1 | + | ++ | ++ |
| 14A2 | + | ++ | ++ |
| 14B | + | ++ | +++ |
| 14B1 | ++ | ++ | ++ |
| 14B2 | ++ | ++ | +++ |
| 15A | + | ++ | ND |
| 15B | + | ++ | + |
| 16A | + | ++ | + |
| 16B | + | ++ | + |
| 17A | + | ++ | + |
| 17B | + | ++ | + |
| 18A | + | ++ | ++ |
| 18B | + | ++ | ++ |
| 19A | + | ++ | + |
| 19B | + | ++ | ++ |
| 20A | + | ++ | + |
| 20B | + | ++ | + |
| 21A | + | ++ | + |
| 21B | + | ++ | ++ |
| 22A | + | ++ | ND |
| 22B | + | ++ | ND |
| 23A | + | ++ | ++ |
| 23B | + | ++ | ++ |
| 24A | + | ++ | + |
| 24B | + | ++ | ++ |
| 25A | + | ND | ++ |
| 25B | + | ND | ++ |
| 26A | + | ++ | ++ |
| 26B | + | ++ | ++ |
| 27A | + | ++ | + |
| 27B | + | ++ | ++ |
| 28A | + | ++ | + |
| 28B | + | ++ | ++ |
| 29A | + | ++ | ++ |
| 29B | + | ++ | ++ |
| 30A | + | ++ | ++ |
| 30B | + | ++ | + |
| 31A | +++ | ++ | ++ |
| 31B | + | ++ | ++ |
| 32A | + | ++ | + |
| 32B | + | ++ | ++ |
| 33A | + | ++ | ND |
| 33B | + | ++ | +++ |
| 34A | + | ++ | + |
| 34B | + | ++ | + |
| 35A | ++++ | ++ | ++ |
| 35A1 | ++++ | ++ | +++ |
| 35A2 | ++++ | ++ | ++ |
| 35B | ++++ | ++ | +++ |
| 35B1 | ++++ | ++ | +++ |
| 35B2 | ++++ | ++ | +++ |
| 36A | +++ | ++ | ND |
| 36B | ++ | ++ | ++ |
| 37A | +++ | ++ | ++ |
| 37B | ++++ | ++ | +++ |
| 38A | ++++ | ++ | + |
| 38B | ++++ | ++ | ++ |
| 39A | ++++ | ++ | ++ |
| 39A1 | ++++ | ++ | +++ |
| 39A2 | ND | ND | ND |
| 39B | ++++ | ++ | +++ |
| 39B1 | ++++ | ++ | +++ |
| 39B2 | +++ | ++ | +++ |
| 40A | + | ++ | + |
| 40B | + | ++ | + |
| 41A | + | ++ | ND |
| 41B | + | ++ | ND |
| 42A | + | ++ | + |
| 42B | + | ++ | + |
| 43A | + | ++ | + |
| 43B | + | ++ | ++ |
| 44A | + | ++ | + |
| 44B | + | ++ | + |
| 45A | ++ | ++ | + |
| 45B | + | ++ | ++ |
| 46A | + | ++ | + |
| 46B | + | ++ | + |
| 47A | + | ++ | ++ |
| 47B | + | ++ | ++ |
| 48A | + | ++ | ND |
| 48B | + | ++ | ND |
| 49A | + | ++ | + |
| 49B | ++ | + | + |
| 50A | + | ++ | + |
| 50B | +++ | ++ | ++ |
| 51A | ++ | ++ | + |
| 51B | ++++ | ++ | ++ |
| 52A | + | ND | + |
| 52B | + | ND | + |
| 53A | + | ND | ND |
| 53B | + | ++ | ND |
| 54A | + | ++ | + |
| 54B | + | ++ | + |
| 55A | + | ND | + |
| 55B | + | ND | + |
| 56A | +++ | ++ | ND |
| 56B | +++ | ++ | ND |
| 57A | ++ | ++ | ++ |
| 57B | ++ | ++ | ND |
| 58A | + | + | + |
| 59A | +++ | ++ | + |
| 59B | ++++ | ++ | + |
| 60A | + | ++ | ND |
| 60B | + | ++ | ND |
| 61A | + | ++ | + |
| 61B | + | ++ | ++ |
| 62A | + | ++ | ND |
| 62B | + | ++ | ND |
| 63A | + | ++ | + |
| 63B | + | ++ | + |
| 64A | +++ | ++ | ND |
| 64B | +++ | ++ | ++ |
| 65A | + | ++ | + |
| 66A | + | ++ | ND |
| 66B | +++ | ++ | ++ |
| 67A | + | ++ | + |
| 68A | + | ++ | + |
| 68B | ++ | ++ | + |
| 69A | + | ++ | + |
| 69B | + | ++ | + |
| 70A | +++ | ++ | + |
| 70B | ++ | ++ | +++ |
| 71A | + | ++ | + |
| 71B | ++ | ++ | ++ |
| 72A | + | ++ | +++ |
| 72B | +++ | ++ | +++ |
| 73A | + | ++ | + |
| 73B | ++ | ++ | +++ |
| 74A1 | + | ++ | ND |
| 74A2 | + | ++ | + |
| 74B | + | ++ | + |
| 75A1 | + | ++ | ND |
| 75A2 | + | ND | ND |
| 76 | ++++ | ++ | ND |
| 77A | ++ | ++ | ++ |
| 77B | ++++ | ++ | + |
| 78A | + | ++ | ++ |
| 78B | ++ | ++ | ++ |
| 79A | ++ | ++ | ++ |
| 79B | ++ | ++ | + |
| 80A | + | ND | + |
| 80B | ++ | ND | + |
| 81A | + | ND | + |
| 81B | + | ND | + |
| 82A | + | ND | + |
| 82B | + | ND | + |
| 83A1 | ++++ | ++ | ND |
| 84A1 | ++++ | ++ | ND |
| 85A2 | ++++ | ++ | ND |
| 86A | ++ | ++ | ND |
| 87A | ++++ | ND | + |
| 87B | ++++ | ND | ND |
| 88A | ++++ | ++ | ND |
| 88B | ++++ | ++ | ND |
| 89A | ND | ND | ND |
| 90A | ND | ND | ND |

¹ EC₅₀ is provided as follows: ++++ ≤ 250 nM, 250 nM < +++ ≤ 1 μM; 1 μM < ++ ≤ 10 μM; and + >10 μM
² CC₅₀ is provided as follows: + ≤ 50 μM and ++ >50 μM
³ TP Mouse AUC0-24 1 mpk is provided as follows: + ≤ 50; 50 < ++ ≤ 150; +++ > 150; and ND = Not Determined

### Uses of the Cyclic Phosphate Substituted Nucleoside Derivatives

### Treatment or Prevention of HCV Infection

The Cyclic Phosphate Substituted Nucleoside Derivatives are useful in the inhibition of HCV, the treatment of HCV infection and/or reduction of the likelihood or severity of symptoms of HCV infection and the inhibition of HCV viral replication and/or HCV viral production in a cell-based system. For example, the Cyclic Phosphate Substituted Nucleoside Derivatives are useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the invention provides methods for treating HCV infection in a patient, the methods comprising administering to the patient an effective amount of at least one Cyclic Phosphate Substituted Nucleoside Derivative or a pharmaceutically acceptable salt thereof. In a specific embodiment, the amount administered is effective to treat or prevent infection by HCV in the patient. In another specific embodiment, the amount administered is effective to inhibit HCV viral replication and/or viral production in the patient.

The Cyclic Phosphate Substituted Nucleoside Derivatives are also useful in the preparation and execution of screening assays for antiviral compounds. For example the Cyclic Phosphate Substituted Nucleoside Derivatives are useful for identifying resistant HCV replicon cell lines harboring mutations within NS5B, which are excellent screening tools for more powerful antiviral compounds. Furthermore, the Cyclic Phosphate Substituted Nucleoside Derivatives are useful in establishing or determining the binding site of other antivirals to the HCV NS5B polymerase.

The compositions and combinations of the present invention may be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b.

In one aspect, the present invention provides for the use of a compound of formula (I), or a pharmaceutically acceptable salt thereof, for inhibiting HCV NS5B activity or for preventing and/or treating infection by HCV in a patient in need thereof.

### Combination Therapy

In another embodiment, the present methods for treating or preventing HCV infection can further comprise the administration of one or more additional therapeutic agents which are not Cyclic Phosphate Substituted Nucleoside Derivatives.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one Cyclic Phosphate Substituted Nucleoside Derivative (which may include two or more different 2′-Substituted Nucleoside Derivatives), or a pharmaceutically acceptable salt thereof, and (ii) at least one additional therapeutic agent that is other than a Cyclic Phosphate Substituted Nucleoside Derivative, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Cyclic Phosphate Substituted Nucleoside Derivative and an additional therapeutic agent may be present in fixed amounts (dosage amounts) in a single dosage unit (e.g., a capsule, a tablet and the like).

In one embodiment, the at least one Cyclic Phosphate Substituted Nucleoside Derivative is administered during a time when the additional therapeutic agent(s) exert their prophylactic or therapeutic effect, or vice versa.

In another embodiment, the at least one Cyclic Phosphate Substituted Nucleoside Derivative and the additional therapeutic agent(s) are administered in doses commonly employed when such agents are used as monotherapy for treating a viral infection.

In another embodiment, the at least one Cyclic Phosphate Substituted Nucleoside Derivative and the additional therapeutic agent(s) are administered in doses lower than the doses commonly employed when such agents are used as monotherapy for treating a viral infection.

In still another embodiment, the at least one Cyclic Phosphate Substituted Nucleoside Derivative and the additional therapeutic agent(s) act synergistically and are administered in doses lower than the doses commonly employed when such agents are used as monotherapy for treating a viral infection.

In one embodiment, the at least one Cyclic Phosphate Substituted Nucleoside Derivative and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that may be treated or prevented using the combination therapy methods of the present invention include, but are not limited to, those listed above.

In one embodiment, the viral infection is HCV infection.

The at least one Cyclic Phosphate Substituted Nucleoside Derivative and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Cyclic Phosphate Substituted Nucleoside Derivative and the additional therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, one or more compounds of the invention are administered with one or more additional therapeutic agents, including but not limited to the therapeutic agents described, supra.

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In still another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In yet another embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In still another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor

In yet another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In a further embodiment, the additional therapeutic agent is an HCV entry inhibitor.

In another embodiment, the additional therapeutic agent is an HCV assembly inhibitor.

In another embodiment, one or more compounds of the present invention are administered with one additional therapeutic agent selected from an HCV protease inhibitor, an interferon, a pegylated interferon and ribavirin. In another embodiment, one or more compounds of the present invention are administered with one additional therapeutic agent selected from an HCV polymerase inhibitor, a viral protease inhibitor, an interferon, and a viral replication inhibitor. In another embodiment, one or more compounds of the present invention are administered with ribavirin, or a pharmaceutically acceptable salt thereof.

In still another embodiment, one or more compounds of the present invention are administered with two additional therapeutic agents selected from an HCV protease inhibitor, an HCV replication inhibitor, a nucleoside, an interferon, a pegylated interferon and ribavirin, or a pharmaceutically acceptable salt thereof.

In another embodiment, one or more compounds of the present invention are administered with an HCV protease inhibitor and ribavirin. In another specific embodiment, one or more compounds of the present invention are administered with a pegylated interferon and ribavirin, or a pharmaceutically acceptable salt thereof.

In another embodiment, one or more compounds of the present invention are administered with three additional therapeutic agents selected from an HCV protease inhibitor, an HCV replication inhibitor, a nucleoside, an interferon, a pegylated interferon and ribavirin, or a pharmaceutically acceptable salt thereof.

In one embodiment, one or more compounds of the present invention are administered with two additional therapeutic agents selected from an HCV polymerase inhibitor, a viral protease inhibitor, an interferon, and a viral replication inhibitor.

In another embodiment, one or more compounds of the present invention are administered with ribavirin, interferon and another therapeutic agent.

In another embodiment, one or more compounds of the present invention are administered with ribavirin, interferon and another therapeutic agent, wherein the additional therapeutic agent is selected from an HCV polymerase inhibitor, a viral protease inhibitor, and a viral replication inhibitor.

In still another embodiment, one or more compounds of the present invention are administered with ribavirin, interferon and a viral protease inhibitor.

In another embodiment, one or more compounds of the present invention are administered with ribavirin, interferon and an HCV protease inhibitor.

In another embodiment, one or more compounds of the present invention are administered with ribavirin, interferon and boceprevir or telaprevir, or a pharmaceutically acceptable salt thereof.

In a further embodiment, one or more compounds of the present invention are administered with ribavirin, interferon and an HCV polymerase inhibitor.

In another embodiment, one or more compounds of the present invention are administered with pegylated-interferon alpha and ribavirin, or a pharmaceutically acceptable salt thereof.

In one embodiment, the additional therapeutic agents comprise a viral protease inhibitor and a viral polymerase inhibitor.

In still another embodiment, the additional therapeutic agents comprise a viral protease inhibitor and an immunomodulatory agent.

In yet another embodiment, the additional therapeutic agents comprise a polymerase inhibitor and an immunomodulatory agent.

In another embodiment, the additional therapeutic agents comprise a viral protease inhibitor and a nucleoside.

In another embodiment, the additional therapeutic agents comprise an immunomodulatory agent and a nucleoside.

In one embodiment, the additional therapeutic agents comprise an HCV protease inhibitor and an HCV polymerase inhibitor.

In one embodiment, the additional therapeutic agents comprise an HCV protease inhibitor and an HCV NS5A inhibitor.

In another embodiment, the additional therapeutic agents comprise a nucleoside and an HCV NS5A inhibitor.

In another embodiment, the additional therapeutic agents comprise a viral protease inhibitor, an immunomodulatory agent and a nucleoside.

In a further embodiment, the additional therapeutic agents comprise a viral protease inhibitor, a viral polymerase inhibitor and an immunomodulatory agent.

In another embodiment, the additional therapeutic agent is ribavirin.

HCV polymerase inhibitors useful in the present compositions and methods include, but are not limited to, VP-19744 (Wyeth/ViroPharma), PSI-7851 (Pharmasset), RG7128 (Roche/Pharmasset), Sofosbuvir (Gilead), PSI-938 (Pharmasset-Gilead), PSI-879 (Pharmasset-Gilead), PSI-661 (Pharmasset), PF-868554/filibuvir (Pfizer), VCH-759/VX-759 (ViroChem Pharma/Vertex), HCV-371 (Wyeth/VirroPharma), HCV-796 (Wyeth/ViroPharma), IDX-184 (Idenix), IDX-375 (Idenix), NM-283 (Idenix/Novartis), MK-3682 (Merck), GL-60667 (Genelabs), JTK-109 (Japan Tobacco), PSI-6130 (Pharmasset), R1479 (Roche), R-1626 (Roche), R-7128 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3682 (Merck), VCH-222/VX-222 (ViroChem/Vertex), VCH-916 (ViroChem), VCH-716 (ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004), as well as pharmaceutically acceptable salts of any of the above agents.

Other HCV polymerase inhibitors useful in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125; and the following compounds:

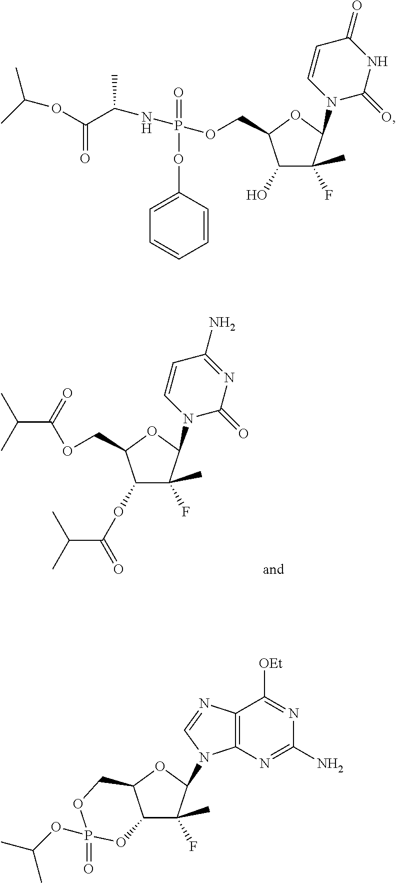

and pharmaceutically acceptable salts thereof.

Interferons useful in the present compositions and methods include, but are not limited to, interferon alfa-2a, interferon alfa-2b, interferon alfacon-1 and petroleum etherG-interferon alpha conjugates. “PEG-interferon alpha conjugates” are interferon alpha molecules covalently attached to a petroleum etherG molecule. Illustrative petroleum etherG-interferon alpha conjugates include interferon alpha-2a (Roferon™, Hoffman La-Roche, Nutley, N.J.) in the form of pegylated interferon alpha-2a (e.g., as sold under the trade name Pegasysm), interferon alpha-2b (Intron™, from Schering-Plough Corporation) in the form of pegylated interferon alpha-2b (e.g., as sold under the trade name petroleum etherG-Intron™ from Schering-Plough Corporation), interferon alpha-2b-XL (e.g., as sold under the trade name petroleum etherG-Intron™), interferon alpha-2c (Berofor Alpha™, Boehringer Ingelheim, Ingelheim, Germany), petroleum etherG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), R-7025 (Roche), IFN-α-2b-XL (Flamel Technologies), belerofon (Nautilus) and consensus interferon as defined by determination of a consensus sequence of naturally occurring interferon alphas (Infergen™, Amgen, Thousand Oaks, Calif.).

Examples of viral protease inhibitors useful in the present compositions and methods include, but are not limited to, an HCV protease inhibitor. Examples of HCV protease inhibitors useful in the present compositions and methods include, but are not limited to, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott/Enanta), TMC-435350 (Medivir), RG7227 (Danoprevir, InterMune/Roche), EA-058 (Abbott/Enanta), EA-063 (Abbott/Enanta), GS-9256 (Gilead), IDX-320 (Idenix), ACH-1625 (Achillion), ACH-2684 (Achillion), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), grazoprevir (Merck), BMS-650032 (Bristol-Myers Squibb), VX-985 (Vertex) and PHX1766 (Phenomix), as well as pharmaceutically acceptable salts of any of the above agents.

Viral replication inhibitors useful in the present compositions and methods include, but are not limited to, HCV replicase inhibitors, IRES inhibitors, NS4A inhibitors, NS3 helicase inhibitors, NS5A inhibitors, NS5B inhibitors, ribavirin, AZD-2836 (Astra Zeneca), viramidine, A-831 (Arrow Therapeutics), EDP-239 (Enanta), ACH-2928 (Achillion), GS-5885 (Gilead); an antisense agent or a therapeutic vaccine, as well as pharmaceutically acceptable salts of any of the above agents.

HCV NS5A inhibitors useful in the present compositions and methods include, but are not limited to, ACH-2928 (Achilon), A-832 (Arrow Therpeutics), AZD-7295 (Astra Zeneca/Arrow), GS-5885 (Gilead), Ledipasvir (Gilead), Velpatasvir (Gilead), Samatasvir (Merck), PPI-461 (Presidio), PPI-1301 (Presidio), BMS-824383 (Bristol-Myers Squibb), BMS-790052 (Bristol-Myers Squibb), elbasvir (Merck) and ruzasvir (Merck), as well as pharmaceutically acceptable salts of any of the above agents. Additional HCV NS5A inhibitors useful as second additional therapeutic agents in the present compositions and methods include, but are not limited to those disclosed in International Publication No. WO 2010/111483.

HCV replicase inhibitors useful in the present compositions and methods include, but are not limited to, those disclosed in U.S. Patent Publication No. US20090081636.

The doses and dosage regimen of the other agents used in the combination therapies of the present invention for the treatment or prevention of HCV infection may be determined using the attending clinician, taking into consideration the approved doses and dosage regimen in the package insert; the age, sex and general health of the patient; and the type and severity of the viral infection or related disease or disorder. When administered in combination, the Cyclic Phosphate Substituted Nucleoside Derivative(s) and the other agent(s) may be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

Generally, a total daily dosage of the at least one Cyclic Phosphate Substituted Nucleoside Derivative(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

### Compositions and Administration

Due to their activity, the Cyclic Phosphate Substituted Nucleoside Derivatives are useful in veterinary and human medicine. As described above, the Cyclic Phosphate Substituted Nucleoside Derivatives are useful for treating or preventing HCV infection in a patient in need thereof.

When administered to a patient, the Cyclic Phosphate Substituted Nucleoside Derivatives may be administered as a component of a composition that comprises a pharmaceutically acceptable carrier or vehicle. The present invention provides pharmaceutical compositions comprising an effective amount of at least one Cyclic Phosphate Substituted Nucleoside Derivative and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules may be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Cyclic Phosphate Substituted Nucleoside Derivatives are administered orally.

In another embodiment, the one or more Cyclic Phosphate Substituted Nucleoside Derivatives are administered intravenously.

In one embodiment, a pharmaceutical preparation comprising a Cyclic Phosphate Substituted Nucleoside Derivative is in unit dosage form. In such form, the preparation is subdivided into unit doses containing effective amounts of the active components.

Compositions may be prepared according to conventional mixing, granulating or coating methods, respectively, and the present compositions can contain, in one embodiment, from about 0.1% to about 99% of the Cyclic Phosphate Substituted Nucleoside Derivative(s) by weight or volume. In various embodiments, the present compositions can contain, in one embodiment, from about 1% to about 70% or from about 5% to about 60% of the Cyclic Phosphate Substituted Nucleoside Derivative(s) by weight or volume.

The quantity of Cyclic Phosphate Substituted Nucleoside Derivative in a unit dose of preparation may be varied or adjusted from about 1 mg to about 2500 mg. In various embodiment, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Cyclic Phosphate Substituted Nucleoside Derivatives will be regulated according to the judgment of the attending clinician considering such factors as age, condition and size of the patient as well as severity of the symptoms being treated. Generally, a total daily dosage of the Cyclic Phosphate Substituted Nucleoside Derivatives range from about 0.1 to about 2000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Cyclic Phosphate Substituted Nucleoside Derivative or a pharmaceutically acceptable salt thereof; (ii) one or more additional therapeutic agents that are not a Cyclic Phosphate Substituted Nucleoside Derivative; and (iii) a pharmaceutically acceptable carrier, wherein the amounts in the composition are together effective to treat HCV infection.

In one embodiment, the present invention provides compositions comprising a Compound of Formula (I) and a pharmaceutically acceptable carrier.

In another embodiment, the present invention provides compositions comprising a Compound of Formula (I), a pharmaceutically acceptable carrier, and a second therapeutic agent selected from the group consisting of HCV antiviral agents, immunomodulators, and anti-infective agents.

In another embodiment, the present invention provides compositions comprising a Compound of Formula (I), a pharmaceutically acceptable carrier, and two additional therapeutic agents, each of which are independently selected from the group consisting of HCV antiviral agents, immunomodulators, and anti-infective agents.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula (I): 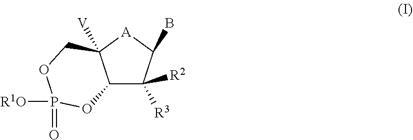
or a pharmaceutically acceptable salt thereof,
wherein:
A is selected from O, S and CH₂;
B is selected from one of the following groups: 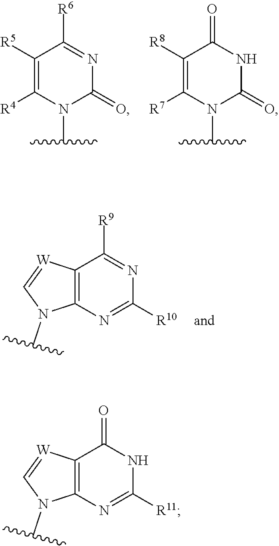
R¹ is —CH(R¹³)—X—Y—Z—R¹⁹;
Q is O or S;
V is H, halo or —N(R¹²)₂;
W is N, CH or CF;
X is a bond or —C(R¹⁴)₂—;
Y is selected from a bond, O, —S(O)₂— and —C(R¹⁵)₂—, such that when Y is O or —S(O)₂—, then X is —C(R¹⁵)₂—;
Z is selected from a bond, —C(R¹⁶)₂— and C₃-C₆ cycloalkylene, such that if X and Y are each a bond, then Z is —C(R¹⁶)₂— or C₃-C₆ cycloalkylene;
R² is selected from H, F, Cl, C₁-C₃ alkyl and C₂-C₃ alkynyl;
R³ is selected from C₁-C₆ alkyl, C₂-C₆ alkenyl, C₂-C₆ alkynyl, —OR¹², F, Cl, —N₃, —CN and —N(R¹²)₂, such that if R² is F or Cl, then R³ is other than F or Cl;
R⁴, R⁵, R⁷ and R⁸ are each independently selected from H, C₁-C₆ alkyl, C₁-C₆ haloalkyl, C₁-C₆ hydroxyalkyl, halo, —OR¹⁸, —SR¹⁸ and —N(R¹⁸)₂;
R⁶, R⁹, R¹⁰ and R¹¹ are each independently selected from H, C₁-C₆ alkyl, C₂-C₆ alkenyl, C₂-C₆ alkynyl, C₃-C₇ cycloalkyl, 5- or 6-membered monocyclic heteroaryl, 9- or 10-membered bicyclic heteroaryl, halo, —OR¹⁸, —SR¹⁸, —S(O)R¹⁸, —S(O)₂R¹⁸, —S(O)₂N(R¹⁸)₂, —NHC(O)OR¹⁸, —NHC(O)N(R¹⁸)₂, C₁-C₆ haloalkyl, C₁-C₆ hydroxyalkyl, —O—(C₁-C₆ haloalkyl), —CN, —NO₂, —N(R¹⁸)₂, —NH(C₁-C₆ alkylene)-(5- or 6-membered monocyclic heteroaryl), —NH(C₁-C₆ alkylene)-(9- or 10-membered bicyclic heteroaryl), —C(O)R¹⁸, —C(O)OR⁸, —C(O)N(R¹⁸)₂ and —NHC(O)R¹⁸, wherein said C₂-C₆ alkenyl group and said C₂-C₆ alkynyl group may be optionally substituted with halo;
each occurrence of R¹² is independently selected from H, C₁-C₆ alkyl, —C(O)R¹⁸ and —C(O)OR¹⁸;
R¹³ is selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)C(O)OR¹⁷ and —C(O)OR¹⁷;
each occurrence of R¹⁴ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)₂, —N(R¹²)C(O)OR¹⁷ and —C(O)OR¹⁷, or both R¹⁴ groups, together with the common carbon atom to which they are attached, can join to form a C₃-C₆ spirocyclic cycloalkyl group;
each occurrence of R¹⁵ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)₂, —N(R¹²)C(O)OR¹⁷ and —C(O)OR¹⁷, or both R¹⁵ groups, together with the common carbon atom to which they are attached, can join to form a C₃-C₆ spirocyclic cycloalkyl group;
each occurrence of R¹⁶ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)C(O)OR¹⁷ and —C(O)OR¹⁷, or both R¹⁶ groups, together with the common carbon atom to which they are attached, can join to form a C₃-C₆ spirocyclic cycloalkyl group;
each occurrence of R¹⁷ is independently selected from H, C₁-C₆ alkyl, C₃-C₇ cycloalkyl and C₆-C₁₀ aryl;
each occurrence of R¹⁸ is independently selected from H, C₁-C₆ alkyl, C₁-C₆ haloalkyl, C₁-C₆ hydroxyalkyl, —(C₁-C₃ alkylene)ₘ-(C₃-C₇ cycloalkyl), —(C₁-C₃ alkylene)ₘ-(C₆-C₁₀ aryl), —(C₁-C₃ alkylene)ₘ-(4 to 7-membered heterocycloalkyl), —(C₁-C₃ alkylene)ₘ-(5- or 6-membered monocyclic heteroaryl) and —(C₁-C₃ alkylene)ₘ-(9- or 10-membered bicyclic heteroaryl);
R¹⁹ is —C(O)OR¹⁷ or: 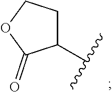 [SMILES: *C1CCOC1=O]
and
each occurrence of m is independently 0 or 1,
such that at least one of R¹³, R¹⁴, R¹⁵ and R¹⁶ is other than H.

2. The compound of claim 1, wherein A is O, or a pharmaceutically acceptable salt thereof.

3. The compound of claim 2, wherein R² is methyl, or a pharmaceutically acceptable salt thereof.

4. The compound of claim 2, wherein R³ is selected from —OH, F, Cl, —N₃, —CN, —C≡CH and —NH₂, or a pharmaceutically acceptable salt thereof.

5. The compound of claim 4, wherein B is guanine, cytosine, adenine or uracil, or a pharmaceutically acceptable salt thereof.

6. The compound of claim 1, having the formula (Ia): 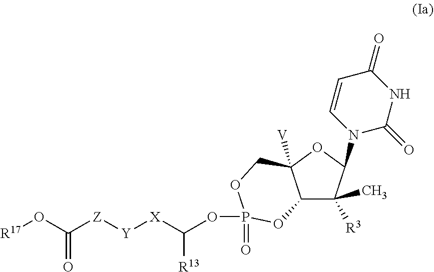
or a pharmaceutically acceptable salt thereof,
wherein:
V is H or F;
X is a bond or —C(R⁴)₂—;
Y is selected from a bond, O, S(O)₂ and —C(R¹⁵)₂—; such that when Y is O or —S(O)₂—, then X is —C(R¹⁵)₂—;
Z is selected from a bond, —C(R¹⁶)₂ and C₃-C₆ cycloalkylene, such that if X and Y are each a bond, then Z is —C(R¹⁶)₂— or C₃-C₆ cycloalkylene;
R³ is selected from —OH, F, Cl, N₃, —CN, —C≡CH and —NH₂;
each occurrence of R¹³ is independently selected from H, phenyl and C₁-C₆ alkyl;
each occurrence of R¹⁴ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)₂, —N(R¹²)C(O)OR¹⁷ and —C(O)OR¹⁷;
each occurrence of R¹⁵ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)₂, —N(R¹²)C(O)OR¹⁷ and —C(O)OR¹⁷;
each occurrence of R¹⁶ is independently selected from H, halo, C₁-C₆ alkyl, C₁-C₆ hydroxylalkyl, C₁-C₆ haloalkyl, C₆-C₁₀ aryl, —OR¹⁷, —O—C(O)R¹⁷, —N(R¹²)C(O)OR¹⁷ and —C(O)OR¹⁷, or two R¹⁶ groups that are attached to the same carbon atom, together with the common carbon atom to which they are attached, can join to form a C₃-C₆ spirocyclic cycloalkyl group;
each occurrence of R¹⁷ is independently selected from H, C₁-C₆ alkyl, C₃-C₇ cycloalkyl and C₆-C₁₀ aryl; and
each occurrence of m is independently 0 or 1.

7. The compound of claim 1, wherein X is a bond, or a pharmaceutically acceptable salt thereof.

8. The compound of claim 7, wherein Y is a bond, or a pharmaceutically acceptable salt thereof.

9. The compound of claim 1, wherein R¹⁷ is methyl, ethyl, isopropyl, t-butyl, n-pentyl, cyclopentyl or cyclohexyl, or a pharmaceutically acceptable salt thereof.

10. The compound of claim 1, wherein R¹ is selected from:

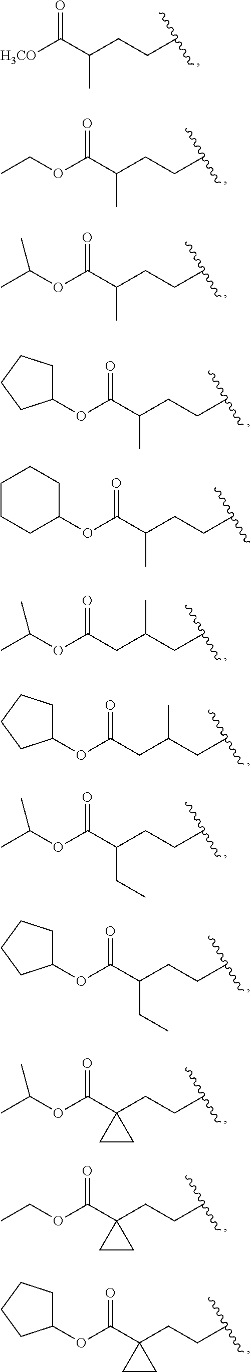

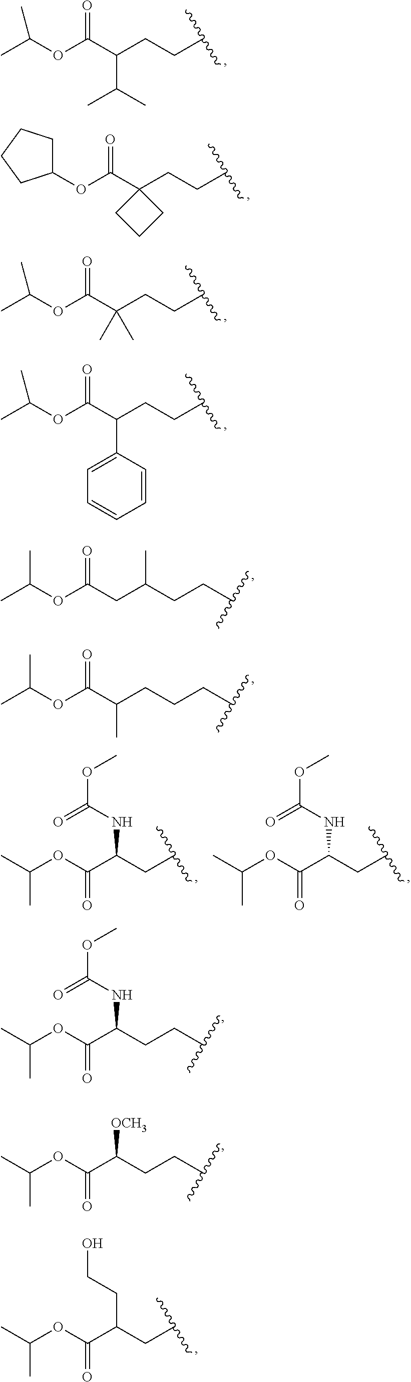

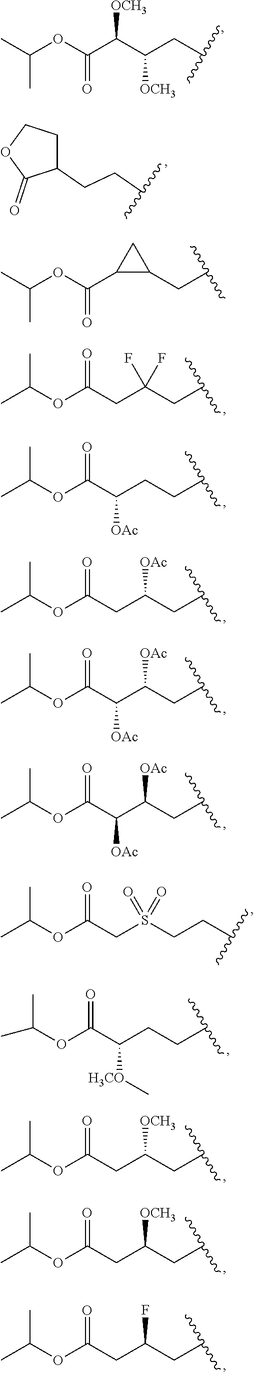

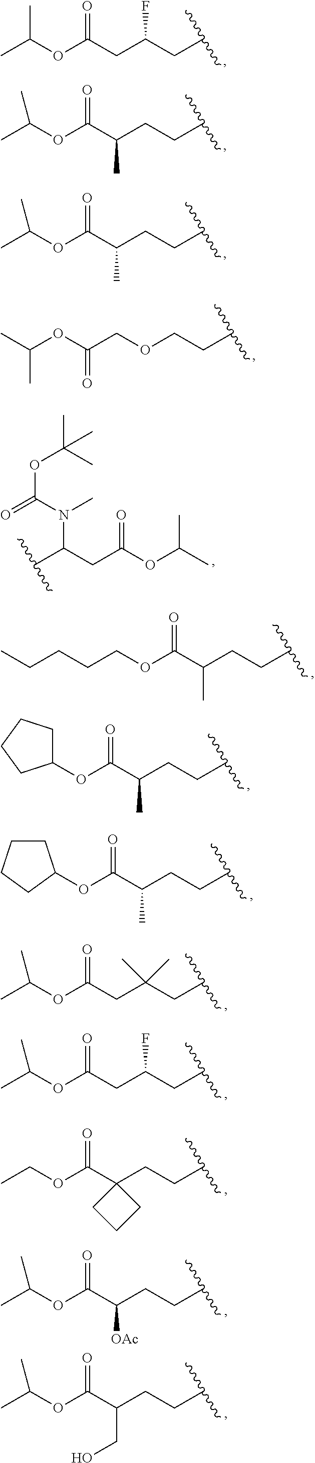

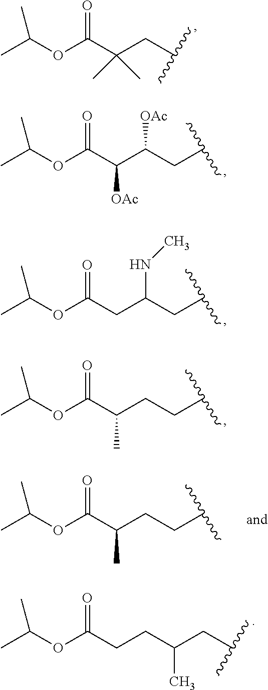

11. The compound of claim 1, wherein X is a bond and Z is —CHR¹⁶—, and wherein R¹⁶ is selected from H, C₁-C₆ alkyl, —O—(C₁-C₆ alkyl), —C(O)O—(C₁-C₆ alkyl) and —O—C(O)—(C₁-C₆ alkyl), or a pharmaceutically acceptable salt thereof.

12. The compound of claim 10, wherein R¹³ is H and Y is —CH₂— or —CH(CH₃)—, or a pharmaceutically acceptable salt thereof.

13. A compound having the structure: 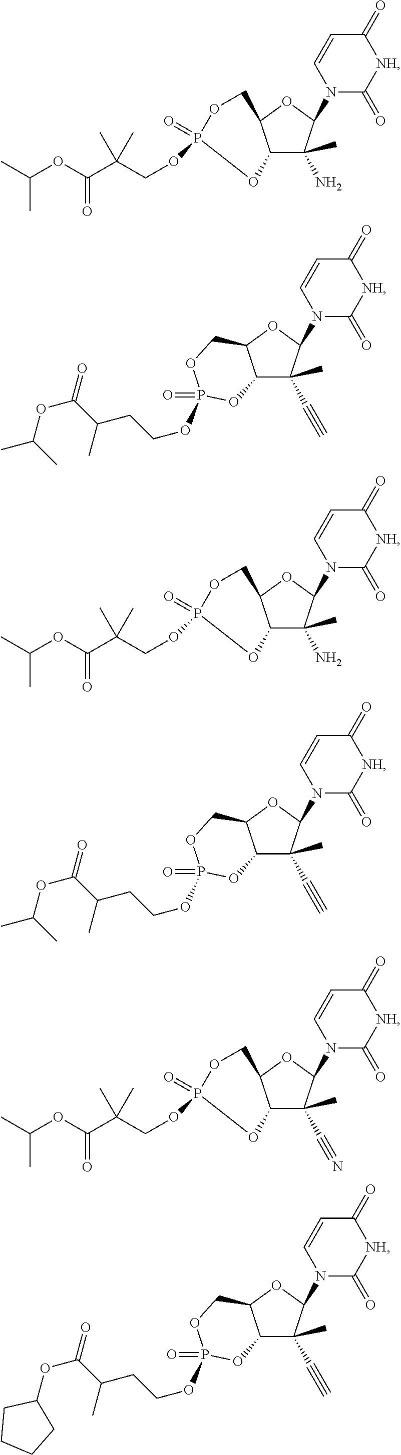 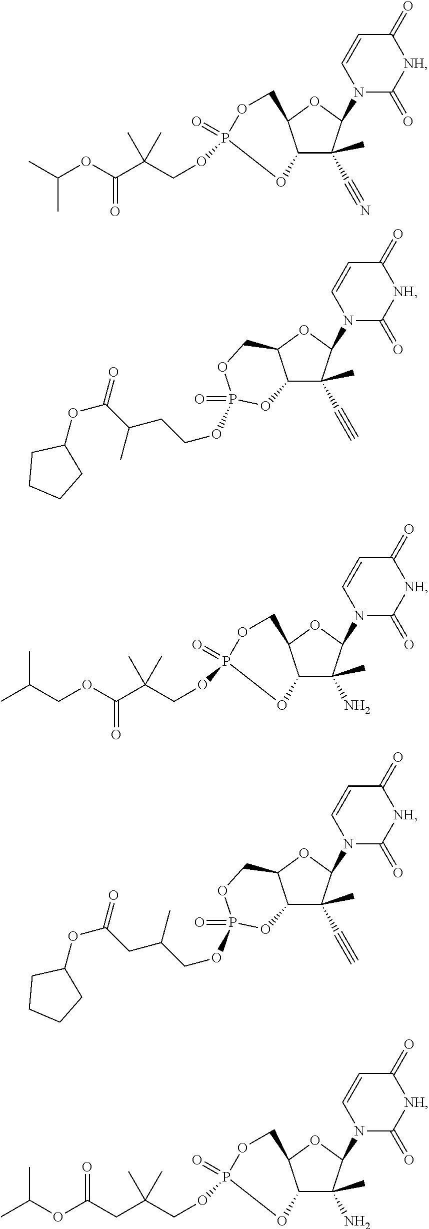 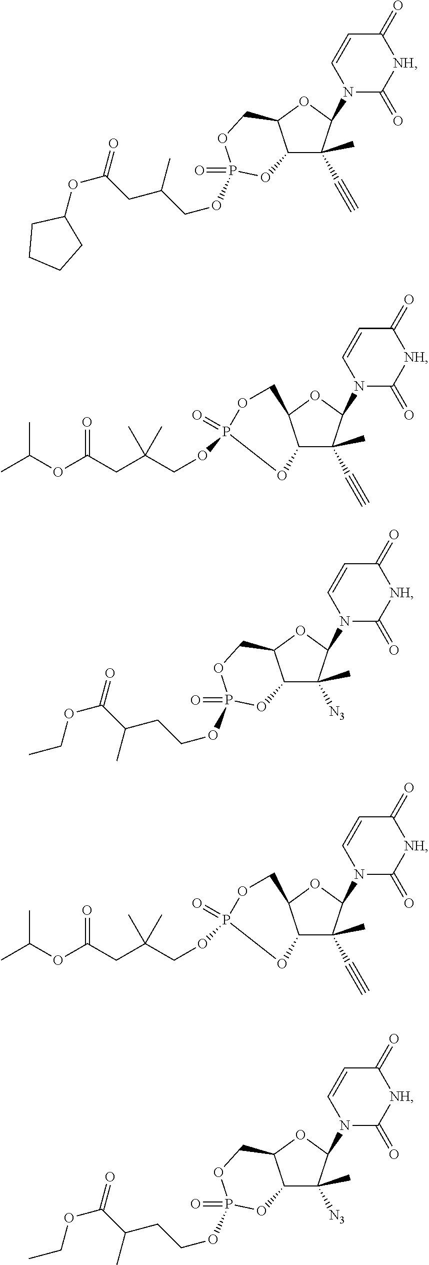 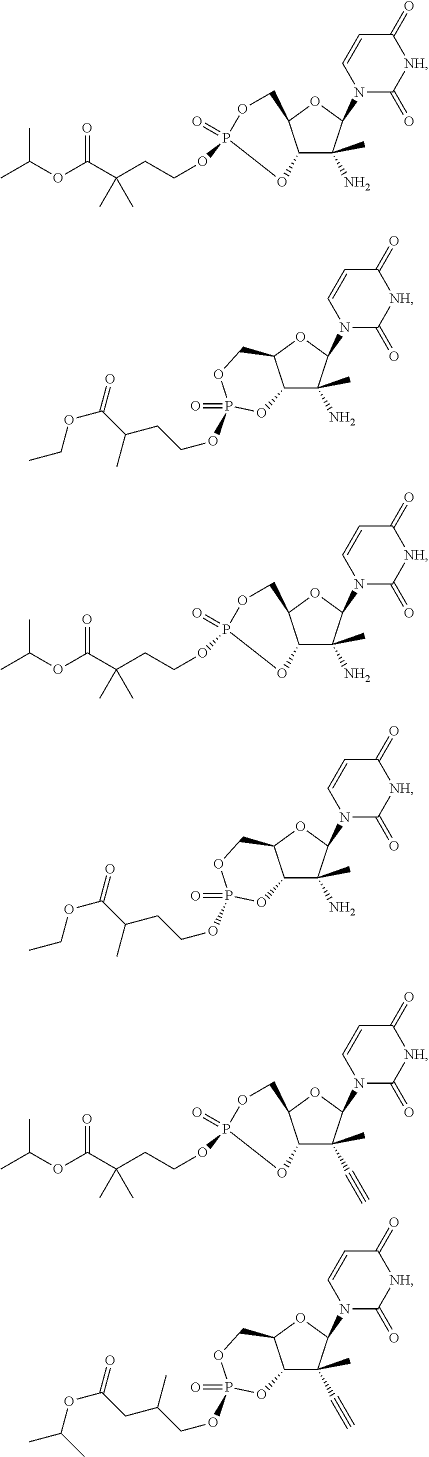 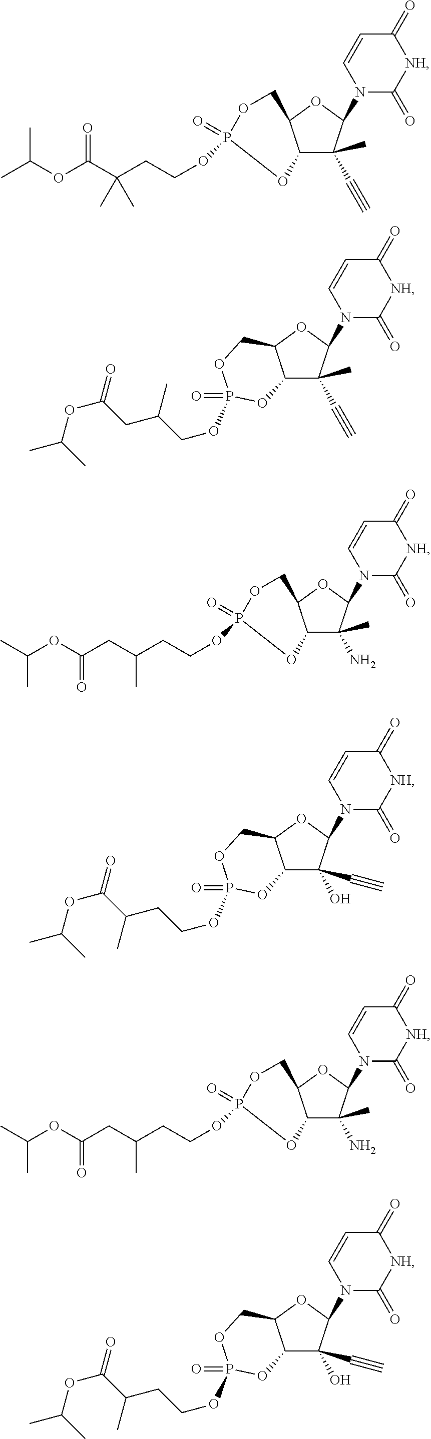 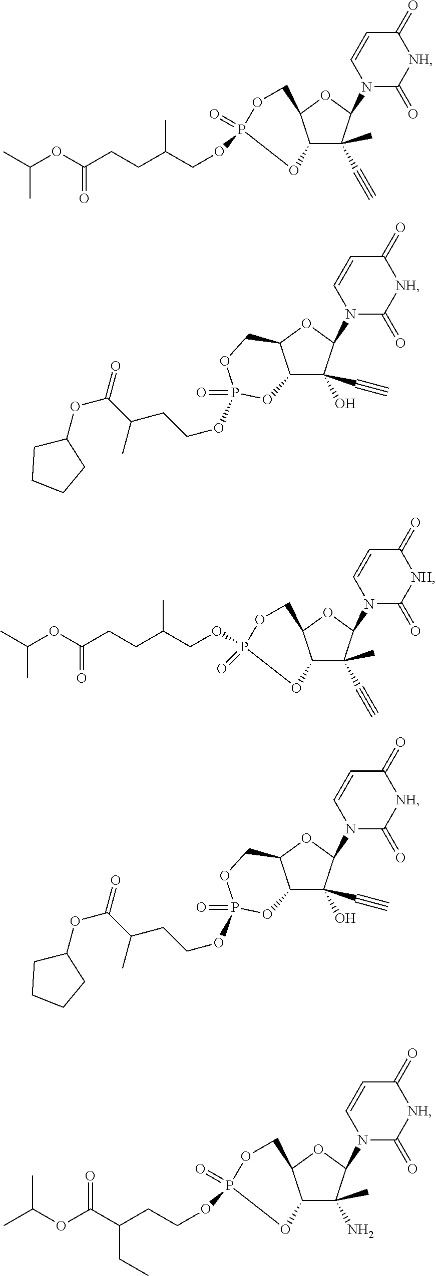 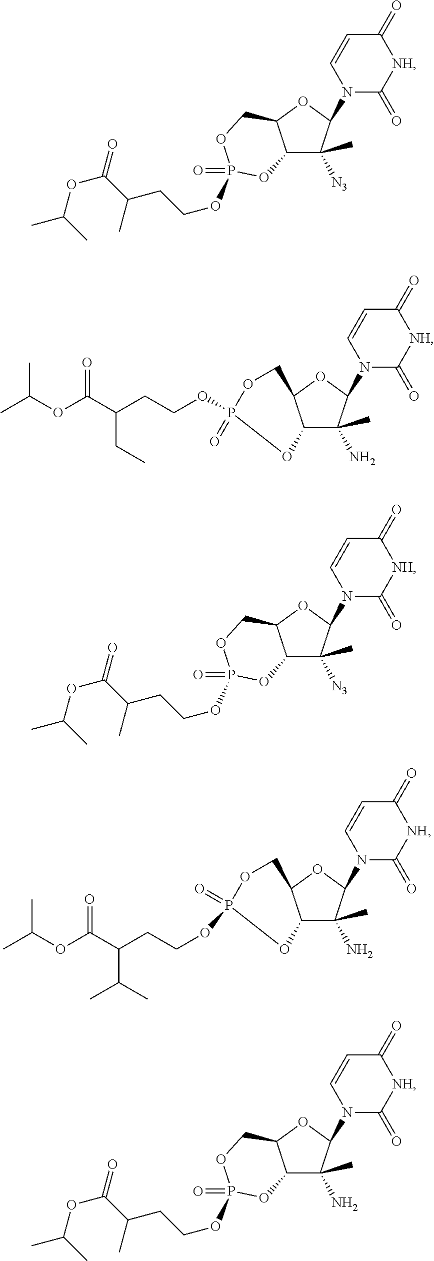 [SMILES: CC(C)OC(=O)C(C)CCO[P@@]1(=O)OC[C@H]2O[C@@H](n3ccc(=O)[nH]c3=O)[C@](C)(N=[N+]=[N-])[C@@H]2O1 ; CCC(CCO[P@]1(=O)OC[C@H]2O[C@@H](n3ccc(=O)[nH]c3=O)[C@](C)(N)[C@@H]2O1)C(=O)OC(C)C ; CC(C)OC(=O)C(C)CCO[P@]1(=O)OC[C@H]2O[C@@H](n3ccc(=O)[nH]c3=O)[C@](C)(N=[N+]=[N-])[C@@H]2O1 ; CC(C)OC(=O)C(CCO[P@@]1(=O)OC[C@H]2O[C@@H](n3ccc(=O)[nH]c3=O)[C@](C)(N)[C@@H]2O1)C(C)C ; CC(C)OC(=O)C(C)CCO[P@@]1(=O)OC[C@H]2O[C@@H](n3ccc(=O)[nH]c3=O)[C@](C)(N)[C@@H]2O1] 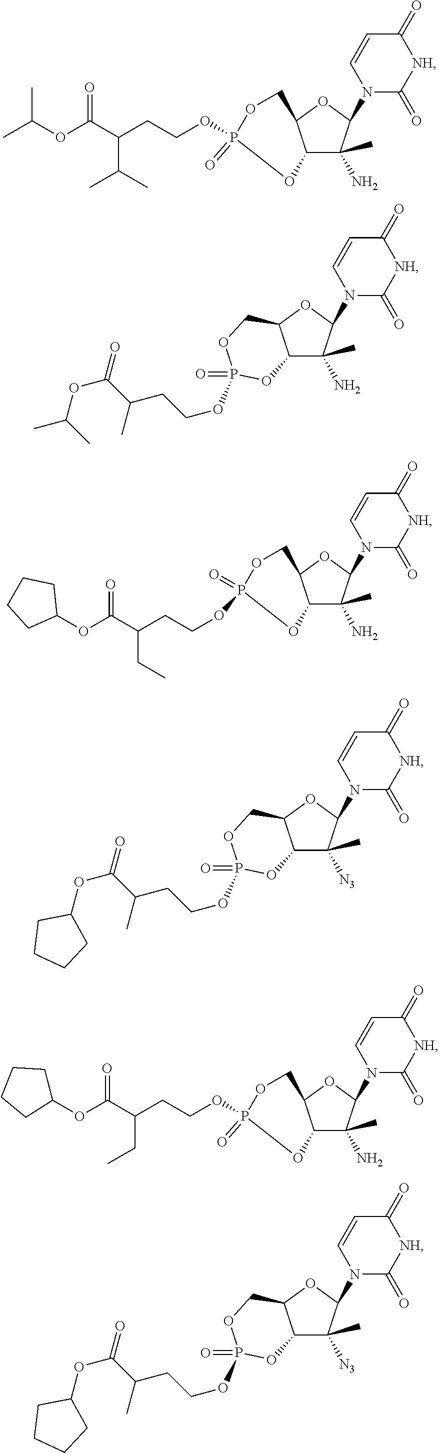 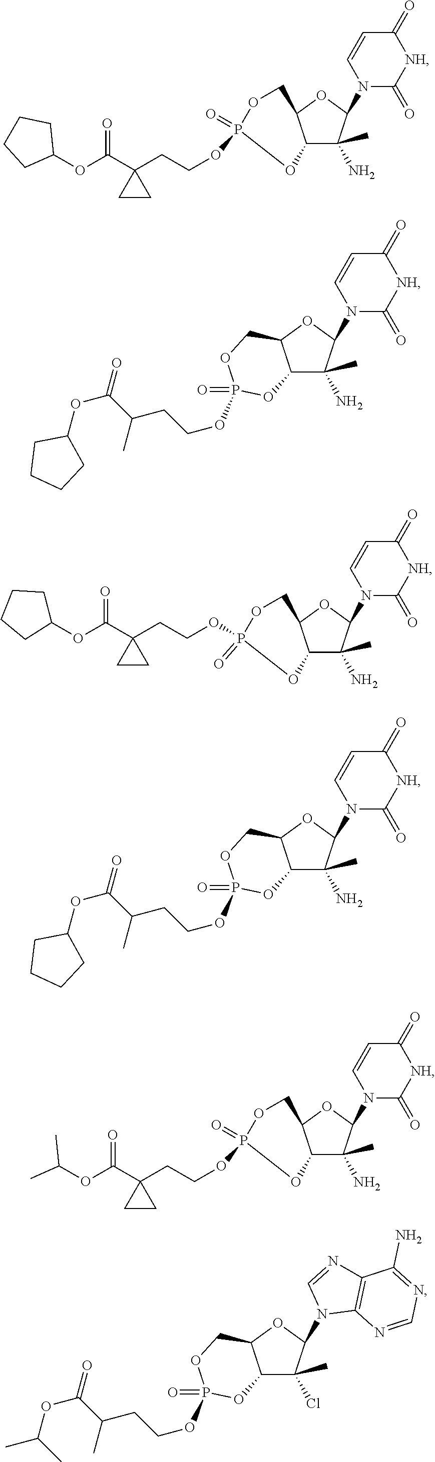 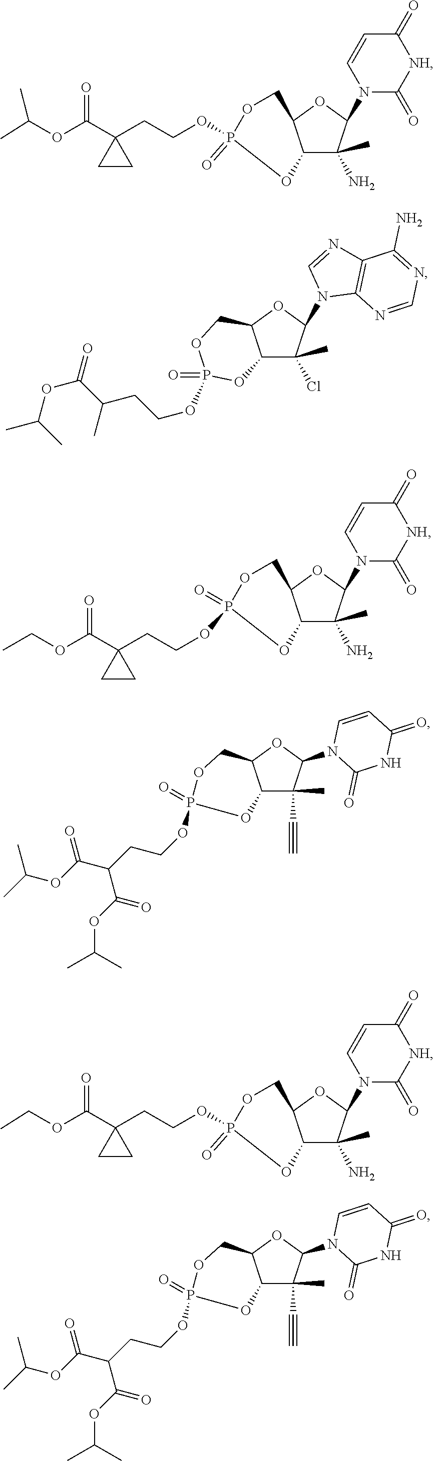 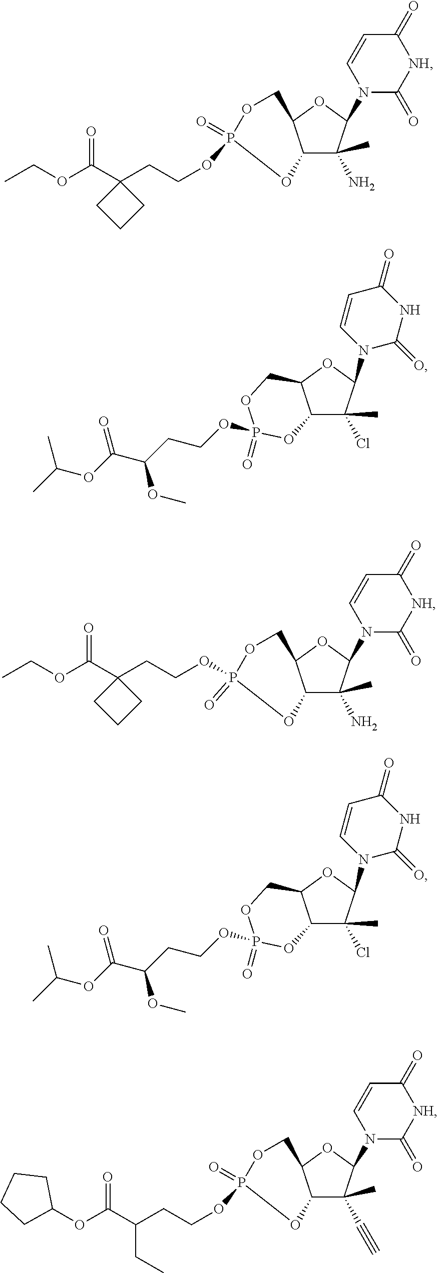 [SMILES: CCOC(=O)C1(CCO[P@@]2(=O)OC[C@H]3O[C@@H](n4ccc(=O)[nH]c4=O)[C@](C)(N)[C@@H]3O2)CCC1 ; CO[C@H](CCO[P@@]1(=O)OC[C@H]2O[C@@H](n3ccc(=O)[nH]c3=O)[C@](C)(Cl)[C@@H]2O1)C(=O)OC(C)C ; CCOC(=O)C1(CCO[P@]2(=O)OC[C@H]3O[C@@H](n4ccc(=O)[nH]c4=O)[C@](C)(N)[C@@H]3O2)CCC1 ; CO[C@H](CCO[P@]1(=O)OC[C@H]2O[C@@H](n3ccc(=O)[nH]c3=O)[C@](C)(Cl)[C@@H]2O1)C(=O)OC(C)C ; C#C[C@]1(C)[C@@H]2O[P@@](=O)(OCCC(CC)C(=O)OC3CCCC3)OC[C@H]2O[C@H]1n1ccc(=O)[nH]c1=O] 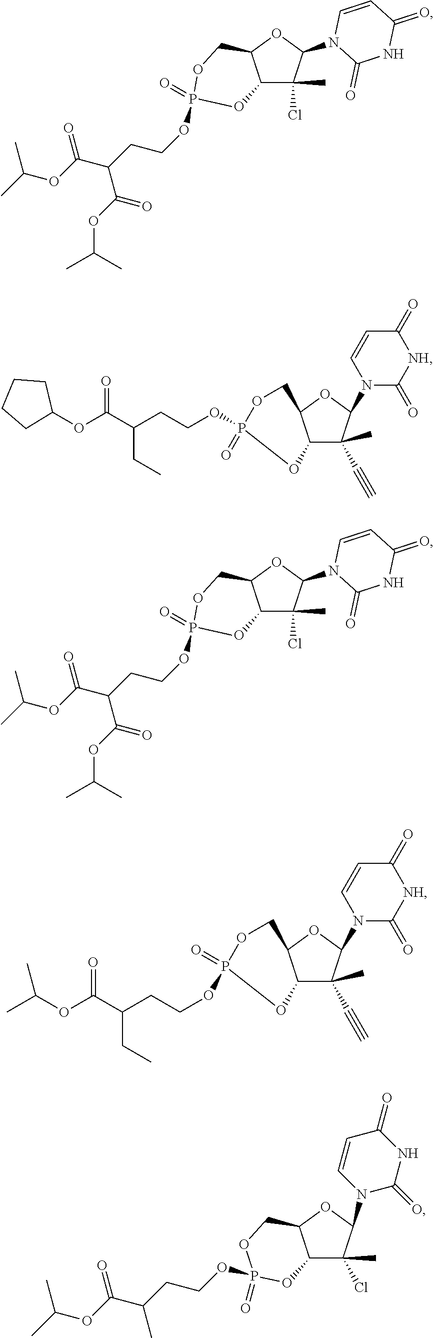 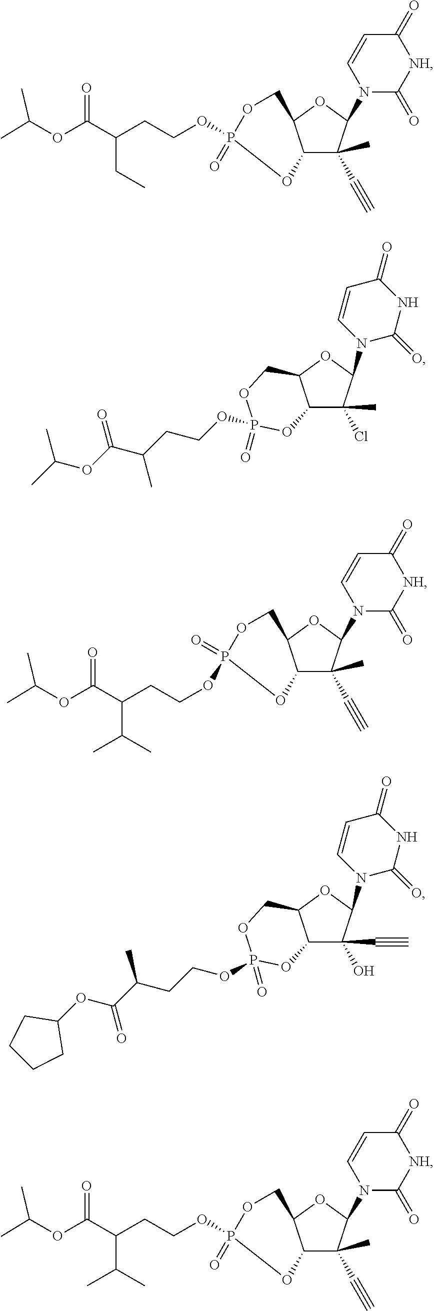 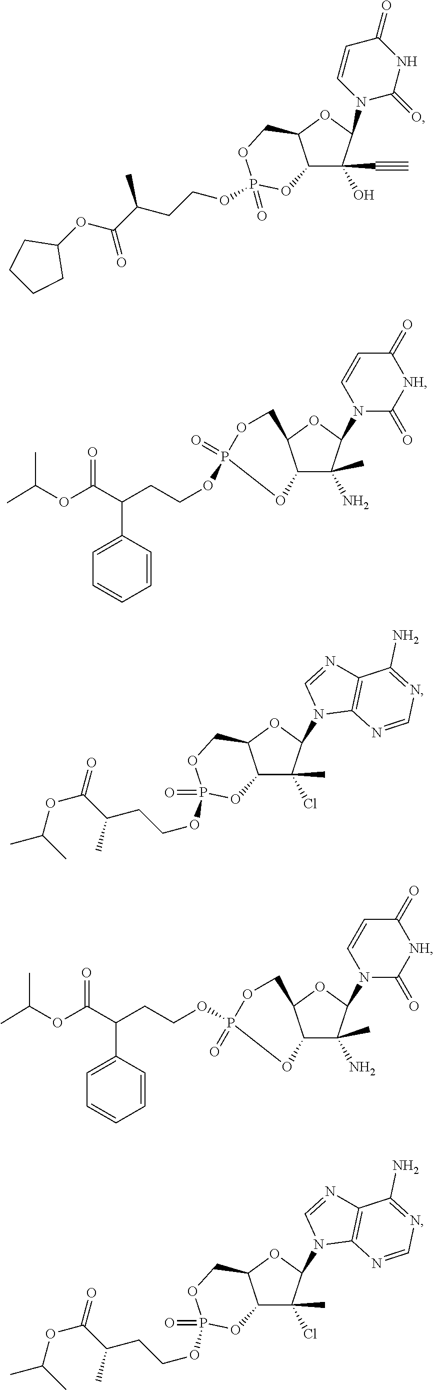 [SMILES: C#C[C@@]1(O)[C@@H]2O[P@](=O)(OCC[C@H](C)C(=O)OC3CCCC3)OC[C@H]2O[C@H]1n1ccc(=O)[nH]c1=O ; CC(C)OC(=O)C(CCO[P@@]1(=O)OC[C@H]2O[C@@H](n3ccc(=O)[nH]c3=O)[C@](C)(N)[C@@H]2O1)c1ccccc1 ; CC(C)OC(=O)[C@@H](C)CCO[P@@]1(=O)OC[C@H]2O[C@@H](n3cnc4c(N)ncnc43)[C@](C)(Cl)[C@@H]2O1 ; CC(C)OC(=O)C(CCO[P@]1(=O)OC[C@H]2O[C@@H](n3ccc(=O)[nH]c3=O)[C@](C)(N)[C@@H]2O1)c1ccccc1 ; CC(C)OC(=O)[C@@H](C)CCO[P@]1(=O)OC[C@H]2O[C@@H](n3cnc4c(N)ncnc43)[C@](C)(Cl)[C@@H]2O1] 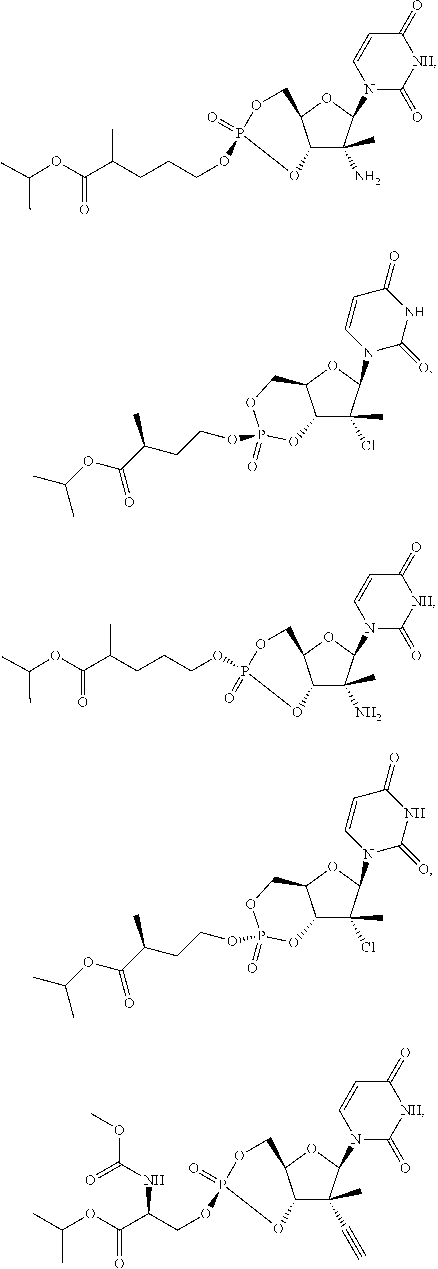 [SMILES: CC(C)OC(=O)C(C)CCCO[P@@]1(=O)OC[C@H]2O[C@@H](n3ccc(=O)[nH]c3=O)[C@](C)(N)[C@@H]2O1 ; CC(C)OC(=O)[C@@H](C)CCO[P@@]1(=O)OC[C@H]2O[C@@H](n3ccc(=O)[nH]c3=O)[C@](C)(Cl)[C@@H]2O1 ; CC(C)OC(=O)C(C)CCCO[P@]1(=O)OC[C@H]2O[C@@H](n3ccc(=O)[nH]c3=O)[C@](C)(N)[C@@H]2O1 ; CC(C)OC(=O)[C@@H](C)CCO[P@]1(=O)OC[C@H]2O[C@@H](n3ccc(=O)[nH]c3=O)[C@](C)(Cl)[C@@H]2O1 ; C#C[C@]1(C)[C@@H]2O[P@@](=O)(OC[C@H](NC(=O)OC)C(=O)OC(C)C)OC[C@H]2O[C@H]1n1ccc(=O)[nH]c1=O] 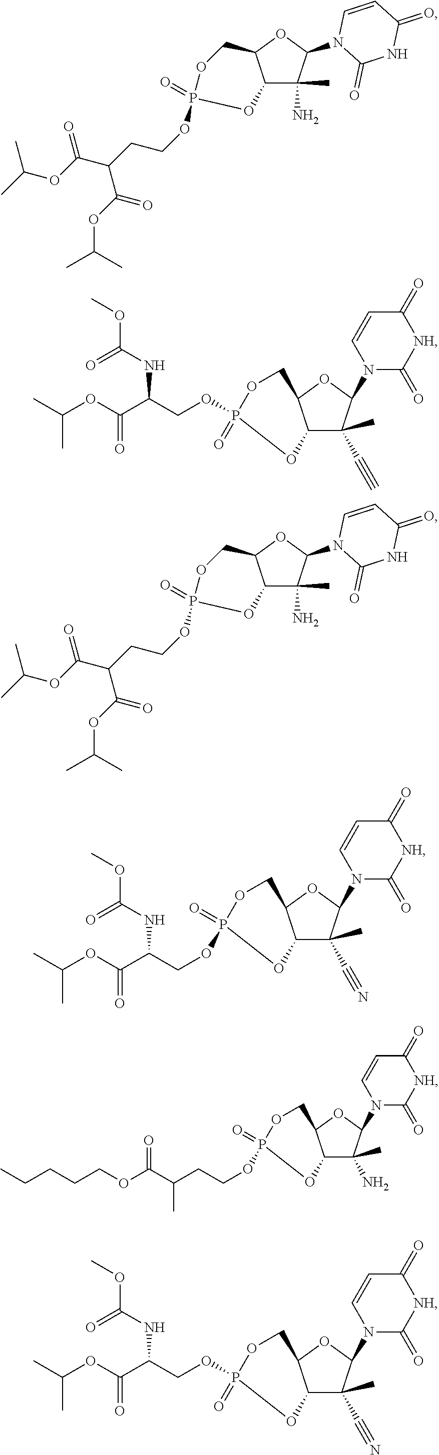 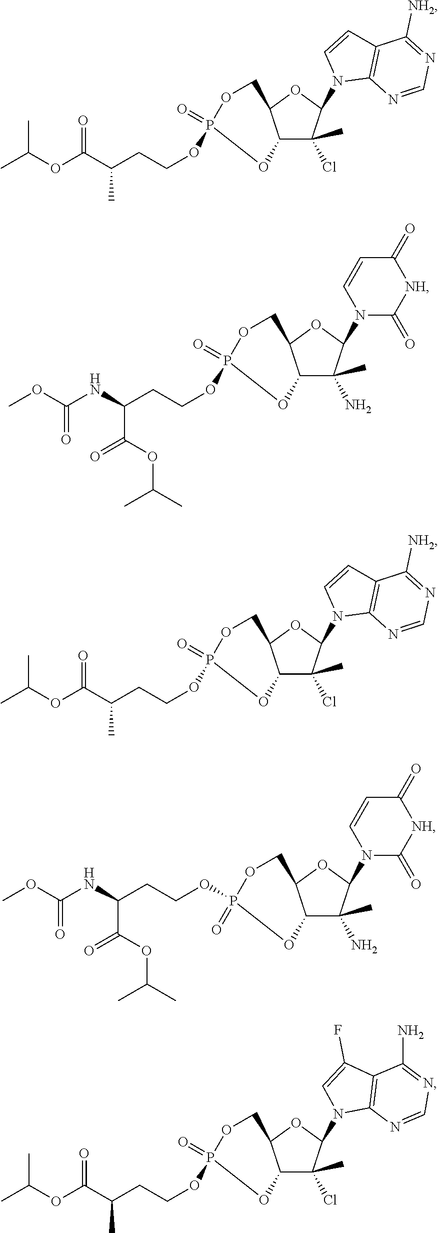 [SMILES: CC(C)OC(=O)[C@@H](C)CCO[P@@]1(=O)OC[C@H]2O[C@@H](n3ccc4c(N)ncnc43)[C@](C)(Cl)[C@@H]2O1 ; COC(=O)N[C@@H](CCO[P@@]1(=O)OC[C@H]2O[C@@H](n3ccc(=O)[nH]c3=O)[C@](C)(N)[C@@H]2O1)C(=O)OC(C)C ; CC(C)OC(=O)[C@@H](C)CCO[P@]1(=O)OC[C@H]2O[C@@H](n3ccc4c(N)ncnc43)[C@](C)(Cl)[C@@H]2O1 ; COC(=O)N[C@@H](CCO[P@]1(=O)OC[C@H]2O[C@@H](n3ccc(=O)[nH]c3=O)[C@](C)(N)[C@@H]2O1)C(=O)OC(C)C ; CC(C)OC(=O)[C@H](C)CCO[P@@]1(=O)OC[C@H]2O[C@@H](n3cc(F)c4c(N)ncnc43)[C@](C)(Cl)[C@@H]2O1] 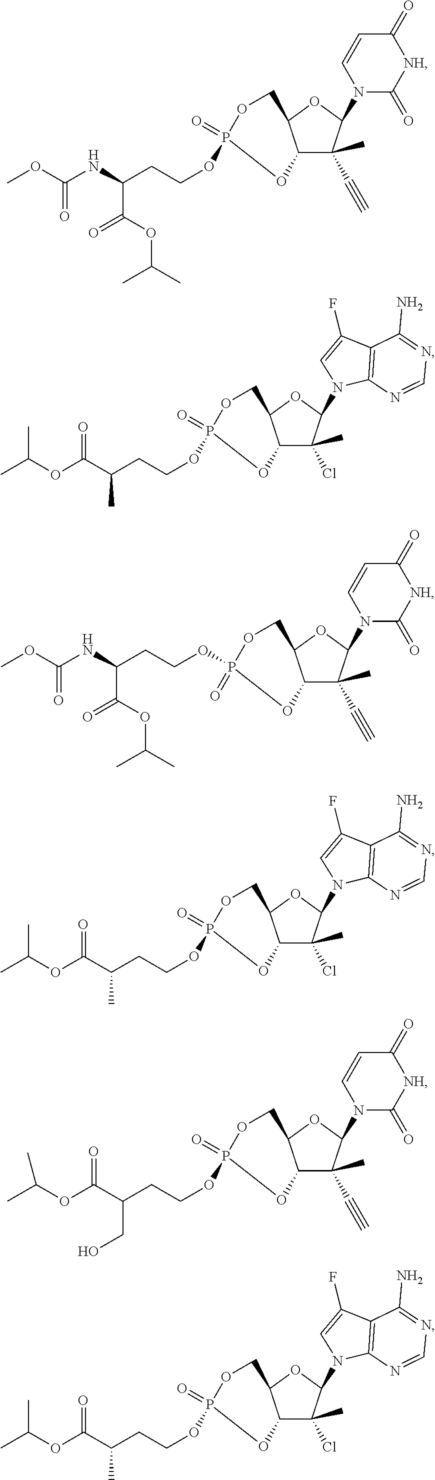 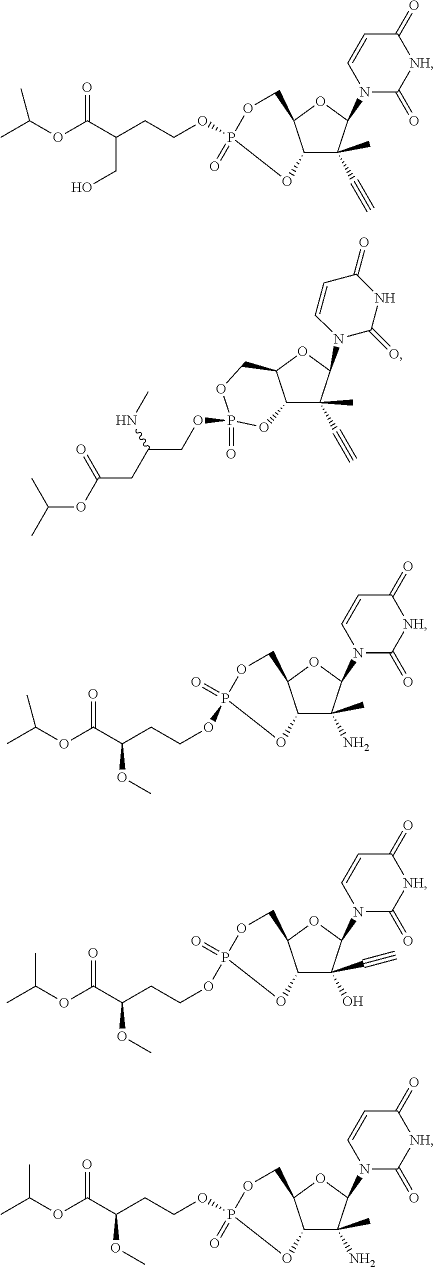 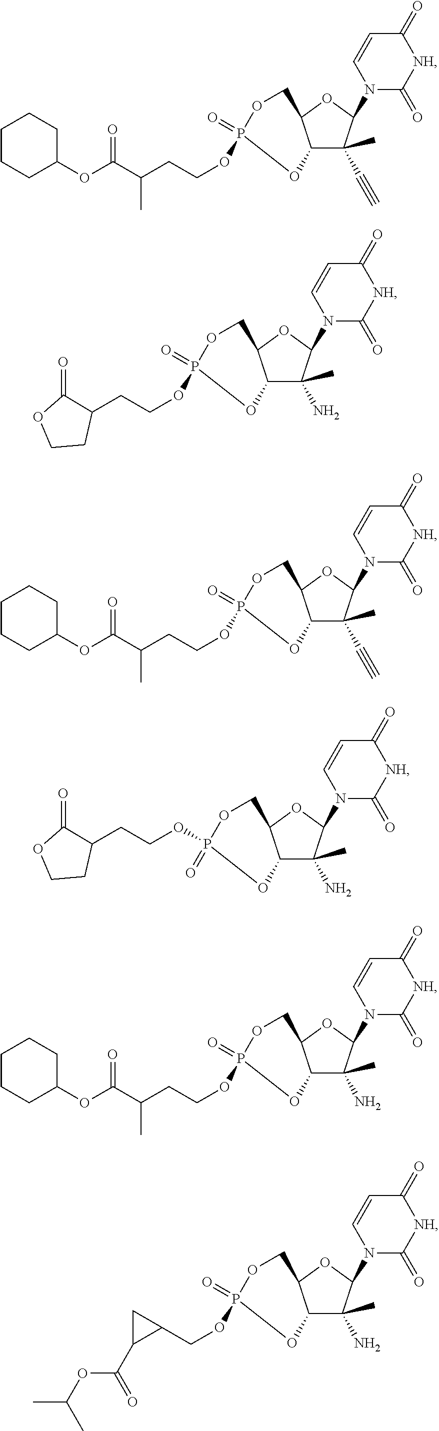 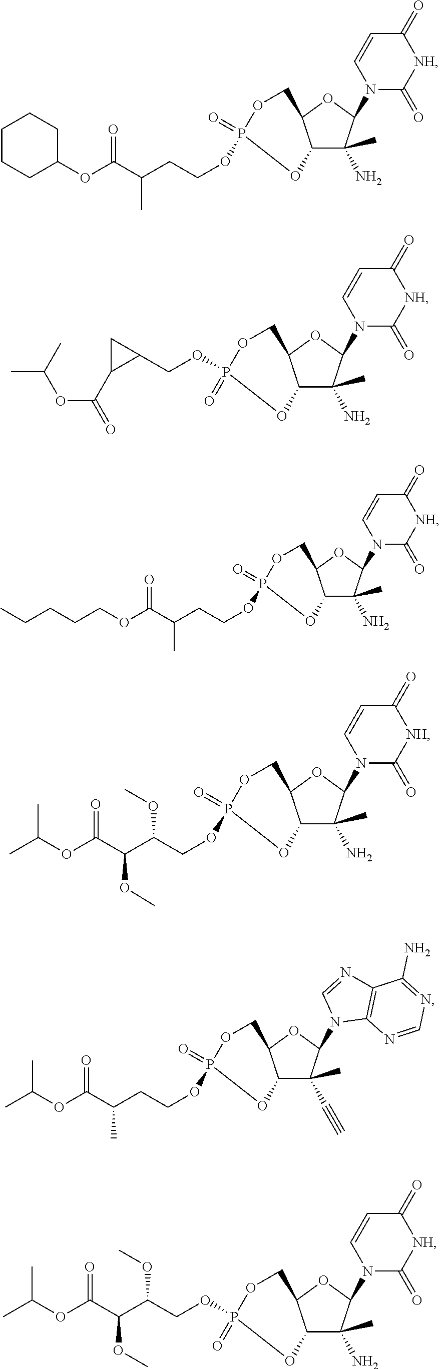 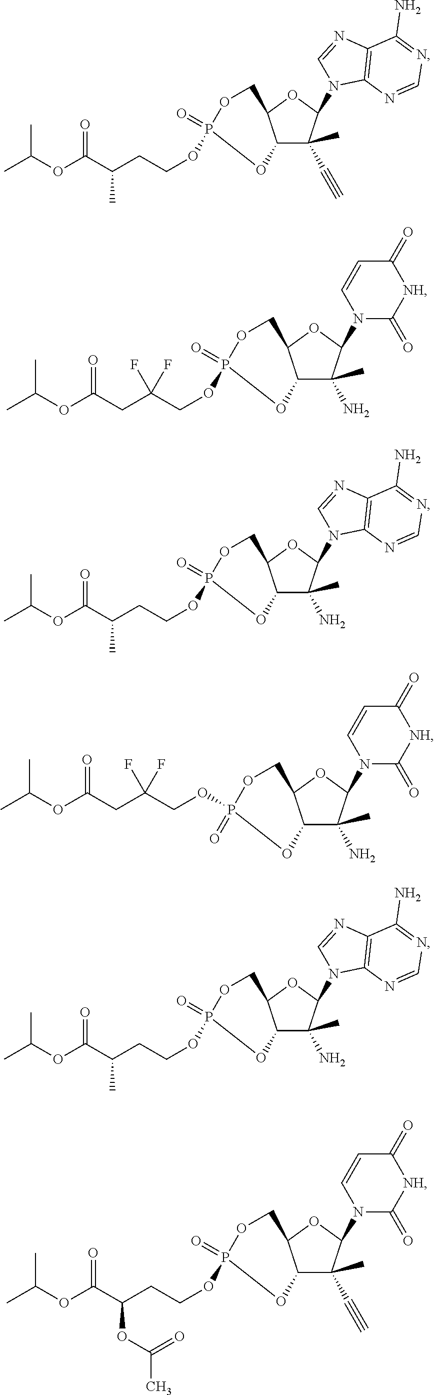 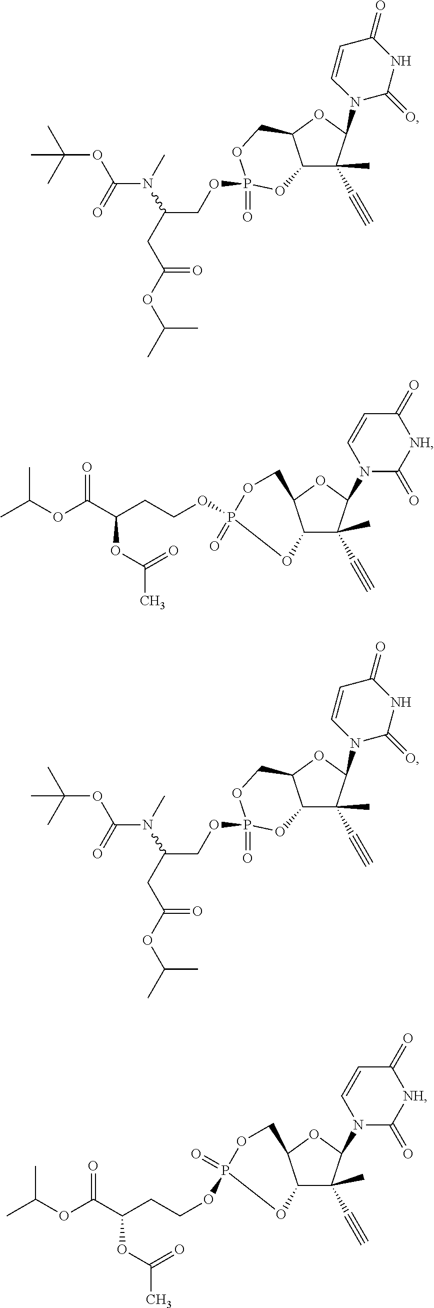 [SMILES: C#C[C@]1(C)[C@@H]2O[P@@](=O)(OCC(CC(=O)OC(C)C)N(C)C(=O)OC(C)(C)C)OC[C@H]2O[C@H]1n1ccc(=O)[nH]c1=O ; C#C[C@]1(C)[C@@H]2O[P@](=O)(OCC[C@@H](OC(C)=O)C(=O)OC(C)C)OC[C@H]2O[C@H]1n1ccc(=O)[nH]c1=O ; C#C[C@]1(C)[C@@H]2O[P@@](=O)(OCC(CC(=O)OC(C)C)N(C)C(=O)OC(C)(C)C)OC[C@H]2O[C@H]1n1ccc(=O)[nH]c1=O ; C#C[C@]1(C)[C@@H]2O[P@@](=O)(OCC[C@H](OC(C)=O)C(=O)OC(C)C)OC[C@H]2O[C@H]1n1ccc(=O)[nH]c1=O] 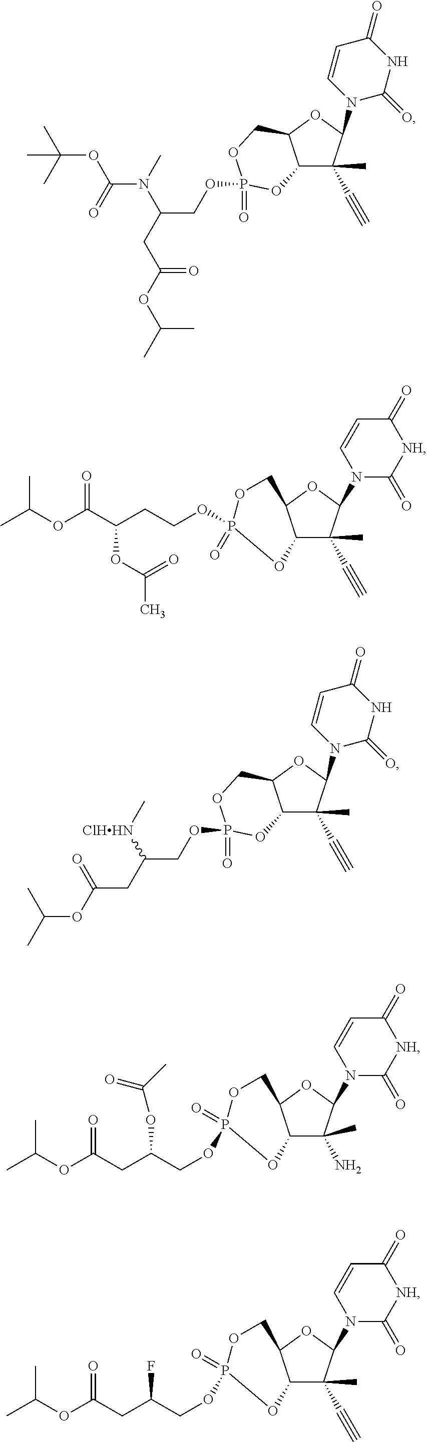 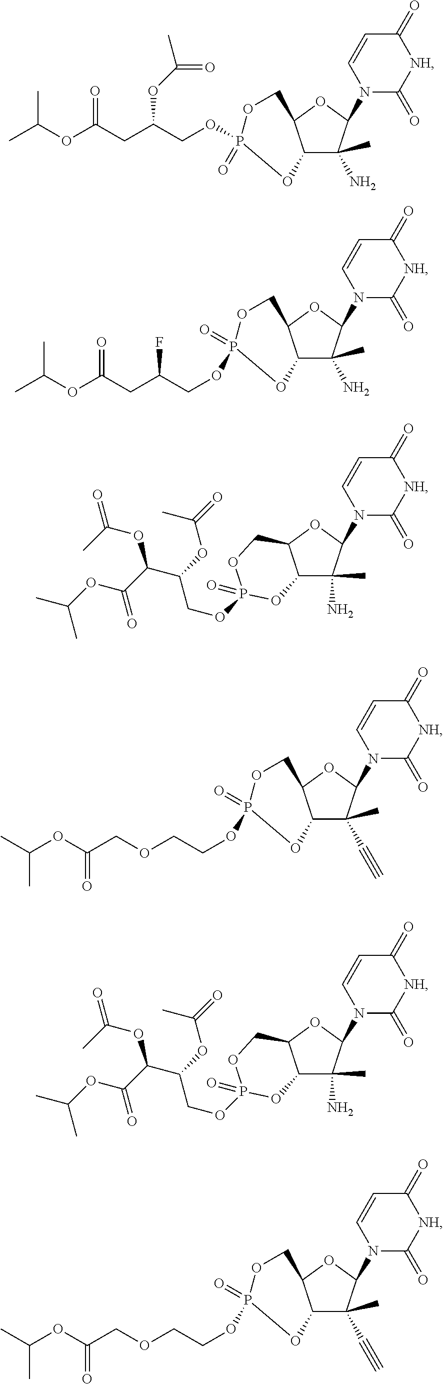 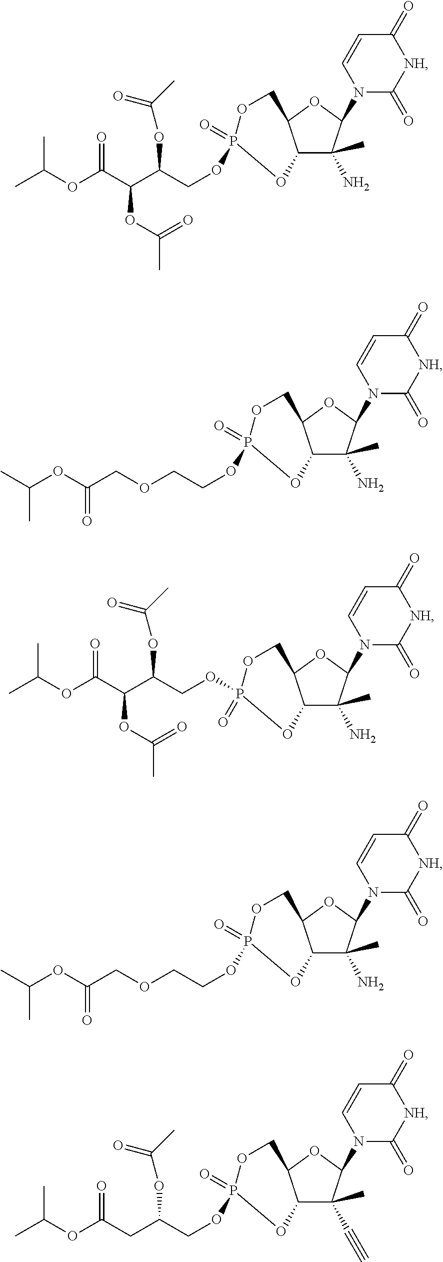 [SMILES: CC(=O)O[C@@H](CO[P@@]1(=O)OC[C@H]2O[C@@H](n3ccc(=O)[nH]c3=O)[C@](C)(N)[C@@H]2O1)[C@@H](OC(C)=O)C(=O)OC(C)C ; CC(C)OC(=O)COCCO[P@@]1(=O)OC[C@H]2O[C@@H](n3ccc(=O)[nH]c3=O)[C@](C)(N)[C@@H]2O1 ; CC(=O)O[C@@H](CO[P@]1(=O)OC[C@H]2O[C@@H](n3ccc(=O)[nH]c3=O)[C@](C)(N)[C@@H]2O1)[C@@H](OC(C)=O)C(=O)OC(C)C ; CC(C)OC(=O)COCCO[P@]1(=O)OC[C@H]2O[C@@H](n3ccc(=O)[nH]c3=O)[C@](C)(N)[C@@H]2O1 ; C#C[C@]1(C)[C@@H]2O[P@@](=O)(OC[C@H](CC(=O)OC(C)C)OC(C)=O)OC[C@H]2O[C@H]1n1ccc(=O)[nH]c1=O] 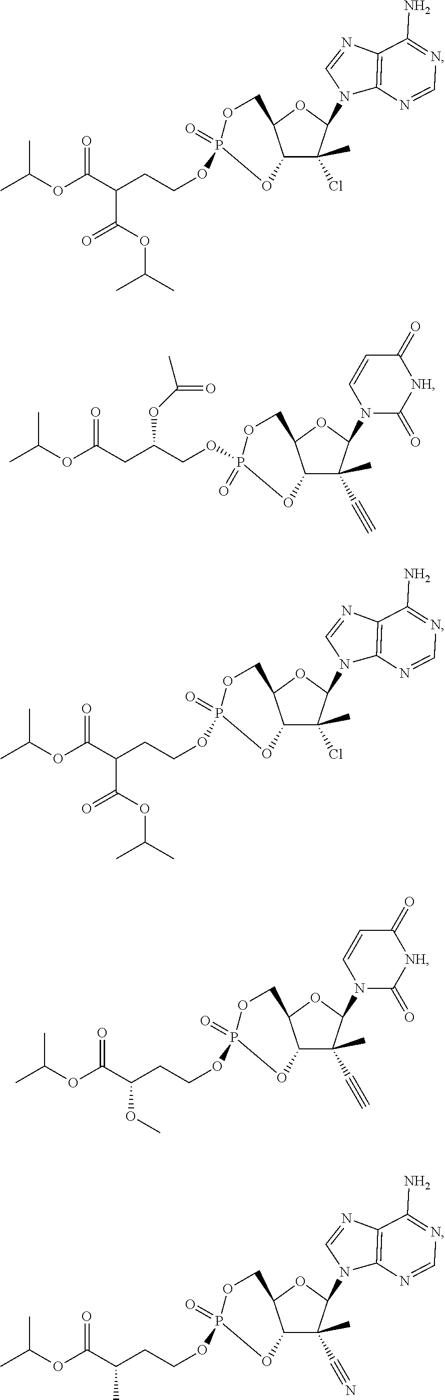 [SMILES: CC(C)OC(=O)C(CCO[P@@]1(=O)OC[C@H]2O[C@@H](n3cnc4c(N)ncnc43)[C@](C)(Cl)[C@@H]2O1)C(=O)OC(C)C ; C#C[C@]1(C)[C@@H]2O[P@](=O)(OC[C@H](CC(=O)OC(C)C)OC(C)=O)OC[C@H]2O[C@H]1n1ccc(=O)[nH]c1=O ; CC(C)OC(=O)C(CCO[P@]1(=O)OC[C@H]2O[C@@H](n3cnc4c(N)ncnc43)[C@](C)(Cl)[C@@H]2O1)C(=O)OC(C)C ; C#C[C@]1(C)[C@@H]2O[P@@](=O)(OCC[C@H](OC)C(=O)OC(C)C)OC[C@H]2O[C@H]1n1ccc(=O)[nH]c1=O ; CC(C)OC(=O)[C@@H](C)CCO[P@@]1(=O)OC[C@H]2O[C@@H](n3cnc4c(N)ncnc43)[C@](C)(C#N)[C@@H]2O1] 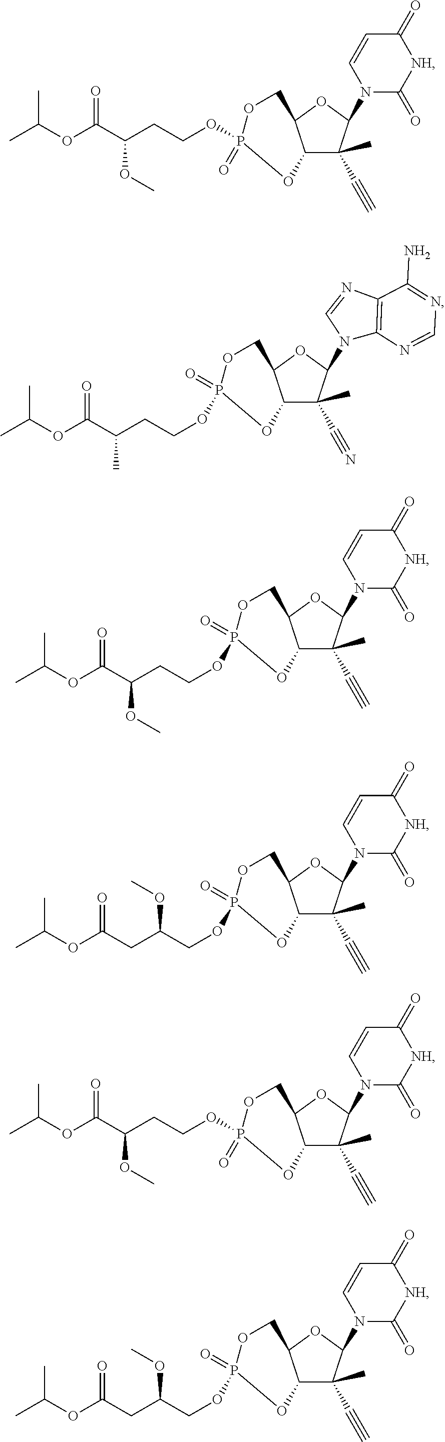 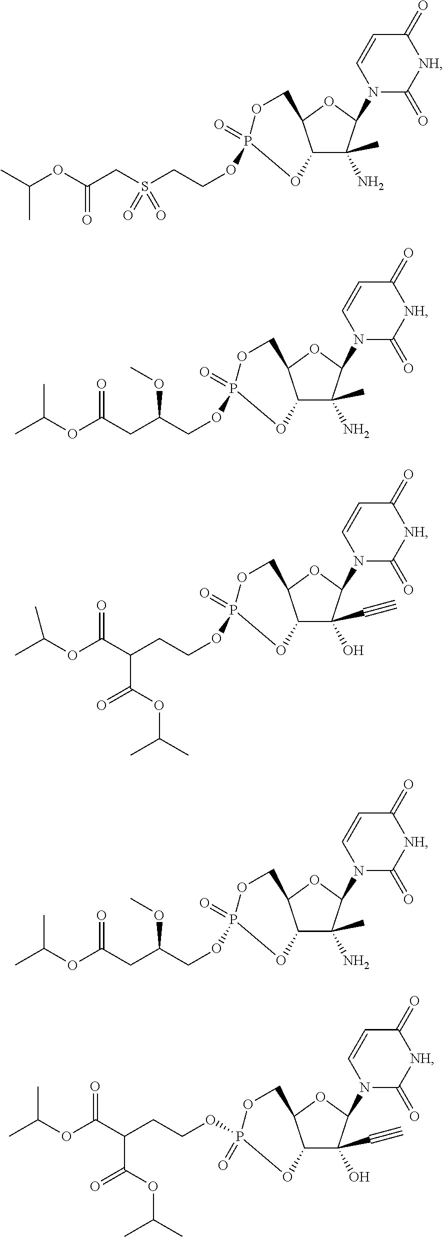 [SMILES: CC(C)OC(=O)CS(=O)(=O)CCO[P@@]1(=O)OC[C@H]2O[C@@H](n3ccc(=O)[nH]c3=O)[C@](C)(N)[C@@H]2O1 ; CO[C@@H](CO[P@@]1(=O)OC[C@H]2O[C@@H](n3ccc(=O)[nH]c3=O)[C@](C)(N)[C@@H]2O1)CC(=O)OC(C)C ; C#C[C@@]1(O)[C@@H]2O[P@@](=O)(OCCC(C(=O)OC(C)C)C(=O)OC(C)C)OC[C@H]2O[C@H]1n1ccc(=O)[nH]c1=O ; CO[C@@H](CO[P@]1(=O)OC[C@H]2O[C@@H](n3ccc(=O)[nH]c3=O)[C@](C)(N)[C@@H]2O1)CC(=O)OC(C)C ; C#C[C@@]1(O)[C@@H]2O[P@](=O)(OCCC(C(=O)OC(C)C)C(=O)OC(C)C)OC[C@H]2O[C@H]1n1ccc(=O)[nH]c1=O] 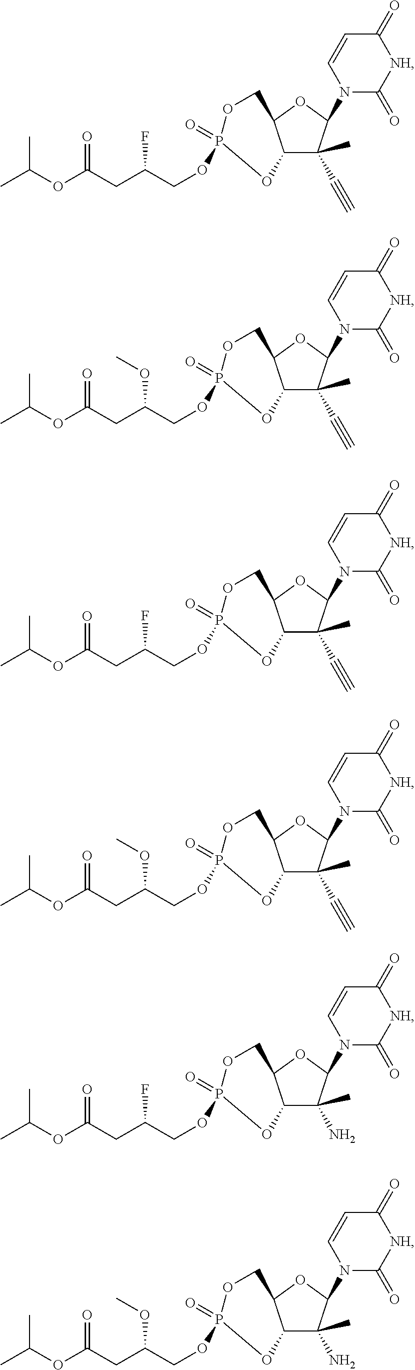 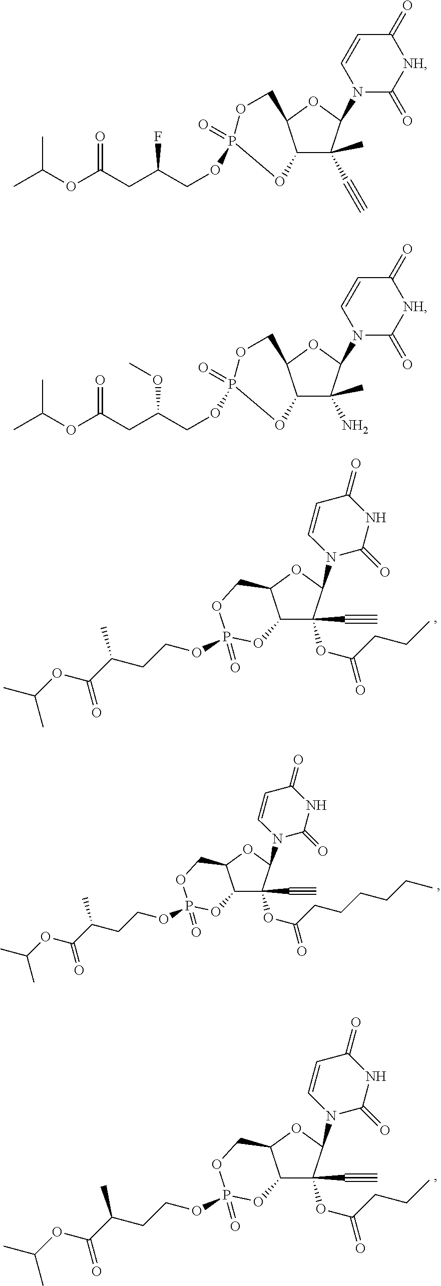 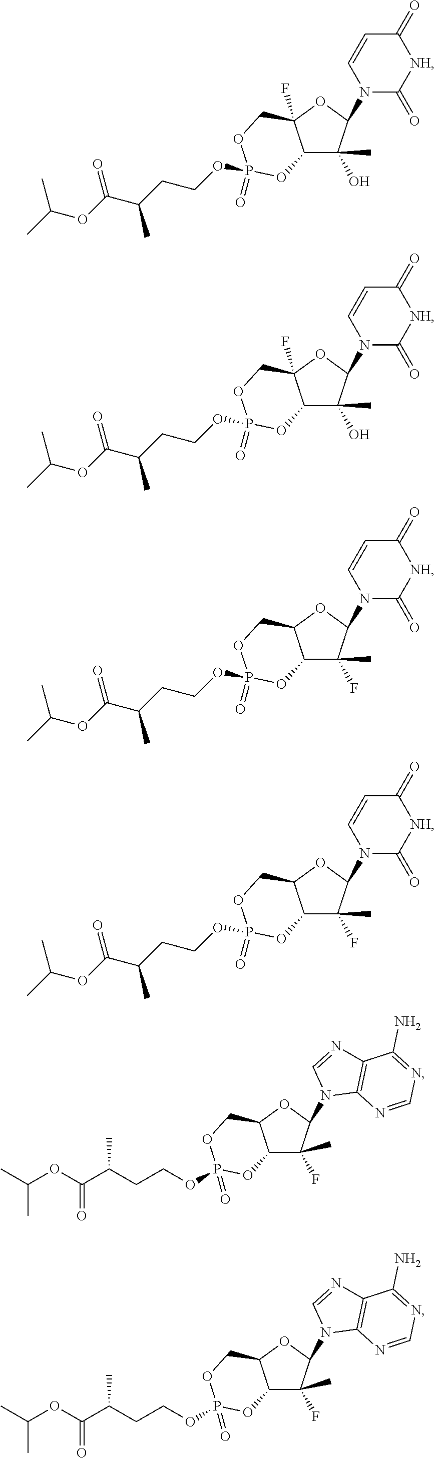 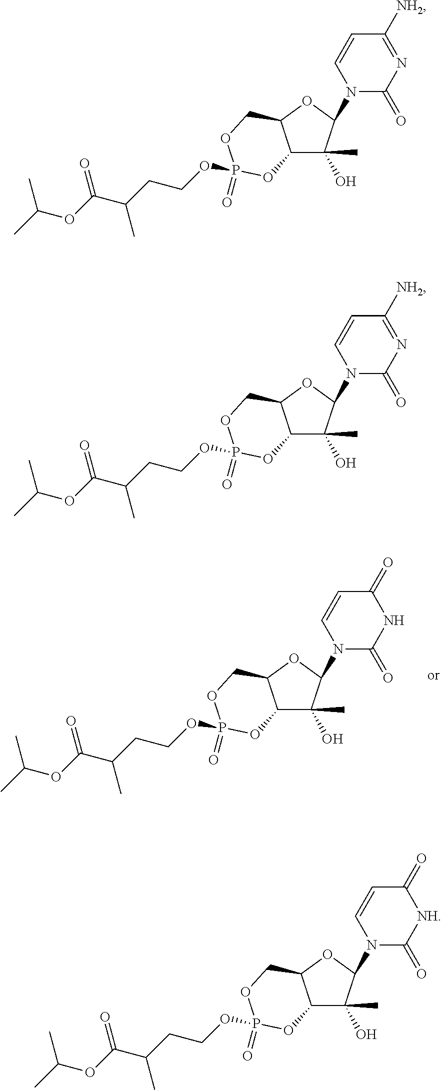 [SMILES: CC(C)OC(=O)C(C)CCO[P@@]1(=O)OC[C@H]2O[C@@H](n3ccc(N)nc3=O)[C@](C)(O)[C@@H]2O1 ; CC(C)OC(=O)C(C)CCO[P@]1(=O)OC[C@H]2O[C@@H](n3ccc(N)nc3=O)[C@](C)(O)[C@@H]2O1 ; CC(C)OC(=O)C(C)CCO[P@@]1(=O)OC[C@H]2O[C@@H](n3ccc(=O)[nH]c3=O)[C@](C)(O)[C@@H]2O1 ; CC(C)OC(=O)C(C)CCO[P@]1(=O)OC[C@H]2O[C@@H](n3ccc(=O)[nH]c3=O)[C@](C)(O)[C@@H]2O1]
or a pharmaceutically acceptable salt thereof.

14. A pharmaceutical composition comprising an effective amount of the compound of claim 1, or a pharmaceutically acceptable salt thereof, and a pharmaceutically acceptable carrier.

15. The pharmaceutical composition of claim 14, further comprising a second therapeutic agent selected from the group consisting of HCV antiviral agents, immunomodulators, and anti-infective agents.

16. The pharmaceutical composition of claim 15, further comprising a third therapeutic agent selected from the group consisting of HCV protease inhibitors, HCV NS5A inhibitors and HCV NS5B polymerase inhibitors.

17. The use of the compound according to claim 1, or a pharmaceutically acceptable salt thereof, in the preparation of a medicament for inhibiting HCV NS5B activity or for treating infection by HCV in a patient in need thereof.

18. A method of treating a patient infected with HCV comprising the step of administering an amount of the compound according to claim 1, or a pharmaceutically acceptable salt thereof, effective to treat infection by HCV in said patient.

19. The method of claim 27, further comprising the step of administering to said patient a second therapeutic agent selected from the group consisting of HCV antiviral agents, immunomodulators, and anti-infective agents.

20. (canceled)

21. The use of the compound of claim 1, or a pharmaceutically acceptable salt thereof, for inhibiting HCV NS5B activity or for preventing and/or treating infection by HCV in a patient in need thereof.
